(19) United States
(12) Patent Application Publication
(10) Pub. No.: US 2026/0131000 A1
(43) Pub. Date: May 14, 2026

(54) ANTIGEN RECOGNIZING RECEPTORS TARGETING L1CAM AND USES THEREOF

(71) Applicants: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US); TRI-INSTITUTIONAL THERAPEUTICS DISCOVERY INSTITUTE, INC., New York, NY (US)

(72) Inventors: Joan MASSAGUE, New York, NY (US); Karuna GANESH, New York, NY (US); Ivo LORENZ, New York, NY (US); Abdul KHAN, New York, NY (US); Sreekumar KODANGATTIL, New York, NY (US); Ziwei LIANG, New York, NY (US); Manuel BACA, New York, NY (US)

(73) Assignees: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US); TRI-INSTITUTIONAL THERAPEUTICS DISCOVERY INSTITUTE, INC., New York, NY (US)

(21) Appl. No.: 19/258,358

(22) Filed: Jul. 2, 2025

Related U.S. Application Data
(63) Continuation of application No. PCT/US2024/010619, filed on Jan. 8, 2024.
(60) Provisional application No. 63/478,809, filed on Jan. 6, 2023.

(51) Int. Cl. A61K 40/42; A61K 40/11; A61K 40/31; A61P 35/00; C07K 14/725; C07K 16/28

(52) U.S. Cl. CPC A61K 40/421; A61K 40/11; A61K 40/31; A61P 35/00; C07K 14/7051; C07K 16/2803; C07K 2317/24; C07K 2317/622; C07K 2317/92

(57) ABSTRACT

The presently disclosed subject matter provides for antigen-recognizing receptors that specifically target L1CAM and cells comprising such L1CAM-targeted antigen-recognizing receptors. The presently disclosed subject matter further provides uses of the L1CAM-targeted antigen-recognizing receptors for treatment.

### CROSS-REFERENCE TO RELATED APPLICATION

This application is a continuation application of International Patent Application No. PCT/US24/10619, filed on Jan. 8, 2024, which claims priority to U.S. Provisional Application No. 63/478,809, filed on Jan. 6, 2023, the contents of each of which are hereby incorporated by reference in their entireties, and to each of which priority is claimed.

### SEQUENCE LISTING

The instant application contains a Sequence Listing which has been submitted herewith and is hereby incorporated by reference in its entirety. Said .xml copy, created on Jul. 2, 2025, is named 0727341532US.xml, and is 174,381 bytes in size.

### INTRODUCTION

The presently disclosed subject matter provides methods and compositions for immunotherapies. It relates to antigen-recognizing receptors (e.g., chimeric antigen receptors (CARs)) that specifically target an L1CAM polypeptide, cells comprising such receptors, and methods of using such cells for treatments.

### BACKGROUND

Advanced solid tumors remain mostly incurable due to the emergence of tumor subclones. Termed Metastasis Stem Cells (MetSCs), these tumor subclones are capable of self-renewal, slow cell-cycling, tumor re-initiation, and therapy resistance. The cell adhesion molecule L1CAM was identified as a critical target specifically on MetSCs. In multiple solid cancers, including colorectal, lung, breast, ovarian, pancreas, prostate, and hepatobiliary cancers, L1CAM+ MetSCs are enriched following therapy, regenerating more aggressive metastatic cancers, which are the principal cause of cancer mortality. High L1CAM expression in tumors is almost universally associated with poor prognosis.

L1CAM is a crucial marker and mediator of quiescence-capable, chemoresistant, stem-like metastasis regenerating cells. Asa result, L1CAM is an attractive target for the treatment of advanced solid tumors. Further, L1CAM is critical for the persistence of multiple solid tumors, including the three types that most prominently cause cancer death (e.g., lung, breast, and colorectal cancer), in multiple metastatic organ sites such as the bone, lung, liver, and brain. L1CAM is critical for the outgrowth of aggressive cancer cells right after freshly seeding a target organ, as well as indolent cancer cells that emerge from dormancy. Thus, new therapeutic approaches are needed to target this dependency of metastasis stem cells and to treat advanced solid tumors.

### SUMMARY

The presently disclosed subject matter provides antigen-recognizing receptors that specifically target an L1CAM polypeptide and cells comprising such L1CAM-targeted antigen-recognizing receptors. The presently disclosed subject matter further provides uses of the L1CAM-targeted antigen-recognizing receptors for treatment.

In certain non-limiting embodiments, the presently disclosed subject matter provides an antigen-recognizing receptor, comprising an extracellular antigen-binding domain, a transmembrane domain, and an intracellular signaling domain, wherein the extracellular antigen-binding domain specifically binds to L1CAM and comprises a heavy chain variable region and a light chain variable region. In certain embodiments,
a) the heavy chain variable region comprises a CDR1 comprising the amino acid sequence set forth in SEQ ID NO: 2 or a conservative modification thereof, a CDR2 comprising the amino acid sequence set forth in SEQ ID NO: 3 or a conservative modification thereof, and a CDR3 comprising the amino acid sequence set forth in SEQ ID NO: 4 or a conservative modification thereof, and the light chain variable region comprises a CDR1 comprising the amino acid sequence set forth in SEQ ID NO: 5 or a conservative modification thereof, a CDR2 comprising the amino acid sequence set forth in SEQ ID NO: 6 or a conservative modification thereof, and a CDR3 comprising the amino acid sequence set forth in SEQ ID NO: 7 or a conservative modification thereof;
b) the heavy chain variable region comprises a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof; and the light chain variable region comprises a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof,
c) the heavy chain variable region comprises a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 26 or a conservative modification thereof; and the light chain variable region comprises a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof,
d) the heavy chain variable region comprises a CDR1 comprising the amino acid sequence set forth in SEQ ID NO: 35 or a conservative modification thereof, a CDR2 comprising the amino acid sequence set forth in SEQ ID NO: 36 or a conservative modification thereof, and a CDR3 comprising the amino acid sequence set forth in SEQ ID NO: 37 or a conservative modification thereof; and the light chain variable region comprises a CDR1 comprising the amino acid sequence set forth in SEQ ID NO: 38 or a conservative modification thereof, a CDR2 comprising the amino acid sequence set forth in SEQ ID NO: 39 or a conservative modification thereof, and a CDR3 comprising the amino acid sequence set forth in SEQ ID NO: 40 or a conservative modification thereof;
e) the heavy chain variable region comprises a CDR1 comprising the amino acid sequence set forth in SEQ ID NO: 43 or a conservative modification thereof, a CDR2 comprising the amino acid sequence set forth in SEQ ID NO: 44 or a conservative modification thereof, and a CDR3 comprising the amino acid sequence set forth in SEQ ID NO: 45 or a conservative modification thereof; and the light chain variable region comprises a CDR1 comprising the amino acid sequence set forth in SEQ ID NO: 46 or a conservative modification thereof, a CDR2 comprising the amino acid sequence set forth in SEQ ID NO: 47 or a conservative modification thereof, and a CDR3 comprising the amino acid sequence set forth in SEQ ID NO: 48 or a conservative modification thereof,
f) the heavy chain variable region comprises a CDR1 comprising the amino acid sequence set forth in SEQ ID NO: 43 or a conservative modification thereof, a CDR2 comprising the amino acid sequence set forth in SEQ ID NO: 44 or a conservative modification thereof, and a CDR3 comprising the amino acid sequence set forth in SEQ ID NO: 45 or a conservative modification thereof; and the light chain variable region comprises a CDR1 comprising the amino acid sequence set forth in SEQ ID NO: 51 or a conservative modification thereof, a CDR2 comprising the amino acid sequence set forth in SEQ ID NO: 52 or a conservative modification thereof, and a CDR3 comprising the amino acid sequence set forth in SEQ ID NO: 40 or a conservative modification thereof,
g) the heavy chain variable region comprises a CDR1 comprising the amino acid sequence set forth in SEQ ID NO: 54 or a conservative modification thereof, a CDR2 comprising the amino acid sequence set forth in SEQ ID NO: 55 or a conservative modification thereof, and a CDR3 comprising the amino acid sequence set forth in SEQ ID NO: 56 or a conservative modification thereof; and the light chain variable region comprises a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 58 or a conservative modification thereof,
h) the heavy chain variable region comprises a CDR1 comprising the amino acid sequence set forth in SEQ ID NO: 62 or a conservative modification thereof, a CDR2 comprising the amino acid sequence set forth in SEQ ID NO: 63 or a conservative modification thereof, and a CDR3 comprising the amino acid sequence set forth in SEQ ID NO: 64 or a conservative modification thereof, and the light chain variable region comprises a CDR1 comprising the amino acid sequence set forth in SEQ ID NO: 65 or a conservative modification thereof, a CDR2 comprising the amino acid sequence set forth in SEQ ID NO: 58 or a conservative modification thereof, and a CDR3 comprising the amino acid sequence set forth in SEQ ID NO: 66 or a conservative modification thereof,
i) the heavy chain variable region comprises a CDR1 comprising the amino acid sequence set forth in SEQ ID NO: 69 or a conservative modification thereof, a CDR2 comprising the amino acid sequence set forth in SEQ ID NO: 70 or a conservative modification thereof, and a CDR3 comprising the amino acid sequence set forth in SEQ ID NO: 71 or a conservative modification thereof, and the light chain variable region comprises a CDR1 comprising the amino acid sequence set forth in SEQ ID NO: 72 or a conservative modification thereof, a CDR2 comprising the amino acid sequence set forth in SEQ ID NO: 73 or a conservative modification thereof, and a CDR3 comprising the amino acid sequence set forth in SEQ ID NO: 74 or a conservative modification thereof,
j) the heavy chain variable region comprises a CDR1 comprising the amino acid sequence set forth in SEQ ID NO: 81 or a conservative modification thereof, a CDR2 comprising the amino acid sequence set forth in SEQ ID NO: 82 or a conservative modification thereof, and a CDR3 comprising the amino acid sequence set forth in SEQ ID NO: 83 or a conservative modification thereof, and the light chain variable region comprises a CDR1 comprising the amino acid sequence set forth in SEQ ID NO: 84 or a conservative modification thereof, a CDR2 comprising the amino acid sequence set forth in SEQ ID NO: 85 or a conservative modification thereof, and a CDR3 comprising the amino acid sequence set forth in SEQ ID NO: 86 or a conservative modification thereof,
k) the heavy chain variable region comprises a CDR1 comprising the amino acid sequence set forth in SEQ ID NO: 93 or a conservative modification thereof, a CDR2 comprising the amino acid sequence set forth in SEQ ID NO: 94 or a conservative modification thereof, and a CDR3 comprising the amino acid sequence set forth in SEQ ID NO: 95 or a conservative modification thereof, and the light chain variable region comprises a CDR1 comprising the amino acid sequence set forth in SEQ ID NO: 96 or a conservative modification thereof, a CDR2 comprising the amino acid sequence set forth in SEQ ID NO: 97 or a conservative modification thereof, and a CDR3 comprising the amino acid sequence set forth in SEQ ID NO: 98 or a conservative modification thereof,
l) the heavy chain variable region comprises a CDR1 comprising the amino acid sequence set forth in SEQ ID NO: 105 or a conservative modification thereof, a CDR2 comprising the amino acid sequence set forth in SEQ ID NO: 106 or a conservative modification thereof, and a CDR3 comprising the amino acid sequence set forth in SEQ ID NO: 107 or a conservative modification thereof; and the light chain variable region comprises a CDR1 comprising the amino acid sequence set forth in SEQ ID NO: 108 or a conservative modification thereof, a CDR2 comprising the amino acid sequence set forth in SEQ ID NO: 39 or a conservative modification thereof, and a CDR3 comprising the amino acid sequence set forth in SEQ ID NO: 109 or a conservative modification thereof; or
m) the heavy chain variable region comprises a CDR1 comprising the amino acid sequence set forth in SEQ ID NO: 116 or a conservative modification thereof, a CDR2 comprising the amino acid sequence set forth in SEQ ID NO: 117 or a conservative modification thereof, and a CDR3 comprising the amino acid sequence set forth in SEQ ID NO: 118 or a conservative modification thereof; and the light chain variable region comprises a CDR1 comprising the amino acid sequence set forth in SEQ ID NO: 119 or a conservative modification thereof, a CDR2 comprising the amino acid sequence set forth in SEQ ID NO: 120 or a conservative modification thereof, and a CDR3 comprising the amino acid sequence set forth in SEQ ID NO: 121 or a conservative modification thereof.

In certain embodiments,
a) the heavy chain variable region comprises a CDR1 comprising the amino acid sequence set forth in SEQ ID NO: 2, a CDR2 comprising the amino acid sequence set forth in SEQ ID NO: 3, and a CDR3 comprising the amino acid sequence set forth in SEQ ID NO: 4; and the light chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7;
b) the heavy chain variable region comprises a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14; and the light chain variable region comprises a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17;
c) the heavy chain variable region comprises a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 26; and the light chain variable region comprises a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17;
d) the heavy chain variable region comprises a CDR1 comprising the amino acid sequence set forth in SEQ ID NO: 35, a CDR2 comprising the amino acid sequence set forth in SEQ ID NO: 36, and a CDR3 comprising the amino acid sequence set forth in SEQ ID NO: 37; and the light chain variable region comprises a CDR1 comprising the amino acid sequence set forth in SEQ ID NO: 38, a CDR2 comprising the amino acid sequence set forth in SEQ ID NO: 39, and a CDR3 comprising the amino acid sequence set forth in SEQ ID NO: 40;
e) the heavy chain variable region comprises a CDR1 comprising the amino acid sequence set forth in SEQ ID NO: 43, a CDR2 comprising the amino acid sequence set forth in SEQ ID NO: 44, and a CDR3 comprising the amino acid sequence set forth in SEQ ID NO: 45; and the light chain variable region comprises a CDR1 comprising the amino acid sequence set forth in SEQ ID NO: 46, a CDR2 comprising the amino acid sequence set forth in SEQ ID NO: 47, and a CDR3 comprising the amino acid sequence set forth in SEQ ID NO: 48;
f) the heavy chain variable region comprises a CDR1 comprising the amino acid sequence set forth in SEQ ID NO: 43, a CDR2 comprising the amino acid sequence set forth in SEQ ID NO: 44, and a CDR3 comprising the amino acid sequence set forth in SEQ ID NO: 45; and the light chain variable region comprises a CDR1 comprising the amino acid sequence set forth in SEQ ID NO: 51, a CDR2 comprising the amino acid sequence set forth in SEQ ID NO: 52, and a CDR3 comprising the amino acid sequence set forth in SEQ ID NO: 40;
g) the heavy chain variable region comprises a CDR1 comprising the amino acid sequence set forth in SEQ ID NO: 54, a CDR2 comprising the amino acid sequence set forth in SEQ ID NO: 55, and a CDR3 comprising the amino acid sequence set forth in SEQ ID NO: 56; and the light chain variable region comprises a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 58;
h) the heavy chain variable region comprises a CDR1 comprising the amino acid sequence set forth in SEQ ID NO: 62, a CDR2 comprising the amino acid sequence set forth in SEQ ID NO: 63, and a CDR3 comprising the amino acid sequence set forth in SEQ ID NO: 64; and the light chain variable region comprises a CDR1 comprising the amino acid sequence set forth in SEQ ID NO: 65, a CDR2 comprising the amino acid sequence set forth in SEQ ID NO: 58, and a CDR3 comprising the amino acid sequence set forth in SEQ ID NO: 66;
i) the heavy chain variable region comprises a CDR1 comprising the amino acid sequence set forth in SEQ ID NO: 69, a CDR2 comprising the amino acid sequence set forth in SEQ ID NO: 70, and a CDR3 comprising the amino acid sequence set forth in SEQ ID NO: 71; and the light chain variable region comprises a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 74;
j) the heavy chain variable region comprises a CDR1 comprising the amino acid sequence set forth in SEQ ID NO: 81, a CDR2 comprising the amino acid sequence set forth in SEQ ID NO: 82, and a CDR3 comprising the amino acid sequence set forth in SEQ ID NO: 83; and the light chain variable region comprises a CDR1 comprising the amino acid sequence set forth in SEQ ID NO: 84, a CDR2 comprising the amino acid sequence set forth in SEQ ID NO: 85, and a CDR3 comprising the amino acid sequence set forth in SEQ ID NO: 86;
k) the heavy chain variable region comprises a CDR1 comprising the amino acid sequence set forth in SEQ ID NO: 93, a CDR2 comprising the amino acid sequence set forth in SEQ ID NO: 94, and a CDR3 comprising the amino acid sequence set forth in SEQ ID NO: 95; and the light chain variable region comprises a CDR1 comprising the amino acid sequence set forth in SEQ ID NO: 96, a CDR2 comprising the amino acid sequence set forth in SEQ ID NO: 97, and a CDR3 comprising the amino acid sequence set forth in SEQ ID NO: 98;
l) the heavy chain variable region comprises a CDR1 comprising the amino acid sequence set forth in SEQ ID NO: 105, a CDR2 comprising the amino acid sequence set forth in SEQ ID NO: 106, and a CDR3 comprising the amino acid sequence set forth in SEQ ID NO: 107; and the light chain variable region comprises a CDR1 comprising the amino acid sequence set forth in SEQ ID NO: 108, a CDR2 comprising the amino acid sequence set forth in SEQ ID NO: 39, and a CDR3 comprising the amino acid sequence set forth in SEQ ID NO: 109; or
m) the heavy chain variable region comprises a CDR1 comprising the amino acid sequence set forth in SEQ ID NO: 116, a CDR2 comprising the amino acid sequence set forth in SEQ ID NO: 117, and a CDR3 comprising the amino acid sequence set forth in SEQ ID NO: 118; and the light chain variable region comprises a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121.

In certain embodiments,
a) the heavy chain variable region comprises a CDR1 comprising the amino acid sequence set forth in SEQ ID NO: 81, a CDR2 comprising the amino acid sequence set forth in SEQ ID NO: 82, and a CDR3 comprising the amino acid sequence set forth in SEQ ID NO: 83; and the light chain variable region comprises a CDR1 comprising the amino acid sequence set forth in SEQ ID NO: 84, a CDR2 comprising the amino acid sequence set forth in SEQ ID NO: 85, and a CDR3 comprising the amino acid sequence set forth in SEQ ID NO: 86;
b) the heavy chain variable region comprises a CDR1 comprising the amino acid sequence set forth in SEQ ID NO: 93, a CDR2 comprising the amino acid sequence set forth in SEQ ID NO: 94, and a CDR3 comprising the amino acid sequence set forth in SEQ ID NO: 95; and the light chain variable region comprises a CDR1 comprising the amino acid sequence set forth in SEQ ID NO: 96, a CDR2 comprising the amino acid sequence set forth in SEQ ID NO: 97, and a CDR3 comprising the amino acid sequence set forth in SEQ ID NO: 98; or
c) the heavy chain variable region comprises a CDR1 comprising the amino acid sequence set forth in SEQ ID NO: 105, a CDR2 comprising the amino acid sequence set forth in SEQ ID NO: 106, and a CDR3 comprising the amino acid sequence set forth in SEQ ID NO: 107; and the light chain variable region comprises a CDR1 comprising the amino acid sequence set forth in SEQ ID NO: 108, a CDR2 comprising the amino acid sequence set forth in SEQ ID NO: 39, and a CDR3 comprising the amino acid sequence set forth in SEQ ID NO: 109.

In certain embodiments, the extracellular antigen-binding domain is a single-chain variable fragment (scFv), a Fab, which is optionally crosslinked, or a F(ab)₂. In certain embodiments, the extracellular antigen-binding domain is a humanized scFv. In certain embodiments, one or more of the scFv, Fab and F(ab)2 are comprised in a fusion protein with a heterologous sequence to form the extracellular antigen-binding domain.

In certain embodiments, the heavy chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 8, SEQ ID NO: 18, SEQ ID NO: 22, SEQ ID NO: 24, SEQ ID NO: 25, SEQ ID NO: 27, SEQ ID NO: 28, SEQ ID NO: 29, SEQ ID NO: 33, SEQ ID NO: 41, SEQ ID NO: 49, SEQ ID NO: 60, SEQ ID NO: 67, SEQ ID NO: 75, SEQ ID NO: 87, SEQ ID NO: 99, SEQ ID NO: 110, SEQ ID NO: 122, SEQ ID NO: 128, SEQ ID NO: 130, SEQ ID NO: 134, or SEQ ID NO: 135. In certain embodiments, the light chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 9, SEQ ID NO: 19, SEQ ID NO: 23, SEQ ID NO: 30, SEQ ID NO: 31, SEQ ID NO: 32, SEQ ID NO: 34, SEQ ID NO: 42, SEQ ID NO: 50, SEQ ID NO: 53, SEQ ID NO: 61, SEQ ID NO: 68, SEQ ID NO: 76, SEQ ID NO: 88, SEQ ID NO: 100, SEQ ID NO: 111, SEQ ID NO: 123, SEQ ID NO: 129, SEQ ID NO: 131, SEQ ID NO: 132, SEQ ID NO: 133, or SEQ ID NO: 136.

In certain embodiments,
a) the heavy chain variable region comprises the amino acid sequence set forth in SEQ ID NO: 8, and the light chain variable region comprises the amino acid sequence set forth in SEQ ID NO: 9;
b) the heavy chain variable region comprises the amino acid sequence set forth in SEQ ID NO: 18, and the light chain variable region comprises the amino acid sequence set forth in SEQ ID NO: 19;
c) the heavy chain variable region comprises the amino acid sequence set forth in SEQ ID NO: 22, and the light chain variable region comprises the amino acid sequence set forth in SEQ ID NO: 23;
d) the heavy chain variable region comprises the amino acid sequence set forth in SEQ ID NO: 24, and the light chain variable region comprises the amino acid sequence set forth in SEQ ID NO: 23;
e) the heavy chain variable region comprises the amino acid sequence set forth in SEQ ID NO: 25, and the light chain variable region comprises the amino acid sequence set forth in SEQ ID NO: 23;
f) the heavy chain variable region comprises the amino acid sequence set forth in SEQ ID NO: 27, and the light chain variable region comprises the amino acid sequence set forth in SEQ ID NO: 23;
g) the heavy chain variable region comprises the amino acid sequence set forth in SEQ ID NO: 28, and the light chain variable region comprises the amino acid sequence set forth in SEQ ID NO: 23;
h) the heavy chain variable region comprises the amino acid sequence set forth in SEQ ID NO: 29, and the light chain variable region comprises the amino acid sequence set forth in SEQ ID NO: 23;
i) the heavy chain variable region comprises the amino acid sequence set forth in SEQ ID NO: 22, and the light chain variable region comprises the amino acid sequence set forth in SEQ ID NO: 30;
j) the heavy chain variable region comprises the amino acid sequence set forth in SEQ ID NO: 24, and the light chain variable region comprises the amino acid sequence set forth in SEQ ID NO: 30;
k) the heavy chain variable region comprises the amino acid sequence set forth in SEQ ID NO: 25, and the light chain variable region comprises the amino acid sequence set forth in SEQ ID NO: 30;
l) the heavy chain variable region comprises the amino acid sequence set forth in SEQ ID NO: 27, and the light chain variable region comprises the amino acid sequence set forth in SEQ ID NO: 30;
m) the heavy chain variable region comprises the amino acid sequence set forth in SEQ ID NO: 28, and the light chain variable region comprises the amino acid sequence set forth in SEQ ID NO: 30;
n) the heavy chain variable region comprises the amino acid sequence set forth in SEQ ID NO: 29, and the light chain variable region comprises the amino acid sequence set forth in SEQ ID NO: 30;
o) the heavy chain variable region comprises the amino acid sequence set forth in SEQ ID NO: 22, and the light chain variable region comprises the amino acid sequence set forth in SEQ ID NO: 31;
p) the heavy chain variable region comprises the amino acid sequence set forth in SEQ ID NO: 24, and the light chain variable region comprises the amino acid sequence set forth in SEQ ID NO: 31;
q) the heavy chain variable region comprises the amino acid sequence set forth in SEQ ID NO: 25, and the light chain variable region comprises the amino acid sequence set forth in SEQ ID NO: 31;
r) the heavy chain variable region comprises the amino acid sequence set forth in SEQ ID NO: 27, and the light chain variable region comprises the amino acid sequence set forth in SEQ ID NO: 31;
s) the heavy chain variable region comprises the amino acid sequence set forth in SEQ ID NO: 28, and the light chain variable region comprises the amino acid sequence set forth in SEQ ID NO: 31;
t) the heavy chain variable region comprises the amino acid sequence set forth in SEQ ID NO: 29, and the light chain variable region comprises the amino acid sequence set forth in SEQ ID NO: 31;
u) the heavy chain variable region comprises the amino acid sequence set forth in SEQ ID NO: 22, and the light chain variable region comprises the amino acid sequence set forth in SEQ ID NO: 32;
v) the heavy chain variable region comprises the amino acid sequence set forth in SEQ ID NO: 24, and the light chain variable region comprises the amino acid sequence set forth in SEQ ID NO: 32;
w) the heavy chain variable region comprises the amino acid sequence set forth in SEQ ID NO: 25, and the light chain variable region comprises the amino acid sequence set forth in SEQ ID NO: 32;
x) the heavy chain variable region comprises the amino acid sequence set forth in SEQ ID NO: 27, and the light chain variable region comprises the amino acid sequence set forth in SEQ ID NO: 32;
y) the heavy chain variable region comprises the amino acid sequence set forth in SEQ ID NO: 28, and the light chain variable region comprises the amino acid sequence set forth in SEQ ID NO: 32;
z) the heavy chain variable region comprises the amino acid sequence set forth in SEQ ID NO: 29, and the light chain variable region comprises the amino acid sequence set forth in SEQ ID NO: 32;
aa) the heavy chain variable region comprises the amino acid sequence set forth in SEQ ID NO: 22, and the light chain variable region comprises the amino acid sequence set forth in SEQ ID NO: 19;
ab) the heavy chain variable region comprises the amino acid sequence set forth in SEQ ID NO: 24, and the light chain variable region comprises the amino acid sequence set forth in SEQ ID NO: 19; ac) the heavy chain variable region comprises the amino acid sequence set forth in SEQ ID NO: 25, and the light chain variable region comprises the amino acid sequence set forth in SEQ ID NO: 19;
ad) the heavy chain variable region comprises the amino acid sequence set forth in SEQ ID NO: 27, and the light chain variable region comprises the amino acid sequence set forth in SEQ ID NO: 19;
ae) the heavy chain variable region comprises the amino acid sequence set forth in SEQ ID NO: 28, and the light chain variable region comprises the amino acid sequence set forth in SEQ ID NO: 19;
af) the heavy chain variable region comprises the amino acid sequence set forth in SEQ ID NO: 29, and the light chain variable region comprises the amino acid sequence set forth in SEQ ID NO: 19;
ag) the heavy chain variable region comprises the amino acid sequence set forth in SEQ ID NO: 33, and the light chain variable region comprises the amino acid sequence set forth in SEQ ID NO: 34;
ah) the heavy chain variable region comprises the amino acid sequence set forth in SEQ ID NO: 41, and the light chain variable region comprises the amino acid sequence set forth in SEQ ID NO: 42;
ai) the heavy chain variable region comprises the amino acid sequence set forth in SEQ ID NO: 49, and the light chain variable region comprises the amino acid sequence set forth in SEQ ID NO: 50;
aj) the heavy chain variable region comprises the amino acid sequence set forth in SEQ ID NO: 49, and the light chain variable region comprises the amino acid sequence set forth in SEQ ID NO: 53;
ak) the heavy chain variable region comprises the amino acid sequence set forth in SEQ ID NO: 60, and the light chain variable region comprises the amino acid sequence set forth in SEQ ID NO: 61;
al) the heavy chain variable region comprises the amino acid sequence set forth in SEQ ID NO: 67, and the light chain variable region comprises the amino acid sequence set forth in SEQ ID NO: 68;
am) the heavy chain variable region comprises the amino acid sequence set forth in SEQ ID NO: 75, and the light chain variable region comprises the amino acid sequence set forth in SEQ ID NO: 76;
an) the heavy chain variable region comprises the amino acid sequence set forth in SEQ ID NO: 87, and the light chain variable region comprises the amino acid sequence set forth in SEQ ID NO: 88;
ao) the heavy chain variable region comprises the amino acid sequence set forth in SEQ ID NO: 99, and the light chain variable region comprises the amino acid sequence set forth in SEQ ID NO: 100;
ap) the heavy chain variable region comprises the amino acid sequence set forth in SEQ ID NO: 110, and the light chain variable region comprises the amino acid sequence set forth in SEQ ID NO: 111;
aq) the heavy chain variable region comprises the amino acid sequence set forth in SEQ ID NO: 122, and the light chain variable region comprises the amino acid sequence set forth in SEQ ID NO: 123;
ar) the heavy chain variable region comprises the amino acid sequence set forth in SEQ ID NO: 128, and the light chain variable region comprises the amino acid sequence set forth in SEQ ID NO: 129;
as) the heavy chain variable region comprises the amino acid sequence set forth in SEQ ID NO: 130, and the light chain variable region comprises the amino acid sequence set forth in SEQ ID NO: 131;
at) the heavy chain variable region comprises the amino acid sequence set forth in SEQ ID NO: 130, and the light chain variable region comprises the amino acid sequence set forth in SEQ ID NO: 132;
au) the heavy chain variable region comprises the amino acid sequence set forth in SEQ ID NO: 130, and the light chain variable region comprises the amino acid sequence set forth in SEQ ID NO: 133;
av) the heavy chain variable region comprises the amino acid sequence set forth in SEQ ID NO: 134, and the light chain variable region comprises the amino acid sequence set forth in SEQ ID NO: 131;
aw) the heavy chain variable region comprises the amino acid sequence set forth in SEQ ID NO: 134, and the light chain variable region comprises the amino acid sequence set forth in SEQ ID NO: 132;
ax) the heavy chain variable region comprises the amino acid sequence set forth in SEQ ID NO: 134, and the light chain variable region comprises the amino acid sequence set forth in SEQ ID NO: 133;
ay) the heavy chain variable region comprises the amino acid sequence set forth in SEQ ID NO: 135, and the light chain variable region comprises the amino acid sequence set forth in SEQ ID NO: 131;
az) the heavy chain variable region comprises the amino acid sequence set forth in SEQ ID NO: 135, and the light chain variable region comprises the amino acid sequence set forth in SEQ ID NO: 132;
ba) the heavy chain variable region comprises the amino acid sequence set forth in SEQ ID NO: 135, and the light chain variable region comprises the amino acid sequence set forth in SEQ ID NO: 133; or
bb) the heavy chain variable region comprises the amino acid sequence set forth in SEQ ID NO: 135, and the light chain variable region comprises the amino acid sequence set forth in SEQ ID NO: 136.

In certain embodiments,
a) the heavy chain variable region comprises the amino acid sequence set forth in SEQ ID NO: 87, and the light chain variable region comprises the amino acid sequence set forth in SEQ ID NO: 88;
b) the heavy chain variable region comprises the amino acid sequence set forth in SEQ ID NO: 99, and the light chain variable region comprises the amino acid sequence set forth in SEQ ID NO: 100; or
c) the heavy chain variable region comprises the amino acid sequence set forth in SEQ ID NO: 110, and the light chain variable region comprises the amino acid sequence set forth in SEQ ID NO: 111.

In certain embodiments, the extracellular antigen-binding domain comprises a linker between the heavy chain variable region and the light chain variable region of the extracellular antigen-binding domain. In certain embodiments, the linker consists of the amino acid sequence set forth in SEQ ID NO: 10, SEQ ID NO: 11, SEQ ID NO: 20, SEQ ID NO: 21, SEQ ID NO: 137, or SEQ ID NO: 138. In certain embodiments, the linker consists of the amino acid sequence set forth in SEQ ID NO: 11. In certain embodiments, a signal peptide is covalently joined to the 5′ terminus of the extracellular antigen-binding domain.

In certain embodiments, the heavy chain variable region and the light chain variable region are positioned from the N- to the C-terminus: VH-VL. In certain embodiments, the heavy chain variable region and the light chain variable region are positioned from the N- to the C-terminus: VL-VH.

In certain non-limiting embodiments, the presently disclosed subject matter provides an antigen-recognizing receptor comprising an extracellular antigen-binding domain, a transmembrane domain, and an intracellular signaling domain, wherein the extracellular antigen-binding domain specifically binds to L1CAM and comprises a heavy chain variable region and a light chain variable region, wherein the heavy chain variable region comprises a CDR1 comprising the amino acid sequence set forth in SEQ ID NO: 81, a CDR2 comprising the amino acid sequence set forth in SEQ ID NO: 82, and a CDR3 comprising the amino acid sequence set forth in SEQ ID NO: 83; and the light chain variable region comprises a CDR1 comprising the amino acid sequence set forth in SEQ ID NO: 84, a CDR2 comprising the amino acid sequence set forth in SEQ ID NO: 85, and a CDR3 comprising the amino acid sequence set forth in SEQ ID NO: 86.

In certain non-limiting embodiments, the presently disclosed subject matter provides an antigen-recognizing receptor comprising an extracellular antigen-binding domain, a transmembrane domain, and an intracellular signaling domain, wherein the extracellular antigen-binding domain specifically binds to L1CAM and comprises a heavy chain variable region and a light chain variable region, wherein the heavy chain variable region comprises a CDR1 comprising the amino acid sequence set forth in SEQ ID NO: 93, a CDR2 comprising the amino acid sequence set forth in SEQ ID NO: 94, and a CDR3 comprising the amino acid sequence set forth in SEQ ID NO: 95; and the light chain variable region comprises a CDR1 comprising the amino acid sequence set forth in SEQ ID NO: 96, a CDR2 comprising the amino acid sequence set forth in SEQ ID NO: 97, and a CDR3 comprising the amino acid sequence set forth in SEQ ID NO: 98.

In certain non-limiting embodiments, the presently disclosed subject matter provides an antigen-recognizing receptor comprising an extracellular antigen-binding domain, a transmembrane domain, and an intracellular signaling domain, wherein the extracellular antigen-binding domain specifically binds to L1CAM and comprises a heavy chain variable region and a light chain variable region, wherein the heavy chain variable region comprises a CDR1 comprising the amino acid sequence set forth in SEQ ID NO: 105, a CDR2 comprising the amino acid sequence set forth in SEQ ID NO: 106, and a CDR3 comprising the amino acid sequence set forth in SEQ ID NO: 107; and the light chain variable region comprises a CDR1 comprising the amino acid sequence set forth in SEQ ID NO: 108, a CDR2 comprising the amino acid sequence set forth in SEQ ID NO: 39, and a CDR3 comprising the amino acid sequence set forth in SEQ ID NO: 109.

In certain non-limiting embodiments, the presently disclosed subject matter provides an antigen-recognizing receptor comprising an extracellular antigen-binding domain, a transmembrane domain, and an intracellular signaling domain, wherein the extracellular antigen-binding domain specifically binds to L1CAM and comprises a heavy chain variable region and a light chain variable region, wherein the heavy chain variable region comprises a CDR1, a CDR2, and a CDR3 of a heavy chain variable region comprising the amino acid sequence set forth in SEQ ID NO: 87, and the light chain variable region comprises a CDR1, a CDR2, and a CDR3 of a light chain variable region comprising the amino acid sequence set forth in SEQ ID NO: 88.

In certain non-limiting embodiments, the presently disclosed subject matter provides an antigen-recognizing receptor comprising an extracellular antigen-binding domain, a transmembrane domain, and an intracellular signaling domain, wherein the extracellular antigen-binding domain specifically binds to L1CAM and comprises a heavy chain variable region and a light chain variable region, wherein the heavy chain variable region comprises a CDR1, a CDR2, and a CDR3 of a heavy chain variable region comprising the amino acid sequence set forth in SEQ ID NO: 99, and the light chain variable region comprises a CDR1, a CDR2, and a CDR3 of a light chain variable region comprising the amino acid sequence set forth in SEQ ID NO: 100.

In certain non-limiting embodiments, the presently disclosed subject matter provides an antigen-recognizing receptor comprising an extracellular antigen-binding domain, a transmembrane domain, and an intracellular signaling domain, wherein the extracellular antigen-binding domain specifically binds to L1CAM and comprises a heavy chain variable region and a light chain variable region, wherein the heavy chain variable region comprises a CDR1, a CDR2, and a CDR3 of a heavy chain variable region comprising the amino acid sequence set forth in SEQ ID NO: 110, and the light chain variable region comprises a CDR1, a CDR2, and a CDR3 of a light chain variable region comprising the amino acid sequence set forth in SEQ ID NO: 111.

In certain non-limiting embodiments, the presently disclosed subject matter provides an antigen-recognizing receptor comprising an extracellular antigen-binding domain, a transmembrane domain, and an intracellular signaling domain, wherein the extracellular antigen-binding domain specifically binds to L1CAM and comprises a single chain a single chain variable region (scFv) comprising the amino acid sequence set forth in SEQ ID NO: 77.

In certain non-limiting embodiments, the presently disclosed subject matter provides an antigen-recognizing receptor comprising an extracellular antigen-binding domain, a transmembrane domain, and an intracellular signaling domain, wherein the extracellular antigen-binding domain specifically binds to L1CAM and comprises a single chain a single chain variable region (scFv) comprising the amino acid sequence set forth in SEQ ID NO: 78.

In certain non-limiting embodiments, the presently disclosed subject matter provides an antigen-recognizing receptor comprising an extracellular antigen-binding domain, a transmembrane domain, and an intracellular signaling domain, wherein the extracellular antigen-binding domain specifically binds to L1CAM and comprises a single chain a single chain variable region (scFv) comprising the amino acid sequence set forth in SEQ ID NO: 89.

In certain non-limiting embodiments, the presently disclosed subject matter provides an antigen-recognizing receptor comprising an extracellular antigen-binding domain, a transmembrane domain, and an intracellular signaling domain, wherein the extracellular antigen-binding domain specifically binds to L1CAM and comprises a single chain a single chain variable region (scFv) comprising the amino acid sequence set forth in SEQ ID NO: 90.

In certain non-limiting embodiments, the presently disclosed subject matter provides an antigen-recognizing receptor comprising an extracellular antigen-binding domain, a transmembrane domain, and an intracellular signaling domain, wherein the extracellular antigen-binding domain specifically binds to L1CAM and comprises a single chain a single chain variable region (scFv) comprising the amino acid sequence set forth in SEQ ID NO: 101.

In certain non-limiting embodiments, the presently disclosed subject matter provides an antigen-recognizing receptor comprising an extracellular antigen-binding domain, a transmembrane domain, and an intracellular signaling domain, wherein the extracellular antigen-binding domain specifically binds to L1CAM and comprises a single chain a single chain variable region (scFv) comprising the amino acid sequence set forth in SEQ ID NO: 102.

In certain embodiments, the transmembrane domain comprises a CD8 polypeptide, a CD28 polypeptide, a CD3ζ polypeptide, a CD4 polypeptide, a 4-1BB polypeptide, an OX40 polypeptide, an ICOS polypeptide, a CTLA-4 polypeptide, a PD-1 polypeptide, a LAG-3 polypeptide, a 2B4 polypeptide, a BTLA polypeptide, or a combination thereof. In certain embodiments, the transmembrane domain comprises a CD28 polypeptide. In certain embodiments, the intracellular signaling domain comprises a CD3ζ polypeptide.

In certain embodiments, the intracellular signaling domain further comprises at least one co-stimulatory signaling region. In certain embodiments, the at least one co-stimulatory signaling region comprises a CD28 polypeptide, a 4-1BB polypeptide, an OX40 polypeptide, an ICOS polypeptide, a DAP-10 polypeptide, or a combination thereof. In certain embodiments, the at least one co-stimulatory signaling region comprises a CD28 polypeptide.

In certain embodiments, the antigen-recognizing receptor is a chimeric antigen receptor (CAR) or a T-cell receptor (TCR) like fusion protein. In certain embodiments, the antigen-recognizing receptor is a CAR. In certain embodiments, the antigen-recognizing receptor is recombinantly expressed or expressed from a vector.

In certain non-limiting embodiments, the presently disclosed subject matter provides a cell comprising the antigen-recognizing receptor disclosed herein. In certain embodiments, the antigen-recognizing receptor is constitutively expressed on the surface of the cell.

In certain embodiments, the cell is an immunoresponsive cell. In certain embodiments, the cell is a cell of the lymphoid lineage or a cell of the myeloid lineage. In certain embodiments, the cell is selected from the group consisting of a T cell, a Natural Killer (NK) cell, and a stem cell from which a lymphoid cell may be differentiated. In certain embodiments, the cell is a T cell. In certain embodiments, the T cell is a cytotoxic T lymphocyte (CTL) or a regulatory T cell.

In certain embodiments, the stem cell is a pluripotent stem cell. In certain embodiments, the pluripotent stem cell is an embryoid stem cell or an induced pluripotent stem cell.

In certain non-limiting embodiments, the presently disclosed subject matter provides nucleic acid molecules encoding the antigen-recognizing receptor disclosed herein. In certain non-limiting embodiments, the presently disclosed subject matter provides vectors, host cells, and lipid nanoparticles comprising the nucleic acids disclosed herein.

In certain non-limiting embodiments, the presently disclosed subject matter provides compositions comprising the cell disclosed herein. In certain embodiments, the composition is a pharmaceutical composition further comprising a pharmaceutically acceptable carrier.

In certain non-limiting embodiments, the presently disclosed subject matter provides methods of treating and/or preventing a tumor in a subject in need thereof, comprising administering to the subject an effective amount of the cells or the compositions disclosed herein. In certain embodiments, the methods reduce the number of tumor cells, reduces tumor size, and/or eradicate the tumor in the subject.

In certain non-limiting embodiments, the presently disclosed subject matter provides methods of increasing or lengthening survival of a subject having a tumor, comprising administering to the subject an effective amount of the cells or the compositions disclosed herein. In certain non-limiting embodiments, the presently disclosed subject matter provides methods of reducing the risk of metastatic spread in a subject in need thereof, comprising administering to the subject an effective amount of the cells or the compositions disclosed herein. In certain non-limiting embodiments, the presently disclosed subject matter provides methods of inhibiting metastatic spread of a primary cancer in a subject in need thereof, comprising administering to the subject an effective amount of the cells or the compositions disclosed herein. In certain non-limiting embodiments, the presently disclosed subject matter provides methods of inhibiting progression of metastatic disease in a subject in need thereof, comprising administering to the subject an effective amount of the cells or the compositions disclosed herein.

In certain embodiments, the tumor is associated with L1CAM. In certain embodiments, the tumor is a cancer. In certain embodiments, the cancer is a metastatic cancer. In certain embodiments, the metastatic cancer localizes in at least one tissue selected from the group consisting of bone, liver, lung, brain, peritoneum, adrenal gland, skin, lymph nodes, pleura, and meninges.

In certain non-limiting embodiments, the presently disclosed subject matter provides methods for producing a cell comprising an antigen-recognizing receptor disclosed herein, comprising introducing into the cell a nucleic acid molecule that encodes the antigen-recognizing receptor.

In certain non-limiting embodiments, the presently disclosed subject matter provides kits for reducing tumor burden in a subject, treating and/or preventing a tumor in a subject, and/or increasing or lengthening survival of a subject having a tumor, comprising the cells disclosed herein. In certain embodiments, the kit further comprises written instructions for using the cell for reducing tumor burden in a subject, treating and/or preventing a tumor or neoplasm in a subject, and/or increasing or lengthening survival of a subject having a tumor.

In certain non-limiting embodiments, the presently disclosed subject matter provides cells or compositions disclosed herein for use in treating and/or preventing a tumor in a subject in need thereof, increasing or lengthening survival of a subject having a tumor, reducing the risk of metastatic spread in a subject in need thereof, inhibiting metastatic spread of a primary cancer in a subject in need thereof, and/or inhibiting progression of metastatic disease in a subject in need thereof.

In certain non-limiting embodiments, the presently disclosed subject matter provides cells or compositions disclosed herein for treating and/or preventing a tumor in a subject in need thereof, increasing or lengthening survival of a subject having a tumor, reducing the risk of metastatic spread in a subject in need thereof, inhibiting metastatic spread of a primary cancer in a subject in need thereof, and/or inhibiting progression of metastatic disease in a subject in need thereof.

In certain non-limiting embodiments, the presently disclosed subject matter provides use of the cells or compositions disclosed herein for the manufacture of a medicament for treating and/or preventing a tumor in a subject in need thereof, increasing or lengthening survival of a subject having a tumor, reducing the risk of metastatic spread in a subject in need thereof, inhibiting metastatic spread of a primary cancer in a subject in need thereof, and/or inhibiting progression of metastatic disease in a subject in need thereof.

### BRIEF DESCRIPTION OF THE FIGURES

The following Detailed Description, given by way of example, but not intended to limit the present disclosure to specific embodiments described, may be understood in conjunction with the accompanying drawings.

FIGS. 1A and 1B illustrate binding of humanized 76H12 Fab variants. FIG. 1A shows flow cytometry for binding to 293T cells engineered to overexpress L1CAM. FIG. 1B shows kinetic data of tested humanized variants binding to L1CAM-Fc fusion protein.

FIGS. 2A and 2B illustrate the generation and activity of the presently disclosed antigen recognizing receptors. FIG. 2A shows an exemplary schematic of the presently disclosed chimeric antigen receptor. FIG. 2B shows the results of an in vitro killing assay using different clones, as detailed in the Example section.

FIGS. 3A-3C illustrate the polyfunctionality of T cells expressing a CAR including the 18H05 scFv (18H05) or the 76H12 scFv (76H12). FIG. 3A shows the results of a killing assay against 293 L1CAM1 cells. FIG. 3B shows the results of a killing assay against MDA MB231 cells. FIG. 3C shows the production of INF gamma in presence of 293 cells expressing L1CAM (+293 L1CAM1) or controls (−293 L1CAM1).

FIGS. 4A-4D show the polyfunctionality of T cells expressing a CAR including the 14A10 scFv (14A10).

FIG. 5 depicts screening funnel and hit summary for hybridoma supernatants. The steps of the screening funnel are shown in sequence on the left. For each step, utilized method, selection criteria and number of hits for wild-type mice (Balb/c & A/J) and transgenic mice (AlivaMab®) are indicated.

FIG. 6 depicts internalization of L1CAM lead candidate mAbs. A Fab-ZAP assay was used to determine the potency of internalization for the L1CAM lead candidate mAbs in HEK293T cells overexpressing human L1CAM. The L1CAM mAbs and a mouse IgG1 isotype control were pre-incubated at various concentrations with a saporin-conjugated Fab fragment that binds mouse Fc. Five days later, cell viability was measured and plotted against mAb concentrations.

FIG. 7 illustrates the polyfunctionality of T cells expressing a CAR including a humanized 76H12 scFv (76H12v3), an scFv derived from the L1CAM-binding antibody CE7 (CE7) or an scFv derived from a non-L1CAM binding antibody (Pali). The relative ability of each of these to induce killing of L1CAM-positive MDA MB 231 cells is plotted as a function of the ratio of effector to target cells.

### DETAILED DESCRIPTION

The presently disclosed subject matter provides antigen-recognizing receptors (e.g., a chimeric antigen receptor (CAR) or a T-cell receptor (TCR) like fusion molecule) that specifically target L1CAM. The presently disclosed subject matter further provides cells comprising such receptors. The cells can be immunoresponsive cells, e.g., genetically modified immunoresponsive cells (e.g., T cells or NK cells). The presently disclosed subject matter also provides methods of using such cells for treatments, e.g., for treating and/or preventing a metastatic cancer (e.g., lung, breast, and colorectal cancer).

Non-limiting embodiments of the present disclosure are described by the present specification and Examples.

For purposes of clarity of disclosure and not by way of limitation, the detailed description is divided into the following subsections:
1. Definitions;
2. L1CAM;
3. Antigen-Recognizing Receptors;
4. Cells;
5. Compositions and Vectors;
6. Polypeptides;
7. Formulations and Administration;
8. Methods of Treatment;
9. Kits; and
10. Exemplary Embodiments.

### 1. DEFINITIONS

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

By “immunoresponsive cell” is meant a cell that functions in an immune response or a progenitor, or progeny thereof. In certain embodiments, the immunoresponsive cell is a cell of lymphoid lineage. Non-limiting examples of cells of lymphoid lineage include T cells, Natural Killer (NK) cells, B cells, and stem cells from which lymphoid cells may be differentiated. In certain embodiments, the immunoresponsive cell is a cell of myeloid lineage.

By “activates an immunoresponsive cell” is meant induction of signal transduction or changes in protein expression in the cell resulting in initiation of an immune response. For example, when CD3 Chains cluster in response to ligand binding and immunoreceptor tyrosine-based inhibition motifs (ITAMs) a signal transduction cascade is produced. In certain embodiments, when a CAR or a TCR like fusion molecule binds to an antigen, a formation of an immunological synapse occurs that includes clustering of many molecules near the bound receptor (e.g., CD4 or CD8, CD3γ/δ/ε/ζ, etc.). This clustering of membrane bound signaling molecules allows for ITAM motifs contained within the CD3 chains to become phosphorylated. This phosphorylation in turn initiates a T cell activation pathway ultimately activating transcription factors, such as NF-κB and AP-1. These transcription factors induce global gene expression of the T cell to increase IL-2 production for proliferation and expression of master regulator T cell proteins in order to initiate a T cell mediated immune response.

By “stimulates an immunoresponsive cell” is meant a signal that results in a robust and sustained immune response. In various embodiments, this occurs after immune cell (e.g., T-cell) activation or concomitantly mediated through receptors including, but not limited to, CD28, CD137 (4-1BB), OX40, CD40 and ICOS. Receiving multiple stimulatory signals can be important to mount a robust and long-term T cell mediated immune response. T cells can quickly become inhibited and unresponsive to antigen. While the effects of these co-stimulatory signals may vary, they generally result in increased gene expression in order to generate long lived, proliferative, and anti-apoptotic T cells that robustly respond to antigen for complete and sustained eradication.

The term “antigen-recognizing receptor” as used herein refers to a receptor that is capable of recognizing a target antigen (e.g., L1CAM). In certain embodiments, the antigen-recognizing receptor is capable of activating an immune or immunoresponsive cell (e.g., a T cell) upon its binding to the target antigen.

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fc fragment of intact antibody, clear more rapidly from the circulation, and may have less non-specific tissue binding of an intact antibody (Wahl et al., Nucl Med (1983); 24:316-325). As used herein, include whole native antibodies, bispecific antibodies; chimeric antibodies; Fab, Fab′, single chain V region fragments (scFv), fusion polypeptides, and unconventional antibodies. In certain embodiments, an antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (C_{H}) region. The heavy chain constant region is comprised of three domains, CH1, CH2 and CH3. Each light chain is comprised of a light chain variable region (abbreviated herein as V_{L}) and a light chain constant C_{L} region. The light chain constant region is comprised of one domain, C_{L}. The V_{H} and V_{L} regions can be further sub-divided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

As used herein, “CDRs” are defined as the complementarity determining region amino acid sequences of an antibody which are the hypervariable regions of immunoglobulin heavy and light chains. See, e.g., Kabat et al., Sequences of Proteins of Immunological Interest, 4th U. S. Department of Health and Human Services, National Institutes of Health (1987), or IMGT numbering system (Lefranc, The Immunologist (1999); 7:132-136; Lefranc et al., Dev. Comp. Immunol. (2003); 27:55-77). The CDRs can also be numbered according to the IMGT numbering system, e.g., the IMGT numbering system accessible at http://www.imgt.org/IMGT_vquest/input. Generally, antibodies comprise three heavy chain and three light chain CDRs or CDR regions in the variable region. CDRs provide the majority of contact residues for the binding of the antibody to the antigen or epitope. In certain embodiments, the CDRs regions are delineated according to the Kabat numbering system.

As used herein, the term “single-chain variable fragment” or “scFv” is a fusion protein of the variable regions of the heavy (V_{H}) and light chains (V_{L}) of an immunoglobulin (e.g., mouse or human) covalently linked to form a V_{H}::VL heterodimer. The heavy (V_{H}) and light chains (V_{L}) are either joined directly or joined by a peptide-encoding linker (e.g., 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. The linker can link the heavy chain variable region and the light chain variable region of the extracellular antigen-binding domain. Non-limiting examples of linkers are disclosed in Shen et al., Anal. Chem. 80(6):1910-1917 (2008) and WO 2014/087010, the contents of which are hereby incorporated by reference in their entireties. In certain embodiments, the linker is a G4S linker.

In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 1380, which is provided below:

[SEQ ID NO: 10]
GGGGSGGGGSGGGSGGGGS

In certain embodiments, the linker comprise the amino acid sequence set forth in SEQ ID NO: 11, which is provided below:

[SEQ ID NO: 11]
GGGGSGGGGSGGGGS

In certain embodiments, the linker comprises the amino acid sequence set forth in SEQ ID NO: 20, which is provided below:

[SEQ ID NO: 20]
GGGGSGGGGSGGGGSGGGSGGGGS

In certain embodiments, the linker comprises the amino acid sequence set forth in SEQ ID NO: 21, which is provided below:

[SEQ ID NO: 21]
GGGGSGGGGSGGGGSGGGGSGGGSGGGGS

In certain embodiments, the linker comprises the amino acid sequence set forth in SEQ ID NO: 137, which is provided below:

[SEQ ID NO: 137]
GGGGS

In certain embodiments, the linker comprises the amino acid sequence set forth in SEQ ID NO: 138, which is provided below:

[SEQ ID NO: 138]
GGGGSGGGGS

Despite removal of the constant regions and the introduction of a linker, scFv proteins retain the specificity of the original immunoglobulin. Single chain Fv polypeptide antibodies can be expressed from a nucleic acid comprising V_{H}- and V_{L}-encoding sequences as described by Huston, et al. Proc. Nat. Acad. Sci. USA, (1988); 85:5879-5883; U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) (2008); 27(6):455-51; Peter et al., J Cachexia Sarcopenia Muscle (2012); August 12; Shieh et al., J Imunol (2009); 183(4):2277-85; Giomarelli et al., Thromb Haemost (2007); 97(6):955-63; Fife et al., J Clin Invst (2006); 116(8):2252-61; Brocks et al., Immunotechnology 1997 3(3):173-84; Moosmayer et al., Ther Immunol 1995 2(10:31-40). Agonistic scFvs having stimulatory activity have been described (Peter et al., J Biol Chem (2003); 25278(38):36740-7; Xie et al., Nat Biotech 1997 15(8):768-71; Ledbetter et al., Crit Rev Immunol (1997); 17(5-6):427-55; Ho et al., BioChim Biophys Acta (2003); 1638(3):257-66).

The term “chimeric antigen receptor” or “CAR” as used herein refers to a molecule comprising an extracellular antigen-binding domain that is fused to an intracellular signaling domain that is capable of activating or stimulating an immunoresponsive cell, and a transmembrane domain. In certain embodiments, the extracellular antigen-binding domain of a CAR comprises a scFv. The scFv can be derived from fusing the variable heavy and light regions of an antibody. In certain embodiments, the scFv may be derived from Fab's (instead of from an antibody, e.g., obtained from Fab libraries). In certain embodiments, the scFv is fused to the transmembrane domain and then to the intracellular signaling domain.

By “substantially identical” or “substantially homologous” is meant a polypeptide or nucleic acid molecule exhibiting at least about 50% homologous or identical to a reference amino acid sequence (for example, any of the amino acid sequences described herein) or a reference nucleic acid sequence (for example, any of the nucleic acid sequences described herein). In certain embodiments, such a sequence is at least about 60%, at least about 65%, at least about 70%, at least about 75%, at least about 80%, at least about 85%, at least about 90%, at least about 95%, at least about 99%, or at least about 100% homologous or identical to the sequence of the amino acid or nucleic acid used for comparison.

Sequence identity can be measured by using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

In certain embodiments, the percent homology between two amino acid sequences is equivalent to the percent identity between the two sequences. The percent identity between the two sequences is a function of the number of identical positions shared by the sequences (i.e., % homology=#of identical positions/total #of positions×100), taking into account the number of gaps, and the length of each gap, which need to be introduced for optimal alignment of the two sequences. The comparison of sequences and determination of percent identity between two sequences can be accomplished using a mathematical algorithm.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the amino acids sequences of the presently disclosed subject matter can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the specified sequences (e.g., heavy and light chain variable region sequences of scFv m903, m904, m905, m906, and m900) disclosed herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

An “effective amount” is an amount sufficient to affect a beneficial or desired clinical result upon treatment. An effective amount can be administered to a subject in one or more doses. In certain embodiments, an effective amount can be an amount that is sufficient to palliate, ameliorate, stabilize, reverse or slow the progression of the disease, or otherwise reduce the pathological consequences of the disease. The effective amount can be determined by a physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the cells administered.

As used herein, the term “endogenous” refers to a nucleic acid molecule or polypeptide that is normally expressed in a cell or tissue.

As used herein, the term “exogenous” refers to a nucleic acid molecule or polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid not present in a native wild-type cell; for example, an exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

As used herein, the term “immunosuppressive activity” is meant induction of signal transduction or changes in protein expression in a cell (e.g., an activated immunoresponsive cell) resulting in a decrease in an immune response. Non-limiting examples of polypeptides suppressing or decreasing an immune response via their binding include CD47, PD-1, CTLA-4, BTLA, LAG-3, 2B4, and their corresponding ligands (including, but not limited to, SIRPa, PD-L1, PD-L2, TNFRSF14, CD48, and FGL-1). Such polypeptides are present in the tumor microenvironment and inhibit immune responses to neoplastic cells. In certain embodiments, inhibiting, blocking, or antagonizing the interaction of immunosuppressive polypeptides and/or their ligands enhances the immune response of the immunoresponsive cell.

As used herein, the term “immunostimulatory activity” is meant induction of signal transduction or changes in protein expression in a cell (e.g., an activated immunoresponsive cell) resulting in an increase in an immune response. Immunostimulatory activity may include pro-inflammatory activity. Non-limiting examples of polypeptides stimulating or increasing an immune response via their binding include CD28, CD40, OX-40, 4-1BB, GITR, and their corresponding ligands, including B7-1, B7-2, CD40L, OX-40L, 4-1BBL, GITRL. Such polypeptides are present in the tumor microenvironment and activate immune responses to neoplastic cells. In certain embodiments, promoting, stimulating, or agonizing pro-inflammatory polypeptides and/or their ligands enhances the immune response of the immunoresponsive cell.

By a “heterologous nucleic acid molecule or polypeptide” is meant a nucleic acid molecule (e.g., a cDNA, DNA or RNA molecule) or polypeptide that is not normally present in a cell or sample obtained from a cell. This nucleic acid may be from another organism, or it may be, for example, an mRNA molecule that is not normally expressed in a cell or sample.

By “modulate” is meant positively or negatively alter. Exemplary modulations include a about 1%, about 2%, about 5%, about 10%, about 25%, about 50%, about 75%, or about 100% change.

By “increase” is meant to alter positively by at least about 5%. An alteration may be by about 5%, about 10%, about 25%, about 30%, about 50%, about 75%, about 100% or more.

By “reduce” is meant to alter negatively by at least about 5%. An alteration may be by about 5%, about 10%, about 25%, about 30%, about 50%, about 75%, or even by about 100%.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated cell” is meant a cell that is separated from the molecular and/or cellular components that naturally accompany the cell.

The term “antigen-binding domain” as used herein refers to a domain capable of specifically binding a particular antigenic determinant or set of antigenic determinants present on a cell.

By “neoplasm” or “neoplasia” is meant a disease characterized by the pathological proliferation of a cell or tissue and its subsequent migration to or invasion of other tissues or organs. Neoplastic growth is typically uncontrolled and progressive, and occurs under conditions that would not elicit, or would cause cessation of, multiplication of normal cells. Neoplasm can affect a variety of cell types, tissues, or organs, including but not limited to an organ selected from the group consisting of bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasia includes cancers, such as sarcomas, carcinomas, or plasmacytomas (malignant tumor of the plasma cells). The neoplasia can a primary tumor or primary cancer. In addition, the neoplasm can be in metastatic status.

By “receptor” is meant a polypeptide, or portion thereof, present on a cell membrane that selectively binds one or more ligand.

By “signal sequence” or “leader sequence” is meant a peptide sequence (e.g., 5, 10, 15, 20, 25 or 30 amino acids) present at the N-terminus of newly synthesized proteins that directs their entry to the secretory pathway

By “specifically binds” or “specifically binds to” or “specifically target” is meant a polypeptide or a fragment thereof that recognizes and/or binds to a biological molecule of interest (e.g., a L1CAM polypeptide), but which does not substantially recognize and/or bind other molecules in a sample, for example, a biological sample, which naturally includes a presently disclosed polypeptide (e.g., a L1CAM polypeptide).

The terms “comprises”, “comprising”, and are intended to have the broad meaning ascribed to them in U.S. Patent Law and can mean “includes”, “including” and the like.

As used herein, “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

An “individual” or “subject” herein is a vertebrate, such as a human or non-human animal, for example, a mammal. Mammals include, but are not limited to, humans, primates, farm animals, sport animals, rodents, and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys. The term “immunocompromised” as used herein refers to a subject who has an immunodeficiency. The subject is very vulnerable to opportunistic infections, infections caused by organisms that usually do not cause disease in a person with a healthy immune system but can affect people with a poorly functioning or suppressed immune system.

Other aspects of the presently disclosed subject matter are described in the following disclosure and are within the ambit of the presently disclosed subject matter.

### 2. L1CAM

L1CAM is a transmembrane protein member of the L1 protein family, encoded by the L1CAM gene. This protein, of about 200 to about 220 kDa, is a neuronal cell adhesion molecule with a strong implication in cell migration, adhesion, neurite outgrowth, myelination and neuronal differentiation (Samatov et al., Prog Histochem Cytochem. 2016 August; 51(2):25-32).

L1CAM is composed of six IgG domains and three fibronectin type III domains and a short cytoplasmic domain. The extracellular domain forms homophilic interactions and heterophilic interactions with integrins and several extracellular matrix proteins. Via its cytoplasmic tail, L1CAM binds ezrin, ankyrin, and other signaling adaptor proteins. L1CAM is predominantly expressed by developing neurons, in which it regulates axon extension and synaptogenesis, and is also found in certain hematological and endothelial cell types. Notably, cancer cells exclusively express non-neuronal splice forms of L1CAM, and it was recently discovered that these isoforms are also naturally expressed in pericytes, the contractile mesenchymal cells that wrap around capillaries to regulate blood flow. L1CAM is involved in metastatic outgrowth of multiple solid tumors, including the three most prominent causes of cancer death (e.g., lung, breast, and colorectal cancer), as well as in metastatic outgrowth in multiple organ sites (e.g., bone, lung, liver, and brain). Further, L1CAM is involved in the outgrowth of aggressive cancer cells right after freshly seeding a target organ as well as indolent cancer cells that emerge from dormancy. L1CAM is required for the initiation of micrometastases, maintenance and expansion of established metastases, and for the re-initiation of metastatic growth by dormant micrometastases.

In certain embodiments, the presently disclosed anti-L1CAM antigen-recognizing receptors bind to human L1CAM. In certain embodiments, the human L1CAM comprises or consists of the amino acid sequence with a UniProt Reference No: P32004 (SEQ ID NO: 1) or a fragment thereof. SEQ ID NO: 1 is provided below. In certain embodiments, the human L1CAM comprises an extracellular domain, a transmembrane domain, and a cytoplasmic domain. In certain embodiments, the extracellular domain comprises or consists of amino acids 20 to 1120 of SEQ ID NO: 1. In certain embodiments, the transmembrane domain comprises or consists of amino acids 1121 to 1143 of SEQ ID NO: 1. In certain embodiments, the cytoplasmic domain comprises or consists of amino acids 1144 to 1257 of SEQ ID NO: 1.

[SEQ ID NO: 1]
MVVALRYVWPLLLCSPCLLIQIPEEYEGHHVMEPPVITEQSPRRLVVFPTDDISLKCEASGKPEVQ
FRWTRDGVHFKPKEELGVTVYQSPHSGSFTITGNNSNFAQRFQGIYRCFASNKLGTAMSHEIRLMA
EGAPKWPKETVKPVEVEEGESVVLPCNPPPSAEPLRIYWMNSKILHIKQDERVTMGQNGNLYFANV
LTSDNHSDYICHAHFPGTRTIIQKEPIDLRVKATNSMIDRKPRLLFPTNSSSHLVALQGQPLVLEC
IAEGFPTPTIKWLRPSGPMPADRVTYQNHNKTLQLLKVGEEDDGEYRCLAENSLGSARHAYYVTVE
AAPYWLHKPQSHLYGPGETARLDCQVQGRPQPEVTWRINGIPVEELAKDQKYRIQRGALILSNVQP
SDTMVTQCEARNRHGLLLANAYIYVVQLPAKILTADNQTYMAVQGSTAYLLCKAFGAPVPSVQWLD
EDGTTVLQDERFFPYANGTLGIRDLQANDTGRYFCLAANDQNNVTIMANLKVKDATQITQGPRSTI
EKKGSRVTFTCQASFDPSLQPSITWRGDGRDLQELGDSDKYFIEDGRLVIHSLDYSDQGNYSCVAS
TELDVVESRAQLLVVGSPGPVPRLVLSDLHLLTQSQVRVSWSPAEDHNAPIEKYDIEFEDKEMAPE
KWYSLGKVPGNQTSTTLKLSPYVHYTFRVTAINKYGPGEPSPVSETVVTPEAAPEKNPVDVKGEGN
ETTNMVITWKPLRWMDWNAPQVQYRVQWRPQGTRGPWQEQIVSDPFLVVSNTSTFVPYEIKVQAVN
SQGKGPEPQVTIGYSGEDYPQAIPELEGIEILNSSAVLVKWRPVDLAQVKGHLRGYNVTYWREGSQ
RKHSKRHIHKDHVVVPANTTSVILSGLRPYSSYHLEVQAFNGRGSGPASEFTFSTPEGVPGHPEAL
HLECQSNTSLLLRWQPPLSHNGVLTGYVLSYHPLDEGGKGQLSFNLRDPELRTHNLTDLSPHLRYR
FQLQATTKEGPGEAIVREGGTMALSGISDFGNISATAGENYSVVSWVPKEGQCNFRFHILFKALGE
EKGGASLSPQYVSYNQSSYTQWDLQPDTDYEIHLFKERMFRHQMAVKINGTGRVRLPPAGFATEGW
FIGFVSAIILLLLVLLILCFIKRSKGGKYSVKDKEDTQVDSEARPMKDETFGEYRSLESDNEEKAF
GSSQPSLNGDIKPLGSDDSLADYGGSVDVQFNEDGSFIGQYSGKKEKEAAGGNDSSGATSPINPAV
ALE

In certain embodiments, the L1CAM comprises or consists of an amino acid sequence that is at least about 80%, at least about 85%, at least about 90%, at least about 95%, at least about 96%, at least about 97%, at least about 98%, or at least about 99%, at least about 100% identical to the amino acid sequence set forth in SEQ ID NO: 1 or a fragment thereof.

In certain embodiments, the presently disclosed anti-L1CAM antigen-recognizing receptors bind to a portion of human L1CAM. In certain embodiments, the presently disclosed anti-L1CAM antigen-recognizing receptors bind to the extracellular domain of L1CAM. In certain embodiments, the presently disclosed anti-L1CAM antigen-recognizing receptors bind to amino acids 20 to 1120 of SEQ ID NO: 1.

### 3. ANTIGEN-RECOGNIZING RECEPTORS

The presently disclosed antigen-recognizing receptors specifically target or binds to a L1CAM polypeptide. In certain embodiments, the antigen-recognizing receptor is a chimeric antigen receptor (CAR). In certain embodiments, the antigen-recognizing receptor is a TCR like fusion molecule.

The presently disclosed subject matter also provides nucleic acid molecules that encode the presently disclosed antigen-recognizing receptors. In certain embodiments, the nucleic acid molecule comprises a nucleotide sequence that encodes a polypeptide of a L1CAM-targeted antigen recognizing receptor disclosed herein.

### 3.1. Chimeric Antigen Receptor (CAR)

In certain embodiments, the antigen-recognizing receptor is a CAR. CARs are engineered receptors, which graft or confer a specificity of interest onto an immune effector cell. CARs can be used to graft the specificity of a monoclonal antibody onto a T cell; with transfer of their coding sequence facilitated by retroviral vectors.

There are three generations of CARs. “First generation” CARs are typically composed of an extracellular antigen-binding domain (e.g., an scFv), which is fused to a transmembrane domain, which is fused to cytoplasmic/intracellular signaling domain. “First generation” CARs can provide de novo antigen recognition and cause activation of both CD4⁺ and CD8⁺ T cells through their CD3ζ chain signaling domain in a single fusion molecule, independent of HLA-mediated antigen presentation. “Second generation” CARs add intracellular signaling domains from various co-stimulatory molecules (e.g., CD28, 4-1BB, ICOS, OX40) to the cytoplasmic tail of the CAR to provide additional signals to the T cell. “Second generation” CARs comprise those that provide both co-stimulation (e.g., CD28 or 4-1BB) and activation (CD3ζ). “Third generation” CARs comprise those that provide multiple co-stimulation (e.g., CD28 and 4-1BB) and activation (CD3ζ). In certain embodiments, the antigen-recognizing receptor is a first-generation CAR. In certain embodiments, the antigen-recognizing receptor is a CAR that does not comprise an intracellular signaling domain of a co-stimulatory molecule or a fragment thereof. In certain embodiments, the antigen-recognizing receptor is a second-generation CAR.

In certain embodiments, the CAR comprises an extracellular antigen-binding domain that specifically binds to a L1CAM polypeptide, a transmembrane domain, and an intracellular signaling domain.

### 3.1.1. Extracellular Antigen-Binding Domain of a CAR

In certain embodiments, the extracellular antigen-binding domain is an scFv. The scFv can be humanized scFv, murine scFv, or human scFv. In certain embodiments, the scFv is a humanized scFv.

In certain embodiments, the extracellular antigen-binding domain is a Fab. In certain embodiments, the Fab is optionally crosslinked. In certain embodiments, the extracellular antigen-binding domain is a F(ab)₂.

Any of the foregoing molecules can be comprised in a fusion protein with a heterologous sequence to form the extracellular antigen-binding domain. In certain non-limiting embodiments, the extracellular antigen-binding domain (embodied, for example, an scFv) binds to a L1CAM polypeptide (e.g., human L1CAM) with a binding affinity, for example with a dissociation constant (K_{D}) of 1×10⁻⁸ M or less, e.g., about 1×10⁻⁸ M or less, about 5×10⁻⁹ M or less, about 1×10⁻⁹ M or less, about 5×10⁻¹⁰ M or less, about 1×10⁻¹⁰ M or less, or about 1×10⁻¹¹ M or less. In certain embodiments, the extracellular antigen-binding domain (embodied, for example, an scFv) binds to a L1CAM polypeptide (e.g., human L1CAM) with a K_{D} of about 1×10⁻¹⁰. In certain embodiments, the extracellular antigen-binding domain (embodied, for example, an scFv) binds to a L1CAM polypeptide (e.g., human L1CAM) with a K_{D} of about 2×10⁻¹¹.

In certain embodiments, a presently disclosed extracellular antigen-binding domain binds to a cell expressing L1CAM (e.g., a metastatic cell expressing L1CAM) with a Half maximal Effective Concentration (EC50) value of from about 1 nM to about 50 nM, from about 5 nM to about 50 nM, from about 10 nM to about 50 nM, from about 20 nM to about 50 nM, from about 30 nM to about 50 nM, from about 40 nM to about 50 nM, or greater than about 50 nM. In certain embodiments, a presently disclosed extracellular antigen-binding domain binds to a cell expressing L1CAM (e.g., a metastatic cell expressing L1CAM) with a EC50 value of about 1 nM. In certain embodiments, a presently disclosed extracellular antigen-binding domain binds to a cell expressing L1CAM (e.g., a metastatic cell expressing L1CAM) with a EC50 value of about 4.8 nM.

In certain embodiments, the extracellular antigen-binding domain (e.g., an scFv) comprises a heavy chain variable region comprising a CDR1 comprising the amino acid sequence set forth in SEQ ID NO: 2 or a conservative modification thereof, a CDR2 comprising the amino acid sequence set forth in SEQ ID NO: 3 or a conservative modification thereof, and a CDR3 comprising the amino acid sequence set forth in SEQ ID NO: 4 or a conservative modification thereof. SEQ ID NOs: 2-4 are provided in Table 1.

In certain embodiments, the extracellular antigen-binding domain (e.g., an scFv) comprises a light chain variable region comprising a CDR1 comprising the amino acid sequence set forth in SEQ ID NO: 5 or a conservative modification thereof, a CDR2 comprising the amino acid sequence set forth in SEQ ID NO: 6 or a conservative modification thereof, and a CDR3 comprising the amino acid sequence set forth in SEQ ID NO: 7 or a conservative modification thereof. SEQ ID NOs: 5-7 are provided in Table 1.

In certain embodiments, the extracellular antigen-binding domain (e.g., an scFv) comprises a heavy chain variable region comprising a CDR1 comprising the amino acid sequence set forth in SEQ ID NO: 2 or a conservative modification thereof, a CDR2 comprising the amino acid sequence set forth in SEQ ID NO: 3 or a conservative modification thereof, and a CDR3 comprising the amino acid sequence set forth in SEQ ID NO: 4 or a conservative modification thereof, and a light chain variable region comprising a CDR1 comprising the amino acid sequence set forth in SEQ ID NO: 5 or a conservative modification thereof, a CDR2 comprising the amino acid sequence set forth in SEQ ID NO: 6 or a conservative modification thereof, and a CDR3 comprising the amino acid sequence set forth in SEQ ID NO: 7 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 2, a CDR2 comprising the amino acid sequence set forth in SEQ ID NO: 3, and a CDR3 comprising the amino acid sequence set forth in SEQ ID NO: 4; and a light chain variable region comprising a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 8. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 8. In certain embodiments, the extracellular antigen-binding domain comprises a V_{H} comprising the amino sequence set forth in SEQ ID NO: 8.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 9. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 9. In certain embodiments, the extracellular antigen-binding domain comprises a V_{L} comprising the amino sequence set forth in SEQ ID NO: 9.

In certain embodiments, the extracellular antigen-binding domain (e.g., an scFv) comprises a V_{H} comprising the amino acid sequence set forth in SEQ ID NO: 8, and a V_{L} comprising the amino acid sequence set forth in SEQ ID NO: 9. In certain embodiments, the extracellular antigen-binding domain is an scFv. In certain embodiments, the scFv is designated as “scFv-Y-005”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 1

| CDRs | 1 | 2 | 3 |
|---|---|---|---|
| V_{H} | GFIFSSYN [SEQ | ISSSSSTT [SEQ | ASLYSGYDYYYYYGMDV [SEQ |
| | ID NO: 2] | ID NO: 3] | ID NO: 4] |
| V_{L} | QSLVHSDGNTY | KIS [SEQ ID NO: | MQATQFPFT [SEQ ID NO: |
| | [SEQ ID NO: 5] | 6] | 7] |

| | |
|---|---|
| Full | EVQMVESGGGLVQPGGSLRLSCAAPGFIFSSYNMNWVRQAPGKGLEWVSYISSSSS |
| V_{H} | TTYYADSVKGRFTISRDNAKNSLYLQMNSLRDEDTAVYYCASLYSGYDYYYYYGMD |
| | VWGQGTTVTVSS [SEQ ID NO: 8] |
| Full | DIVMTQTPLSSPVSLGQPASISCRSSQSLVHSDGNTYLSWLQQRPGQPPRVLIYKI |
| V_{L} | SKRFSGVPDRFSGSGAGTDFTLKISRVEAEDVGVYYCMQATQFPFTFGPGTKVDIK |
| | [SEQ ID NO: 9] |

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof. SEQ ID NOs: 12-14 are provided in Table 2.

In certain embodiments, the extracellular antigen-binding domain (e.g., an scFv) comprises a light chain variable region comprising a CDR1 comprising the amino acid sequence set forth in SEQ ID NO: 176 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 2.

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof; and a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 18. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 18. In certain embodiments, the extracellular antigen-binding domain comprises a V_{H} comprising the amino sequence set forth in SEQ ID NO: 18.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 19. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 19. In certain embodiments, the extracellular antigen-binding domain comprises a V_{L} comprising the amino sequence set forth in SEQ ID NO: 19.

In certain embodiments, the extracellular antigen-binding domain (e.g., an scFv) comprises a V_{H} comprising the amino acid sequence set forth in SEQ ID NO: 18, and a V_{L} comprising the amino acid sequence set forth in SEQ ID NO: 19. In certain embodiments, the extracellular antigen-binding domain is an scFv. In certain embodiments, the scFv is designated as “scFv-Y-004”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 2

| CDRs | 1 | 2 | 3 |
|---|---|---|---|
| V_{H} | GYTFTNYY [SEQ | IGPGSGSP [SEQ | ARWLLPFDY [SEQ ID NO: |
| | ID NO: 12] | ID NO: 13] | 14] |
| V_{L} | SSVNY [SEQ ID | ATS [SEQ ID NO: | QQFSSDPLT [SEQ ID NO: |
| | NO: 176] | 16] | 17] |

| | |
|---|---|
| Full | QVQLVQSGAEVKKPGSSVKVSCKASGYTFTNYYIHWVRQAPGQGLEWMGKIGPGSG |
| V_{H} | SPYYQGMFKDRVTITADESTSTAYMELSSLRSEDTAVYYCARWLLPFDYWGQGTLV |
| | TVSS [SEQ ID NO: 18] |
| Full | EIVLTQSPGTLSLSPGERATLSCRASSSVNYMHWYQQKPGQAPRLLIYATSILASG |
| V_{L} | IPDRFSGSGSGTDYTLTISRLEPEDFAVYYCQQFSSDPLTFGGGTKVEIK [SEQ |
| | ID NO: 19] |

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof. SEQ ID NOs: 12-14 are provided in Table 3.

In certain embodiments, the extracellular antigen-binding domain (e.g., an scFv) comprises a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 3.

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof; and a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 22. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 22. In certain embodiments, the extracellular antigen-binding domain comprises a V_{H} comprising the amino sequence set forth in SEQ ID NO: 22.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 23. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 23. In certain embodiments, the extracellular antigen-binding domain comprises a V_{L} comprising the amino sequence set forth in SEQ ID NO: 23.

In certain embodiments, the extracellular antigen-binding domain (e.g., an scFv) comprises a V_{H} comprising the amino acid sequence set forth in SEQ ID NO: 22, and a V_{L} comprising the amino acid sequence set forth in SEQ ID NO: 23. In certain embodiments, the extracellular antigen-binding domain is an scFv. In certain embodiments, the scFv is designated as “k1-h1”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 3

| CDRs | 1 | 2 | 3 |
|---|---|---|---|
| V_{H} | GYTFTNYY [SEQ | IGPGSGSP [SEQ | ARWLLPFDY [SEQ ID NO: |
| | ID NO: 12] | ID NO: 13] | 14] |
| V_{L} | SSVNY [SEQ ID | ATS [SEQ ID NO: | QQFSSDPLT [SEQ ID NO: |
| | NO: 15] | 16] | 17] |

| | |
|---|---|
| Full | QVQLVQSGAEVKKPGASVKVSCKASGYTFTNYYIHWVRQAPGQGLEWMGKIGPGSG |
| V_{H} | SPYYNGMFKDRVTMTRDTSTSTVYMELSSLRSEDTAVYYCARWLLPFDYWGQGTLV |
| | TVSS [SEQ ID NO: 22] |
| Full | EIVLTQSPATLSLSPGERATLSCRASSSVNYMHWYQQKPGQAPRLLIYATSILASG |
| V_{L} | IPARFSGSGSGTDYTLTISSLEPEDFAVYYCQQFSSDPLTFGGGTKVEIK [SEQ |
| | ID NO: 23] |

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof. SEQ ID NOs: 12-14 are provided in Table 4.

In certain embodiments, the extracellular antigen-binding domain (e.g., an scFv) comprises a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 4.

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof; and a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 24. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 24. In certain embodiments, the extracellular antigen-binding domain comprises a V_{H} comprising the amino sequence set forth in SEQ ID NO: 24.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 23. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 23. In certain embodiments, the extracellular antigen-binding domain comprises a V_{L} comprising the amino sequence set forth in SEQ ID NO: 23.

In certain embodiments, the extracellular antigen-binding domain (e.g., an scFv) comprises a V_{H} comprising the amino acid sequence set forth in SEQ ID NO: 24, and a V_{L} comprising the amino acid sequence set forth in SEQ ID NO: 23. In certain embodiments, the extracellular antigen-binding domain is an scFv. In certain embodiments, the scFv is designated as “k1-h2”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 4

| CDRs | 1 | 2 | 3 |
|---|---|---|---|
| V_{H} | GYTFTNYY [SEQ | IGPGSGSP [SEQ | ARWLLPFDY [SEQ ID NO: |
| | ID NO: 12] | ID NO: 13] | 14] |
| V_{L} | SSVNY [SEQ ID | ATS [SEQ ID NO: | QQFSSDPLT [SEQ ID NO: |
| | NO: 15] | 16] | 17] |

| | |
|---|---|
| Full | QVQLVQSGAEVKKPGASVKVSCKASGYTFTNYYIHWVRQAPGQGLEWMGKIGPGSG |
| V_{H} | SPYYNGMFKDRVTSTRDTSISTAYMELSRLRSDDTVVYYCARWLLPFDYWGQGTLV |
| | TVSS [SEQ ID NO: 24] |
| Full | EIVLTQSPATLSLSPGERATLSCRASSSVNYMHWYQQKPGQAPRLLIYATSILASG |
| V_{L} | IPARFSGSGSGTDYTLTISSLEPEDFAVYYCQQFSSDPLTFGGGTKVEIK [SEQ |
| | ID NO: 23] |

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof. SEQ ID NOs: 12-14 are provided in Table 5.

In certain embodiments, the extracellular antigen-binding domain (e.g., an scFv) comprises a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 5.

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof; and a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 25. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 25. In certain embodiments, the extracellular antigen-binding domain comprises a V_{H} comprising the amino sequence set forth in SEQ ID NO: 25.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 23. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 23. In certain embodiments, the extracellular antigen-binding domain comprises a V_{L} comprising the amino sequence set forth in SEQ ID NO: 23.

In certain embodiments, the extracellular antigen-binding domain (e.g., an scFv) comprises a V_{H} comprising the amino acid sequence set forth in SEQ ID NO: 25, and a V_{L} comprising the amino acid sequence set forth in SEQ ID NO: 23. In certain embodiments, the extracellular antigen-binding domain is an scFv. In certain embodiments, the scFv is designated as “k1-h3”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 5

| CDRs | 1 | 2 | 3 |
|---|---|---|---|
| V_{H} | GYTFTNYY | IGPGSGSP | ARWLLPFDY |
| | [SEQ ID | [SEQ ID | [SEQ ID |
| | NO: 12] | NO: 13] | NO: 14] |
| V_{L} | SSVNY | ATS | QQFSSDPLT |
| | [SEQ ID | [SEQ ID | [SEQ ID |
| | NO: 15] | NO: 16] | NO: 17] |

| | |
|---|---|
| Full | QVQLVESGGGVVQPGRSLRLSCAASGYTFTNYYIHWVRQAPG |
| V_{H} | KGLEWVAKIGPGSGSPYYNGMFKDRFTISRDNSKNTLYLQMN |
| | SLRAEDTAVYYCARWLLPFDYWGQGTLVTVSS |
| | [SEQ ID NO: 25] |
| Full | EIVLTQSPATLSLSPGERATLSCRASSSVNYMHWYQQKPGQA |
| V_{L} | PRLLIYATSILASGIPARFSGSGSGTDYTLTISSLEPEDFAV |
| | YYCQQFSSDPLTFGGGTKVEIK |
| | [SEQ ID NO: 23] |

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 26 or a conservative modification thereof. SEQ ID NOs: 12, 13, and 26 are provided in Table 6.

In certain embodiments, the extracellular antigen-binding domain (e.g., an scFv) comprises a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 6.

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 26 or a conservative modification thereof; and a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 26; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 27. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 27. In certain embodiments, the extracellular antigen-binding domain comprises a V_{H} comprising the amino sequence set forth in SEQ ID NO: 27.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 23. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 23. In certain embodiments, the extracellular antigen-binding domain comprises a V_{L} comprising the amino sequence set forth in SEQ ID NO: 23.

In certain embodiments, the extracellular antigen-binding domain (e.g., an scFv) comprises a V_{H} comprising the amino acid sequence set forth in SEQ ID NO: 27, and a V_{L} comprising the amino acid sequence set forth in SEQ ID NO: 23. In certain embodiments, the extracellular antigen-binding domain is an scFv. In certain embodiments, the scFv is designated as “k1-h4”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 6

| CDRs | 1 | 2 | 3 |
|---|---|---|---|
| V_{H} | GYTFTNYY | IGPGSGSP | AKWLLPFDY |
| | [SEQ ID | [SEQ ID | [SEQ ID |
| | NO: 12] | NO: 13] | NO: 26] |
| V_{L} | SSVN | ATS | QQFSSDPLT |
| | [SEQ ID | [SEQ ID | [SEQ ID |
| | NO: 15] | NO: 16] | NO: 17] |

| | |
|---|---|
| Full | EVQLLESGGGLVQPGGSLRLSCAASGYTFTNYYIHWVRQAPG |
| V_{H} | KGLEWVSKIGPGSGSPYYNGMFKDRFTISVDNSKNTLYLQMN |
| | SLRAEDTAVYYCAKWLLPFDYWGQGTLVTVSS |
| | [SEQ ID NO: 27] |
| Full | EIVLTQSPATLSLSPGERATLSCRASSSVNYMHWYQQKPGQA |
| V_{L} | PRLLIYATSILASGIPARFSGSGSGTDYTLTISSLEPEDFAV |
| | YYCQQFSSDPLTFGGGTKVEIK |
| | [SEQ ID NO: 23] |

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof. SEQ ID NOs: 12-14 are provided in Table 7.

In certain embodiments, the extracellular antigen-binding domain (e.g., an scFv) comprises a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 7.

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof; and a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 28. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 28. In certain embodiments, the extracellular antigen-binding domain comprises a V_{H} comprising the amino sequence set forth in SEQ ID NO: 28.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 23. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 23. In certain embodiments, the extracellular antigen-binding domain comprises a V_{L} comprising the amino sequence set forth in SEQ ID NO: 23.

In certain embodiments, the extracellular antigen-binding domain (e.g., an scFv) comprises a V_{H} comprising the amino acid sequence set forth in SEQ ID NO: 28, and a V_{L} comprising the amino acid sequence set forth in SEQ ID NO: 23. In certain embodiments, the extracellular antigen-binding domain is an scFv. In certain embodiments, the scFv is designated as “k1-h5”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 7

| CDRs | 1 | 2 | 3 |
|---|---|---|---|
| V_{H} | GYTFTNYY | IGPGSGSP | ARWLLPFDY |
| | [SEQ ID | [SEQ ID | [SEQ ID |
| | NO: 12] | NO: 13] | NO: 14] |
| V_{L} | SSVNY | ATS | QQFSSDPLT |
| | [SEQ ID | [SEQ ID | [SEQ ID |
| | NO: 15] | NO: 16] | NO: 17] |

| | |
|---|---|
| Full | QVQLVQSGAEVKKPGSSVKVSCKASGYTFTNYYIHWVRQAPG |
| V_{H} | QGLEWMGKIGPGSGSPYYNGMFKDRVTITADESTSTAYMELS |
| | SLRSEDTAVYYCARWLLPFDYWGQGTLVTVSS |
| | [SEQ ID NO: 28] |
| Full | EIVLTQSPATLSLSPGERATLSCRASSSVNYMHWYQQKPGQA |
| V_{L} | PRLLIYATSILASGIPARFSGSGSGTDYTLTISSLEPEDFAV |
| | YYCQQFSSDPLTFGGGTKVEIK[ |
| | [SEQ ID NO: 23] |

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 26 or a conservative modification thereof. SEQ ID NOs: 12, 13, and 26 are provided in Table 8.

In certain embodiments, the extracellular antigen-binding domain (e.g., an scFv) comprises a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 8.

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 26 or a conservative modification thereof; and a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 26; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 29. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 29. In certain embodiments, the extracellular antigen-binding domain comprises a V_{H} comprising the amino sequence set forth in SEQ ID NO: 29.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 23. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 23. In certain embodiments, the extracellular antigen-binding domain comprises a V_{L} comprising the amino sequence set forth in SEQ ID NO: 23.

In certain embodiments, the extracellular antigen-binding domain (e.g., an scFv) comprises a V_{H} comprising the amino acid sequence set forth in SEQ ID NO: 29, and a V_{L} comprising the amino acid sequence set forth in SEQ ID NO: 23. In certain embodiments, the extracellular antigen-binding domain is an scFv. In certain embodiments, the scFv is designated as “k1-h6”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 8

| CDRs | 1 | 2 | 3 |
|---|---|---|---|
| V_{H} | GYTFTNYY | IGPGSGSP | AKWLLPFDY |
| | [SEQ ID | [SEQ ID | [SEQ ID |
| | NO: 12] | NO: 13] | NO: 26] |
| V_{L} | SSVNY | ATS | QQFSSDPLT |
| | [SEQ ID | [SEQ ID | [SEQ ID |
| | NO: 15] | NO: 16] | NO: 17] |

| | |
|---|---|
| Full | EVQLLESGGGLVQPGGSLRLSCAASGYTFTNYYIHWVRQAPG |
| V_{H} | KGLEWVSKIGPGSGSPYYADSVKGRFTISVDNSKNTLYLQMN |
| | SLRAEDTAVYYCAKWLLPFDYWGQGTLVTVSS |
| | [SEQ ID NO: 29] |
| Full | EIVLTQSPATLSLSPGERATLSCRASSSVNYMHWYQQKPGQA |
| V_{L} | PRLLIYATSILASGIPARFSGSGSGTDYTLTISSLEPEDFAV |
| | YYCQQFSSDPLTFGGGTKVEIK |
| | [SEQ ID NO: 23] |

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof. SEQ ID NOs: 12-14 are provided in Table 9.

In certain embodiments, the extracellular antigen-binding domain (e.g., an scFv) comprises a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 9.

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof; and a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 22. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 22. In certain embodiments, the extracellular antigen-binding domain comprises a V_{H} comprising the amino sequence set forth in SEQ ID NO: 22.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 30. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 30. In certain embodiments, the extracellular antigen-binding domain comprises a V_{L} comprising the amino sequence set forth in SEQ ID NO: 30.

In certain embodiments, the extracellular antigen-binding domain (e.g., an scFv) comprises a V_{H} comprising the amino acid sequence set forth in SEQ ID NO: 22, and a V_{L} comprising the amino acid sequence set forth in SEQ ID NO: 30. In certain embodiments, the extracellular antigen-binding domain is an scFv. In certain embodiments, the scFv is designated as “k2-h1”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 9

| CDRs | 1 | 2 | 3 |
|---|---|---|---|
| V_{H} | GYTFTNYY | IGPGSGSP | ARWLLPFDY |
| | [SEQ ID | [SEQ ID | [SEQ ID |
| | NO: 12] | NO: 13] | NO: 14] |
| V_{L} | SSVNY | ATS | QQFSSDPLT |
| | [SEQ ID | [SEQ ID | [SEQ ID |
| | NO: 15] | NO: 16] | NO: 17] |

| | |
|---|---|
| Full | QVQLVQSGAEVKKPGASVKVSCKASGYTFTNYYIHWVRQAPG |
| V_{H} | QGLEWMGKIGPGSGSPYYNGMFKDRVTMTRDTSTSTVYMELS |
| | SLRSEDTAVYYCARWLLPFDYWGQGTLVTVSS |
| | [SEQ ID NO: 22] |
| Full | EIVMTQSPATLSVSPGERATLSCRASSSVNYMHWYQQKPGQA |
| V_{L} | PRLLIYATSILASGIPARFSGSGSGTEYTLTISSLQSEDFAV |
| | YYCQQFSSDPLTFGGGTKVEIK |
| | [SEQ ID NO: 30] |

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof. SEQ ID NOs: 12-14 are provided in Table 10.

In certain embodiments, the extracellular antigen-binding domain (e.g., an scFv) comprises a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 10.

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof; and a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 24. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 24. In certain embodiments, the extracellular antigen-binding domain comprises a V_{H} comprising the amino sequence set forth in SEQ ID NO: 24.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 30. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 30. In certain embodiments, the extracellular antigen-binding domain comprises a V_{L} comprising the amino sequence set forth in SEQ ID NO: 30.

In certain embodiments, the extracellular antigen-binding domain (e.g., an scFv) comprises a V_{H} comprising the amino acid sequence set forth in SEQ ID NO: 24, and a V_{L} comprising the amino acid sequence set forth in SEQ ID NO: 30. In certain embodiments, the extracellular antigen-binding domain is an scFv. In certain embodiments, the scFv is designated as “k2-h2”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 10

| CDRs | 1 | 2 | 3 |
|---|---|---|---|
| V_{H} | GYTFTNYY | IGPGSGSP | ARWLLPFDY |
| | [SEQ ID | [SEQ ID | [SEQ ID |
| | NO: 12] | NO: 13] | NO: 14] |
| V_{L} | SSVNY | ATS | QQFSSDPLT |
| | [SEQ ID | [SEQ ID | [SEQ ID |
| | NO: 15] | NO: 16] | NO: 17] |

| | |
|---|---|
| Full | QVQLVQSGAEVKKPGASVKVSCKASGYTFTNYYIHWVRQAPG |
| V_{H} | QGLEWMGKIGPGSGSPYYNGMFKDRVTSTRDTSISTAYMELS |
| | RLRSDDTVVYYCARWLLPFDYWGQGTLVTVSS |
| | [SEQ ID NO: 24] |
| Full | EIVMTQSPATLSVSPGERATLSCRASSSVNYMHWYQQKPGQA |
| V_{L} | PRLLIYATSILASGIPARFSGSGSGTEYTLTISSLQSEDFAV |
| | YYCQQFSSDPLTFGGGTKVEIK |
| | [SEQ ID NO: 30] |

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof. SEQ ID NOs: 12-14 are provided in Table 11.

In certain embodiments, the extracellular antigen-binding domain (e.g., an scFv) comprises a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 11.

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof; and a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 25. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90°/, about 91%, about 92%, about 93%, about 94%, about 95%, about 96%, about 97%, about 98%, about 99% or about 100% homologous or identical to SEQ ID NO: 25. In certain embodiments, the extracellular antigen-binding domain comprises a V_{H} comprising the amino sequence set forth in SEQ ID NO: 25.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 30. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 30. In certain embodiments, the extracellular antigen-binding domain comprises a V_{L} comprising the amino sequence set forth in SEQ ID NO: 30.

In certain embodiments, the extracellular antigen-binding domain (e.g., an scFv) comprises a V_{H} comprising the amino acid sequence set forth in SEQ ID NO: 25, and a V_{L} comprising the amino acid sequence set forth in SEQ ID NO: 30. In certain embodiments, the extracellular antigen-binding domain is an scFv. In certain embodiments, the scFv is designated as “k2-h3”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 11

| CDRs | 1 | 2 | 3 |
|---|---|---|---|
| V_{H} | GYTFTNYY | IGPGSGSP | ARWLLPFDY |
| | [SEQ ID | [SEQ ID | [SEQ ID |
| | NO: 12] | NO: 13] | NO: 14] |
| V_{L} | SSVNY | ATS | QQFSSDPLT |
| | [SEQ ID | [SEQ ID | [SEQ ID |
| | NO: 15] | NO: 16] | NO: 17] |

| | |
|---|---|
| Full | QVQLVESGGGVVQPGRSLRLSCAASGYTFTNYYIHWVRQAPG |
| V_{H} | KGLEWVAKIGPGSGSPYYNGMFKDRFTISRDNSKNTLYLQMN |
| | SLRAEDTAVYYCARWLLPFDYWGQGTLVTVSS |
| | [SEQ ID NO: 25] |
| Full | EIVMTQSPATLSVSPGERATLSCRASSSVNYMHWYQQKPGQA |
| V_{L} | PRLLIYATSILASGIPARFSGSGSGTEYTLTISSLQSEDFAV |
| | YYCQQFSSDPLTFGGGTKVEIK |
| | [SEQ ID NO: 30] |

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 26 or a conservative modification thereof. SEQ ID NOs: 12, 13, and 26 are provided in Table 12.

In certain embodiments, the extracellular antigen-binding domain (e.g., an scFv) comprises a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 12.

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 26 or a conservative modification thereof; and a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 26; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 27. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 27. In certain embodiments, the extracellular antigen-binding domain comprises a V_{H} comprising the amino sequence set forth in SEQ ID NO: 27.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 30. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 30. In certain embodiments, the extracellular antigen-binding domain comprises a V_{L} comprising the amino sequence set forth in SEQ ID NO: 30.

In certain embodiments, the extracellular antigen-binding domain (e.g., an scFv) comprises a V_{H} comprising the amino acid sequence set forth in SEQ ID NO: 27, and a V_{L} comprising the amino acid sequence set forth in SEQ ID NO: 30. In certain embodiments, the extracellular antigen-binding domain is an scFv. In certain embodiments, the scFv is designated as “k2-h4”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 12

| CDRs | 1 | 2 | 3 |
|---|---|---|---|
| V_{H} | GYTFTNYY | IGPGSGSP | AKWLLPFDY |
| | [SEQ ID | [SEQ ID | [SEQ ID |
| | NO: 12] | NO: 13] | NO: 26] |
| V_{L} | SSVNY | ATS | QQFSSDPLT |
| | [SEQ ID | [SEQ ID | [SEQ ID |
| | NO: 15] | NO: 16] | NO: 17] |

| | |
|---|---|
| Full | EVQLLESGGGLVQPGGSLRLSCAASGYTFTNYYIHWVRQAPG |
| V_{H} | KGLEWVSKIGPGSGSPYYNGMFKDRFTISVDNSKNTLYLQMN |
| | SLRAEDTAVYYCAKWLLPFDYWGQGTLVTVSS |
| | [SEQ ID NO: 27] |
| Full | EIVMTQSPATLSVSPGERATLSCRASSSVNYMHWYQQKPGQA |
| V_{L} | PRLLIYATSILASGIPARFSGSGSGTEYTLTISSLQSEDFAV |
| | YYCQQFSSDPLTFGGGTKVEIK |
| | [SEQ ID NO: 30] |

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof. SEQ ID NOs: 12-14 are provided in Table 13.

In certain embodiments, the extracellular antigen-binding domain (e.g., an scFv) comprises a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 13.

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof; and a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 28. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 28. In certain embodiments, the extracellular antigen-binding domain comprises a V_{H} comprising the amino sequence set forth in SEQ ID NO: 28.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 30. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 30. In certain embodiments, the extracellular antigen-binding domain comprises a V_{L} comprising the amino sequence set forth in SEQ ID NO: 30.

In certain embodiments, the extracellular antigen-binding domain (e.g., an scFv) comprises a V_{H} comprising the amino acid sequence set forth in SEQ ID NO: 28, and a V_{L} comprising the amino acid sequence set forth in SEQ ID NO: 30. In certain embodiments, the extracellular antigen-binding domain is an scFv. In certain embodiments, the scFv is designated as “k2-h5”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 13

| CDRs | 1 | 2 | 3 |
|---|---|---|---|
| V_{H} | GYTFTNYY | IGPGSGSP | ARWLLPFDY |
| | [SEQ ID | [SEQ ID | [SEQ ID |
| | NO: 12] | NO: 13] | NO: 14] |
| V_{L} | SSVNY | ATS | QQFSSDPLT |
| | [SEQ ID | [SEQ ID | [SEQ ID |
| | NO: 15] | NO: 16] | NO: 17] |

| | |
|---|---|
| Full | QVQLVQSGAEVKKPGSSVKVSCKASGYTFTNYYIHWVRQAPG |
| V_{H} | QGLEWMGKIGPGSGSPYYNGMFKDRVTITADESTSTAYMELS |
| | SLRSEDTAVYYCARWLLPFDYWGQGTLVTVSS |
| | [SEQ ID NO: 28] |
| Full | EIVMTQSPATLSVSPGERATLSCRASSSVNYMHWYQQKPGQA |
| V_{L} | PRLLIYATSILASGIPARFSGSGSGTEYTLTISSLQSEDFAV |
| | YYCQQFSSDPLTFGGGTKVEIK |
| | [SEQ ID NO: 30] |

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 26 or a conservative modification thereof. SEQ ID NOs: 12, 13, and 26 are provided in Table 14.

In certain embodiments, the extracellular antigen-binding domain (e.g., an scFv) comprises a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 14.

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 26 or a conservative modification thereof, and a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 26; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 29. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 29. In certain embodiments, the extracellular antigen-binding domain comprises a V_{H} comprising the amino sequence set forth in SEQ ID NO: 29.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 30. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 30. In certain embodiments, the extracellular antigen-binding domain comprises a V_{L} comprising the amino sequence set forth in SEQ ID NO: 30.

In certain embodiments, the extracellular antigen-binding domain (e.g., an scFv) comprises a V_{H} comprising the amino acid sequence set forth in SEQ ID NO: 29, and a V_{L} comprising the amino acid sequence set forth in SEQ ID NO: 30. In certain embodiments, the extracellular antigen-binding domain is an scFv. In certain embodiments, the scFv is designated as “k2-h6”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 14

| CDRs | 1 | 2 | 3 |
|---|---|---|---|
| VH | GYTFTNYY | IGPGSGSP | AKWLLPFDY |
| | [SEQ ID | [SEQ ID | [SEQ ID |
| | NO: 12] | NO: 13] | NO: 26] |
| VL | SSVNY | ATS | QQFSSDPLT |
| | [SEQ ID | [SEQ ID | [SEQ ID |
| | NO: 15] | NO: 16] | NO: 17] |

| | |
|---|---|
| Full | EVQLLESGGGLVQPGGSLRLSCAASGYTFTNYYIHWVRQAPG |
| VH | KGLEWVSKIGPGSGSPYYADSVKGRFTISVDNSKNTLYLQMN |
| | SLRAEDTAVYYCAKWLLPFDYWGQGTLVTVSS |
| | [SEQ ID NO: 29] |
| Full | EIVMTQSPATLSVSPGERATLSCRASSSVNYMHWYQQKPGQA |
| VL | PRLLIYATSILASGIPARFSGSGSGTEYTLTISSLQSEDFAV |
| | YYCQQFSSDPLTFGGGTKVEIK |
| | [SEQ ID NO: 30] |

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof. SEQ ID NOs: 12-14 are provided in Table 15.

In certain embodiments, the extracellular antigen-binding domain (e.g., an scFv) comprises a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 15.

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof; and a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 22. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 22. In certain embodiments, the extracellular antigen-binding domain comprises a V_{H} comprising the amino sequence set forth in SEQ ID NO: 22.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 31. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100 homologous or identical to SEQ ID NO: 31. In certain embodiments, the extracellular antigen-binding domain comprises a V_{L} comprising the amino sequence set forth in SEQ ID NO: 31.

In certain embodiments, the extracellular antigen-binding domain (e.g., an scFv) comprises a V_{H} comprising the amino acid sequence set forth in SEQ ID NO: 22, and a V_{L} comprising the amino acid sequence set forth in SEQ ID NO: 31. In certain embodiments, the extracellular antigen-binding domain is an scFv. In certain embodiments, the scFv is designated as “k3-h1”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 15

| CDRs | 1 | 2 | 3 |
|---|---|---|---|
| VH | GYTFTNYY | IGPGSGSP | ARWLLPFDY |
| | [SEQ ID | [SEQ ID | [SEQ ID |
| | NO: 12] | NO: 13] | NO: 14] |
| VL | SSVNY | ATS | QQFSSDPLT |
| | [SEQ ID | [SEQ ID | [SEQ ID |
| | NO: 15] | NO: 16] | NO: 17] |

| | |
|---|---|
| Full | QVQLVQSGAEVKKPGASVKVSCKASGYTFTNYYIHWVRQAPG |
| VH | QGLEWMGKIGPGSGSPYYNGMFKDRVTMTRDTSTSTVYMELS |
| | SLRSEDTAVYYCARWLLPFDYWGQGTLVTVSS |
| | [SEQ ID NO: 22] |
| Full | DIQMTQSPSTLSASVGDRVTITCRASSSVNYMHWYQQKPGKA |
| VL | PKLLIYATSILASGVPSRFSGSGSGTEYTLTISSLQPDDFAT |
| | YYCQQFSSDPLTFGGGTKVEIK |
| | [SEQ ID NO: 31] |

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof. SEQ ID NOs: 12-14 are provided in Table 16.

In certain embodiments, the extracellular antigen-binding domain (e.g., an scFv) comprises a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 16.

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof; and a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 24. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 24. In certain embodiments, the extracellular antigen-binding domain comprises a V_{H} comprising the amino sequence set forth in SEQ ID NO: 24.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 31. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 31. In certain embodiments, the extracellular antigen-binding domain comprises a V_{L} comprising the amino sequence set forth in SEQ ID NO: 31.

In certain embodiments, the extracellular antigen-binding domain (e.g., an scFv) comprises a V_{H} comprising the amino acid sequence set forth in SEQ ID NO: 24, and a V_{L} comprising the amino acid sequence set forth in SEQ ID NO: 31. In certain embodiments, the extracellular antigen-binding domain is an scFv. In certain embodiments, the scFv is designated as “k3-h2”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 16

| CDRs | 1 | 2 | 3 |
|---|---|---|---|
| VH | GYTFTNYY | IGPGSGSP | ARWLLPFDY |
| | [SEQ ID | [SEQ ID | [SEQ ID |
| | NO: 12] | NO: 13] | NO: 14] |
| VL | SSVNY | ATS | QQFSSDPLT |
| | [SEQ ID | [SEQ ID | [SEQ ID |
| | NO: 15] | NO: 16] | NO: 17] |

| | |
|---|---|
| Full | QVQLVQSGAEVKKPGASVKVSCKASGYTFTNYYIHWVRQAPG |
| VH | QGLEWMGKIGPGSGSPYYNGMFKDRVTSTRDTSISTAYMELS |
| | RLRSDDTVVYYCARWLLPFDYWGQGTLVTVSS |
| | [SEQ ID NO: 24] |
| Full | DIQMTQSPSTLSASVGDRVTITCRASSSVNYMHWYQQKPGKA |
| VL | PKLLIYATSILASGVPSRFSGSGSGTEYTLTISSLQPDDFAT |
| | YYCQQFSSDPLTFGGGTKVEIK |
| | [SEQ ID NO: 31] |

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof. SEQ ID NOs: 12-14 are provided in Table 17.

In certain embodiments, the extracellular antigen-binding domain (e.g., an scFv) comprises a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 17.

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof; and a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 25. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 25. In certain embodiments, the extracellular antigen-binding domain comprises a V_{H} comprising the amino sequence set forth in SEQ ID NO: 25.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 31. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 31. In certain embodiments, the extracellular antigen-binding domain comprises a V_{L} comprising the amino sequence set forth in SEQ ID NO: 31.

In certain embodiments, the extracellular antigen-binding domain (e.g., an scFv) comprises a V_{H} comprising the amino acid sequence set forth in SEQ ID NO: 25, and a V_{L} comprising the amino acid sequence set forth in SEQ ID NO: 31. In certain embodiments, the extracellular antigen-binding domain is an scFv. In certain embodiments, the scFv is designated as “k3-h3”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 17

| CDRs | 1 | 2 | 3 |
|---|---|---|---|
| VH | GYTFTNYY | IGPGSGSP | ARWLLPFDY |
| | [SEQ ID | [SEQ ID | [SEQ ID |
| | NO: 12] | NO: 13] | NO: 14] |
| VL | SSVNY | ATS | QQFSSDPLT |
| | [SEQ ID | [SEQ ID | [SEQ ID |
| | NO: 15] | NO: 16] | NO: 17] |

| | |
|---|---|
| Full | QVQLVESGGGVVQPGRSLRLSCAASGYTFTNYYIHWVRQAPG |
| VH | KGLEWVAKIGPGSGSPYYNGMFKDRFTISRDNSKNTLYLQMN |
| | SLRAEDTAVYYCARWLLPFDYWGQGTLVTVSS |
| | [SEQ ID NO: 25] |
| Full | DIQMTQSPSTLSASVGDRVTITCRASSSVNYMHWYQQKPGKA |
| VL | PKLLIYATSILASGVPSRFSGSGSGTEYTLTISSLQPDDFAT |
| | YYCQQFSSDPLTFGGGTKVEIK |
| | [SEQ ID NO: 31] |

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 26 or a conservative modification thereof. SEQ ID NOs: 12, 13, and 26 are provided in Table 18.

In certain embodiments, the extracellular antigen-binding domain (e.g., an scFv) comprises a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 18.

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 26 or a conservative modification thereof; and a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 26; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 27. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 27. In certain embodiments, the extracellular antigen-binding domain comprises a V_{H} comprising the amino sequence set forth in SEQ ID NO: 27.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 31. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 31. In certain embodiments, the extracellular antigen-binding domain comprises a V_{L} comprising the amino sequence set forth in SEQ ID NO: 31.

In certain embodiments, the extracellular antigen-binding domain (e.g., an scFv) comprises a V_{H} comprising the amino acid sequence set forth in SEQ ID NO: 27, and a V_{L} comprising the amino acid sequence set forth in SEQ ID NO: 31. In certain embodiments, the extracellular antigen-binding domain is an scFv. In certain embodiments, the scFv is designated as “k3-h4”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 18

| CDRs | 1 | 2 | 3 |
|---|---|---|---|
| VH | GYTFTNYY | IGPGSGSP | AKWLLPFDY |
| | [SEQ ID | [SEQ ID | [SEQ ID |
| | NO: 12] | NO: 13] | NO: 26] |
| VL | SSVNY | ATS | QQFSSDPLT |
| | [SEQ ID | [SEQ ID | [SEQ ID |
| | NO: 15] | NO: 16] | NO: 17] |

| | |
|---|---|
| Full | EVQLLESGGGLVQPGGSLRLSCAASGYTFTNYYIHWVRQAPG |
| VH | KGLEWVSKIGPGSGSPYYNGMFKDRFTISVDNSKNTLYLQMN |
| | SLRAEDTAVYYCAKWLLPFDYWGQGTLVTVSS |
| | [SEQ ID NO: 27] |
| Full | DIQMTQSPSTLSASVGDRVTITCRASSSVNYMHWYQQKPGKA |
| VL | PKLLIYATSILASGVPSRFSGSGSGTEYTLTISSLQPDDFAT |
| | YYCQQFSSDPLTFGGGTKVEIK |
| | [SEQ ID NO: 31] |

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof. SEQ ID NOs: 12-14 are provided in Table 19.

In certain embodiments, the extracellular antigen-binding domain (e.g., an scFv) comprises a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 19.

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof, and a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 28. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 28. In certain embodiments, the extracellular antigen-binding domain comprises a V_{H} comprising the amino sequence set forth in SEQ ID NO: 28.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 31. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90°/a, about 91%, about 92%, about 93%, about 94%, about 95%, about 96%, about 97%, about 98%, about 99% or about 100% homologous or identical to SEQ ID NO: 31. In certain embodiments, the extracellular antigen-binding domain comprises a V_{L} comprising the amino sequence set forth in SEQ ID NO: 31.

In certain embodiments, the extracellular antigen-binding domain (e.g., an scFv) comprises a V_{H} comprising the amino acid sequence set forth in SEQ ID NO: 28, and a V_{L} comprising the amino acid sequence set forth in SEQ ID NO: 31. In certain embodiments, the extracellular antigen-binding domain is an scFv. In certain embodiments, the scFv is designated as “k3-h5”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 19

| CDRs | 1 | 2 | 3 |
|---|---|---|---|
| VH | GYTFTNYY | IGPGSGSP | ARWLLPFDY |
| | [SEQ ID | [SEQ ID | [SEQ ID |
| | NO: 12] | NO: 13] | NO: 14] |
| VL | SSVNY | ATS | QQFSSDPLT |
| | [SEQ ID | [SEQ ID | [SEQ ID |
| | NO: 15] | NO: 16] | NO: 17] |

| | |
|---|---|
| Full | QVQLVQSGAEVKKPGSSVKVSCKASGYTFTNYYIHWVRQAPG |
| VH | QGLEWMGKIGPGSGSPYYNGMFKDRVTITADESTSTAYMELS |
| | SLRSEDTAVYYCARWLLPFDYWGQGTLVTVSS |
| | [SEQ ID NO: 28] |
| Full | DIQMTQSPSTLSASVGDRVTITCRASSSVNYMHWYQQKPGKA |
| VL | PKLLIYATSILASGVPSRFSGSGSGTEYTLTISSLQPDDFAT |
| | YYCQQFSSDPLTFGGGTKVEIK |
| | [SEQ ID NO: 31] |

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 26 or a conservative modification thereof. SEQ ID NOs: 12, 13, and 26 are provided in Table 20.

In certain embodiments, the extracellular antigen-binding domain (e.g., an scFv) comprises a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 20.

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 26 or a conservative modification thereof; and a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 26; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 29. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 29. In certain embodiments, the extracellular antigen-binding domain comprises a V_{H} comprising the amino sequence set forth in SEQ ID NO: 29.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 31. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100 homologous or identical to SEQ ID NO: 31. In certain embodiments, the extracellular antigen-binding domain comprises a V_{L} comprising the amino sequence set forth in SEQ ID NO: 31.

In certain embodiments, the extracellular antigen-binding domain (e.g., an scFv) comprises a V_{H} comprising the amino acid sequence set forth in SEQ ID NO: 29, and a V_{L} comprising the amino acid sequence set forth in SEQ ID NO: 31. In certain embodiments, the extracellular antigen-binding domain is an scFv. In certain embodiments, the scFv is designated as “k3-h6”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 20

| CDRs | 1 | 2 | 3 |
|---|---|---|---|
| VH | GYTFTNYY | IGPGSGSP | AKWLLPFDY |
| | [SEQ ID NO: 12] | [SEQ ID NO: 13] | [SEQ ID NO: 26] |
| VL | SSVNY | ATS | QQFSSDPLT |
| | [SEQ ID NO: 15] | [SEQ ID NO: 16] | [SEQ ID NO: 17] |

| | |
|---|---|
| Full | EVQLLESGGGLVQPGGSLRLSCAASGYTFTNYYIHWVRQAPGKGLEWVSKIGPGSG |
| VH | SPYYADSVKGRFTISVDNSKNTLYLQMNSLRAEDTAVYYCAKWLLPFDYWGQGTLV |
| | TVSS |
| | [SEQ ID NO: 29] |
| Full | DIQMTQSPSTLSASVGDRVTITCRASSSVNYMHWYQQKPGKAPKLLIYATSILASG |
| VL | VPSRFSGSGSGTEYTLTISSLQPDDFATYYCQQFSSDPLTFGGGTKVEIK |
| | [SEQ ID NO: 31] |

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof. SEQ ID NOs: 12-14 are provided in Table 21.

In certain embodiments, the extracellular antigen-binding domain (e.g., an scFv) comprises a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 21.

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof, and a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 22. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 22. In certain embodiments, the extracellular antigen-binding domain comprises a V_{H} comprising the amino sequence set forth in SEQ ID NO: 22.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 32. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 32. In certain embodiments, the extracellular antigen-binding domain comprises a V_{L} comprising the amino sequence set forth in SEQ ID NO: 32.

In certain embodiments, the extracellular antigen-binding domain (e.g., an scFv) comprises a V_{H} comprising the amino acid sequence set forth in SEQ ID NO: 22, and a V_{L} comprising the amino acid sequence set forth in SEQ ID NO: 32. In certain embodiments, the extracellular antigen-binding domain is an scFv. In certain embodiments, the scFv is designated as “k4-h1”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 21

| CDRS | 1 | 2 | 3 |
|---|---|---|---|
| V_{H} | GYTFTNYY | IGPGSGSP | ARWLLPFDY |
| | [SEQ ID NO: 12] | [SEQ ID NO: 13] | [SEQ ID NO: 14] |
| V_{L} | SSVNY | ATS | QQFSSDPLT |
| | [SEQ ID NO: 15] | [SEQ ID NO: 16] | [SEQ ID NO: 17] |

| | |
|---|---|
| Full | QVQLVQSGAEVKKPGASVKVSCKASGYTFTNYYIHWVRQAPGQGLEWMGKIGPGSG |
| V_{H} | SPYYNGMFKDRVTMTRDTSTSTVYMELSSLRSEDTAVYYCARWLLPFDYWGQGTLV |
| | TVSS |
| | [SEQ ID NO: 22] |
| Full | DIQMTQSPSSVSASVGDRVTITCRASSSVNYMHWYQQKPGKAPKPLIYATSILASG |
| V_{L} | VPSRFSGSGSGTDYTLTISSLQPEDFATYYCQQFSSDPLTFGGGTKVEIK |
| | [SEQ ID NO: 32] |

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof. SEQ ID NOs: 12-14 are provided in Table 22.

In certain embodiments, the extracellular antigen-binding domain (e.g., an scFv) comprises a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 22.

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof, and a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 24. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 24. In certain embodiments, the extracellular antigen-binding domain comprises a V_{H} comprising the amino sequence set forth in SEQ ID NO: 24.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 32. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 32. In certain embodiments, the extracellular antigen-binding domain comprises a V_{L} comprising the amino sequence set forth in SEQ ID NO: 32.

In certain embodiments, the extracellular antigen-binding domain (e.g., an scFv) comprises a V_{H} comprising the amino acid sequence set forth in SEQ ID NO: 24, and a V_{L} comprising the amino acid sequence set forth in SEQ ID NO: 32. In certain embodiments, the extracellular antigen-binding domain is an scFv. In certain embodiments, the scFv is designated as “k4-h2”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 22

| CDRs | 1 | 2 | 3 |
|---|---|---|---|
| V_{H} | GYTFTNYY | IGPGSGSP | ARWLLPFDY |
| | [SEQ ID NO: 12] | [SEQ ID NO: 13] | [SEQ ID NO: 14] |
| V_{L} | SSVNY | ATS | QQFSSDPLT |
| | [SEQ ID NO: 15] | [SEQ ID NO: 16] | [SEQ ID NO: 17] |

| | |
|---|---|
| Full | QVQLVQSGAEVKKPGASVKVSCKASGYTFTNYYIHWVRQAPGQGLEWMGKIGPGSG |
| V_{H} | SPYYNGMFKDRVTSTRDTSISTAYMELSRLRSDDTVVYYCARWLLPFDYWGQGTLV |
| | TVSS |
| | [SEQ ID NO: 24] |
| Full | DIQMTQSPSSVSASVGDRVTITCRASSSVNYMHWYQQKPGKAPKPLIYATSILASG |
| V_{L} | VPSRESGSGSGTDYTLTISSLQPEDFATYYCQQFSSDPLTFGGGTKVEIK |
| | [SEQ ID NO: 32] |

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof. SEQ ID NOs: 12-14 are provided in Table 23.

In certain embodiments, the extracellular antigen-binding domain (e.g., an scFv) comprises a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 23.

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof; and a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 25. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 25. In certain embodiments, the extracellular antigen-binding domain comprises a V_{H} comprising the amino sequence set forth in SEQ ID NO: 25.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 32. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 32. In certain embodiments, the extracellular antigen-binding domain comprises a V_{L} comprising the amino sequence set forth in SEQ ID NO: 32.

In certain embodiments, the extracellular antigen-binding domain (e.g., an scFv) comprises a V_{H} comprising the amino acid sequence set forth in SEQ ID NO: 25, and a V_{L} comprising the amino acid sequence set forth in SEQ ID NO: 32. In certain embodiments, the extracellular antigen-binding domain is an scFv. In certain embodiments, the scFv is designated as “k4-h3”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 23

| CDRS | 1 | 2 | 3 |
|---|---|---|---|
| V_{H} | GYTFTNYY | IGPGSGSP | ARWLLPFDY |
| | [SEQ ID NO: 12] | [SEQ ID NO: 13] | [SEQ ID NO: 14] |
| V_{L} | SSVNY | ATS | QQFSSDPLT |
| | [SEQ ID NO: 15] | [SEQ ID NO: 16] | [SEQ ID NO: 17] |

| | |
|---|---|
| Full | QVQLVESGGGVVQPGRSLRLSCAASGYTFTNYYIHWVRQAPGKGLEWVAKIGPGSG |
| V_{H} | SPYYNGMFKDRFTI SRDNSKNTLYLQMNSLRAEDTAVYYCARWLLPFDYWGQGTLV |
| | TVSS |
| | [SEQ ID NO: 25] |
| Full | DIQMTQSPSSVSASVGDRVTITCRASSSVNYMHWYQQKPGKAPKPLIYATSILASG |
| V_{L} | VPSRESGSGSGTDYTLTISSLQPEDFATYYCQQFSSDPLTFGGGTKVEIK |
| | [SEQ ID NO: 32] |

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 26 or a conservative modification thereof. SEQ ID NOs: 12, 13, and 26 are provided in Table 24.

In certain embodiments, the extracellular antigen-binding domain (e.g., an scFv) comprises a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 24.

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 26 or a conservative modification thereof, and a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 26; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 27. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 27. In certain embodiments, the extracellular antigen-binding domain comprises a V_{H} comprising the amino sequence set forth in SEQ ID NO: 27.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 32. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90°/a, about 91%, about 92%, about 93%, about 94%, about 95%, about 96%, about 97%, about 98%, about 99% or about 100% homologous or identical to SEQ ID NO: 32. In certain embodiments, the extracellular antigen-binding domain comprises a V_{L} comprising the amino sequence set forth in SEQ ID NO: 32.

In certain embodiments, the extracellular antigen-binding domain (e.g., an scFv) comprises a V_{H} comprising the amino acid sequence set forth in SEQ ID NO: 27, and a V_{L} comprising the amino acid sequence set forth in SEQ ID NO: 32. In certain embodiments, the extracellular antigen-binding domain is an scFv. In certain embodiments, the scFv is designated as “k4-h4”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 24

| CDRs | 1 | 2 | 3 |
|---|---|---|---|
| V_{H} | GYTFTNYY | IGPGSGSP | AKWLLPFDY |
| | [SEQ ID NO: 12] | [SEQ ID NO: 13] | [SEQ ID NO: 26] |
| V_{L} | SSVNY | ATS | QQFSSDPLT |
| | [SEQ ID NO: 15] | [SEQ ID NO: 16] | [SEQ ID NO: 17] |

| | |
|---|---|
| Full | EVQLLESGGGLVQPGGSLRLSCAASGYTFTNYYIHWVRQAPGKGLEWVSKIGPGSG |
| V_{H} | SPYYNGMFKDRFTISVDNSKNTLYLQMNSLRAEDTAVYYCAKWLLPFDYWGQGTLV |
| | TVSS |
| | [SEQ ID NO: 27] |
| Full | DIQMTQSPSSVSASVGDRVTITCRASSSVNYMHWYQQKPGKAPKPLIYATSILASG |
| V_{L} | VPSRESGSGSGTDYTLTISSLQPEDFATYYCQQFSSDPLTFGGGTKVEIK |
| | [SEQ ID NO: 32] |

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof. SEQ ID NOs: 12-14 are provided in Table 25.

In certain embodiments, the extracellular antigen-binding domain (e.g., an scFv) comprises a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 25.

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof; and a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 28. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 28. In certain embodiments, the extracellular antigen-binding domain comprises a V_{H} comprising the amino sequence set forth in SEQ ID NO: 28.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 32. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100 homologous or identical to SEQ ID NO: 32. In certain embodiments, the extracellular antigen-binding domain comprises a V_{L} comprising the amino sequence set forth in SEQ ID NO: 32.

In certain embodiments, the extracellular antigen-binding domain (e.g., an scFv) comprises a V_{H} comprising the amino acid sequence set forth in SEQ ID NO: 28, and a V_{L} comprising the amino acid sequence set forth in SEQ ID NO: 32. In certain embodiments, the extracellular antigen-binding domain is an scFv. In certain embodiments, the scFv is designated as “k4-h5”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 25

| CDRS | 1 | 2 | 3 |
|---|---|---|---|
| V_{H} | GYTFTNYY | IGPGSGSP | ARWLLPFDY |
| | [SEQ ID NO: 12] | [SEQ ID NO: 13] | [SEQ ID NO: 14] |
| V_{L} | SSVNY | ATS | QQFSSDPLT |
| | [SEQ ID NO: 15] | [SEQ ID NO: 16] | [SEQ ID NO: 17] |

| | |
|---|---|
| Full | QVQLVQSGAEVKKPGSSVKVSCKASGYTFTNYYIHWVRQAPGQGLEWMGKIGPGSG |
| V_{H} | SPYYNGMFKDRVTITADESTSTAYMELSSLRSEDTAVYYCARWLLPFDYWGQGTLV |
| | TVSS |
| | [SEQ ID NO: 28] |
| Full | DIQMTQSPSSVSASVGDRVTITCRASSSVNYMHWYQQKPGKAPKPLIYATSILASG |
| V_{L} | VPSRFSGSGSGTDYTLTISSLQPEDFATYYCQQFSSDPLTFGGGTKVEIK |
| | [SEQ ID NO: 32] |

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 26 or a conservative modification thereof. SEQ ID NOs: 12, 13, and 26 are provided in Table 26.

In certain embodiments, the extracellular antigen-binding domain (e.g., an scFv) comprises a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 26.

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 26 or a conservative modification thereof; and a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 26; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 29. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 29. In certain embodiments, the extracellular antigen-binding domain comprises a V_{H} comprising the amino sequence set forth in SEQ ID NO: 29.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 32. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 32. In certain embodiments, the extracellular antigen-binding domain comprises a V_{L} comprising the amino sequence set forth in SEQ ID NO: 32.

In certain embodiments, the extracellular antigen-binding domain (e.g., an scFv) comprises a V_{H} comprising the amino acid sequence set forth in SEQ ID NO: 29, and a V_{L} comprising the amino acid sequence set forth in SEQ ID NO: 32. In certain embodiments, the extracellular antigen-binding domain is an scFv. In certain embodiments, the scFv is designated as “k4-h6”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 26

| CDRs | 1 | 2 | 3 |
|---|---|---|---|
| V_{H} | GYTFTNYY | IGPGSGSP | AKWLLPFDY |
| | [SEQ ID NO: 12] | [SEQ ID NO: 13] | [SEQ ID NO: 26] |
| V_{L} | SSVNY | ATS | QQFSSDPLT |
| | [SEQ ID NO: 15] | [SEQ ID NO: 16] | [SEQ ID NO: 17] |

| | |
|---|---|
| Full | EVQLLESGGGLVQPGGSLRLSCAASGYTFTNYYIHWVRQAPGKGLEWVSKIGPGSG |
| V_{H} | SPYYADSVKGRFTISVDNSKNTLYLQMNSLRAEDTAVYYCAKWLLPFDYWGQGTLV |
| | TVSS |
| | [SEQ ID NO: 29] |
| Full | DIQMTQSPSSVSASVGDRVTITCRASSSVNYMHWYQQKPGKAPKPLIYATSILASG |
| V_{L} | VPSRFSGSGSGTDYTLTISSLQPEDFATYYCQQFSSDPLTFGGGTKVEIK |
| | [SEQ ID NO: 32] |

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof. SEQ ID NOs: 12-14 are provided in Table 27.

In certain embodiments, the extracellular antigen-binding domain (e.g., an scFv) comprises a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 27.

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof; and a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 22. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 22. In certain embodiments, the extracellular antigen-binding domain comprises a V_{H} comprising the amino sequence set forth in SEQ ID NO: 22.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 19. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 19. In certain embodiments, the extracellular antigen-binding domain comprises a V_{L} comprising the amino sequence set forth in SEQ ID NO: 19.

In certain embodiments, the extracellular antigen-binding domain (e.g., an scFv) comprises a V_{H} comprising the amino acid sequence set forth in SEQ ID NO: 22, and a V_{L} comprising the amino acid sequence set forth in SEQ ID NO: 19. In certain embodiments, the extracellular antigen-binding domain is an scFv. In certain embodiments, the scFv is designated as “k5-h1”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 27

| CDRs | 1 | 2 | 3 |
|---|---|---|---|
| V_{H} | GYTFTNYY | IGPGSGSP | ARWLLPFDY |
| | [SEQ ID NO: 12] | [SEQ ID NO: 13] | [SEQ ID NO: 14] |
| V_{L} | SSVNY | ATS | QQFSSDPLT |
| | [SEQ ID NO: 15] | [SEQ ID NO: 16] | [SEQ ID NO: 17] |

| | |
|---|---|
| Full | QVQLVQSGAEVKKPGASVKVSCKASGYTFTNYYIHWVRQAPGQGLEWMGKIGPGSG |
| V_{H} | SPYYNGMFKDRVTMTRDTSTSTVYMELSSLRSEDTAVYYCARWLLPFDYWGQGTLV |
| | TVSS |
| | [SEQ ID NO: 22] |
| Full | EIVLTQSPGTLSLSPGERATLSCRASSSVNYMHWYQQKPGQAPRLLIYATSILASG |
| V_{L} | IPDRESGSGSGTDYTLTISRLEPEDFAVYYCQQFSSDPLTFGGGTKVEIK |
| | [SEQ ID NO: 19] |

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof. SEQ ID NOs: 12-14 are provided in Table 28.

In certain embodiments, the extracellular antigen-binding domain (e.g., an scFv) comprises a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 28.

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof; and a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 24. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 24. In certain embodiments, the extracellular antigen-binding domain comprises a V_{H} comprising the amino sequence set forth in SEQ ID NO: 24.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 19. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 19. In certain embodiments, the extracellular antigen-binding domain comprises a V_{L} comprising the amino sequence set forth in SEQ ID NO: 19.

In certain embodiments, the extracellular antigen-binding domain (e.g., an scFv) comprises a V_{H} comprising the amino acid sequence set forth in SEQ ID NO: 24, and a V_{L} comprising the amino acid sequence set forth in SEQ ID NO: 19. In certain embodiments, the extracellular antigen-binding domain is an scFv. In certain embodiments, the scFv is designated as “k5-h2”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 28

| CDRS | 1 | 2 | 3 |
|---|---|---|---|
| V_{H} | GYTFTNYY | IGPGSGSP | ARWLLPFDY |
| | [SEQ ID NO: 12] | [SEQ ID NO: 13] | [SEQ ID NO: 14] |
| V_{L} | SSVNY | ATS | QQFSSDPLT |
| | [SEQ ID NO: 15] | [SEQ ID NO: 16] | [SEQ ID NO: 17] |

| | |
|---|---|
| Full | QVQLVQSGAEVKKPGASVKVSCKASGYTFTNYYIHWVRQAPGQGLEWMGKIGPGSG |
| V_{H} | SPYYNGMFKDRVTSTRDTSISTAYMELSRLRSDDTVVYYCARWLLPFDYWGQGTLV |
| | TVSS |
| | [SEQ ID NO: 24] |
| Full | EIVLTQSPGTLSLSPGERATLSCRASSSVNYMHWYQQKPGQAPRLLIYATSILASG |
| V_{L} | IPDRESGSGSGTDYTLTISRLEPEDFAVYYCQQFSSDPLTFGGGTKVEIK |
| | [SEQ ID NO: 19] |

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof. SEQ ID NOs: 12-14 are provided in Table 29.

In certain embodiments, the extracellular antigen-binding domain (e.g., an scFv) comprises a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 29.

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof, and a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 25. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 25. In certain embodiments, the extracellular antigen-binding domain comprises a V_{H} comprising the amino sequence set forth in SEQ ID NO: 25.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 19. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90°/a, about 91%, about 92%, about 93%, about 94%, about 95%, about 96%, about 97%, about 98%, about 99% or about 100% homologous or identical to SEQ ID NO: 19. In certain embodiments, the extracellular antigen-binding domain comprises a V_{L} comprising the amino sequence set forth in SEQ ID NO: 19.

In certain embodiments, the extracellular antigen-binding domain (e.g., an scFv) comprises a V_{H} comprising the amino acid sequence set forth in SEQ ID NO: 25, and a V_{L} comprising the amino acid sequence set forth in SEQ ID NO: 19. In certain embodiments, the extracellular antigen-binding domain is an scFv. In certain embodiments, the scFv is designated as “k5-h”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 29

| CDRS | 1 | 2 | 3 |
|---|---|---|---|
| V_{H} | GYTFTNYY | IGPGSGSP | ARWLLPFDY |
| | [SEQ ID NO: 12] | [SEQ ID NO: 13] | [SEQ ID NO: 14] |
| V_{L} | SSVNY | ATS | QQFSSDPLT |
| | [SEQ ID NO: 15] | [SEQ ID NO: 16] | [SEQ ID NO: 17] |

| | |
|---|---|
| Full | QVQLVESGGGVVQPGRSLRLSCAASGYTFTNYYIHWVRQAPGKGLEWVAKIGPGSG |
| V_{H} | SPYYNGMFKDRFTISRDNSKNTLYLQMNSLRAEDTAVYYCARWLLPFDYWGQGTLV |
| | TVSS |
| | [SEQ ID NO: 25] |
| Full | EIVLTQSPGTLSLSPGERATLSCRASSSVNYMHWYQQKPGQAPRLLIYATSILASG |
| V_{L} | IPDRFSGSGSGTDYTLTISRLEPEDFAVYYCQQFSSDPLTFGGGTKVEIK |
| | [SEQ ID NO: 19] |

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 26 or a conservative modification thereof. SEQ ID NOs: 12, 13, and 26 are provided in Table 30.

In certain embodiments, the extracellular antigen-binding domain (e.g., an scFv) comprises a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 30.

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 26 or a conservative modification thereof; and a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 26; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 27. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 27. In certain embodiments, the extracellular antigen-binding domain comprises a V_{H} comprising the amino sequence set forth in SEQ ID NO: 27.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 19. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100 homologous or identical to SEQ ID NO: 19. In certain embodiments, the extracellular antigen-binding domain comprises a V_{L} comprising the amino sequence set forth in SEQ ID NO: 19.

In certain embodiments, the extracellular antigen-binding domain (e.g., an scFv) comprises a V_{H} comprising the amino acid sequence set forth in SEQ ID NO: 27, and a V_{L} comprising the amino acid sequence set forth in SEQ ID NO: 19. In certain embodiments, the extracellular antigen-binding domain is an scFv. In certain embodiments, the scFv is designated as “k5-h4”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 30

| CDRS | 1 | 2 | 3 |
|---|---|---|---|
| V_{H} | GYTFTNYY | IGPGSGSP | AKWLLPFDY |
| | [SEQ ID NO: 12] | [SEQ ID NO: 13] | [SEQ ID NO: 26] |
| V_{L} | SSVNY | ATS | QQFSSDPLT |
| | [SEQ ID NO: 15] | [SEQ ID NO: 16] | [SEQ ID NO: 17] |

| | |
|---|---|
| Full | EVQLLESGGGLVQPGGSLRLSCAASGYTFTNYYIHWVRQAPGKGLEWVSKIGPGSG |
| V_{H} | SPYYNGMFKDRFTISVDNSKNTLYLQMNSLRAEDTAVYYCAKWLLPFDYWGQGTLV |
| | TVSS |
| | [SEQ ID NO: 27] |
| Full | EIVLTQSPGTLSLSPGERATLSCRASSSVNYMHWYQQKPGQAPRLLIYATSILASG |
| V_{L} | IPDRESGSGSGTDYTLTI SRLEPEDFAVYYCQQFSSDPLTFGGGTKVEIK |
| | [SEQ ID NO: 19] |

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof. SEQ ID NOs: 12-14 are provided in Table 31.

In certain embodiments, the extracellular antigen-binding domain (e.g., an scFv) comprises a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 31.

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof; and a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 28. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 28. In certain embodiments, the extracellular antigen-binding domain comprises a V_{H} comprising the amino sequence set forth in SEQ ID NO: 28.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 19. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 19. In certain embodiments, the extracellular antigen-binding domain comprises a V_{L} comprising the amino sequence set forth in SEQ ID NO: 19.

In certain embodiments, the extracellular antigen-binding domain (e.g., an scFv) comprises a V_{H} comprising the amino acid sequence set forth in SEQ ID NO: 28, and a V_{L} comprising the amino acid sequence set forth in SEQ ID NO: 19. In certain embodiments, the extracellular antigen-binding domain is an scFv. In certain embodiments, the scFv is designated as “k5-h5”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 31

| CDRs | 1 | 2 | 3 |
|---|---|---|---|
| VH | GYTFTNYY | IGPGSGSP | ARWLLPFDY |
| | [SEQ ID NO: 12] | [SEQ ID NO: 13] | [SEQ ID NO: 14] |
| VL | SSVNY | ATS | QQFSSDPLT |
| | [SEQ ID NO: 15] | [SEQ ID NO: 16] | [SEQ ID NO: 17] |

| | |
|---|---|
| Full | QVQLVQSGAEVKKPGSSVKVSCKASGYTFTNYYIHWVRQAPGQGLEWMGKIGPGSG |
| VH | SPYYNGMFKDRVTITADESTSTAYMELSSLRSEDTAVYYCARWLLPFDYWGQGTLV |
| | TVSS |
| | [SEQ ID NO: 28] |
| Full | EIVLTQSPGTLSLSPGERATLSCRASSSVNYMHWYQQKPGQAPRLLIYATSILASG |
| VL | IPDRFSGSGSGTDYTLTISRLEPEDFAVYYCQQFSSDPLTFGGGTKVEIK |
| | [SEQ ID NO: 19] |

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 26 or a conservative modification thereof. SEQ ID NOs: 12, 13, and 26 are provided in Table 32.

In certain embodiments, the extracellular antigen-binding domain (e.g., an scFv) comprises a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 32.

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 26 or a conservative modification thereof; and a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 26; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 29. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 29. In certain embodiments, the extracellular antigen-binding domain comprises a V_{H} comprising the amino sequence set forth in SEQ ID NO: 29.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 19. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 19. In certain embodiments, the extracellular antigen-binding domain comprises a V_{L} comprising the amino sequence set forth in SEQ ID NO: 19.

In certain embodiments, the extracellular antigen-binding domain (e.g., an scFv) comprises a V_{H} comprising the amino acid sequence set forth in SEQ ID NO: 29, and a V_{L} comprising the amino acid sequence set forth in SEQ ID NO: 19. In certain embodiments, the extracellular antigen-binding domain is an scFv. In certain embodiments, the scFv is designated as “k5-h6”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 32

| CDRS | 1 | 2 | 3 |
|---|---|---|---|
| VH | GYTFTNYY | IGPGSGSP | AKWLLPFDY |
| | [SEQ ID NO: 12] | [SEQ ID NO: 13] | [SEQ ID NO: 26] |
| VL | SSVNY | ATS | QQFSSDPLT |
| | [SEQ ID NO: 15] | [SEQ ID NO: 16] | [SEQ ID NO: 17] |

| | |
|---|---|
| Full | EVQLLESGGGLVQPGGSLRLSCAASGYTFTNYYIHWVRQAPGKGLEWVSKIGPGSG |
| VH | SPYYADSVKGRFTISVDNSKNTLYLQMNSLRAEDTAVYYCAKWLLPFDYWGQGTLV |
| | TVSS |
| | [SEQ ID NO: 29] |
| Full | EIVLTQSPGTLSLSPGERATLSCRASSSVNYMHWYQQKPGQAPRLLIYATSILASG |
| VL | IPDRESGSGSGTDYTLTISRLEPEDFAVYYCQQFSSDPLTFGGGTKVEIK |
| | [SEQ ID NO: 19] |

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 26 or a conservative modification thereof. SEQ ID NOs: 12, 13, and 26 are provided in Table 33.

In certain embodiments, the extracellular antigen-binding domain (e.g., an scFv) comprises a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 33.

In certain embodiments, the extracellular antigen-binding domain (e.g., an scFv) comprises a heavy chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 26 or a conservative modification thereof; and a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 26; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 33. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 33. In certain embodiments, the extracellular antigen-binding domain comprises a V_{H} comprising the amino sequence set forth in SEQ ID NO: 33.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 34. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 34. In certain embodiments, the extracellular antigen-binding domain comprises a V_{L} comprising the amino sequence set forth in SEQ ID NO: 34.

In certain embodiments, the extracellular antigen-binding domain (e.g., an scFv) comprises a V_{H} comprising the amino acid sequence set forth in SEQ ID NO: 33, and a V_{L} comprising the amino acid sequence set forth in SEQ ID NO: 34. In certain embodiments, the extracellular antigen-binding domain is an scFv. In certain embodiments, the scFv is designated as “143G03”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 33

| CDRS | 1 | 2 | 3 |
|---|---|---|---|
| VH | GYTFTNYY | IGPGSGSP | AKWLLPFDY |
| | [SEQ ID NO: 12] | [SEQ ID NO: 13] | [SEQ ID NO: 26] |
| VL | SSVNY | ATS | QQFSSDPLT |
| | [SEQ ID NO: 15] | [SEQ ID NO: 16] | [SEQ ID NO: 17] |

| | |
|---|---|
| Full | QVQLRQSGAELVRPGTSVKMSCKASGYTFTNYYIHWVKQRPGQGLEWIGKIGPGSG |
| VH | SPYYNGMFKDKATLTVDTSSSTAYMQLSSLTSEDSAVYFCARWLLPFDYWGQGTTL |
| | TVSS |
| | [SEQ ID NO: 33] |
| Full | QIVLSQSPGILSASPGEKVTMTCRASSSVNYMHWYQQKSGSSPKPWIFATSILASG |
| VL | VPPRFSGSGSGTSYSLTISSVEAEDTATYYCQQFSSDPLTFGAGTKLELK |
| | [SEQ ID NO: 34] |

In certain embodiments, the extracellular antigen-binding domain (e.g., an scFv) comprises a heavy chain variable region comprising a CDR1 comprising the amino acid sequence set forth in SEQ ID NO: 35 or a conservative modification thereof, a CDR2 comprising the amino acid sequence set forth in SEQ ID NO: 36 or a conservative modification thereof, and a CDR3 comprising the amino acid sequence set forth in SEQ ID NO: 37 or a conservative modification thereof. SEQ ID NOs: 35-37 are provided in Table 34.

In certain embodiments, the extracellular antigen-binding domain (e.g., an scFv) comprises a light chain variable region comprising a CDR1 comprising the amino acid sequence set forth in SEQ ID NO: 38 or a conservative modification thereof, a CDR2 comprising the amino acid sequence set forth in SEQ ID NO: 39 or a conservative modification thereof, and a CDR3 comprising the amino acid sequence set forth in SEQ ID NO: 40 or a conservative modification thereof. SEQ ID NOs: 38-40 are provided in Table 34.

In certain embodiments, the extracellular antigen-binding domain (e.g., an scFv) comprises a heavy chain variable region comprising a CDR1 comprising the amino acid sequence set forth in SEQ ID NO: 35 or a conservative modification thereof, a CDR2 comprising the amino acid sequence set forth in SEQ ID NO: 36 or a conservative modification thereof, and a CDR3 comprising the amino acid sequence set forth in SEQ ID NO: 37 or a conservative modification thereof, and a light chain variable region comprising a CDR1 comprising the amino acid sequence set forth in SEQ ID NO: 38 or a conservative modification thereof, a CDR2 comprising the amino acid sequence set forth in SEQ ID NO: 39 or a conservative modification thereof, and a CDR3 comprising the amino acid sequence set forth in SEQ ID NO: 40 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 35, a CDR2 comprising the amino acid sequence set forth in SEQ ID NO: 36, and a CDR3 comprising the amino acid sequence set forth in SEQ ID NO: 37; and a light chain variable region comprising a CDR1 comprising the amino acid sequence set forth in SEQ ID NO: 38, a CDR2 comprising the amino acid sequence set forth in SEQ ID NO: 39, and a CDR3 comprising the amino acid sequence set forth in SEQ ID NO: 40.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 41. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90°/, about 91%, about 92%, about 93%, about 94%, about 95%, about 96%, about 97%, about 98%, about 99% or about 100% homologous or identical to SEQ ID NO: 41. In certain embodiments, the extracellular antigen-binding domain comprises a V_{H} comprising the amino sequence set forth in SEQ ID NO: 41.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 42. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90°/a, about 91%, about 92%, about 93%, about 94%, about 95%, about 96%, about 97%, about 98%, about 99% or about 100% homologous or identical to SEQ ID NO: 42. In certain embodiments, the extracellular antigen-binding domain comprises a V_{L} comprising the amino sequence set forth in SEQ ID NO: 42.

In certain embodiments, the extracellular antigen-binding domain (e.g., an scFv) comprises a V_{H} comprising the amino acid sequence set forth in SEQ ID NO: 41, and a V_{L} comprising the amino acid sequence set forth in SEQ ID NO: 42. In certain embodiments, the extracellular antigen-binding domain is an scFv. In certain embodiments, the scFv is designated as “04B06”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 34

| CDRs | 1 | 2 | 3 |
|---|---|---|---|
| VH | GGSINSYY | FYTSGST | AREWSGS FYGGDAFDI |
| | [SEQ ID NO: 35] | [SEQ ID NO: 36] | [SEQ ID NO: 37] |
| VL | QSVLNSSNNKNY | WAS | QQYYSTPYT |
| | [SEQ ID NO: 38] | [SEQ ID NO: 39] | [SEQ ID NO: 40] |

| | |
|---|---|
| Full | QVQLQESGPGLVKPSETLSLTCTVSGGSINSYYWSWIRQPAGKGLEWIGRFYTSGS |
| VH | TNHNPSLKSRVTMSVDTSKNQFSLKLTSVTAADTAVYYCAREWSGSFYGGDAFDIW |
| | GQGTMVTVSS |
| | [SEQ ID NO: 41] |
| Full | DIVMTQSPDSLAVSLGERATINCKSSQSVLNSSNNKNYLTWYQQRPGQPPNLLIYW |
| VL | ASTRESGVPDRESGSGSGTDFTLTISSLQAEDVAVYYCQQYYSTPYTFGQGTKLEI |
| | E |
| | [SEQ ID NO: 42] |

In certain embodiments, the extracellular antigen-binding domain (e.g., an scFv) comprises a heavy chain variable region comprising a CDR1 comprising the amino acid sequence set forth in SEQ ID NO: 43 or a conservative modification thereof, a CDR2 comprising the amino acid sequence set forth in SEQ ID NO: 44 or a conservative modification thereof, and a CDR3 comprising the amino acid sequence set forth in SEQ ID NO: 45 or a conservative modification thereof. SEQ ID NOs: 43-45 are provided in Table 35.

In certain embodiments, the extracellular antigen-binding domain (e.g., an scFv) comprises a light chain variable region comprising a CDR1 comprising the amino acid sequence set forth in SEQ ID NO: 46 or a conservative modification thereof, a CDR2 comprising the amino acid sequence set forth in SEQ ID NO: 47 or a conservative modification thereof, and a CDR3 comprising the amino acid sequence set forth in SEQ ID NO: 48 or a conservative modification thereof. SEQ ID NOs: 46-48 are provided in Table 35.

In certain embodiments, the extracellular antigen-binding domain (e.g., an scFv) comprises a heavy chain variable region comprising a CDR1 comprising the amino acid sequence set forth in SEQ ID NO: 43 or a conservative modification thereof, a CDR2 comprising the amino acid sequence set forth in SEQ ID NO: 44 or a conservative modification thereof, and a CDR3 comprising the amino acid sequence set forth in SEQ ID NO: 45 or a conservative modification thereof; and a light chain variable region comprising a CDR1 comprising the amino acid sequence set forth in SEQ ID NO: 46 or a conservative modification thereof, a CDR2 comprising the amino acid sequence set forth in SEQ ID NO: 47 or a conservative modification thereof, and a CDR3 comprising the amino acid sequence set forth in SEQ ID NO: 48 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 43, a CDR2 comprising the amino acid sequence set forth in SEQ ID NO: 44, and a CDR3 comprising the amino acid sequence set forth in SEQ ID NO: 45; and a light chain variable region comprising a CDR1 comprising the amino acid sequence set forth in SEQ ID NO: 46, a CDR2 comprising the amino acid sequence set forth in SEQ ID NO: 47, and a CDR3 comprising the amino acid sequence set forth in SEQ ID NO: 48.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 49. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 49. In certain embodiments, the extracellular antigen-binding domain comprises a V_{H} comprising the amino sequence set forth in SEQ ID NO: 49.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 50. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 50. In certain embodiments, the extracellular antigen-binding domain comprises a V_{L} comprising the amino sequence set forth in SEQ ID NO: 50.

In certain embodiments, the extracellular antigen-binding domain (e.g., an scFv) comprises a V_{H} comprising the amino acid sequence set forth in SEQ ID NO: 49, and a V_{L} comprising the amino acid sequence set forth in SEQ ID NO: 50. In certain embodiments, the extracellular antigen-binding domain is an scFv. In certain embodiments, the scFv is designated as “12D10v1”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 35

| CDRS | 1 | 2 | 3 |
|---|---|---|---|
| VH | GYPFTGYY | INPNSGDT | AREMGTTKGYFDL |
| | [SEQ ID NO: 43] | [SEQ ID NO: 44] | [SEQ ID NO: 45] |
| VL | QGISSY | AAS | QQLNSYPDT |
| | [SEQ ID NO: 46] | [SEQ ID NO: 47] | [SEQ ID NO: 48] |

| | |
|---|---|
| Full | QVQLVQSGAEVKKPGASVRVSCKASGYPFTGYYMHWVRQAPGQGLEWMGWINPNSG |
| VH | DTDYVQKFQGRVTMTRDTSISTAYMELSRLRSDDTAVYYCAREMGTTKGYFDLWGR |
| | GTLVTVSS |
| | [SEQ ID NO: 49] |
| Full | DIQLTQSPSFLSASVGDRVTITCRASQGISSYLAWFQQKPGKAPRLLIYAASTLQS |
| VL | GVPSRESGSRSGTEFTLTISSLQPEDFATYYCQQLNSYPDTFGQGTKVDIK |
| | [SEQ ID NO: 50] |

In certain embodiments, the extracellular antigen-binding domain (e.g., an scFv) comprises a heavy chain variable region comprising a CDR1 comprising the amino acid sequence set forth in SEQ ID NO: 43 or a conservative modification thereof, a CDR2 comprising the amino acid sequence set forth in SEQ ID NO: 44 or a conservative modification thereof, and a CDR3 comprising the amino acid sequence set forth in SEQ ID NO: 45 or a conservative modification thereof. SEQ ID NOs: 43-45 are provided in Table 36.

In certain embodiments, the extracellular antigen-binding domain (e.g., an scFv) comprises a light chain variable region comprising a CDR1 comprising the amino acid sequence set forth in SEQ ID NO: 51 or a conservative modification thereof, a CDR2 comprising the amino acid sequence set forth in SEQ ID NO: 52 or a conservative modification thereof, and a CDR3 comprising the amino acid sequence set forth in SEQ ID NO: 40 or a conservative modification thereof. SEQ ID NOs: 51, 52, and 40 are provided in Table 36.

In certain embodiments, the extracellular antigen-binding domain (e.g., an scFv) comprises a heavy chain variable region comprising a CDR1 comprising the amino acid sequence set forth in SEQ ID NO: 43 or a conservative modification thereof, a CDR2 comprising the amino acid sequence set forth in SEQ ID NO: 44 or a conservative modification thereof, and a CDR3 comprising the amino acid sequence set forth in SEQ ID NO: 45 or a conservative modification thereof; and a light chain variable region comprising a CDR1 comprising the amino acid sequence set forth in SEQ ID NO: 51 or a conservative modification thereof, a CDR2 comprising the amino acid sequence set forth in SEQ ID NO: 52 or a conservative modification thereof, and a CDR3 comprising the amino acid sequence set forth in SEQ ID NO: 40 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 43, a CDR2 comprising the amino acid sequence set forth in SEQ ID NO: 44, and a CDR3 comprising the amino acid sequence set forth in SEQ ID NO: 45; and a light chain variable region comprising a CDR1 comprising the amino acid sequence set forth in SEQ ID NO: 51, a CDR2 comprising the amino acid sequence set forth in SEQ ID NO: 52, and a CDR3 comprising the amino acid sequence set forth in SEQ ID NO: 40.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 49. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 49. In certain embodiments, the extracellular antigen-binding domain comprises a V_{H} comprising the amino sequence set forth in SEQ ID NO: 49.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 53. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 53. In certain embodiments, the extracellular antigen-binding domain comprises a V_{L} comprising the amino sequence set forth in SEQ ID NO: 53.

In certain embodiments, the extracellular antigen-binding domain (e.g., an scFv) comprises a V_{H} comprising the amino acid sequence set forth in SEQ ID NO: 49, and a V_{L} comprising the amino acid sequence set forth in SEQ ID NO: 53. In certain embodiments, the extracellular antigen-binding domain is an scFv. In certain embodiments, the scFv is designated as “12D10v2”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 36

| CDRs | 1 | 2 | 3 |
|---|---|---|---|
| VH | GYPFTGYY | INPNSGDT | AREMGTTKGYFDL |
| | [SEQ ID NO: 43] | [SEQ ID NO: 44] | [SEQ ID NO: 45] |
| VL | QTILYSPNNKNY | LTS | QQYYSTPYT |
| | [SEQ ID NO: 51] | [SEQ ID NO: 52] | [SEQ ID NO: 40] |

| | |
|---|---|
| Full | QVQLVQSGAEVKKPGASVRVSCKASGYPFTGYYMHWVRQAPGQGLEWMGWINPNSG |
| VH | DTDYVQKFQGRVTMTRDTSISTAYMELSRLRSDDTAVYYCAREMGTTKGYFDLWGR |
| | GTLVTVSS |
| | [SEQ ID NO: 49] |
| Full | DIVMTQSPDSLAVSLGERATINCKSSQTILYSPNNKNYLAWYQQKPGQPPKLLIYL |
| VL | TSTRESGVPDRESGSGSGTDFTLTISSLQAEDVAVYYCQQYYSTPYTFGQGTKLEI |
| | K |
| | [SEQ ID NO: 53] |

In certain embodiments, the extracellular antigen-binding domain (e.g., an scFv) comprises a heavy chain variable region comprising a CDR1 comprising the amino acid sequence set forth in SEQ ID NO: 54 or a conservative modification thereof, a CDR2 comprising the amino acid sequence set forth in SEQ ID NO: 55 or a conservative modification thereof, and a CDR3 comprising the amino acid sequence set forth in SEQ ID NO: 56 or a conservative modification thereof. SEQ ID NOs: 54-56 are provided in Table 37.

In certain embodiments, the extracellular antigen-binding domain (e.g., an scFv) comprises a light chain variable region comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 59 or a conservative modification thereof. SEQ ID NOs: 57-59 are provided in Table 37.

In certain embodiments, the extracellular antigen-binding domain (e.g., an scFv) comprises a heavy chain variable region comprising a CDR1 comprising the amino acid sequence set forth in SEQ ID NO: 54 or a conservative modification thereof, a CDR2 comprising the amino acid sequence set forth in SEQ ID NO: 55 or a conservative modification thereof, and a CDR3 comprising the amino acid sequence set forth in SEQ ID NO: 56 or a conservative modification thereof; and a light chain variable region comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 59 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 54, a CDR2 comprising the amino acid sequence set forth in SEQ ID NO: 55, and a CDR3 comprising the amino acid sequence set forth in SEQ ID NO: 56; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 59.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 60. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 60. In certain embodiments, the extracellular antigen-binding domain comprises a V_{H} comprising the amino sequence set forth in SEQ ID NO: 60.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 61. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 61. In certain embodiments, the extracellular antigen-binding domain comprises a V_{L} comprising the amino sequence set forth in SEQ ID NO: 61.

In certain embodiments, the extracellular antigen-binding domain (e.g., an scFv) comprises a V_{H} comprising the amino acid sequence set forth in SEQ ID NO: 60, and a V_{L} comprising the amino acid sequence set forth in SEQ ID NO: 61. In certain embodiments, the extracellular antigen-binding domain is an scFv. In certain embodiments, the scFv is designated as “13G04”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 37

| CDRs | 1 | 2 | 3 |
|---|---|---|---|
| VH | GYTFIGYY | INPNSGGT | ARGGLERRDAFDI |
| | [SEQ ID NO: 54] | [SEQ ID NO: 55] | [SEQ ID NO: 56] |
| VL | QSISNW | KAS | QQYDTYRT |
| | [SEQ ID NO: 57] | [SEQ ID NO: 58] | [SEQ ID NO: 59] |

| | |
|---|---|
| Full | QVQLVQSGAEVKKPGASVKVSCKASGYTFIGYYLHWVRQAPGQGLEWMGWINPNSG |
| VH | GTNSAQKFQGRVTMTRDTSISTTYMDLSRLRSDDTAVYYCARGGLERRDAFDIWGQ |
| | GTLVTVSS |
| | [SEQ ID NO: 60] |
| Full | DIQMTQSPSTLSASVGDRVTITCRASQSISNWLAWYQQKPGKAPNLLIYKASSLES |
| VL | GVPSRFSGSGSGTEFTLTISSLQPDDFATYYCQQYDTYRTFGQGTKVEIK |
| | [SEQ ID NO: 61] |

In certain embodiments, the extracellular antigen-binding domain (e.g., an scFv) comprises a heavy chain variable region comprising a CDR1 comprising the amino acid sequence set forth in SEQ ID NO: 62 or a conservative modification thereof, a CDR2 comprising the amino acid sequence set forth in SEQ ID NO: 63 or a conservative modification thereof, and a CDR3 comprising the amino acid sequence set forth in SEQ ID NO: 64 or a conservative modification thereof. SEQ ID NOs: 62-64 are provided in Table 38.

In certain embodiments, the extracellular antigen-binding domain (e.g., an scFv) comprises a light chain variable region comprising a CDR1 comprising the amino acid sequence set forth in SEQ ID NO: 65 or a conservative modification thereof, a CDR2 comprising the amino acid sequence set forth in SEQ ID NO: 58 or a conservative modification thereof, and a CDR3 comprising the amino acid sequence set forth in SEQ ID NO: 66 or a conservative modification thereof. SEQ ID NOs: 65, 58, and 66 are provided in Table 38.

In certain embodiments, the extracellular antigen-binding domain (e.g., an scFv) comprises a heavy chain variable region comprising a CDR1 comprising the amino acid sequence set forth in SEQ ID NO: 62 or a conservative modification thereof, a CDR2 comprising the amino acid sequence set forth in SEQ ID NO: 63 or a conservative modification thereof, and a CDR3 comprising the amino acid sequence set forth in SEQ ID NO: 64 or a conservative modification thereof; and a light chain variable region comprising a CDR1 comprising the amino acid sequence set forth in SEQ ID NO: 65 or a conservative modification thereof, a CDR2 comprising the amino acid sequence set forth in SEQ ID NO: 58 or a conservative modification thereof, and a CDR3 comprising the amino acid sequence set forth in SEQ ID NO: 66 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 62, a CDR2 comprising the amino acid sequence set forth in SEQ ID NO: 63, and a CDR3 comprising the amino acid sequence set forth in SEQ ID NO: 64; and a light chain variable region comprising a CDR1 comprising the amino acid sequence set forth in SEQ ID NO: 65, a CDR2 comprising the amino acid sequence set forth in SEQ ID NO: 58, and a CDR3 comprising the amino acid sequence set forth in SEQ ID NO: 66.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 67. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 67. In certain embodiments, the extracellular antigen-binding domain comprises a V_{H} comprising the amino sequence set forth in SEQ ID NO: 67.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 68. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 68. In certain embodiments, the extracellular antigen-binding domain comprises a V_{L} comprising the amino sequence set forth in SEQ ID NO: 68.

In certain embodiments, the extracellular antigen-binding domain (e.g., an scFv) comprises a V_{H} comprising the amino acid sequence set forth in SEQ ID NO: 67, and a V_{L} comprising the amino acid sequence set forth in SEQ ID NO: 68. In certain embodiments, the extracellular antigen-binding domain is an scFv. In certain embodiments, the scFv is designated as “15G02”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 38

| CDRs | 1 | 2 | 3 |
|---|---|---|---|
| VH | GFTFSHYW | IMQDGSEK | ARDRGVTTLFDY |
| | [SEQ ID NO: 62] | [SEQ ID NO: 63] | [SEQ ID NO: 64] |
| VL | QSISSW | KAS | QQYYSYGT |
| | [SEQ ID NO: 65] | [SEQ ID NO: 58] | [SEQ ID NO: 66] |

| | |
|---|---|
| Full | EVQLVESGGGLVQPGGSLRLSCGVSGFTFSHYWMNWVRQAPGKGLEWVANIMQDGS |
| VH | EKYYVDSVKGRFTISRDNAKNSLYLQMNSLRVEDTAVYYCARDRGVTTLFDYWGQG |
| | ILVTVSS |
| | [SEQ ID NO: 67] |
| Full | DIQMTQSPSTLSASVGDRVTITCRASQSISSWLAWYQQKPGKAPKLLIYKASSLES |
| VL | GVPSRFSGSGSGTEFTLTISSLQPDDFATYYCQQYYSYGTFGQGTKVEIK |
| | [SEQ ID NO: 68] |

In certain embodiments, the extracellular antigen-binding domain (e.g., an scFv) comprises a heavy chain variable region comprising a CDR1 comprising the amino acid sequence set forth in SEQ ID NO: 69 or a conservative modification thereof, a CDR2 comprising the amino acid sequence set forth in SEQ ID NO: 70 or a conservative modification thereof, and a CDR3 comprising the amino acid sequence set forth in SEQ ID NO: 71 or a conservative modification thereof. SEQ ID NOs: 69-71 are provided in Table 39.

In certain embodiments, the extracellular antigen-binding domain (e.g., an scFv) comprises a light chain variable region comprising a CDR1 comprising the amino acid sequence set forth in SEQ ID NO: 72 or a conservative modification thereof, a CDR2 comprising the amino acid sequence set forth in SEQ ID NO: 73 or a conservative modification thereof, and a CDR3 comprising the amino acid sequence set forth in SEQ ID NO: 74 or a conservative modification thereof. SEQ ID NOs: 72-74 are provided in Table 39.

In certain embodiments, the extracellular antigen-binding domain (e.g., an scFv) comprises a heavy chain variable region comprising a CDR1 comprising the amino acid sequence set forth in SEQ ID NO: 69 or a conservative modification thereof, a CDR2 comprising the amino acid sequence set forth in SEQ ID NO: 70 or a conservative modification thereof, and a CDR3 comprising the amino acid sequence set forth in SEQ ID NO: 71 or a conservative modification thereof, and a light chain variable region comprising a CDR1 comprising the amino acid sequence set forth in SEQ ID NO: 72 or a conservative modification thereof, a CDR2 comprising the amino acid sequence set forth in SEQ ID NO: 73 or a conservative modification thereof, and a CDR3 comprising the amino acid sequence set forth in SEQ ID NO: 74 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 69, a CDR2 comprising the amino acid sequence set forth in SEQ ID NO: 70, and a CDR3 comprising the amino acid sequence set forth in SEQ ID NO: 71; and a light chain variable region comprising a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 74.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 75. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 75. In certain embodiments, the extracellular antigen-binding domain comprises a V_{H} comprising the amino sequence set forth in SEQ ID NO: 75.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 76. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90°/a, about 91%, about 92%, about 93%, about 94%, about 95%, about 96%, about 97%, about 98%, about 99% or about 100% homologous or identical to SEQ ID NO: 76. In certain embodiments, the extracellular antigen-binding domain comprises a V_{L} comprising the amino sequence set forth in SEQ ID NO: 76.

In certain embodiments, the extracellular antigen-binding domain (e.g., an scFv) comprises a V_{H} comprising the amino acid sequence set forth in SEQ ID NO: 75, and a V_{L} comprising the amino acid sequence set forth in SEQ ID NO: 76. In certain embodiments, the extracellular antigen-binding domain is an scFv. In certain embodiments, the scFv is designated as “13F04”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}. In certain embodiments, the extracellular antigen-binding domain is an scFv comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 77. In certain embodiments, the extracellular antigen-binding domain is an scFv comprising the amino sequence set forth in SEQ ID NO: 77. An exemplary nucleotide sequence encoding the amino acid sequence of SEQ ID NO: 77 is set forth in SEQ ID NO: 79. SEQ ID NOs: 77 and 79 are provided in Table 39 below.

In certain embodiments, the variable regions within the extracellular antigen-binding domain have to be linked one after another such that at the N-terminus of the extracellular antigen-binding domain, a light chain variable region (V_{L}) is positioned. In certain embodiments, the extracellular antigen-binding domain is an scFv, the variable regions are positioned from the N- to the C-terminus: V_{L}-V_{H}. In certain embodiments, the extracellular antigen-binding domain is an scFv comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 78. In certain embodiments, the extracellular antigen-binding domain is an scFv comprising the amino sequence set forth in SEQ ID NO: 78. An exemplary nucleotide sequence encoding the amino acid sequence of SEQ ID NO: 78 is set forth in SEQ ID NO: 80. SEQ ID NOs: 78 and 80 are provided in Table 39 below.

TABLE 39

| CDRs | 1 | 2 | 3 |
|---|---|---|---|
| VH | GYIFISYW | INPRNGRT | ARGGDYGGSFDY |
| | [SEQ ID NO: 69] | [SEQ ID NO: 70] | [SEQ ID NO: 71] |
| VL | ESVDSYGNSF | RAS | QQSNEDPWT |
| | [SEQ ID NO: 72] | [SEQ ID NO: 73] | [SEQ ID NO: 74] |

| | |
|---|---|
| Full VH | QVQLQQPGAELVKPGASVKLSCKASGYIFISYWMHWVKQRPGQGLEWIGEINPRNG |
| | RTNYNEKFKSKATLTVDKSSSTAYMQLTSLTSEDSAVYYCARGGDYGGSFDYWGQG |
| | TTLTVSS |
| | [SEQ ID NO: 75] |
| Full VL | DIVLTQSPASLAVSLGQRATISCRASESVDSYGNSFMHWYQQKPGQPPKLLIYRAS |
| | NLESGIPARFSGSGSRTDFTLTVNPVEADDVATYYCQQSNEDPWTFGGGTKLEIK |
| | [SEQ ID NO: 76] |
| ScFv | QVQLQQPGAELVKPGASVKLSCKASGYIFISYWMHWVKQRPGQGLEWIGEINPRNG |
| VH-VL | RTNYNEKFKSKATLTVDKSSSTAYMQLTSLTSEDSAVYYCARGGDYGGSFDYWGQG |
| | TTLTVSSGGGGSGGGGSGGGGSDIVLTQSPASLAVSLGQRATISCRASESVDSYGN |
| | SFMHWYQQKPGQPPKLLIYRASNLESGIPARFSGSGSRTDFTLTVNPVEADDVATY |
| | YCQQSNEDPWTFGGGTKLEIK |
| | [SEQ ID NO: 77] |
| ScFv | DIVLTQSPASLAVSLGQRATISCRASESVDSYGNSFMHWYQQKPGQPPKLLIYRAS |
| VL-VH | NLESGIPARFSGSGSRTDFTLTVNPVEADDVATYYCQQSNEDPWTFGGGTKLEIKG |
| | GGGSGGGGSGGGGSQVQLQQPGAELVKPGASVKLSCKASGYIFISYWMHWVKQRPG |
| | QGLEWIGEINPRNGRTNYNEKFKSKATLTVDKSSSTAYMQLTSLTSEDSAVYYCAR |
| | GGDYGGSFDYWGQGTTLTVSS |
| | [SEQ ID NO: 78] |
| DNA ScFv | CAGGTACAGCTCCAGCAACCGGGGGCGGAGTTGGTAAAGCCGGGTGCGTCCGTAAA |
| VH-VL | ATTGTCATGCAAGGCCAGCGGCTATATCTTCATTAGCTACTGGATGCACTGGGTTA |
| | AGCAACGGCCCGGCCAGGGTCTTGAGTGGATCGGGGAAATTAATCCGAGGAACGGC |
| | CGGACGAACTACAACGAAAAATTCAAATCTAAAGCTACTCTCACCGTGGATAAGTC |
| | TTCTTCTACAGCTTATATGCAGTTGACCAGTCTCACCAGTGAAGACAGTGCGGTAT |
| | ATTACTGTGCAAGGGGTGGAGACTATGGGGGCTCATTCGATTATTGGGGGCAAGGT |
| | ACTACACTGACTGTATCTTCCGGCGGTGGTGGCTCAGGCGGCGGAGGATCCGGGGG |
| | GGGGGGATCCGACATAGTACTCACTCAGAGCCCAGCTTCACTGGCTGTGTCTTTGG |
| | GACAACGCGCAACTATATCCTGCCGGGCGTCAGAATCTGTAGATAGCTATGGAAAC |
| | AGTTTTATGCATTGGTATCAGCAAAAACCCGGTCAGCCCCCTAAGTTGCTCATTTA |
| | TAGAGCCTCAAACCTCGAATCAGGGATTCCGGCACGCTTCAGTGGGTCTGGTAGTA |
| | GAACGGATTTCACACTTACAGTCAATCCAGTCGAGGCCGACGATGTGGCTACATAC |
| | TACTGCCAGCAATCCAATGAAGATCCTTGGACATTTGGTGGCGGGACAAAGCTGGA |
| | AATAAAG |
| | [SEQ ID NO: 79] |
| DNA ScFv | GACATAGTACTCACTCAGAGCCCAGCTTCACTGGCTGTGTCTTTGGGACAACGCGC |
| VL-VH | AACTATATCCTGCCGGGCGTCAGAATCTGTAGATAGCTATGGAAACAGTTTTATGC |
| | ATTGGTATCAGCAAAAACCCGGTCAGCCCCCTAAGTTGCTCATTTATAGAGCCTCA |
| | AACCTCGAATCAGGGATTCCGGCACGCTTCAGTGGGTCTGGTAGTAGAACGGATTT |
| | CACACTTACAGTCAATCCAGTCGAGGCCGACGATGTGGCTACATACTACTGCCAGC |
| | AATCCAATGAAGATCCTTGGACATTTGGTGGCGGGACAAAGCTGGAAATAAAGGGC |
| | GGTGGTGGCTCAGGCGGCGGAGGATCCGGGGGGGGGGGATCCCAGGTACAGCTCCA |
| | GCAACCGGGGGCGGAGTTGGTAAAGCCGGGTGCGTCCGTAAAATTGTCATGCAAGG |
| | CCAGCGGCTATATCTTCATTAGCTACTGGATGCACTGGGTTAAGCAACGGCCCGGC |
| | CAGGGTCTTGAGTGGATCGGGGAAATTAATCCGAGGAACGGCCGGACGAACTACAA |
| | CGAAAAATTCAAATCTAAAGCTACTCTCACCGTGGATAAGTCTTCTTCTACAGCTT |
| | ATATGCAGTTGACCAGTCTCACCAGTGAAGACAGTGCGGTATATTACTGTGCAAGG |
| | GGTGGAGACTATGGGGGCTCATTCGATTATTGGGGGCAAGGTACTACACTGACTGT |
| | ATCTTCC |
| | [SEQ ID NO: 80] |

In certain embodiments, the extracellular antigen-binding domain (e.g., an scFv) comprises a heavy chain variable region comprising a CDR1 comprising the amino acid sequence set forth in SEQ ID NO: 81 or a conservative modification thereof, a CDR2 comprising the amino acid sequence set forth in SEQ ID NO: 82 or a conservative modification thereof, and a CDR3 comprising the amino acid sequence set forth in SEQ ID NO: 83 or a conservative modification thereof. SEQ ID NOs: 81-83 are provided in Table 40.

In certain embodiments, the extracellular antigen-binding domain (e.g., an scFv) comprises a light chain variable region comprising a CDR1 comprising the amino acid sequence set forth in SEQ ID NO: 84 or a conservative modification thereof, a CDR2 comprising the amino acid sequence set forth in SEQ ID NO: 85 or a conservative modification thereof, and a CDR3 comprising the amino acid sequence set forth in SEQ ID NO: 86 or a conservative modification thereof. SEQ ID NOs: 84-86 are provided in Table 40.

In certain embodiments, the extracellular antigen-binding domain (e.g., an scFv) comprises a heavy chain variable region comprising a CDR1 comprising the amino acid sequence set forth in SEQ ID NO: 81 or a conservative modification thereof, a CDR2 comprising the amino acid sequence set forth in SEQ ID NO: 82 or a conservative modification thereof, and a CDR3 comprising the amino acid sequence set forth in SEQ ID NO: 83 or a conservative modification thereof; and a light chain variable region comprising a CDR1 comprising the amino acid sequence set forth in SEQ ID NO: 84 or a conservative modification thereof, a CDR2 comprising the amino acid sequence set forth in SEQ ID NO: 85 or a conservative modification thereof, and a CDR3 comprising the amino acid sequence set forth in SEQ ID NO: 86 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 81, a CDR2 comprising the amino acid sequence set forth in SEQ ID NO: 82, and a CDR3 comprising the amino acid sequence set forth in SEQ ID NO: 83; and a light chain variable region comprising a CDR1 comprising the amino acid sequence set forth in SEQ ID NO: 84, a CDR2 comprising the amino acid sequence set forth in SEQ ID NO: 85, and a CDR3 comprising the amino acid sequence set forth in SEQ ID NO: 86.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 87. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90°/, about 91%, about 92%, about 93%, about 94%, about 95%, about 96%, about 97%, about 98%, about 99% or about 100% homologous or identical to SEQ ID NO: 87. In certain embodiments, the extracellular antigen-binding domain comprises a V_{H} comprising the amino sequence set forth in SEQ ID NO: 87.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 88. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90°/a, about 91%, about 92%, about 93%, about 94%, about 95%, about 96%, about 97%, about 98%, about 99% or about 100% homologous or identical to SEQ ID NO: 88. In certain embodiments, the extracellular antigen-binding domain comprises a V_{L} comprising the amino sequence set forth in SEQ ID NO: 88.

In certain embodiments, the extracellular antigen-binding domain (e.g., an scFv) comprises a V_{H} comprising the amino acid sequence set forth in SEQ ID NO: 87, and a V_{L} comprising the amino acid sequence set forth in SEQ ID NO: 88. In certain embodiments, the extracellular antigen-binding domain is an scFv. In certain embodiments, the scFv is designated as “14A10”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}. In certain embodiments, the extracellular antigen-binding domain is an scFv comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 89. In certain embodiments, the extracellular antigen-binding domain is an scFv comprising the amino sequence set forth in SEQ ID NO: 89. An exemplary nucleotide sequence encoding the amino acid sequence of SEQ ID NO: 89 is set forth in SEQ ID NO: 91. SEQ ID NOs: 89 and 91 are provided in Table 40 below.

In certain embodiments, the variable regions within the extracellular antigen-binding domain have to be linked one after another such that at the N-terminus of the extracellular antigen-binding domain, a light chain variable region (V_{L}) is positioned. In certain embodiments, the extracellular antigen-binding domain is an scFv, the variable regions are positioned from the N- to the C-terminus: V_{L}-V_{H}. In certain embodiments, the extracellular antigen-binding domain is an scFv comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 90. In certain embodiments, the extracellular antigen-binding domain is an scFv comprising the amino sequence set forth in SEQ ID NO: 90. An exemplary nucleotide sequence encoding the amino acid sequence of SEQ ID NO: 90 is set forth in SEQ ID NO: 92. SEQ ID NOs: 90 and 92 are provided in Table 40 below.

TABLE 40

| CDRs | 1 | 2 | 3 |
|---|---|---|---|
| VH | GFIFSSYN | ISSSSSTT | ASLYSGYDYYYYYGMDV |
| | [SEQ ID NO: 81] | [SEQ ID NO: 82] | [SEQ ID NO: 83] |
| VL | QSLVHSDGNTY | KIS | MQATQFPFT |
| | [SEQ ID NO: 84] | [SEQ ID NO: 85] | [SEQ ID NO: 86] |

| | |
|---|---|
| Full | EVQMVESGGGLVQPGGSLRLSCAAPGFIFSSYNMNWVRQAPGKGLEWVSYISSSSS |
| VH | TTYYADSVKGRFTISRDNAKNSLYLQMNSLRDEDTAVYYCASLYSGYDYYYYYGMD |
| | VWGQGTTVTVSS |
| | [SEQ ID NO: 87] |
| Full | DIVMTQTPLSSPVSLGQPASISCRSSQSLVHSDGNTYLSWLQQRPGQPPRVLIYKI |
| VL | SKRFSGVPDRFSGSGAGTDFTLKISRVEAEDVGVYYCMQATQFPFTFGPGTKVDIK |
| | [SEQ ID NO: 88] |
| ScFv | EVQMVESGGGLVQPGGSLRLSCAAPGFIFSSYNMNWVRQAPGKGLEWVSYISSSSS |
| VH-VL | TTYYADSVKGRFTISRDNAKNSLYLQMNSLRDEDTAVYYCASLYSGYDYYYYYGMD |
| | VWGQGTTVTVSSGGGGSGGGGSGGGGSDIVMTQTPLSSPVSLGQPASISCRSSQSL |
| | VHSDGNTYLSWLQQRPGQPPRVLIYKISKRFSGVPDRESGSGAGTDFTLKISRVEA |
| | EDVGVYYCMQATQFPFTFGPGTKVDIK |
| | [SEQ ID NO: 89] |
| ScFv | DIVMTQTPLSSPVSLGQPASISCRSSQSLVHSDGNTYLSWLQQRPGQPPRVLIYKI |
| VL-VH | SKRFSGVPDRESGSGAGTDFTLKISRVEAEDVGVYYCMQATQFPFTFGPGTKVDIK |
| | GGGGSGGGGSGGGGSEEVQMVESGGGLVQPGGSLRLSCAAPGFIFSSYNMNWVRQA |
| | PGKGLEWVSYISSSSSTTYYADSVKGRFTISRDNAKNSLYLQMNSLRDEDTAVYYC |
| | ASLYSGYDYYYYYGMDVWGQGTTVTVSS |
| | [SEQ ID NO: 90] |
| DNA | GAAGTCCAGATGGTGGAGTCAGGAGGGGGTCTGGTCCAACCGGGGGGTAGCCTGAG |
| ScFv | GTTGTCCTGCGCGGCACCAGGATTTATCTTCTCAAGCTATAACATGAACTGGGTAC |
| VH-VL | GCCAAGCACCTGGCAAAGGACTGGAATGGGTAAGTTACATTTCTTCCAGTTCATCA |
| | ACAACTTACTATGCAGACAGTGTGAAAGGCCGGTTTACTATAAGCCGGGACAATGC |
| | TAAGAACTCACTCTATCTCCAAATGAACTCCCTCCGCGACGAGGACACAGCTGTCT |
| | ATTACTGTGCTAGTCTCTACTCTGGCTACGATTACTATTACTATTATGGAATGGAT |
| | GTGTGGGGGCAAGGCACTACTGTCACAGTCTCAAGCGGCGGTGGTGGCTCAGGCGG |
| | CGGAGGATCCGGGGGGGGGGGATCCGATATTGTTATGACGCAAACGCCCTTGTCAT |
| | CACCTGTATCATTGGGTCAGCCGGCTTCCATATCCTGCCGATCTAGTCAATCCTTG |
| | GTGCACTCAGACGGGAATACCTACCTTTCATGGCTCCAGCAGAGACCCGGGCAACC |
| | ACCGAGAGTGTTGATCTATAAAATTTCTAAACGGTTTAGCGGGGTCCCTGACAGAT |
| | TCTCCGGTTCCGGGGCTGGAACTGATTTTACACTTAAAATATCCCGAGTCGAAGCG |
| | GAAGACGTGGGGGTGTATTATTGCATGCAAGCAACTCAATTTCCCTTCACATTTGG |
| | GCCTGGTACAAAAGTGGACATCAAG |
| | [SEQ ID NO: 91] |
| DNA | GATATTGTTATGACGCAAACGCCCTTGTCATCACCTGTATCATTGGGTCAGCCGGC |
| SCFv | TTCCATATCCTGCCGATCTAGTCAATCCTTGGTGCACTCAGACGGGAATACCTACC |
| VL-VH | TTTCATGGCTCCAGCAGAGACCCGGGCAACCACCGAGAGTGTTGATCTATAAAATT |
| | TCTAAACGGTTTAGCGGGGTCCCTGACAGATTCTCCGGTTCCGGGGCTGGAACTGA |
| | TTTTACACTTAAAATATCCCGAGTCGAAGCGGAAGACGTGGGGGTGTATTATTGCA |
| | TGCAAGCAACTCAATTTCCCTTCACATTTGGGCCTGGTACAAAAGTGGACATCAAG |
| | GGCGGTGGTGGCTCAGGCGGCGGAGGATCCGGGGGGGGGGGATCCGAAGTCCAGAT |
| | GGTGGAGTCAGGAGGGGGTCTGGTCCAACCGGGGGGTAGCCTGAGGTTGTCCTGCG |
| | CGGCACCAGGATTTATCTTCTCAAGCTATAACATGAACTGGGTACGCCAAGCACCT |
| | GGCAAAGGACTGGAATGGGTAAGTTACATTTCTTCCAGTTCATCAACAACTTACTA |
| | TGCAGACAGTGTGAAAGGCCGGTTTACTATAAGCCGGGACAATGCTAAGAACTCAC |
| | TCTATCTCCAAATGAACTCCCTCCGCGACGAGGACACAGCTGTCTATTACTGTGCT |
| | AGTCTCTACTCTGGCTACGATTACTATTACTATTATGGAATGGATGTGTGGGGGCA |
| | AGGCACTACTGTCACAGTCTCAAGC |
| | [SEQ ID NO: 92] |

In certain embodiments, the extracellular antigen-binding domain (e.g., an scFv) comprises a heavy chain variable region comprising a CDR1 comprising the amino acid sequence set forth in SEQ ID NO: 93 or a conservative modification thereof, a CDR2 comprising the amino acid sequence set forth in SEQ ID NO: 94 or a conservative modification thereof, and a CDR3 comprising the amino acid sequence set forth in SEQ ID NO: 95 or a conservative modification thereof. SEQ ID NOs: 93-95 are provided in Table 41.

In certain embodiments, the extracellular antigen-binding domain (e.g., an scFv) comprises a light chain variable region comprising a CDR1 comprising the amino acid sequence set forth in SEQ ID NO: 96 or a conservative modification thereof, a CDR2 comprising the amino acid sequence set forth in SEQ ID NO: 97 or a conservative modification thereof, and a CDR3 comprising the amino acid sequence set forth in SEQ ID NO: 98 or a conservative modification thereof. SEQ ID NOs: 96-98 are provided in Table 41.

In certain embodiments, the extracellular antigen-binding domain (e.g., an scFv) comprises a heavy chain variable region comprising a CDR1 comprising the amino acid sequence set forth in SEQ ID NO: 93 or a conservative modification thereof, a CDR2 comprising the amino acid sequence set forth in SEQ ID NO: 94 or a conservative modification thereof, and a CDR3 comprising the amino acid sequence set forth in SEQ ID NO: 95 or a conservative modification thereof; and a light chain variable region comprising a CDR1 comprising the amino acid sequence set forth in SEQ ID NO: 96 or a conservative modification thereof, a CDR2 comprising the amino acid sequence set forth in SEQ ID NO: 97 or a conservative modification thereof, and a CDR3 comprising the amino acid sequence set forth in SEQ ID NO: 98 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 93, a CDR2 comprising the amino acid sequence set forth in SEQ ID NO: 94, and a CDR3 comprising the amino acid sequence set forth in SEQ ID NO: 95; and a light chain variable region comprising a CDR1 comprising the amino acid sequence set forth in SEQ ID NO: 96, a CDR2 comprising the amino acid sequence set forth in SEQ ID NO: 97, and a CDR3 comprising the amino acid sequence set forth in SEQ ID NO: 98.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 99. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 99. In certain embodiments, the extracellular antigen-binding domain comprises a V_{H} comprising the amino sequence set forth in SEQ ID NO: 99.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 100. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 100. In certain embodiments, the extracellular antigen-binding domain comprises a V_{L} comprising the amino sequence set forth in SEQ ID NO: 100.

In certain embodiments, the extracellular antigen-binding domain (e.g., an scFv) comprises a V_{H} comprising the amino acid sequence set forth in SEQ ID NO: 99, and a V_{L} comprising the amino acid sequence set forth in SEQ ID NO: 100. In certain embodiments, the extracellular antigen-binding domain is an scFv. In certain embodiments, the scFv is designated as “76H05”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}. In certain embodiments, the extracellular antigen-binding domain is an scFv comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 101. In certain embodiments, the extracellular antigen-binding domain is an scFv comprising the amino sequence set forth in SEQ ID NO: 101. An exemplary nucleotide sequence encoding the amino acid sequence of SEQ ID NO: 101 is set forth in SEQ ID NO: 103. SEQ ID NOs: 101 and 103 are provided in Table 41 below.

In certain embodiments, the variable regions within the extracellular antigen-binding domain have to be linked one after another such that at the N-terminus of the extracellular antigen-binding domain, a light chain variable region (V_{L}) is positioned. In certain embodiments, the extracellular antigen-binding domain is an scFv, the variable regions are positioned from the N- to the C-terminus: V_{L}-V_{H}. In certain embodiments, the extracellular antigen-binding domain is an scFv comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 102. In certain embodiments, the extracellular antigen-binding domain is an scFv comprising the amino sequence set forth in SEQ ID NO: 102. An exemplary nucleotide sequence encoding the amino acid sequence of SEQ ID NO: 102 is set forth in SEQ ID NO: 104. SEQ ID NOs: 102 and 104 are provided in Table 41 below.

TABLE 41

| CDRs | 1 | 2 | 3 |
|---|---|---|---|
| VH | GFSLTSYG | IWGDGST | ATSTVGYAVDY |
| | [SEQ ID NO: 93] | [SEQ ID NO: 94] | [SEQ ID NO: 95] |
| VL | ENIYSN | AAT | QHFWGSPYT |
| | [SEQ ID NO: 96] | [SEQ ID NO: 97] | [SEQ ID NO: 98] |

| | |
|---|---|
| Full | QVQLKESGPGLVAPSQSLSITCTISGFSLTSYGVHWVRQPPGKGLEWLVVIWGDGS |
| VH | TSYNSALKSRLSISKDNSKSQVFLKMNSLQTDDTAMYFCATSTVGYAVDYWGQGTS |
| | VTVSS |
| | [SEQ ID NO: 99] |
| Full | DIQMTQSPASLSVSVGETVTITCRASENIYSNLAWYQQRQGKSPQLLVYAATNLAD |
| VL | GVPSRFSGSGSGTQYSLKINSLQSEDFGSYYCQHFWGSPYTFGGGTKLEIK |
| | [SEQ ID NO: 100] |
| ScFv | QVQLKESGPGLVAPSQSLSITCTISGFSLTSYGVHWVRQPPGKGLEWLVVIWGDGS |
| VH-VL | TSYNSALKSRLSISKDNSKSQVFLKMNSLQTDDTAMYFCATSTVGYAVDYWGQGTS |
| | VTVSSGGGGSGGGGSGGGGSDIQMTQSPASLSVSVGETVTITCRASENIYSNLAWY |
| | QQRQGKSPQLLVYAATNLADGVPSRESGSGSGTQYSLKINSLQSEDEGSYYCQHEW |
| | GSPYTFGGGTKLEIK |
| | [SEQ ID NO: 101] |
| ScFv | DIQMTQSPASLSVSVGETVTITCRASENIYSNLAWYQQRQGKSPQLLVYAATNLAD |
| VL-VH | GVPSRFSGSGSGTQYSLKINSLQSEDFGSYYCQHFWGSPYTFGGGTKLEIKGGGGS |
| | GGGGSGGGGSQVQLKESGPGLVAPSQSLSITCTISGESLTSYGVHWVRQPPGKGLE |
| | WLVVIWGDGSTSYNSALKSRLSISKDNSKSQVFLKMNSLQTDDTAMYFCATSTVGY |
| | AVDYWGQGTSVTVSS |
| | [SEQ ID NO: 102] |
| DNA | CAAGTCCAGCTCAAGGAGAGCGGTCCAGGACTCGTAGCACCCTCTCAATCTTTGTC |
| SCFv | TATCACTTGTACCATAAGCGGTTTTTCCTTGACAAGTTATGGCGTACATTGGGTGA |
| VH-VL | GACAACCGCCAGGTAAAGGACTCGAATGGTTGGTTGTTATCTGGGGGGATGGGAGT |
| | ACCAGCTACAACAGCGCACTCAAGTCTCGCCTTTCAATTTCTAAGGACAACAGTAA |
| | ATCACAGGTTTTTCTGAAGATGAATAGCCTGCAAACCGACGACACGGCGATGTATT |
| | TCTGTGCGACTTCTACGGTAGGTTATGCCGTCGACTATTGGGGTCAGGGTACGTCA |
| | GTTACCGTAAGTAGCGGCGGTGGTGGCTCAGGCGGCGGAGGATCCGGGGGGGGGGG |
| | ATCCGACATCCAGATGACCCAGTCACCGGCCAGTTTGTCCGTTTCCGTAGGCGAGA |
| | CCGTAACCATCACATGCCGAGCAAGCGAGAATATCTATTCCAACCTGGCCTGGTAT |
| | CAGCAGAGACAAGGCAAAAGTCCGCAACTTTTGGTTTATGCTGCCACTAATTTGGC |
| | CGATGGGGTCCCGAGTCGATTTTCTGGCTCTGGCTCCGGGACGCAGTATTCCCTGA |
| | AGATAAACTCTTTGCAGTCAGAAGATTTTGGTTCATATTATTGTCAGCATTTCTGG |
| | GGCAGCCCGTACACATTTGGAGGTGGGACCAAATTGGAAATAAAA |
| | [SEQ ID NO: 103] |
| DNA | GACATCCAGATGACCCAGTCACCGGCCAGTTTGTCCGTTTCCGTAGGCGAGACCGT |
| ScFv | AACCATCACATGCCGAGCAAGCGAGAATATCTATTCCAACCTGGCCTGGTATCAGC |
| VL-VH | AGAGACAAGGCAAAAGTCCGCAACTTTTGGTTTATGCTGCCACTAATTTGGCCGAT |
| | GGGGTCCCGAGTCGATTTTCTGGCTCTGGCTCCGGGACGCAGTATTCCCTGAAGAT |
| | AAACTCTTTGCAGTCAGAAGATTTTGGTTCATATTATTGTCAGCATTTCTGGGGCA |
| | GCCCGTACACATTTGGAGGTGGGACCAAATTGGAAATAAAAGGCGGTGGTGGCTCA |
| | GGCGGCGGAGGATCCGGGGGGGGGGGATCCCAAGTCCAGCTCAAGGAGAGCGGTCC |
| | AGGACTCGTAGCACCCTCTCAATCTTTGTCTATCACTTGTACCATAAGCGGTTTTT |
| | CCTTGACAAGTTATGGCGTACATTGGGTGAGACAACCGCCAGGTAAAGGACTCGAA |
| | TGGTTGGTTGTTATCTGGGGGGATGGGAGTACCAGCTACAACAGCGCACTCAAGTC |
| | TCGCCTTTCAATTTCTAAGGACAACAGTAAATCACAGGTTTTTCTGAAGATGAATA |
| | GCCTGCAAACCGACGACACGGCGATGTATTTCTGTGCGACTTCTACGGTAGGTTAT |
| | GCCGTCGACTATTGGGGTCAGGGTACGTCAGTTACCGTAAGTAGC |
| | [SEQ ID NO: 104] |

In certain embodiments, the extracellular antigen-binding domain (e.g., an scFv) comprises a heavy chain variable region comprising a CDR1 comprising the amino acid sequence set forth in SEQ ID NO: 105 or a conservative modification thereof, a CDR2 comprising the amino acid sequence set forth in SEQ ID NO: 106 or a conservative modification thereof, and a CDR3 comprising the amino acid sequence set forth in SEQ ID NO: 107 or a conservative modification thereof. SEQ ID NOs: 105-107 are provided in Table 42.

In certain embodiments, the extracellular antigen-binding domain (e.g., an scFv) comprises a light chain variable region comprising a CDR1 comprising the amino acid sequence set forth in SEQ ID NO: 108 or a conservative modification thereof, a CDR2 comprising the amino acid sequence set forth in SEQ ID NO: 39 or a conservative modification thereof, and a CDR3 comprising the amino acid sequence set forth in SEQ ID NO: 109 or a conservative modification thereof. SEQ ID NOs: 108, 39, and 109 are provided in Table 42.

In certain embodiments, the extracellular antigen-binding domain (e.g., an scFv) comprises a heavy chain variable region comprising a CDR1 comprising the amino acid sequence set forth in SEQ ID NO: 105 or a conservative modification thereof, a CDR2 comprising the amino acid sequence set forth in SEQ ID NO: 106 or a conservative modification thereof, and a CDR3 comprising the amino acid sequence set forth in SEQ ID NO: 107 or a conservative modification thereof, and a light chain variable region comprising a CDR1 comprising the amino acid sequence set forth in SEQ ID NO: 108 or a conservative modification thereof, a CDR2 comprising the amino acid sequence set forth in SEQ ID NO: 39 or a conservative modification thereof, and a CDR3 comprising the amino acid sequence set forth in SEQ ID NO: 109 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 105, a CDR2 comprising the amino acid sequence set forth in SEQ ID NO: 106, and a CDR3 comprising the amino acid sequence set forth in SEQ ID NO: 107; and a light chain variable region comprising a CDR1 comprising the amino acid sequence set forth in SEQ ID NO: 108, a CDR2 comprising the amino acid sequence set forth in SEQ ID NO: 39, and a CDR3 comprising the amino acid sequence set forth in SEQ ID NO: 109.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 110. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 110. In certain embodiments, the extracellular antigen-binding domain comprises a V_{H} comprising the amino sequence set forth in SEQ ID NO: 110.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 111. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 111. In certain embodiments, the extracellular antigen-binding domain comprises a V_{L} comprising the amino sequence set forth in SEQ ID NO: 111.

In certain embodiments, the extracellular antigen-binding domain (e.g., an scFv) comprises a V_{H} comprising the amino acid sequence set forth in SEQ ID NO: 110, and a V_{L} comprising the amino acid sequence set forth in SEQ ID NO: 111. In certain embodiments, the extracellular antigen-binding domain is an scFv. In certain embodiments, the scFv is designated as “18H05”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}. In certain embodiments, the extracellular antigen-binding domain is an scFv comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 112. In certain embodiments, the extracellular antigen-binding domain is an scFv comprising the amino sequence set forth in SEQ ID NO: 112. An exemplary nucleotide sequence encoding the amino acid sequence of SEQ ID NO: 112 is set forth in SEQ ID NO: 114. SEQ ID NOs: 112 and 114 are provided in Table 42 below.

In certain embodiments, the variable regions within the extracellular antigen-binding domain have to be linked one after another such that at the N-terminus of the extracellular antigen-binding domain, a light chain variable region (V_{L}) is positioned. In certain embodiments, the extracellular antigen-binding domain is an scFv, the variable regions are positioned from the N- to the C-terminus: V_{L}-V_{H}. In certain embodiments, the extracellular antigen-binding domain is an scFv comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 113. In certain embodiments, the extracellular antigen-binding domain is an scFv comprising the amino sequence set forth in SEQ ID NO: 113. An exemplary nucleotide sequence encoding the amino acid sequence of SEQ ID NO: 113 is set forth in SEQ ID NO: 115. SEQ ID NOs: 113 and 115 are provided in Table 42 below.

TABLE 42

| CDRS | 1 | 2 | 3 |
|---|---|---|---|
| VH | GDSISSYY | IYTSGTT | ASHSSGWYDAFDI |
| | [SEQ ID NO: | [SEQ ID NO: | [SEQ ID NO: 107] |
| | 105] | 106] | |
| VL | QSVLYSSNNKNY | WAS | QQYYSTPPT |
| | [SEQ ID NO: | [SEQ ID NO: 39] | [SEQ ID NO: 109] |
| | 108] | | |

| | |
|---|---|
| Full | QVQLQESGPGLVKPSETLSLTCTVSGDSISSYYWTWIRQPAGKGLEWIGRIYTSGT |
| VH | TNYNPSLKSRVTMSVDTSKNQFSLKLSSVTAADTAVYYCASHSSGWYDAFDIWGQG |
| | TMVTVSS |
| | [SEQ ID NO: 110] |
| Full | DIVMTQSPDSLAVSLGERATINCKSSQSVLYSSNNKNYLAWYQQKPGQPPKLFIYW |
| VL | ASTRESGVPDRESGSGSGTDFTLTISSLQAEDVAVYYCQQYYSTPPTFGQGTKVEI |
| | K |
| | [SEQ ID NO: 111] |
| ScFv | QVQLQESGPGLVKPSETLSLTCTVSGDSISSYYWTWIRQPAGKGLEWIGRIYTSGT |
| VH-VL | TNYNPSLKSRVTMSVDTSKNQFSLKLSSVTAADTAVYYCASHSSGWYDAFDIWGQG |
| | TMVTVSSGGGGSGGGGSGGGGSDIVMTQSPDSLAVSLGERATINCKSSQSVLYSSN |
| | NKNYLAWYQQKPGQPPKLFIYWASTRESGVPDRESGSGSGTDFTLTISSLQAEDVA |
| | VYYCQQYYSTPPTFGQGTKVEIK |
| | [SEQ ID NO: 112] |
| ScFv | DIVMTQSPDSLAVSLGERATINCKSSQSVLYSSNNKNYLAWYQQKPGQPPKLFIYW |
| VL-VH | ASTRESGVPDRFSGSGSGTDFTLTISSLQAEDVAVYYCQQYYSTPPTFGQGTKVEI |
| | KGGGGSGGGGSGGGGSQVQLQESGPGLVKPSETLSLTCTVSGDSISSYYWTWIRQP |
| | AGKGLEWIGRIYTSGTTNYNPSLKSRVTMSVDTSKNQFSLKLSSVTAADTAVYYCA |
| | SHSSGWYDAFDIWGQGTMVTVSS |
| | [SEQ ID NO: 113] |
| DNA | CAGGTACAGCTTCAAGAAAGCGGTCCAGGACTTGTGAAGCCGAGCGAGACGCTTTC |
| ScFv | ACTGACTTGCACAGTCAGTGGGGACAGTATTAGTTCCTATTACTGGACATGGATAC |
| VH-VL | GACAACCAGCAGGGAAAGGTCTGGAATGGATTGGGCGCATTTACACCAGTGGAACT |
| | ACAAATTACAACCCATCCCTCAAATCCAGGGTTACGATGAGCGTCGACACGAGTAA |
| | GAATCAGTTTTCCCTGAAGCTCTCCAGTGTGACGGCTGCAGATACTGCAGTGTATT |
| | ATTGCGCTTCCCACTCAAGTGGTTGGTACGATGCTTTCGACATCTGGGGGCAGGGT |
| | ACTATGGTGACCGTGTCCTCAGGCGGTGGTGGCTCAGGCGGCGGAGGATCCGGGGG |
| | GGGGGGATCCGACATCGTAATGACCCAATCACCTGATTCTCTGGCTGTGAGCCTTG |
| | GTGAGAGGGCAACAATAAACTGCAAGAGCTCTCAGTCCGTACTTTATAGTAGCAAC |
| | AACAAGAACTATTTGGCGTGGTACCAGCAGAAGCCGGGTCAGCCGCCAAAGCTGTT |
| | TATATATTGGGCCTCTACCCGCGAATCTGGAGTACCGGACAGGTTTAGTGGTAGTG |
| | GCTCAGGTACGGATTTTACGCTCACCATTAGCTCTCTGCAAGCAGAGGACGTAGCC |
| | GTATATTATTGCCAGCAATATTATTCTACGCCACCCACCTTTGGTCAGGGGACGAA |
| | AGTTGAGATCAAG |
| | [SEQ ID NO: 114] |
| DNA | GACATCGTAATGACCCAATCACCTGATTCTCTGGCTGTGAGCCTTGGTGAGAGGGC |
| ScFv | AACAATAAACTGCAAGAGCTCTCAGTCCGTACTTTATAGTAGCAACAACAAGAACT |
| VL-VH | ATTTGGCGTGGTACCAGCAGAAGCCGGGTCAGCCGCCAAAGCTGTTTATATATTGG |
| | GCCTCTACCCGCGAATCTGGAGTACCGGACAGGTTTAGTGGTAGTGGCTCAGGTAC |
| | GGATTTTACGCTCACCATTAGCTCTCTGCAAGCAGAGGACGTAGCCGTATATTATT |
| | GCCAGCAATATTATTCTACGCCACCCACCTTTGGTCAGGGGACGAAAGTTGAGATC |
| | AAGGGCGGTGGTGGCTCAGGCGGCGGAGGATCCGGGGGGGGGGGATCCCAGGTACA |
| | GCTTCAAGAAAGCGGTCCAGGACTTGTGAAGCCGAGCGAGACGCTTTCACTGACTT |
| | GCACAGTCAGTGGGGACAGTATTAGTTCCTATTACTGGACATGGATACGACAACCA |
| | GCAGGGAAAGGTCTGGAATGGATTGGGCGCATTTACACCAGTGGAACTACAAATTA |
| | CAACCCATCCCTCAAATCCAGGGTTACGATGAGCGTCGACACGAGTAAGAATCAGT |
| | TTTCCCTGAAGCTCTCCAGTGTGACGGCTGCAGATACTGCAGTGTATTATTGCGCT |
| | TCCCACTCAAGTGGTTGGTACGATGCTTTCGACATCTGGGGGCAGGGTACTATGGT |
| | GACCGTGTCCTCA |
| | [SEQ ID NO: 115] |

In certain embodiments, the extracellular antigen-binding domain (e.g., an scFv) comprises a heavy chain variable region comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, and a CDR3 comprising the amino acid sequence set forth in SEQ ID NO: 118 or a conservative modification thereof. SEQ ID NOs: 116-118 are provided in Table 43.

In certain embodiments, the extracellular antigen-binding domain (e.g., an scFv) comprises a light chain variable region comprising a CDR1 comprising the amino acid sequence set forth in SEQ ID NO: 119 or a conservative modification thereof, a CDR2 comprising the amino acid sequence set forth in SEQ ID NO: 120 or a conservative modification thereof, and a CDR3 comprising the amino acid sequence set forth in SEQ ID NO: 121 or a conservative modification thereof. SEQ ID NOs: 119-121 are provided in Table 43.

In certain embodiments, the extracellular antigen-binding domain (e.g., an scFv) comprises a heavy chain variable region comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, and a CDR3 comprising the amino acid sequence set forth in SEQ ID NO: 118 or a conservative modification thereof; and a light chain variable region comprising a CDR1 comprising the amino acid sequence set forth in SEQ ID NO: 119 or a conservative modification thereof, a CDR2 comprising the amino acid sequence set forth in SEQ ID NO: 120 or a conservative modification thereof, and a CDR3 comprising the amino acid sequence set forth in SEQ ID NO: 121 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 116, a CDR2 comprising the amino acid sequence set forth in SEQ ID NO: 117, and a CDR3 comprising the amino acid sequence set forth in SEQ ID NO: 118; and a light chain variable region comprising a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 122. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 122. In certain embodiments, the extracellular antigen-binding domain comprises a V_{H} comprising the amino sequence set forth in SEQ ID NO: 122.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 123. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 123. In certain embodiments, the extracellular antigen-binding domain comprises a V_{L} comprising the amino sequence set forth in SEQ ID NO: 123.

In certain embodiments, the extracellular antigen-binding domain (e.g., an scFv) comprises a V_{H} comprising the amino acid sequence set forth in SEQ ID NO: 122, and a V_{L} comprising the amino acid sequence set forth in SEQ ID NO: 123. In certain embodiments, the extracellular antigen-binding domain is an scFv. In certain embodiments, the scFv is designated as “76H12”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}. In certain embodiments, the extracellular antigen-binding domain is an scFv comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 124. In certain embodiments, the extracellular antigen-binding domain is an scFv comprising the amino sequence set forth in SEQ ID NO: 124. An exemplary nucleotide sequence encoding the amino acid sequence of SEQ ID NO: 124 is set forth in SEQ ID NO: 126. SEQ ID NOs: 124 and 126 are provided in Table 43 below.

In certain embodiments, the variable regions within the extracellular antigen-binding domain have to be linked one after another such that at the N-terminus of the extracellular antigen-binding domain, a light chain variable region (V_{L}) is positioned. In certain embodiments, the extracellular antigen-binding domain is an scFv, the variable regions are positioned from the N- to the C-terminus: V_{L}-V_{H}. In certain embodiments, the extracellular antigen-binding domain is an scFv comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 125. In certain embodiments, the extracellular antigen-binding domain is an scFv comprising the amino sequence set forth in SEQ ID NO: 125. An exemplary nucleotide sequence encoding the amino acid sequence of SEQ ID NO: 125 is set forth in SEQ ID NO: 127. SEQ ID NOs: 125 and 127 are provided in Table 43 below.

TABLE 43

| CDRs | 1 | 2 | 3 |
|---|---|---|---|
| VH | GYTFTSYW | VYPGSGST | TRPGNYEGFAY |
| | [SEQ ID NO: | [SEQ ID NO: | [SEQ ID NO: 118] |
| | 116] | 117] | |
| VL | ESVDSYGSSF | LAS | QQNNEDPWT |
| | [SEQ ID NO: | [SEQ ID NO: | [SEQ ID NO: 121] |
| | 119] | 120] | |

| | |
|---|---|
| Full | QVQLQQPGSDLVRPGTSVKLSCKASGYTFTSYWMHWVKQRPGQGLEWIGNVYPGSG |
| VH | STKYDEKFKTKATLTVDTSSNTAYMELSSLTSEDSAVYYCTRPGNYEGFAYWGQGT |
| | LVTVSA |
| | [SEQ ID NO: 122] |
| Full | NIVLTQSPASLAVSLGQRATISCRASESVDSYGSSFMHWYQQKPGQPPKLLIYLAS |
| VL | NLESGVPARFSGSGSRTDFTLTIDPVEADDAATYYCQQNNEDPWTFGGGTKLEIK |
| | [SEQ ID NO: 123] |
| ScFv | QVQLQQPGSDLVRPGTSVKLSCKASGYTFTSYWMHWVKQRPGQGLEWIGNVYPGSG |
| VH-VL | STKYDEKFKTKATLTVDTSSNTAYMELSSLTSEDSAVYYCTRPGNYEGFAYWGQGT |
| | LVTVSAGGGGSGGGGSGGGGSNIVLTQSPASLAVSLGQRATISCRASESVDSYGSS |
| | FMHWYQQKPGQPPKLLIYLASNLESGVPARFSGSGSRTDFTLTIDPVEADDAATYY |
| | CQQNNEDPWTFGGGTKLEIK |
| | [SEQ ID NO: 124] |
| SCFv | NIVLTQSPASLAVSLGQRATISCRASESVDSYGSSFMHWYQQKPGQPPKLLIYLAS |
| VL-VH | NLESGVPARFSGSGSRTDFTLTIDPVEADDAATYYCQQNNEDPWTFGGGTKLEIKG |
| | GGGSGGGGSGGGGSQVQLQQPGSDLVRPGTSVKLSCKASGYTFTSYWMHWVKQRPG |
| | QGLEWIGNVYPGSGSTKYDEKFKTKATLTVDTSSNTAYMELSSLTSEDSAVYYCTR |
| | PGNYEGFAYWGQGTLVTVSA |
| | [SEQ ID NO: 125] |
| DNA | CAGGTGCAACTTCAGCAGCCCGGCTCTGATCTTGTGCGCCCTGGTACTTCTGTAAA |
| ScFv | GCTTAGCTGTAAAGCTTCTGGTTATACGTTCACGAGTTATTGGATGCACTGGGTAA |
| VH-VL | AGCAGCGGCCTGGACAGGGATTGGAGTGGATCGGAAACGTGTACCCTGGCAGTGGT |
| | TCTACAAAGTATGACGAGAAATTTAAGACAAAAGCAACACTCACTGTCGATACGTC |
| | TTCAAACACAGCGTACATGGAGTTGAGTTCATTGACCTCCGAGGACAGCGCTGTAT |
| | ATTACTGCACACGCCCTGGAAACTACGAAGGTTTCGCGTATTGGGGGCAGGGGACC |
| | TTGGTCACAGTGTCAGCCGGCGGTGGTGGCTCAGGCGGCGGAGGATCCGGGGGGGG |
| | GGGATCCAACATAGTCCTTACGCAAAGTCCAGCTTCACTGGCAGTATCTCTGGGTC |
| | AACGCGCTACCATTAGTTGTCGCGCAAGCGAATCCGTCGATAGCTATGGGTCTTCC |
| | TTTATGCACTGGTATCAGCAGAAGCCGGGACAGCCGCCCAAGCTGCTGATCTACCT |
| | CGCGTCCAACTTGGAGTCAGGCGTTCCCGCACGGTTTAGTGGAAGCGGAAGTAGAA |
| | CTGATTTTACTCTTACGATTGATCCTGTCGAGGCTGATGATGCTGCTACATATTAT |
| | TGTCAGCAGAATAATGAGGATCCTTGGACCTTCGGCGGGGGAACCAAATTGGAAAT |
| | TAAA |
| | [SEQ ID NO: 126] |
| DNA | AACATAGTCCTTACGCAAAGTCCAGCTTCACTGGCAGTATCTCTGGGTCAACGCGC |
| ScFv | TACCATTAGTTGTCGCGCAAGCGAATCCGTCGATAGCTATGGGTCTTCCTTTATGC |
| VL-VH | ACTGGTATCAGCAGAAGCCGGGACAGCCGCCCAAGCTGCTGATCTACCTCGCGTCC |
| | AACTTGGAGTCAGGCGTTCCCGCACGGTTTAGTGGAAGCGGAAGTAGAACTGATTT |
| | TACTCTTACGATTGATCCTGTCGAGGCTGATGATGCTGCTACATATTATTGTCAGC |
| | AGAATAATGAGGATCCTTGGACCTTCGGCGGGGGAACCAAATTGGAAATTAAAGGC |
| | GGTGGTGGCTCAGGCGGCGGAGGATCCGGGGGGGGGGGATCCCAGGTGCAACTTCA |
| | GCAGCCCGGCTCTGATCTTGTGCGCCCTGGTACTTCTGTAAAGCTTAGCTGTAAAG |
| | CTTCTGGTTATACGTTCACGAGTTATTGGATGCACTGGGTAAAGCAGCGGCCTGGA |
| | CAGGGATTGGAGTGGATCGGAAACGTGTACCCTGGCAGTGGTTCTACAAAGTATGA |
| | CGAGAAATTTAAGACAAAAGCAACACTCACTGTCGATACGTCTTCAAACACAGCGT |
| | ACATGGAGTTGAGTTCATTGACCTCCGAGGACAGCGCTGTATATTACTGCACACGC |
| | CCTGGAAACTACGAAGGTTTCGCGTATTGGGGGCAGGGGACCTTGGTCACAGTGTC |
| | AGCC |
| | [SEQ ID NO: 127] |

In certain embodiments, the extracellular antigen-binding domain (e.g., an scFv) comprises a heavy chain variable region comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, and a CDR3 comprising the amino acid sequence set forth in SEQ ID NO: 118 or a conservative modification thereof. SEQ ID NOs: 116-118 are provided in Table 44.

In certain embodiments, the extracellular antigen-binding domain (e.g., an scFv) comprises a light chain variable region comprising a CDR1 comprising the amino acid sequence set forth in SEQ ID NO: 119 or a conservative modification thereof, a CDR2 comprising the amino acid sequence set forth in SEQ ID NO: 120 or a conservative modification thereof, and a CDR3 comprising the amino acid sequence set forth in SEQ ID NO: 121 or a conservative modification thereof. SEQ ID NOs: 119-121 are provided in Table 44.

In certain embodiments, the extracellular antigen-binding domain (e.g., an scFv) comprises a heavy chain variable region comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, and a CDR3 comprising the amino acid sequence set forth in SEQ ID NO: 118 or a conservative modification thereof, and a light chain variable region comprising a CDR1 comprising the amino acid sequence set forth in SEQ ID NO: 119 or a conservative modification thereof, a CDR2 comprising the amino acid sequence set forth in SEQ ID NO: 120 or a conservative modification thereof, and a CDR3 comprising the amino acid sequence set forth in SEQ ID NO: 121 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 116, a CDR2 comprising the amino acid sequence set forth in SEQ ID NO: 117, and a CDR3 comprising the amino acid sequence set forth in SEQ ID NO: 118; and a light chain variable region comprising a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 128. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 128. In certain embodiments, the extracellular antigen-binding domain comprises a V_{H} comprising the amino sequence set forth in SEQ ID NO: 128.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 129. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 129. In certain embodiments, the extracellular antigen-binding domain comprises a V_{L} comprising the amino sequence set forth in SEQ ID NO: 129.

In certain embodiments, the extracellular antigen-binding domain (e.g., an scFv) comprises a V_{H} comprising the amino acid sequence set forth in SEQ ID NO: 128, and a V_{L} comprising the amino acid sequence set forth in SEQ ID NO: 129. In certain embodiments, the extracellular antigen-binding domain is an scFv. In certain embodiments, the scFv is designated as “76H12v2”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 44

| CDRS | 1 | 2 | 3 |
|---|---|---|---|
| VH | GYTFTSYW | VYPGSGST | TRPGNYEGFAY |
| | [SEQ ID NO: | [SEQ ID NO: | [SEQ ID NO: 118] |
| | 116] | 117] | |
| VL | ESVDSYGSSF | LAS | QQNNEDPWT |
| | [SEQ ID NO: | [SEQ ID NO: | [SEQ ID NO: 121] |
| | 119] | 120] | |

| | |
|---|---|
| Full | QVQLVQSGAEVKKPGASVKVSCKASGYTFTSYWMHWVRQAPGQGLEWMGNVYPGSG |
| | STKYAQKFQGRVTMTRDTSTSTVYMELSSLRSEDTAVYYCARPGNYEGFAYWGQGT |
| | LVTVSS |
| VH | [SEQ ID NO: 128] |
| Full | NIQLTQSPSSLSASVGDRVTITCRASESVDSYGSSFMHWYQQKPGKPPKLLIYLAS |
| VL | NLESGVPSRFSGSGSGTDFTLTISSLQPEDFATYYCQQNNEDPWTFGQGTKLEIK |
| | [SEQ ID NO: 129] |

In certain embodiments, the extracellular antigen-binding domain (e.g., an scFv) comprises a heavy chain variable region comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, and a CDR3 comprising the amino acid sequence set forth in SEQ ID NO: 118 or a conservative modification thereof. SEQ ID NOs: 116-118 are provided in Table 45.

In certain embodiments, the extracellular antigen-binding domain (e.g., an scFv) comprises a light chain variable region comprising a CDR1 comprising the amino acid sequence set forth in SEQ ID NO: 119 or a conservative modification thereof, a CDR2 comprising the amino acid sequence set forth in SEQ ID NO: 120 or a conservative modification thereof, and a CDR3 comprising the amino acid sequence set forth in SEQ ID NO: 121 or a conservative modification thereof. SEQ ID NOs: 119-121 are provided in Table 45.

In certain embodiments, the extracellular antigen-binding domain (e.g., an scFv) comprises a heavy chain variable region comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, and a CDR3 comprising the amino acid sequence set forth in SEQ ID NO: 118 or a conservative modification thereof, and a light chain variable region comprising a CDR1 comprising the amino acid sequence set forth in SEQ ID NO: 119 or a conservative modification thereof, a CDR2 comprising the amino acid sequence set forth in SEQ ID NO: 120 or a conservative modification thereof, and a CDR3 comprising the amino acid sequence set forth in SEQ ID NO: 121 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 116, a CDR2 comprising the amino acid sequence set forth in SEQ ID NO: 117, and a CDR3 comprising the amino acid sequence set forth in SEQ ID NO: 118; and a light chain variable region comprising a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 130. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 130. In certain embodiments, the extracellular antigen-binding domain comprises a V_{H} comprising the amino sequence set forth in SEQ ID NO: 130.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 131. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 131. In certain embodiments, the extracellular antigen-binding domain comprises a V_{L} comprising the amino sequence set forth in SEQ ID NO: 131.

In certain embodiments, the extracellular antigen-binding domain (e.g., an scFv) comprises a V_{H} comprising the amino acid sequence set forth in SEQ ID NO: 130, and a V_{L} comprising the amino acid sequence set forth in SEQ ID NO: 131. In certain embodiments, the extracellular antigen-binding domain is an scFv. In certain embodiments, the scFv is designated as “76H12v3”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 45

| CDRs | 1 | 2 | 3 |
|---|---|---|---|
| VH | GYTFTSYW | VYPGSGST | TRPGNYEGFAY |
| | [SEQ ID NO: | [SEQ ID NO: | [SEQ ID NO: 118] |
| | 116] | 117] | |
| VL | ESVDSYGSSF | LAS | QQNNEDPWT |
| | [SEQ ID NO: | [SEQ ID NO: | [SEQ ID NO: 121] |
| | 119] | 120] | |

| | |
|---|---|
| Full | QVQLVQSGAEVKKPGASVKVSCKASGYTFTSYWMHWVRQAPGQGLEWIGNVYPGSG |
| VH | STKYAQKFQGRATLTVDTSTSTVYMELSSLRSEDTAVYYCARPGNYEGFAYWGQGT |
| | LVTVSS |
| | [SEQ ID NO: 130] |
| Full | DIQLTQSPSSLSASVGDRVTITCRASESVDSYGSSFMHWYQQKPGKPPKLLIYLAS |
| VL | NLESGVPSRFSGSGSGTDFTLTISSLQPEDFATYYCQQNNEDPWTFGQGTKLEIK |
| | [SEQ ID NO: 131] |

In certain embodiments, the extracellular antigen-binding domain (e.g., an scFv) comprises a heavy chain variable region comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, and a CDR3 comprising the amino acid sequence set forth in SEQ ID NO: 118 or a conservative modification thereof. SEQ ID NOs: 116-118 are provided in Table 46.

In certain embodiments, the extracellular antigen-binding domain (e.g., an scFv) comprises a light chain variable region comprising a CDR1 comprising the amino acid sequence set forth in SEQ ID NO: 119 or a conservative modification thereof, a CDR2 comprising the amino acid sequence set forth in SEQ ID NO: 120 or a conservative modification thereof, and a CDR3 comprising the amino acid sequence set forth in SEQ ID NO: 121 or a conservative modification thereof. SEQ ID NOs: 119-121 are provided in Table 46.

In certain embodiments, the extracellular antigen-binding domain (e.g., an scFv) comprises a heavy chain variable region comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, and a CDR3 comprising the amino acid sequence set forth in SEQ ID NO: 118 or a conservative modification thereof, and a light chain variable region comprising a CDR1 comprising the amino acid sequence set forth in SEQ ID NO: 119 or a conservative modification thereof, a CDR2 comprising the amino acid sequence set forth in SEQ ID NO: 120 or a conservative modification thereof, and a CDR3 comprising the amino acid sequence set forth in SEQ ID NO: 121 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 116, a CDR2 comprising the amino acid sequence set forth in SEQ ID NO: 117, and a CDR3 comprising the amino acid sequence set forth in SEQ ID NO: 118; and a light chain variable region comprising a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 130. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 130. In certain embodiments, the extracellular antigen-binding domain comprises a V_{H} comprising the amino sequence set forth in SEQ ID NO: 130.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 132. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 132. In certain embodiments, the extracellular antigen-binding domain comprises a V_{L} comprising the amino sequence set forth in SEQ ID NO: 132.

In certain embodiments, the extracellular antigen-binding domain (e.g., an scFv) comprises a V_{H} comprising the amino acid sequence set forth in SEQ ID NO: 130, and a V_{L} comprising the amino acid sequence set forth in SEQ ID NO: 132. In certain embodiments, the extracellular antigen-binding domain is an scFv. In certain embodiments, the scFv is designated as “76H12v4”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 46

| CDRs | 1 | 2 | 3 |
|---|---|---|---|
| VH | GYTFTSYW | VYPGSGST | TRPGNYEGFAY |
| | [SEQ ID NO: | [SEQ ID NO: | [SEQ ID NO: 118] |
| | 116] | 117] | |
| VL | ESVDSYGSSF | LAS | QQNNEDPWT |
| | [SEQ ID NO: | [SEQ ID NO: | [SEQ ID NO: 121] |
| | 119] | 120] | |

| | |
|---|---|
| Full | QVQLVQSGAEVKKPGASVKVSCKASGYTFTSYWMHWVRQAPGQGLEWIGNVYPGSG |
| VH | STKYAQKFQGRATLTVDTSTSTVYMELSSLRSEDTAVYYCARPGNYEGFAYWGQGT |
| | LVTVSS |
| | [SEQ ID NO: 130] |
| Full | DIQLTQSPSSLSASVGDRVTITCRASESVDSYGSSFMHWYQQKPGKPPKLLIYLAS |
| VL | NLESGVPSRFSGSGSRTDFTLTISSLQPEDFATYYCQQNNEDPWTFGQGTKLEIK |
| | [SEQ ID NO: 132] |

In certain embodiments, the extracellular antigen-binding domain (e.g., an scFv) comprises a heavy chain variable region comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, and a CDR3 comprising the amino acid sequence set forth in SEQ ID NO: 118 or a conservative modification thereof. SEQ ID NOs: 116-118 are provided in Table 47.

In certain embodiments, the extracellular antigen-binding domain (e.g., an scFv) comprises a light chain variable region comprising a CDR1 comprising the amino acid sequence set forth in SEQ ID NO: 119 or a conservative modification thereof, a CDR2 comprising the amino acid sequence set forth in SEQ ID NO: 120 or a conservative modification thereof, and a CDR3 comprising the amino acid sequence set forth in SEQ ID NO: 121 or a conservative modification thereof. SEQ ID NOs: 119-121 are provided in Table 47.

In certain embodiments, the extracellular antigen-binding domain (e.g., an scFv) comprises a heavy chain variable region comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, and a CDR3 comprising the amino acid sequence set forth in SEQ ID NO: 118 or a conservative modification thereof, and a light chain variable region comprising a CDR1 comprising the amino acid sequence set forth in SEQ ID NO: 119 or a conservative modification thereof, a CDR2 comprising the amino acid sequence set forth in SEQ ID NO: 120 or a conservative modification thereof, and a CDR3 comprising the amino acid sequence set forth in SEQ ID NO: 121 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 116, a CDR2 comprising the amino acid sequence set forth in SEQ ID NO: 117, and a CDR3 comprising the amino acid sequence set forth in SEQ ID NO: 118; and a light chain variable region comprising a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 130. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 130. In certain embodiments, the extracellular antigen-binding domain comprises a V_{H} comprising the amino sequence set forth in SEQ ID NO: 130.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 133. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 133. In certain embodiments, the extracellular antigen-binding domain comprises a V_{L} comprising the amino sequence set forth in SEQ ID NO: 133.

In certain embodiments, the extracellular antigen-binding domain (e.g., an scFv) comprises a V_{H} comprising the amino acid sequence set forth in SEQ ID NO: 130, and a V_{L} comprising the amino acid sequence set forth in SEQ ID NO: 133. In certain embodiments, the extracellular antigen-binding domain is an scFv. In certain embodiments, the scFv is designated as “76H12v5”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 47

| CDRs | 1 | 2 | 3 |
|---|---|---|---|
| VH | GYTFTSYW | VYPGSGST | TRPGNYEGFAY |
| | [SEQ ID NO: | [SEQ ID NO: | [SEQ ID NO: 118] |
| | 116] | 117] | |
| VL | ESVDSYGSSF | LAS | QQNNEDPWT |
| | [SEQ ID NO: | [SEQ ID NO: | [SEQ ID NO: 121] |
| | 119] | 120] | |

| | |
|---|---|
| Full | QVQLVQSGAEVKKPGASVKVSCKASGYTFTSYWMHWVRQAPGQGLEWIGNVYPGSG |
| VH | STKYAQKFQGRATLTVDTSTSTVYMELSSLRSEDTAVYYCARPGNYEGFAYWGQGT |
| | LVTVSS |
| | [SEQ ID NO: 130] |
| Full | NIQLTQSPSSLSASVGDRVTITCRASESVDSYGSSFMHWYQQKPGKPPKLLIYLAS |
| VL | NLESGVPSRFSGSGSGTDFTLTISSLQPEDFATYYCQQNNEDPWTFGQGTKLEIK |
| | [SEQ ID NO: 133] |

In certain embodiments, the extracellular antigen-binding domain (e.g., an scFv) comprises a heavy chain variable region comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, and a CDR3 comprising the amino acid sequence set forth in SEQ ID NO: 118 or a conservative modification thereof. SEQ ID NOs: 116-118 are provided in Table 48.

In certain embodiments, the extracellular antigen-binding domain (e.g., an scFv) comprises a light chain variable region comprising a CDR1 comprising the amino acid sequence set forth in SEQ ID NO: 119 or a conservative modification thereof, a CDR2 comprising the amino acid sequence set forth in SEQ ID NO: 120 or a conservative modification thereof, and a CDR3 comprising the amino acid sequence set forth in SEQ ID NO: 121 or a conservative modification thereof. SEQ ID NOs: 119-121 are provided in Table 48.

In certain embodiments, the extracellular antigen-binding domain (e.g., an scFv) comprises a heavy chain variable region comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, and a CDR3 comprising the amino acid sequence set forth in SEQ ID NO: 118 or a conservative modification thereof, and a light chain variable region comprising a CDR1 comprising the amino acid sequence set forth in SEQ ID NO: 119 or a conservative modification thereof, a CDR2 comprising the amino acid sequence set forth in SEQ ID NO: 120 or a conservative modification thereof, and a CDR3 comprising the amino acid sequence set forth in SEQ ID NO: 121 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 116, a CDR2 comprising the amino acid sequence set forth in SEQ ID NO: 117, and a CDR3 comprising the amino acid sequence set forth in SEQ ID NO: 118; and a light chain variable region comprising a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 134. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 134. In certain embodiments, the extracellular antigen-binding domain comprises a V_{H} comprising the amino sequence set forth in SEQ ID NO: 134.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 131. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 131. In certain embodiments, the extracellular antigen-binding domain comprises a V_{L} comprising the amino sequence set forth in SEQ ID NO: 131.

In certain embodiments, the extracellular antigen-binding domain (e.g., an scFv) comprises a V_{H} comprising the amino acid sequence set forth in SEQ ID NO: 134, and a V_{L} comprising the amino acid sequence set forth in SEQ ID NO: 131. In certain embodiments, the extracellular antigen-binding domain is an scFv. In certain embodiments, the scFv is designated as “76H12v6”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 48

| CDRs | 1 | 2 | 3 |
|---|---|---|---|
| VH | GYTFTSYW | VYPGSGST | TRPGNYEGFAY |
| | [SEQ ID NO: | [SEQ ID NO: | [SEQ ID NO: 118] |
| | 116] | 117] | |
| VL | ESVDSYGSSF | LAS | QQNNEDPWT |
| | [SEQ ID NO: | [SEQ ID NO: | [SEQ ID NO: 121] |
| | 119] | 120] | |

| | |
|---|---|
| Full | QVQLVQSGAEVKKPGASVKVSCKASGYTFTSYWMHWVRQAPGQGLEWIGNVYPGSG |
| VH | STKYAQKFQGRATLTVDTSTSTAYMELSSLRSEDTAVYYCTRPGNYEGFAYWGQGT |
| | LVTVSS |
| | [SEQ ID NO: 134] |
| Full | DIQLTQSPSSLSASVGDRVTITCRASESVDSYGSSFMHWYQQKPGKPPKLLIYLAS |
| VL | NLESGVPSRFSGSGSGTDFTLTISSLQPEDFATYYCQQNNEDPWTFGQGTKLEIK |
| | [SEQ ID NO: 131] |

In certain embodiments, the extracellular antigen-binding domain (e.g., an scFv) comprises a heavy chain variable region comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, and a CDR3 comprising the amino acid sequence set forth in SEQ ID NO: 118 or a conservative modification thereof. SEQ ID NOs: 116-118 are provided in Table 49.

In certain embodiments, the extracellular antigen-binding domain (e.g., an scFv) comprises a light chain variable region comprising a CDR1 comprising the amino acid sequence set forth in SEQ ID NO: 119 or a conservative modification thereof, a CDR2 comprising the amino acid sequence set forth in SEQ ID NO: 120 or a conservative modification thereof, and a CDR3 comprising the amino acid sequence set forth in SEQ ID NO: 121 or a conservative modification thereof. SEQ ID NOs: 119-121 are provided in Table 49.

In certain embodiments, the extracellular antigen-binding domain (e.g., an scFv) comprises a heavy chain variable region comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, and a CDR3 comprising the amino acid sequence set forth in SEQ ID NO: 118 or a conservative modification thereof, and a light chain variable region comprising a CDR1 comprising the amino acid sequence set forth in SEQ ID NO: 119 or a conservative modification thereof, a CDR2 comprising the amino acid sequence set forth in SEQ ID NO: 120 or a conservative modification thereof, and a CDR3 comprising the amino acid sequence set forth in SEQ ID NO: 121 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 116, a CDR2 comprising the amino acid sequence set forth in SEQ ID NO: 117, and a CDR3 comprising the amino acid sequence set forth in SEQ ID NO: 118; and a light chain variable region comprising a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 134. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 134. In certain embodiments, the extracellular antigen-binding domain comprises a V_{H} comprising the amino sequence set forth in SEQ ID NO: 134.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 132. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 132. In certain embodiments, the extracellular antigen-binding domain comprises a V_{L} comprising the amino sequence set forth in SEQ ID NO: 132.

In certain embodiments, the extracellular antigen-binding domain (e.g., an scFv) comprises a V_{H} comprising the amino acid sequence set forth in SEQ ID NO: 134, and a V_{L} comprising the amino acid sequence set forth in SEQ ID NO: 132. In certain embodiments, the extracellular antigen-binding domain is an scFv. In certain embodiments, the scFv is designated as “76H12v7”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 49

| CDRs | 1 | 2 | 3 |
|---|---|---|---|
| VH | GYTFTSYW | VYPGSGST | TRPGNYEGFAY |
| | [SEQ ID NO: | [SEQ ID NO: | [SEQ ID NO: 118] |
| | 116] | 117] | |
| VL | ESVDSYGSSF | LAS | QQNNEDPWT |
| | [SEQ ID NO: | [SEQ ID NO: | [SEQ ID NO: 121] |
| | 119] | 120] | |

| | |
|---|---|
| Full | QVQLVQSGAEVKKPGASVKVSCKASGYTFTSYWMHWVRQAPGQGLEWIGNVYPGSG |
| VH | STKYAQKFQGRATLTVDTSTSTAYMELSSLRSEDTAVYYCTRPGNYEGFAYWGQGT |
| | LVTVSS |
| | [SEQ ID NO: 134] |
| Full | DIQLTQSPSSLSASVGDRVTITCRASESVDSYGSSFMHWYQQKPGKPPKLLIYLAS |
| VL | NLESGVPSRFSGSGSRTDFTLTISSLQPEDFATYYCQQNNEDPWTFGQGTKLEIK |
| | [SEQ ID NO: 132] |

In certain embodiments, the extracellular antigen-binding domain (e.g., an scFv) comprises a heavy chain variable region comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, and a CDR3 comprising the amino acid sequence set forth in SEQ ID NO: 118 or a conservative modification thereof. SEQ ID NOs: 116-118 are provided in Table 50.

In certain embodiments, the extracellular antigen-binding domain (e.g., an scFv) comprises a light chain variable region comprising a CDR1 comprising the amino acid sequence set forth in SEQ ID NO: 119 or a conservative modification thereof, a CDR2 comprising the amino acid sequence set forth in SEQ ID NO: 120 or a conservative modification thereof, and a CDR3 comprising the amino acid sequence set forth in SEQ ID NO: 121 or a conservative modification thereof. SEQ ID NOs: 119-121 are provided in Table 50.

In certain embodiments, the extracellular antigen-binding domain (e.g., an scFv) comprises a heavy chain variable region comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, and a CDR3 comprising the amino acid sequence set forth in SEQ ID NO: 118 or a conservative modification thereof, and a light chain variable region comprising a CDR1 comprising the amino acid sequence set forth in SEQ ID NO: 119 or a conservative modification thereof, a CDR2 comprising the amino acid sequence set forth in SEQ ID NO: 120 or a conservative modification thereof, and a CDR3 comprising the amino acid sequence set forth in SEQ ID NO: 121 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 116, a CDR2 comprising the amino acid sequence set forth in SEQ ID NO: 117, and a CDR3 comprising the amino acid sequence set forth in SEQ ID NO: 118; and a light chain variable region comprising a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 134. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90°/, about 91%, about 92%, about 93%, about 94%, about 95%, about 96%, about 97%, about 98%, about 99% or about 100% homologous or identical to SEQ ID NO: 134. In certain embodiments, the extracellular antigen-binding domain comprises a V_{H} comprising the amino sequence set forth in SEQ ID NO: 134.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 133. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 133. In certain embodiments, the extracellular antigen-binding domain comprises a V_{L} comprising the amino sequence set forth in SEQ ID NO: 133.

In certain embodiments, the extracellular antigen-binding domain (e.g., an scFv) comprises a V_{H} comprising the amino acid sequence set forth in SEQ ID NO: 134, and a V_{L} comprising the amino acid sequence set forth in SEQ ID NO: 133. In certain embodiments, the extracellular antigen-binding domain is an scFv. In certain embodiments, the scFv is designated as “76H12v8”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 50

| CDRs | 1 | 2 | 3 |
|---|---|---|---|
| VH | GYTFTSYW | VYPGSGST | TRPGNYEGFAY |
| | [SEQ ID NO: | [SEQ ID NO: | [SEQ ID NO: 118] |
| | 116] | 117] | |
| VL | ESVDSYGSSF | LAS | QQNNEDPWT |
| | [SEQ ID NO: | [SEQ ID NO: | [SEQ ID NO: 121] |
| | 119] | 120] | |

| | |
|---|---|
| Full | QVQLVQSGAEVKKPGASVKVSCKASGYTFTSYWMHWVRQAPGQGLEWIGNVYPGSG |
| VH | STKYAQKFQGRATLTVDTSTSTAYMELSSLRSEDTAVYYCTRPGNYEGFAYWGQGT |
| | LVTVSS |
| | [SEQ ID NO: 134] |
| Full | NIQLTQSPSSLSASVGDRVTITCRASESVDSYGSSFMHWYQQKPGKPPKLLIYLAS |
| VL | NLESGVPSRFSGSGSGTDFTLTISSLQPEDFATYYCQQNNEDPWTFGQGTKLEIK |
| | [SEQ ID NO: 133] |

In certain embodiments, the extracellular antigen-binding domain (e.g., an scFv) comprises a heavy chain variable region comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, and a CDR3 comprising the amino acid sequence set forth in SEQ ID NO: 118 or a conservative modification thereof. SEQ ID NOs: 116-118 are provided in Table 51.

In certain embodiments, the extracellular antigen-binding domain (e.g., an scFv) comprises a light chain variable region comprising a CDR1 comprising the amino acid sequence set forth in SEQ ID NO: 119 or a conservative modification thereof, a CDR2 comprising the amino acid sequence set forth in SEQ ID NO: 120 or a conservative modification thereof, and a CDR3 comprising the amino acid sequence set forth in SEQ ID NO: 121 or a conservative modification thereof. SEQ ID NOs: 119-121 are provided in Table 51.

In certain embodiments, the extracellular antigen-binding domain (e.g., an scFv) comprises a heavy chain variable region comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, and a CDR3 comprising the amino acid sequence set forth in SEQ ID NO: 118 or a conservative modification thereof, and a light chain variable region comprising a CDR1 comprising the amino acid sequence set forth in SEQ ID NO: 119 or a conservative modification thereof, a CDR2 comprising the amino acid sequence set forth in SEQ ID NO: 120 or a conservative modification thereof, and a CDR3 comprising the amino acid sequence set forth in SEQ ID NO: 121 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 116, a CDR2 comprising the amino acid sequence set forth in SEQ ID NO: 117, and a CDR3 comprising the amino acid sequence set forth in SEQ ID NO: 118; and a light chain variable region comprising a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 135. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 135. In certain embodiments, the extracellular antigen-binding domain comprises a V_{H} comprising the amino sequence set forth in SEQ ID NO: 135.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 131. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 131. In certain embodiments, the extracellular antigen-binding domain comprises a V_{L} comprising the amino sequence set forth in SEQ ID NO: 131.

In certain embodiments, the extracellular antigen-binding domain (e.g., an scFv) comprises a V_{H} comprising the amino acid sequence set forth in SEQ ID NO: 135, and a V_{L} comprising the amino acid sequence set forth in SEQ ID NO: 131. In certain embodiments, the extracellular antigen-binding domain is an scFv. In certain embodiments, the scFv is designated as “76H12v9”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 51

| CDRs | 1 | 2 | 3 |
|---|---|---|---|
| VH | GYTFTSYW | VYPGSGST | TRPGNYEGFAY |
| | [SEQ ID NO: | [SEQ ID NO: | [SEQ ID NO: 118] |
| | 116] | 117] | |
| VL | ESVDSYGSSF | LAS | QQNNEDPWT |
| | [SEQ ID NO: | [SEQ ID NO: | [SEQ ID NO: 121] |
| | 119] | 120] | |

| | |
|---|---|
| Full | QVQLVQSGAEVKKPGASVKVSCKASGYTFTSYWMHWVRQAPGQGLEWIGNVYPGSG |
| VH | STKYAQKFQGRATLTVDTSTNTAYMELSSLRSEDTAVYYCTRPGNYEGFAYWGQGT |
| | LVTVSS |
| | [SEQ ID NO: 135] |
| Full | DIQLTQSPSSLSASVGDRVTITCRASESVDSYGSSFMHWYQQKPGKPPKLLIYLAS |
| VL | NLESGVPSRFSGSGSGTDFTLTISSLQPEDFATYYCQQNNEDPWTFGQGTKLEIK |
| | [SEQ ID NO: 131] |

In certain embodiments, the extracellular antigen-binding domain (e.g., an scFv) comprises a heavy chain variable region comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, and a CDR3 comprising the amino acid sequence set forth in SEQ ID NO: 118 or a conservative modification thereof. SEQ ID NOs: 116-118 are provided in Table 52.

In certain embodiments, the extracellular antigen-binding domain (e.g., an scFv) comprises a light chain variable region comprising a CDR1 comprising the amino acid sequence set forth in SEQ ID NO: 119 or a conservative modification thereof, a CDR2 comprising the amino acid sequence set forth in SEQ ID NO: 120 or a conservative modification thereof, and a CDR3 comprising the amino acid sequence set forth in SEQ ID NO: 121 or a conservative modification thereof. SEQ ID NOs: 119-121 are provided in Table 52.

In certain embodiments, the extracellular antigen-binding domain (e.g., an scFv) comprises a heavy chain variable region comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, and a CDR3 comprising the amino acid sequence set forth in SEQ ID NO: 118 or a conservative modification thereof; and a light chain variable region comprising a CDR1 comprising the amino acid sequence set forth in SEQ ID NO: 119 or a conservative modification thereof, a CDR2 comprising the amino acid sequence set forth in SEQ ID NO: 120 or a conservative modification thereof, and a CDR3 comprising the amino acid sequence set forth in SEQ ID NO: 121 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 116, a CDR2 comprising the amino acid sequence set forth in SEQ ID NO: 117, and a CDR3 comprising the amino acid sequence set forth in SEQ ID NO: 118; and a light chain variable region comprising a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 135. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 135. In certain embodiments, the extracellular antigen-binding domain comprises a V_{H} comprising the amino sequence set forth in SEQ ID NO: 135.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 132. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 132. In certain embodiments, the extracellular antigen-binding domain comprises a V_{L} comprising the amino sequence set forth in SEQ ID NO: 132.

In certain embodiments, the extracellular antigen-binding domain (e.g., an scFv) comprises a V_{H} comprising the amino acid sequence set forth in SEQ ID NO: 135, and a V_{L} comprising the amino acid sequence set forth in SEQ ID NO: 132. In certain embodiments, the extracellular antigen-binding domain is an scFv. In certain embodiments, the scFv is designated as “76H12v10”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 52

| CDRs | 1 | 2 | 3 |
|---|---|---|---|
| VH | GYTFTSYW | VYPGSGST | TRPGNYEGFAY |
| | [SEQ ID NO: | [SEQ ID NO: | [SEQ ID NO: 118] |
| | 116] | 117] | |
| VL | ESVDSYGSSF | LAS | QQNNEDPWT |
| | [SEQ ID NO: | [SEQ ID NO: | [SEQ ID NO: 121] |
| | 119] | 120] | |

| | |
|---|---|
| Full | QVQLVQSGAEVKKPGASVKVSCKASGYTFTSYWMHWVRQAPGQGLEWIGNVYPGSG |
| VH | STKYAQKFQGRATLTVDTSTNTAYMELSSLRSEDTAVYYCTRPGNYEGFAYWGQGT |
| | LVTVSS |
| | [SEQ ID NO: 135] |
| Full | DIQLTQSPSSLSASVGDRVTITCRASESVDSYGSSFMHWYQQKPGKPPKLLIYLAS |
| VL | NLESGVPSRFSGSGSRTDFTLTISSLQPEDFATYYCQQNNEDPWTFGQGTKLEIK |
| | [SEQ ID NO: 132] |

In certain embodiments, the extracellular antigen-binding domain (e.g., an scFv) comprises a heavy chain variable region comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, and a CDR3 comprising the amino acid sequence set forth in SEQ ID NO: 118 or a conservative modification thereof. SEQ ID NOs: 116-118 are provided in Table 53.

In certain embodiments, the extracellular antigen-binding domain (e.g., an scFv) comprises a light chain variable region comprising a CDR1 comprising the amino acid sequence set forth in SEQ ID NO: 119 or a conservative modification thereof, a CDR2 comprising the amino acid sequence set forth in SEQ ID NO: 120 or a conservative modification thereof, and a CDR3 comprising the amino acid sequence set forth in SEQ ID NO: 121 or a conservative modification thereof. SEQ ID NOs: 119-121 are provided in Table 53.

In certain embodiments, the extracellular antigen-binding domain (e.g., an scFv) comprises a heavy chain variable region comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, and a CDR3 comprising the amino acid sequence set forth in SEQ ID NO: 118 or a conservative modification thereof, and a light chain variable region comprising a CDR1 comprising the amino acid sequence set forth in SEQ ID NO: 119 or a conservative modification thereof, a CDR2 comprising the amino acid sequence set forth in SEQ ID NO: 120 or a conservative modification thereof, and a CDR3 comprising the amino acid sequence set forth in SEQ ID NO: 121 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 116, a CDR2 comprising the amino acid sequence set forth in SEQ ID NO: 117, and a CDR3 comprising the amino acid sequence set forth in SEQ ID NO: 118; and a light chain variable region comprising a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 135. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 135. In certain embodiments, the extracellular antigen-binding domain comprises a V_{H} comprising the amino sequence set forth in SEQ ID NO: 135.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 133. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 133. In certain embodiments, the extracellular antigen-binding domain comprises a V_{L} comprising the amino sequence set forth in SEQ ID NO: 133.

In certain embodiments, the extracellular antigen-binding domain (e.g., an scFv) comprises a V_{H} comprising the amino acid sequence set forth in SEQ ID NO: 135, and a V_{L} comprising the amino acid sequence set forth in SEQ ID NO: 133. In certain embodiments, the extracellular antigen-binding domain is an scFv. In certain embodiments, the scFv is designated as “76H12v11”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 53

| CDRS | 1 | 2 | 3 |
|---|---|---|---|
| VH | GYTFTSYW | VYPGSGST | TRPGNYEGFAY |
| | [SEQ ID NO: | [SEQ ID NO: | [SEQ ID NO: 118] |
| | 116] | 117] | |
| VL | ESVDSYGSSF | LAS | QQNNEDPWT |
| | [SEQ ID NO: | [SEQ ID NO: | [SEQ ID NO: 121] |
| | 119] | 120] | |

| | |
|---|---|
| Full | QVQLVQSGAEVKKPGASVKVSCKASGYTFTSYWMHWVRQAPGQGLEWIGNVYPGSG |
| VH | STKYAQKFQGRATLTVDTSTNTAYMELSSLRSEDTAVYYCTRPGNYEGFAYWGQGT |
| | LVTVSS |
| | [SEQ ID NO: 135] |
| Full | NIQLTQSPSSLSASVGDRVTITCRASESVDSYGSSFMHWYQQKPGKPPKLLIYLAS |
| VL | NLESGVPSRFSGSGSGTDFTLTISSLQPEDFATYYCQQNNEDPWTFGQGTKLEIK |
| | [SEQ ID NO: 133] |

In certain embodiments, the extracellular antigen-binding domain (e.g., an scFv) comprises a heavy chain variable region comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, and a CDR3 comprising the amino acid sequence set forth in SEQ ID NO: 118 or a conservative modification thereof. SEQ ID NOs: 116-118 are provided in Table 54.

In certain embodiments, the extracellular antigen-binding domain (e.g., an scFv) comprises a light chain variable region comprising a CDR1 comprising the amino acid sequence set forth in SEQ ID NO: 119 or a conservative modification thereof, a CDR2 comprising the amino acid sequence set forth in SEQ ID NO: 120 or a conservative modification thereof, and a CDR3 comprising the amino acid sequence set forth in SEQ ID NO: 121 or a conservative modification thereof. SEQ ID NOs: 119-121 are provided in Table 54.

In certain embodiments, the extracellular antigen-binding domain (e.g., an scFv) comprises a heavy chain variable region comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, and a CDR3 comprising the amino acid sequence set forth in SEQ ID NO: 118 or a conservative modification thereof, and a light chain variable region comprising a CDR1 comprising the amino acid sequence set forth in SEQ ID NO: 119 or a conservative modification thereof, a CDR2 comprising the amino acid sequence set forth in SEQ ID NO: 120 or a conservative modification thereof, and a CDR3 comprising the amino acid sequence set forth in SEQ ID NO: 121 or a conservative modification thereof.

In certain embodiments, the extracellular antigen-binding domain (e.g., an scFv) comprises a heavy chain variable region comprising a CDR1 comprising the amino acid sequence set forth in SEQ ID NO: 116, a CDR2 comprising the amino acid sequence set forth in SEQ ID NO: 117, and a CDR3 comprising the amino acid sequence set forth in SEQ ID NO: 118; and a light chain variable region comprising a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121.

In certain embodiments, the extracellular antigen-binding domain (e.g., an scFv) comprises a heavy chain variable region (V_{H}) comprising an amino acid sequence that is at least about 80% (e.g., at least about 85%, at least about 90%, or at least about 95%) homologous or identical to the amino sequence set forth in SEQ ID NO: 135. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90°/, about 91%, about 92%, about 93%, about 94%, about 95%, about 96%, about 97%, about 98%, about 99% or about 100% homologous or identical to SEQ ID NO: 135. In certain embodiments, the extracellular antigen-binding domain comprises a V_{H} comprising the amino sequence set forth in SEQ ID NO: 135.

In certain embodiments, the extracellular antigen-binding domain (e.g., an scFv) comprises a light chain variable region (V_{L}) comprising an amino acid sequence that is at least about 80% (e.g., at least about 85%, at least about 90%, or at least about 95%) homologous or identical to the amino sequence set forth in SEQ ID NO: 136. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 136. In certain embodiments, the extracellular antigen-binding domain comprises a V_{L} comprising the amino sequence set forth in SEQ ID NO: 136.

In certain embodiments, the extracellular antigen-binding domain (e.g., an scFv) comprises a V_{H} comprising the amino acid sequence set forth in SEQ ID NO: 135, and a V_{L} comprising the amino acid sequence set forth in SEQ ID NO: 136. In certain embodiments, the extracellular antigen-binding domain is an scFv. In certain embodiments, the scFv is designated as “76H12v12”. In certain embodiments, the V_{H} and V_{L} are linked via a linker. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138.

TABLE 54

| CDRs | 1 | 2 | 3 |
|---|---|---|---|
| VH | GYTFTSYW | VYPGSGST | TRPGNYEGFAY |
| | [SEQ ID NO: | [SEQ ID NO: | [SEQ ID NO: 118] |
| | 116] | 117] | |
| VL | ESVDSYGSSF | LAS | QQNNEDPWT |
| | [SEQ ID NO: | [SEQ ID NO: | [SEQ ID NO: 121] |
| | 119] | 120] | |

| | |
|---|---|
| Full | QVQLVQSGAEVKKPGASVKVSCKASGYTFTSYWMHWVRQAPGQGLEWIGNVYPGSG |
| VH | STKYAQKFQGRATLTVDTSTNTAYMELSSLRSEDTAVYYCTRPGNYEGFAYWGQGT |
| | LVTVSS |
| | [SEQ ID NO: 135] |
| Full | DIQMTQSPSSLSASVGDRVTITCRASESVDSYGSSFMHWYQQKPGKAPKLLIYLAS |
| VL | NLESGVPSRFSGSGSGTDFTLTISSLQPEDFATYYCQQNNEDPWTFGQGTKLEIK |
| | [SEQ ID NO: 136] |

As used herein, the term “a conservative sequence modification” refers to an amino acid modification that does not significantly affect or alter the binding characteristics of the presently disclosed L1CAM-targeted CAR (e.g., the extracellular antigen-binding domain of the CAR) comprising the amino acid sequence. Conservative modifications can include amino acid substitutions, additions and deletions. Modifications can be introduced into the extracellular antigen-binding domain of the presently disclosed CAR by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered antibody can be tested for retained function (i.e., the functions set forth in (c) through (l) above) using the functional assays described herein. In certain embodiments, no more than one, no more than two, no more than three, no more than four, no more than five residues within a specified sequence or a CDR region are altered.

The V_{H} and/or V_{L} amino acid sequences comprising or consisting of at least about 80%, at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homology or identity to a specific sequence (e.g., SEQ ID NO: 8, SEQ ID NO: 9, SEQ ID NO: 18, SEQ ID NO: 19, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 41, SEQ ID NO: 42, SEQ ID NO: 49, SEQ ID NO: 50, SEQ ID NO: 53, SEQ ID NO: 60, SEQ ID NO: 61, SEQ ID NO: 67, SEQ ID NO: 68, SEQ ID NO: 75, SEQ ID NO: 76, SEQ ID NO: 87, SEQ ID NO: 88, SEQ ID NO: 99, SEQ ID NO: 100, SEQ ID NO: 110, SEQ ID NO: 111, SEQ ID NO: 122, SEQ ID NO: 123, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, or SEQ ID NO: 136) may contain substitutions (e.g., conservative substitutions), insertions, or deletions relative to the specified sequence(s), but retain the ability to bind to a target antigen (e.g., mesothelin). In certain embodiments, a total of 1 to 10 amino acids are substituted, inserted and/or deleted in a specific sequence (e.g., SEQ ID NO: 8, SEQ ID NO: 9, SEQ ID NO: 18, SEQ ID NO: 19, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 41, SEQ ID NO: 42, SEQ ID NO: 49, SEQ ID NO: 50, SEQ ID NO: 53, SEQ ID NO: 60, SEQ ID NO: 61, SEQ ID NO: 67, SEQ ID NO: 68, SEQ ID NO: 75, SEQ ID NO: 76, SEQ ID NO: 87, SEQ ID NO: 88, SEQ ID NO: 99, SEQ ID NO: 100, SEQ ID NO: 110, SEQ ID NO: 111, SEQ ID NO: 122, SEQ ID NO: 123, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, or SEQ ID NO: 136). In certain embodiments, substitutions, insertions, or deletions occur in regions outside the CDRs (e.g., in the FRs) of the extracellular antigen-binding domain. In certain embodiments, the extracellular antigen-binding domain comprises V_{H} and/or V_{L} sequence selected from SEQ ID NO: 8, SEQ ID NO: 9, SEQ ID NO: 18, SEQ ID NO: 19, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 41, SEQ ID NO: 42, SEQ ID NO: 49, SEQ ID NO: 50, SEQ ID NO: 53, SEQ ID NO: 60, SEQ ID NO: 61, SEQ ID NO: 67, SEQ ID NO: 68, SEQ ID NO: 75, SEQ ID NO: 76, SEQ ID NO: 87, SEQ ID NO: 88, SEQ ID NO: 99, SEQ ID NO: 100, SEQ ID NO: 110, SEQ ID NO: 111, SEQ ID NO: 122, SEQ ID NO: 123, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, or SEQ ID NO: 136, including post-translational modifications of that sequence (e.g., SEQ ID NO: 8, SEQ ID NO: 9, SEQ ID NO: 18, SEQ ID NO: 19, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 41, SEQ ID NO: 42, SEQ ID NO: 49, SEQ ID NO: 50, SEQ ID NO: 53, SEQ ID NO: 60, SEQ ID NO: 61, SEQ ID NO: 67, SEQ ID NO: 68, SEQ ID NO: 75, SEQ ID NO: 76, SEQ ID NO: 87, SEQ ID NO: 88, SEQ ID NO: 99, SEQ ID NO: 100, SEQ ID NO: 110, SEQ ID NO: 111, SEQ ID NO: 122, SEQ ID NO: 123, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, or SEQ ID NO: 136).

In certain embodiments, the extracellular antigen-binding domain of a presently disclosed CAR cross-competes for binding to a L1CAM polypeptide with a reference antibody or an antigen-binding portion thereof comprising a heavy chain variable region comprising a CDR1 comprising the amino acid sequence set forth in SEQ ID NO: 2, a CDR2 comprising the amino acid sequence set forth in SEQ ID NO: 3, a CDR3 comprising the amino acid sequence set forth in SEQ ID NO: 4; and a light chain variable region comprising a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7. In certain embodiments, the extracellular antigen-binding domain of a presently disclosed CAR cross-competes for binding to an L1CAM antibody with a reference antibody or an antigen-binding portion thereof comprising a V_{H} comprising the amino acid sequence set forth in SEQ ID NO: 8, and a V_{L} comprising the amino acid sequence set forth in SEQ ID NO: 9.

In certain embodiments, the extracellular antigen-binding domain of a presently disclosed CAR cross-competes for binding to a L1CAM polypeptide with a reference antibody or an antigen-binding portion thereof comprising a heavy chain variable region comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17. In certain embodiments, the extracellular antigen-binding domain of a presently disclosed CAR cross-competes for binding to an L1CAM antibody with a reference antibody or an antigen-binding portion thereof comprising a V_{H} comprising the amino acid sequence set forth in SEQ ID NO: 18, and a V_{L} comprising the amino acid sequence set forth in SEQ ID NO: 19.

In certain embodiments, the extracellular antigen-binding domain of a presently disclosed CAR cross-competes for binding to a L1CAM polypeptide with a reference antibody or an antigen-binding portion thereof comprising a heavy chain variable region comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17. In certain embodiments, the extracellular antigen-binding domain of a presently disclosed CAR cross-competes for binding to an L1CAM antibody with a reference antibody or an antigen-binding portion thereof comprising a V_{H} comprising the amino acid sequence set forth in SEQ ID NO: 22, and a V_{L} comprising the amino acid sequence set forth in SEQ ID NO: 23.

In certain embodiments, the extracellular antigen-binding domain of a presently disclosed CAR cross-competes for binding to a L1CAM polypeptide with a reference antibody or an antigen-binding portion thereof comprising a heavy chain variable region comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17. In certain embodiments, the extracellular antigen-binding domain of a presently disclosed CAR cross-competes for binding to an L1CAM antibody with a reference antibody or an antigen-binding portion thereof comprising a V_{H} comprising the amino acid sequence set forth in SEQ ID NO: 24, and a V_{L} comprising the amino acid sequence set forth in SEQ ID NO: 23.

In certain embodiments, the extracellular antigen-binding domain of a presently disclosed CAR cross-competes for binding to a L1CAM polypeptide with a reference antibody or an antigen-binding portion thereof comprising a heavy chain variable region comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17. In certain embodiments, the extracellular antigen-binding domain of a presently disclosed CAR cross-competes for binding to an L1CAM antibody with a reference antibody or an antigen-binding portion thereof comprising a V_{H} comprising the amino acid sequence set forth in SEQ ID NO: 25, and a V_{L} comprising the amino acid sequence set forth in SEQ ID NO: 23.

In certain embodiments, the extracellular antigen-binding domain of a presently disclosed CAR cross-competes for binding to a L1CAM polypeptide with a reference antibody or an antigen-binding portion thereof comprising a heavy chain variable region comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 26; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17. In certain embodiments, the extracellular antigen-binding domain of a presently disclosed CAR cross-competes for binding to an L1CAM antibody with a reference antibody or an antigen-binding portion thereof comprising a V_{H} comprising the amino acid sequence set forth in SEQ ID NO: 27, and a V_{L} comprising the amino acid sequence set forth in SEQ ID NO: 23.

In certain embodiments, the extracellular antigen-binding domain of a presently disclosed CAR cross-competes for binding to a L1CAM polypeptide with a reference antibody or an antigen-binding portion thereof comprising a heavy chain variable region comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17. In certain embodiments, the extracellular antigen-binding domain of a presently disclosed CAR cross-competes for binding to an L1CAM antibody with a reference antibody or an antigen-binding portion thereof comprising a V_{H} comprising the amino acid sequence set forth in SEQ ID NO: 28, and a V_{L} comprising the amino acid sequence set forth in SEQ ID NO: 23.

In certain embodiments, the extracellular antigen-binding domain of a presently disclosed CAR cross-competes for binding to a L1CAM polypeptide with a reference antibody or an antigen-binding portion thereof comprising a heavy chain variable region comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 26; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17. In certain embodiments, the extracellular antigen-binding domain of a presently disclosed CAR cross-competes for binding to an L1CAM antibody with a reference antibody or an antigen-binding portion thereof comprising a V_{H} comprising the amino acid sequence set forth in SEQ ID NO: 29, and a V_{L} comprising the amino acid sequence set forth in SEQ ID NO: 23.

In certain embodiments, the extracellular antigen-binding domain of a presently disclosed CAR cross-competes for binding to a L1CAM polypeptide with a reference antibody or an antigen-binding portion thereof comprising a heavy chain variable region comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17. In certain embodiments, the extracellular antigen-binding domain of a presently disclosed CAR cross-competes for binding to an L1CAM antibody with a reference antibody or an antigen-binding portion thereof comprising a V_{H} comprising the amino acid sequence set forth in SEQ ID NO: 22, and a V_{L} comprising the amino acid sequence set forth in SEQ ID NO: 30.

In certain embodiments, the extracellular antigen-binding domain of a presently disclosed CAR cross-competes for binding to a L1CAM polypeptide with a reference antibody or an antigen-binding portion thereof comprising a heavy chain variable region comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17. In certain embodiments, the extracellular antigen-binding domain of a presently disclosed CAR cross-competes for binding to an L1CAM antibody with a reference antibody or an antigen-binding portion thereof comprising a V_{H} comprising the amino acid sequence set forth in SEQ ID NO: 24, and a V_{L} comprising the amino acid sequence set forth in SEQ ID NO: 30.

In certain embodiments, the extracellular antigen-binding domain of a presently disclosed CAR cross-competes for binding to a L1CAM polypeptide with a reference antibody or an antigen-binding portion thereof comprising a heavy chain variable region comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17. In certain embodiments, the extracellular antigen-binding domain of a presently disclosed CAR cross-competes for binding to an L1CAM antibody with a reference antibody or an antigen-binding portion thereof comprising a V_{H} comprising the amino acid sequence set forth in SEQ ID NO: 25, and a V_{L} comprising the amino acid sequence set forth in SEQ ID NO: 30.

In certain embodiments, the extracellular antigen-binding domain of a presently disclosed CAR cross-competes for binding to a L1CAM polypeptide with a reference antibody or an antigen-binding portion thereof comprising a heavy chain variable region comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 26; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17. In certain embodiments, the extracellular antigen-binding domain of a presently disclosed CAR cross-competes for binding to an L1CAM antibody with a reference antibody or an antigen-binding portion thereof comprising a V_{H} comprising the amino acid sequence set forth in SEQ ID NO: 27, and a V_{L} comprising the amino acid sequence set forth in SEQ ID NO: 30.

In certain embodiments, the extracellular antigen-binding domain of a presently disclosed CAR cross-competes for binding to a L1CAM polypeptide with a reference antibody or an antigen-binding portion thereof comprising a heavy chain variable region comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17. In certain embodiments, the extracellular antigen-binding domain of a presently disclosed CAR cross-competes for binding to an L1CAM antibody with a reference antibody or an antigen-binding portion thereof comprising a V_{H} comprising the amino acid sequence set forth in SEQ ID NO: 28, and a V_{L} comprising the amino acid sequence set forth in SEQ ID NO: 30.

In certain embodiments, the extracellular antigen-binding domain of a presently disclosed CAR cross-competes for binding to a L1CAM polypeptide with a reference antibody or an antigen-binding portion thereof comprising a heavy chain variable region comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 26; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17. In certain embodiments, the extracellular antigen-binding domain of a presently disclosed CAR cross-competes for binding to an L1CAM antibody with a reference antibody or an antigen-binding portion thereof comprising a V_{H} comprising the amino acid sequence set forth in SEQ ID NO: 29, and a V_{L} comprising the amino acid sequence set forth in SEQ ID NO: 30.

In certain embodiments, the extracellular antigen-binding domain of a presently disclosed CAR cross-competes for binding to a L1CAM polypeptide with a reference antibody or an antigen-binding portion thereof comprising a heavy chain variable region comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17. In certain embodiments, the extracellular antigen-binding domain of a presently disclosed CAR cross-competes for binding to an L1CAM antibody with a reference antibody or an antigen-binding portion thereof comprising a V_{H} comprising the amino acid sequence set forth in SEQ ID NO: 22, and a V_{L} comprising the amino acid sequence set forth in SEQ ID NO: 31.

In certain embodiments, the extracellular antigen-binding domain of a presently disclosed CAR cross-competes for binding to a L1CAM polypeptide with a reference antibody or an antigen-binding portion thereof comprising a heavy chain variable region comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17. In certain embodiments, the extracellular antigen-binding domain of a presently disclosed CAR cross-competes for binding to an L1CAM antibody with a reference antibody or an antigen-binding portion thereof comprising a V_{H} comprising the amino acid sequence set forth in SEQ ID NO: 24, and a V_{L} comprising the amino acid sequence set forth in SEQ ID NO: 31.

In certain embodiments, the extracellular antigen-binding domain of a presently disclosed CAR cross-competes for binding to a L1CAM polypeptide with a reference antibody or an antigen-binding portion thereof comprising a heavy chain variable region comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17. In certain embodiments, the extracellular antigen-binding domain of a presently disclosed CAR cross-competes for binding to an L1CAM antibody with a reference antibody or an antigen-binding portion thereof comprising a V_{H} comprising the amino acid sequence set forth in SEQ ID NO: 25, and a V_{L} comprising the amino acid sequence set forth in SEQ ID NO: 31.

In certain embodiments, the extracellular antigen-binding domain of a presently disclosed CAR cross-competes for binding to a L1CAM polypeptide with a reference antibody or an antigen-binding portion thereof comprising a heavy chain variable region comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 26; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17. In certain embodiments, the extracellular antigen-binding domain of a presently disclosed CAR cross-competes for binding to an L1CAM antibody with a reference antibody or an antigen-binding portion thereof comprising a V_{H} comprising the amino acid sequence set forth in SEQ ID NO: 27, and a V_{L} comprising the amino acid sequence set forth in SEQ ID NO: 31.

In certain embodiments, the extracellular antigen-binding domain of a presently disclosed CAR cross-competes for binding to a L1CAM polypeptide with a reference antibody or an antigen-binding portion thereof comprising a heavy chain variable region comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17. In certain embodiments, the extracellular antigen-binding domain of a presently disclosed CAR cross-competes for binding to an L1CAM antibody with a reference antibody or an antigen-binding portion thereof comprising a V_{H} comprising the amino acid sequence set forth in SEQ ID NO: 28, and a V_{L} comprising the amino acid sequence set forth in SEQ ID NO: 31.

In certain embodiments, the extracellular antigen-binding domain of a presently disclosed CAR cross-competes for binding to a L1CAM polypeptide with a reference antibody or an antigen-binding portion thereof comprising a heavy chain variable region comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 26; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17. In certain embodiments, the extracellular antigen-binding domain of a presently disclosed CAR cross-competes for binding to an L1CAM antibody with a reference antibody or an antigen-binding portion thereof comprising a V_{H} comprising the amino acid sequence set forth in SEQ ID NO: 29, and a V_{L} comprising the amino acid sequence set forth in SEQ ID NO: 31.

In certain embodiments, the extracellular antigen-binding domain of a presently disclosed CAR cross-competes for binding to a L1CAM polypeptide with a reference antibody or an antigen-binding portion thereof comprising a heavy chain variable region comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17. In certain embodiments, the extracellular antigen-binding domain of a presently disclosed CAR cross-competes for binding to an L1CAM antibody with a reference antibody or an antigen-binding portion thereof comprising a V_{H} comprising the amino acid sequence set forth in SEQ ID NO: 22, and a V_{L} comprising the amino acid sequence set forth in SEQ ID NO: 32.

In certain embodiments, the extracellular antigen-binding domain of a presently disclosed CAR cross-competes for binding to a L1CAM polypeptide with a reference antibody or an antigen-binding portion thereof comprising a heavy chain variable region comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17. In certain embodiments, the extracellular antigen-binding domain of a presently disclosed CAR cross-competes for binding to an L1CAM antibody with a reference antibody or an antigen-binding portion thereof comprising a V_{H} comprising the amino acid sequence set forth in SEQ ID NO: 24, and a V_{L} comprising the amino acid sequence set forth in SEQ ID NO: 32.

In certain embodiments, the extracellular antigen-binding domain of a presently disclosed CAR cross-competes for binding to a L1CAM polypeptide with a reference antibody or an antigen-binding portion thereof comprising a heavy chain variable region comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17. In certain embodiments, the extracellular antigen-binding domain of a presently disclosed CAR cross-competes for binding to an L1CAM antibody with a reference antibody or an antigen-binding portion thereof comprising a V_{H} comprising the amino acid sequence set forth in SEQ ID NO: 25, and a V_{L} comprising the amino acid sequence set forth in SEQ ID NO: 32.

In certain embodiments, the extracellular antigen-binding domain of a presently disclosed CAR cross-competes for binding to a L1CAM polypeptide with a reference antibody or an antigen-binding portion thereof comprising a heavy chain variable region comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 26; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17. In certain embodiments, the extracellular antigen-binding domain of a presently disclosed CAR cross-competes for binding to an L1CAM antibody with a reference antibody or an antigen-binding portion thereof comprising a V_{H} comprising the amino acid sequence set forth in SEQ ID NO: 27, and a V_{L} comprising the amino acid sequence set forth in SEQ ID NO: 32.

In certain embodiments, the extracellular antigen-binding domain of a presently disclosed CAR cross-competes for binding to a L1CAM polypeptide with a reference antibody or an antigen-binding portion thereof comprising a heavy chain variable region comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17. In certain embodiments, the extracellular antigen-binding domain of a presently disclosed CAR cross-competes for binding to an L1CAM antibody with a reference antibody or an antigen-binding portion thereof comprising a V_{H} comprising the amino acid sequence set forth in SEQ ID NO: 28, and a V_{L} comprising the amino acid sequence set forth in SEQ ID NO: 32.

In certain embodiments, the extracellular antigen-binding domain of a presently disclosed CAR cross-competes for binding to a L1CAM polypeptide with a reference antibody or an antigen-binding portion thereof comprising a heavy chain variable region comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 26; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17. In certain embodiments, the extracellular antigen-binding domain of a presently disclosed CAR cross-competes for binding to an L1CAM antibody with a reference antibody or an antigen-binding portion thereof comprising a V_{H} comprising the amino acid sequence set forth in SEQ ID NO: 29, and a V_{L} comprising the amino acid sequence set forth in SEQ ID NO: 32.

In certain embodiments, the extracellular antigen-binding domain of a presently disclosed CAR cross-competes for binding to a L1CAM polypeptide with a reference antibody or an antigen-binding portion thereof comprising a heavy chain variable region comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17. In certain embodiments, the extracellular antigen-binding domain of a presently disclosed CAR cross-competes for binding to an L1CAM antibody with a reference antibody or an antigen-binding portion thereof comprising a V_{H} comprising the amino acid sequence set forth in SEQ ID NO: 22, and a V_{L} comprising the amino acid sequence set forth in SEQ ID NO: 19.

In certain embodiments, the extracellular antigen-binding domain of a presently disclosed CAR cross-competes for binding to a L1CAM polypeptide with a reference antibody or an antigen-binding portion thereof comprising a heavy chain variable region comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17. In certain embodiments, the extracellular antigen-binding domain of a presently disclosed CAR cross-competes for binding to an L1CAM antibody with a reference antibody or an antigen-binding portion thereof comprising a V_{H} comprising the amino acid sequence set forth in SEQ ID NO: 24, and a V_{L} comprising the amino acid sequence set forth in SEQ ID NO: 19.

In certain embodiments, the extracellular antigen-binding domain of a presently disclosed CAR cross-competes for binding to a L1CAM polypeptide with a reference antibody or an antigen-binding portion thereof comprising a heavy chain variable region comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17. In certain embodiments, the extracellular antigen-binding domain of a presently disclosed CAR cross-competes for binding to an L1CAM antibody with a reference antibody or an antigen-binding portion thereof comprising a V_{H} comprising the amino acid sequence set forth in SEQ ID NO: 25, and a V_{L} comprising the amino acid sequence set forth in SEQ ID NO: 19.

In certain embodiments, the extracellular antigen-binding domain of a presently disclosed CAR cross-competes for binding to a L1CAM polypeptide with a reference antibody or an antigen-binding portion thereof comprising a heavy chain variable region comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 26; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17. In certain embodiments, the extracellular antigen-binding domain of a presently disclosed CAR cross-competes for binding to an L1CAM antibody with a reference antibody or an antigen-binding portion thereof comprising a V_{H} comprising the amino acid sequence set forth in SEQ ID NO: 27, and a V_{L} comprising the amino acid sequence set forth in SEQ ID NO: 19.

In certain embodiments, the extracellular antigen-binding domain of a presently disclosed CAR cross-competes for binding to a L1CAM polypeptide with a reference antibody or an antigen-binding portion thereof comprising a heavy chain variable region comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17. In certain embodiments, the extracellular antigen-binding domain of a presently disclosed CAR cross-competes for binding to an L1CAM antibody with a reference antibody or an antigen-binding portion thereof comprising a V_{H} comprising the amino acid sequence set forth in SEQ ID NO: 28, and a V_{L} comprising the amino acid sequence set forth in SEQ ID NO: 19.

In certain embodiments, the extracellular antigen-binding domain of a presently disclosed CAR cross-competes for binding to a L1CAM polypeptide with a reference antibody or an antigen-binding portion thereof comprising a heavy chain variable region comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 26; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17. In certain embodiments, the extracellular antigen-binding domain of a presently disclosed CAR cross-competes for binding to an L1CAM antibody with a reference antibody or an antigen-binding portion thereof comprising a V_{H} comprising the amino acid sequence set forth in SEQ ID NO: 29, and a V_{L} comprising the amino acid sequence set forth in SEQ ID NO: 19.

In certain embodiments, the extracellular antigen-binding domain of a presently disclosed CAR cross-competes for binding to a L1CAM polypeptide with a reference antibody or an antigen-binding portion thereof comprising a heavy chain variable region comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17. In certain embodiments, the extracellular antigen-binding domain of a presently disclosed CAR cross-competes for binding to an L1CAM antibody with a reference antibody or an antigen-binding portion thereof comprising a V_{H} comprising the amino acid sequence set forth in SEQ ID NO: 33, and a V_{L} comprising the amino acid sequence set forth in SEQ ID NO: 34. In certain embodiments, the extracellular antigen-binding domain of a presently disclosed CAR cross-competes for binding to a L1CAM polypeptide with a reference antibody or an antigen-binding portion thereof comprising a heavy chain variable region comprising a CDR1 comprising the amino acid sequence set forth in SEQ ID NO: 35, a CDR2 comprising the amino acid sequence set forth in SEQ ID NO: 36, a CDR3 comprising the amino acid sequence set forth in SEQ ID NO: 37; and a light chain variable region comprising a CDR1 comprising the amino acid sequence set forth in SEQ ID NO: 38, a CDR2 comprising the amino acid sequence set forth in SEQ ID NO: 39, and a CDR3 comprising the amino acid sequence set forth in SEQ ID NO: 40. In certain embodiments, the extracellular antigen-binding domain of a presently disclosed CAR cross-competes for binding to an L1CAM antibody with a reference antibody or an antigen-binding portion thereof comprising a V_{H} comprising the amino acid sequence set forth in SEQ ID NO: 41, and a V_{L} comprising the amino acid sequence set forth in SEQ ID NO: 42.

In certain embodiments, the extracellular antigen-binding domain of a presently disclosed CAR cross-competes for binding to a L1CAM polypeptide with a reference antibody or an antigen-binding portion thereof comprising a heavy chain variable region comprising a CDR1 comprising the amino acid sequence set forth in SEQ ID NO: 43, a CDR2 comprising the amino acid sequence set forth in SEQ ID NO: 44, a CDR3 comprising the amino acid sequence set forth in SEQ ID NO: 45; and a light chain variable region comprising a CDR1 comprising the amino acid sequence set forth in SEQ ID NO: 46, a CDR2 comprising the amino acid sequence set forth in SEQ ID NO: 47, and a CDR3 comprising the amino acid sequence set forth in SEQ ID NO: 48. In certain embodiments, the extracellular antigen-binding domain of a presently disclosed CAR cross-competes for binding to an L1CAM antibody with a reference antibody or an antigen-binding portion thereof comprising a V_{H} comprising the amino acid sequence set forth in SEQ ID NO: 49, and a V_{L} comprising the amino acid sequence set forth in SEQ ID NO: 50.

In certain embodiments, the extracellular antigen-binding domain of a presently disclosed CAR cross-competes for binding to a L1CAM polypeptide with a reference antibody or an antigen-binding portion thereof comprising a heavy chain variable region comprising a CDR1 comprising the amino acid sequence set forth in SEQ ID NO: 43, a CDR2 comprising the amino acid sequence set forth in SEQ ID NO: 44, a CDR3 comprising the amino acid sequence set forth in SEQ ID NO: 45; and a light chain variable region comprising a CDR1 comprising the amino acid sequence set forth in SEQ ID NO: 51, a CDR2 comprising the amino acid sequence set forth in SEQ ID NO: 52, and a CDR3 comprising the amino acid sequence set forth in SEQ ID NO: 40. In certain embodiments, the extracellular antigen-binding domain of a presently disclosed CAR cross-competes for binding to an L1CAM antibody with a reference antibody or an antigen-binding portion thereof comprising a V_{H} comprising the amino acid sequence set forth in SEQ ID NO: 49, and a V_{L} comprising the amino acid sequence set forth in SEQ ID NO: 53.

In certain embodiments, the extracellular antigen-binding domain of a presently disclosed CAR cross-competes for binding to a L1CAM polypeptide with a reference antibody or an antigen-binding portion thereof comprising a heavy chain variable region comprising a CDR1 comprising the amino acid sequence set forth in SEQ ID NO: 54, a CDR2 comprising the amino acid sequence set forth in SEQ ID NO: 55, a CDR3 comprising the amino acid sequence set forth in SEQ ID NO: 56; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 59. In certain embodiments, the extracellular antigen-binding domain of a presently disclosed CAR cross-competes for binding to an L1CAM antibody with a reference antibody or an antigen-binding portion thereof comprising a V_{H} comprising the amino acid sequence set forth in SEQ ID NO: 60, and a V_{L} comprising the amino acid sequence set forth in SEQ ID NO: 61.

In certain embodiments, the extracellular antigen-binding domain binds to the same or substantially the same epitope on a L1CAM polypeptide with a reference antibody or an antigen-binding portion thereof comprising a heavy chain variable region comprising a CDR1 comprising the amino acid sequence set forth in SEQ ID NO: 62, a CDR2 comprising the amino acid sequence set forth in SEQ ID NO: 63, a CDR3 comprising the amino acid sequence set forth in SEQ ID NO: 64; and a light chain variable region comprising a CDR1 comprising the amino acid sequence set forth in SEQ ID NO: 65, a CDR2 comprising the amino acid sequence set forth in SEQ ID NO: 58, and a CDR3 comprising the amino acid sequence set forth in SEQ ID NO: 66. In certain embodiments, the extracellular antigen-binding domain of a presently disclosed CAR binds to the same or substantially the same epitope on an L1CAM antibody with a reference antibody or an antigen-binding portion thereof comprising a V_{H} comprising the amino acid sequence set forth in SEQ ID NO: 67, and a V_{L} comprising the amino acid sequence set forth in SEQ ID NO: 68.

In certain embodiments, the extracellular antigen-binding domain binds to the same or substantially the same epitope on a L1CAM polypeptide with a reference antibody or an antigen-binding portion thereof comprising a heavy chain variable region comprising a CDR1 comprising the amino acid sequence set forth in SEQ ID NO: 69, a CDR2 comprising the amino acid sequence set forth in SEQ ID NO: 70, a CDR3 comprising the amino acid sequence set forth in SEQ ID NO: 71; and a light chain variable region comprising a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 74. In certain embodiments, the extracellular antigen-binding domain of a presently disclosed CAR binds to the same or substantially the same epitope on an L1CAM antibody with a reference antibody or an antigen-binding portion thereof comprising a V_{H} comprising the amino acid sequence set forth in SEQ ID NO: 75, and a V_{L} comprising the amino acid sequence set forth in SEQ ID NO: 76.

In certain embodiments, the extracellular antigen-binding domain binds to the same or substantially the same epitope on a L1CAM polypeptide with a reference antibody or an antigen-binding portion thereof comprising a heavy chain variable region comprising a CDR1 comprising the amino acid sequence set forth in SEQ ID NO: 81, a CDR2 comprising the amino acid sequence set forth in SEQ ID NO: 82, a CDR3 comprising the amino acid sequence set forth in SEQ ID NO: 83; and a light chain variable region comprising a CDR1 comprising the amino acid sequence set forth in SEQ ID NO: 84, a CDR2 comprising the amino acid sequence set forth in SEQ ID NO: 85, and a CDR3 comprising the amino acid sequence set forth in SEQ ID NO: 86. In certain embodiments, the extracellular antigen-binding domain of a presently disclosed CAR binds to the same or substantially the same epitope on an L1CAM antibody with a reference antibody or an antigen-binding portion thereof comprising a V_{H} comprising the amino acid sequence set forth in SEQ ID NO: 87, and a V_{L} comprising the amino acid sequence set forth in SEQ ID NO: 88.

In certain embodiments, the extracellular antigen-binding domain binds to the same or substantially the same epitope on a L1CAM polypeptide with a reference antibody or an antigen-binding portion thereof comprising a heavy chain variable region comprising a CDR1 comprising the amino acid sequence set forth in SEQ ID NO: 93, a CDR2 comprising the amino acid sequence set forth in SEQ ID NO: 94, a CDR3 comprising the amino acid sequence set forth in SEQ ID NO: 95; and a light chain variable region comprising a CDR1 comprising the amino acid sequence set forth in SEQ ID NO: 96, a CDR2 comprising the amino acid sequence set forth in SEQ ID NO: 97, and a CDR3 comprising the amino acid sequence set forth in SEQ ID NO: 98. In certain embodiments, the extracellular antigen-binding domain of a presently disclosed CAR binds to the same or substantially the same epitope on an L1CAM antibody with a reference antibody or an antigen-binding portion thereof comprising a V_{H} comprising the amino acid sequence set forth in SEQ ID NO: 99, and a V_{L} comprising the amino acid sequence set forth in SEQ ID NO: 100.

In certain embodiments, the extracellular antigen-binding domain binds to the same or substantially the same epitope on a L1CAM polypeptide with a reference antibody or an antigen-binding portion thereof comprising a heavy chain variable region comprising a CDR1 comprising the amino acid sequence set forth in SEQ ID NO: 105, a CDR2 comprising the amino acid sequence set forth in SEQ ID NO: 106, a CDR3 comprising the amino acid sequence set forth in SEQ ID NO: 107; and a light chain variable region comprising a CDR1 comprising the amino acid sequence set forth in SEQ ID NO: 108, a CDR2 comprising the amino acid sequence set forth in SEQ ID NO: 39, and a CDR3 comprising the amino acid sequence set forth in SEQ ID NO: 109. In certain embodiments, the extracellular antigen-binding domain of a presently disclosed CAR binds to the same or substantially the same epitope on an L1CAM antibody with a reference antibody or an antigen-binding portion thereof comprising a V_{H} comprising the amino acid sequence set forth in SEQ ID NO: 110, and a V_{L} comprising the amino acid sequence set forth in SEQ ID NO: 111.

In certain embodiments, the extracellular antigen-binding domain binds to the same or substantially the same epitope on a L1CAM polypeptide with a reference antibody or an antigen-binding portion thereof comprising a heavy chain variable region comprising a CDR1 comprising the amino acid sequence set forth in SEQ ID NO: 116, a CDR2 comprising the amino acid sequence set forth in SEQ ID NO: 117, a CDR3 comprising the amino acid sequence set forth in SEQ ID NO: 118; and a light chain variable region comprising a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121. In certain embodiments, the extracellular antigen-binding domain of a presently disclosed CAR binds to the same or substantially the same epitope on an L1CAM antibody with a reference antibody or an antigen-binding portion thereof comprising a V_{H} comprising the amino acid sequence set forth in SEQ ID NO: 122, and a V_{L} comprising the amino acid sequence set forth in SEQ ID NO: 123.

In certain embodiments, the extracellular antigen-binding domain binds to the same or substantially the same epitope on a L1CAM polypeptide with a reference antibody or an antigen-binding portion thereof comprising a heavy chain variable region comprising a CDR1 comprising the amino acid sequence set forth in SEQ ID NO: 116, a CDR2 comprising the amino acid sequence set forth in SEQ ID NO: 117, a CDR3 comprising the amino acid sequence set forth in SEQ ID NO: 118; and a light chain variable region comprising a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121. In certain embodiments, the extracellular antigen-binding domain of a presently disclosed CAR binds to the same or substantially the same epitope on an L1CAM antibody with a reference antibody or an antigen-binding portion thereof comprising a V_{H} comprising the amino acid sequence set forth in SEQ ID NO: 128, and a V_{L} comprising the amino acid sequence set forth in SEQ ID NO: 129.

In certain embodiments, the extracellular antigen-binding domain binds to the same or substantially the same epitope on a L1CAM polypeptide with a reference antibody or an antigen-binding portion thereof comprising a heavy chain variable region comprising a CDR1 comprising the amino acid sequence set forth in SEQ ID NO: 116, a CDR2 comprising the amino acid sequence set forth in SEQ ID NO: 117, a CDR3 comprising the amino acid sequence set forth in SEQ ID NO: 118; and a light chain variable region comprising a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121. In certain embodiments, the extracellular antigen-binding domain of a presently disclosed CAR binds to the same or substantially the same epitope on an L1CAM antibody with a reference antibody or an antigen-binding portion thereof comprising a V_{H} comprising the amino acid sequence set forth in SEQ ID NO: 130, and a V_{L} comprising the amino acid sequence set forth in SEQ ID NO: 131.

In certain embodiments, the extracellular antigen-binding domain binds to the same or substantially the same epitope on a L1CAM polypeptide with a reference antibody or an antigen-binding portion thereof comprising a heavy chain variable region comprising a CDR1 comprising the amino acid sequence set forth in SEQ ID NO: 116, a CDR2 comprising the amino acid sequence set forth in SEQ ID NO: 117, a CDR3 comprising the amino acid sequence set forth in SEQ ID NO: 118; and a light chain variable region comprising a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121. In certain embodiments, the extracellular antigen-binding domain of a presently disclosed CAR binds to the same or substantially the same epitope on an L1CAM antibody with a reference antibody or an antigen-binding portion thereof comprising a V_{H} comprising the amino acid sequence set forth in SEQ ID NO: 130, and a V_{L} comprising the amino acid sequence set forth in SEQ ID NO: 132.

In certain embodiments, the extracellular antigen-binding domain binds to the same or substantially the same epitope on a L1CAM polypeptide with a reference antibody or an antigen-binding portion thereof comprising a heavy chain variable region comprising a CDR1 comprising the amino acid sequence set forth in SEQ ID NO: 116, a CDR2 comprising the amino acid sequence set forth in SEQ ID NO: 117, a CDR3 comprising the amino acid sequence set forth in SEQ ID NO: 118; and a light chain variable region comprising a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121. In certain embodiments, the extracellular antigen-binding domain of a presently disclosed CAR binds to the same or substantially the same epitope on an L1CAM antibody with a reference antibody or an antigen-binding portion thereof comprising a V_{H} comprising the amino acid sequence set forth in SEQ ID NO: 130, and a V_{L} comprising the amino acid sequence set forth in SEQ ID NO: 133.

In certain embodiments, the extracellular antigen-binding domain binds to the same or substantially the same epitope on a L1CAM polypeptide with a reference antibody or an antigen-binding portion thereof comprising a heavy chain variable region comprising a CDR1 comprising the amino acid sequence set forth in SEQ ID NO: 116, a CDR2 comprising the amino acid sequence set forth in SEQ ID NO: 117, a CDR3 comprising the amino acid sequence set forth in SEQ ID NO: 118; and a light chain variable region comprising a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121. In certain embodiments, the extracellular antigen-binding domain of a presently disclosed CAR binds to the same or substantially the same epitope on an L1CAM antibody with a reference antibody or an antigen-binding portion thereof comprising a V_{H} comprising the amino acid sequence set forth in SEQ ID NO: 134, and a V_{L} comprising the amino acid sequence set forth in SEQ ID NO: 131.

In certain embodiments, the extracellular antigen-binding domain binds to the same or substantially the same epitope on a L1CAM polypeptide with a reference antibody or an antigen-binding portion thereof comprising a heavy chain variable region comprising a CDR1 comprising the amino acid sequence set forth in SEQ ID NO: 116, a CDR2 comprising the amino acid sequence set forth in SEQ ID NO: 117, a CDR3 comprising the amino acid sequence set forth in SEQ ID NO: 118; and a light chain variable region comprising a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121. In certain embodiments, the extracellular antigen-binding domain of a presently disclosed CAR binds to the same or substantially the same epitope on an L1CAM antibody with a reference antibody or an antigen-binding portion thereof comprising a V_{H} comprising the amino acid sequence set forth in SEQ ID NO: 134, and a V_{L} comprising the amino acid sequence set forth in SEQ ID NO: 132.

In certain embodiments, the extracellular antigen-binding domain binds to the same or substantially the same epitope on a L1CAM polypeptide with a reference antibody or an antigen-binding portion thereof comprising a heavy chain variable region comprising a CDR1 comprising the amino acid sequence set forth in SEQ ID NO: 116, a CDR2 comprising the amino acid sequence set forth in SEQ ID NO: 117, a CDR3 comprising the amino acid sequence set forth in SEQ ID NO: 118; and a light chain variable region comprising a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121. In certain embodiments, the extracellular antigen-binding domain of a presently disclosed CAR binds to the same or substantially the same epitope on an L1CAM antibody with a reference antibody or an antigen-binding portion thereof comprising a V_{H} comprising the amino acid sequence set forth in SEQ ID NO: 134, and a V_{L} comprising the amino acid sequence set forth in SEQ ID NO: 133.

In certain embodiments, the extracellular antigen-binding domain binds to the same or substantially the same epitope on a L1CAM polypeptide with a reference antibody or an antigen-binding portion thereof comprising a heavy chain variable region comprising a CDR1 comprising the amino acid sequence set forth in SEQ ID NO: 116, a CDR2 comprising the amino acid sequence set forth in SEQ ID NO: 117, a CDR3 comprising the amino acid sequence set forth in SEQ ID NO: 118; and a light chain variable region comprising a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121. In certain embodiments, the extracellular antigen-binding domain of a presently disclosed CAR binds to the same or substantially the same epitope on an L1CAM antibody with a reference antibody or an antigen-binding portion thereof comprising a V_{H} comprising the amino acid sequence set forth in SEQ ID NO: 135, and a V_{L} comprising the amino acid sequence set forth in SEQ ID NO: 131.

In certain embodiments, the extracellular antigen-binding domain binds to the same or substantially the same epitope on a L1CAM polypeptide with a reference antibody or an antigen-binding portion thereof comprising a heavy chain variable region comprising a CDR1 comprising the amino acid sequence set forth in SEQ ID NO: 116, a CDR2 comprising the amino acid sequence set forth in SEQ ID NO: 117, a CDR3 comprising the amino acid sequence set forth in SEQ ID NO: 118; and a light chain variable region comprising a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121. In certain embodiments, the extracellular antigen-binding domain of a presently disclosed CAR binds to the same or substantially the same epitope on an L1CAM antibody with a reference antibody or an antigen-binding portion thereof comprising a V_{H} comprising the amino acid sequence set forth in SEQ ID NO: 135, and a V_{L} comprising the amino acid sequence set forth in SEQ ID NO: 132.

In certain embodiments, the extracellular antigen-binding domain binds to the same or substantially the same epitope on a L1CAM polypeptide with a reference antibody or an antigen-binding portion thereof comprising a heavy chain variable region comprising a CDR1 comprising the amino acid sequence set forth in SEQ ID NO: 116, a CDR2 comprising the amino acid sequence set forth in SEQ ID NO: 117, a CDR3 comprising the amino acid sequence set forth in SEQ ID NO: 118; and a light chain variable region comprising a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121. In certain embodiments, the extracellular antigen-binding domain of a presently disclosed CAR binds to the same or substantially the same epitope on an L1CAM antibody with a reference antibody or an antigen-binding portion thereof comprising a V_{H} comprising the amino acid sequence set forth in SEQ ID NO: 135, and a V_{L} comprising the amino acid sequence set forth in SEQ ID NO: 133.

In certain embodiments, the extracellular antigen-binding domain binds to the same or substantially the same epitope on a L1CAM polypeptide with a reference antibody or an antigen-binding portion thereof comprising a heavy chain variable region comprising a CDR1 comprising the amino acid sequence set forth in SEQ ID NO: 116, a CDR2 comprising the amino acid sequence set forth in SEQ ID NO: 117, a CDR3 comprising the amino acid sequence set forth in SEQ ID NO: 118; and a light chain variable region comprising a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121. In certain embodiments, the extracellular antigen-binding domain of a presently disclosed CAR binds to the same or substantially the same epitope on an L1CAM antibody with a reference antibody or an antigen-binding portion thereof comprising a V_{H} comprising the amino acid sequence set forth in SEQ ID NO: 135, and a V_{L} comprising the amino acid sequence set forth in SEQ ID NO: 136. Extracellular antigen-binding domains that cross-compete or compete with the reference antibody or antigen-binding portions thereof for binding to a L1CAM polypeptide can be identified by using routine methods known in the art, including, but not limited to, ELISAs, radioimmunoassays (RIAs), Biacore, flow cytometry, Western blotting, and any other suitable quantitative or qualitative antibody-binding assays. Competition ELISA is described in Morris, “Epitope Mapping of Protein Antigens by Competition ELISA”, The Protein Protocols Handbook (1996), pp 595-600, edited by J. Walker, which is incorporated by reference in its entirety. In certain embodiments, the antibody-binding assay comprises measuring an initial binding of a reference antibody to a L1CAM polypeptide, admixing the reference antibody with a test extracellular antigen-binding domain, measuring a second binding of the reference antibody to the L1CAM polypeptide in the presence of the test extracellular antigen-binding domain, and comparing the initial binding with the second binding of the reference antibody, wherein a decreased second binding of the reference antibody to the L1CAM polypeptide in comparison to the initial binding indicates that the test extracellular antigen-binding domain cross-competes with the reference antibody for binding to L1CAM polypeptide, e.g., one that recognizes the same or substantially the same epitope, an overlapping epitope, or an adjacent epitope. In certain embodiments, the reference antibody is labeled, e.g., with a fluorochrome, biotin, or peroxidase. In certain embodiments, the L1CAM polypeptide is expressed in cells, e.g., in a flow cytometry test. In certain embodiments, the L1CAM polypeptide is immobilized onto a surface, including a Biacore ship (e.g., in a Biacore test), or other media suitable for surface plasmon resonance analysis. The binding of the reference antibody in the presence of a completely irrelevant antibody (that does not bind to L1CAM polypeptide) can serve as the control high value. The control low value can be obtained by incubating a labeled reference antibody with an unlabeled reference antibody, where competition and reduced binding of the labeled reference antibody would occur. In certain embodiments, a test extracellular antigen-binding domain that reduces the binding of the reference antibody to L1CAM polypeptide by at least about 20%, at least about 30%, at least about 40%, at least about 50%, at least about 60%, at least about 70%, at least about 80%, at least about 90%, or at least about 95% is considered to be an extracellular antigen-binding domain that cross-competes with the reference antibody for binding to L1CAM polypeptide. In certain embodiments, the assays are performed at room temperature.

In certain embodiments, the antibody-binding assay comprises measuring an initial binding of a test extracellular antigen-binding domain to L1CAM polypeptide, admixing the test extracellular antigen-binding domain with a reference antibody, measuring a second binding of the test extracellular antigen-binding domain to L1CAM polypeptide in the presence of the reference antibody, and comparing the initial binding with the second binding of the test extracellular antigen-binding domain, where a decreased second binding of the test extracellular antigen-binding domain to L1CAM polypeptide in comparison to the initial binding indicates that the test extracellular antigen-binding domain cross-competes with the reference antibody for binding to L1CAM polypeptide, e.g., one that recognizes the same or substantially the same epitope, an overlapping epitope, or an adjacent epitope. In certain embodiments, the test extracellular antigen-binding domain is labeled, e.g., with a fluorochrome, biotin, or peroxidase. In certain embodiments, the L1CAM polypeptide is expressed in cells, e.g., in a flow cytometry test. In certain embodiments, the L1CAM polypeptide is immobilized onto a surface, including a Biacore ship (e.g., in a Biacore test), or other media suitable for surface plasmon resonance analysis. The binding of the test extracellular antigen-binding domain in the presence of a completely irrelevant antibody (that does not bind to L1CAM polypeptide) can serve as the control high value. The control low value can be obtained by incubating a labeled test extracellular antigen-binding domain with an unlabeled test extracellular antigen-binding domain, where competition and reduced binding of the labeled test extracellular antigen-binding domain would occur. In certain embodiments, a test extracellular antigen-binding domain, whose binding to L1CAM polypeptide is decreased by at least about 20%, at least about 30%, at least about 40%, at least about 50%, at least about 60%, at least about 70%, at least about 80%, at least about 90%, or at least about 95% in the presence of a reference antibody, is considered to be an extracellular antigen-binding domain that cross-competes with the reference antibody for binding to L1CAM polypeptide. In certain embodiments, the assays are performed at room temperature.

In certain non-limiting embodiments, the extracellular antigen-binding domain of the presently disclosed CAR comprises a linker connecting the heavy chain variable region and light chain variable region of the extracellular antigen-binding domain. In certain embodiments, the linker comprises the amino acid sequence set forth in SEQ ID NO: 10, SEQ ID NO: 11, SEQ ID NO: 20, SEQ ID NO: 21, SEQ ID NO: 137, or SEQ ID NO: 138. In certain embodiments, the linker comprises the amino acid sequence set forth in SEQ ID NO: 10. In certain embodiments, the linker comprises the amino acid sequence set forth in SEQ ID NO: 11. In certain embodiments, the linker comprises the amino acid sequence set forth in SEQ ID NO: 20. In certain embodiments, the linker comprises the amino acid sequence set forth in SEQ ID NO: 21. In certain embodiments, the linker comprises the amino acid sequence set forth in SEQ ID NO: 137. In certain embodiments, the linker comprises the amino acid sequence set forth in SEQ ID NO: 138.

In certain embodiments, the variable regions within the extracellular antigen-binding domain of the CAR have to be linked one after another such that at the N-terminus of the extracellular antigen-binding domain, a heavy chain variable region (V_{H}) is positioned. In certain embodiments, if the extracellular antigen-binding domain of the CAR is an scFv, the variable regions are positioned from the N- to the C-terminus: V_{H}-V_{L}.

In certain embodiments, the variable regions within the extracellular antigen-binding domain of the CAR have to be linked one after another such that at the N-terminus of the extracellular antigen-binding domain, a light chain variable region (V_{L}) is positioned. In certain embodiments, if the extracellular antigen-binding domain of the CAR is an scFv, the variable regions are positioned from the N- to the C-terminus: V_{L}-V_{H}.

In addition, the extracellular antigen-binding domain can comprise a leader or a signal peptide that directs the nascent protein into the endoplasmic reticulum. Signal peptide or leader can be essential if the CAR is to be glycosylated and anchored in the cell membrane. The signal sequence or leader can be a peptide sequence (about 5, about 10, about 15, about 20, about 25, or about 30 amino acids long) present at the N-terminus of newly synthesized proteins that directs their entry to the secretory pathway. In certain embodiments, the signal peptide is covalently joined to the 5′ terminus of the extracellular antigen-binding domain. In certain embodiments, the signal peptide comprises a CD8 polypeptide, e.g., the CAR comprises a truncated CD8 signal peptide. In certain embodiments, the truncated CD8 signal peptide comprises or consists of the amino acid sequence set forth in SEQ ID NO: 139. An exemplary nucleotide sequence encoding the truncated CD8 signal peptide of SEQ ID NO: 139 is set forth in SEQ ID NO: 140. SEQ ID NOs: 139 and 140 are provided below.

[SEQ ID NO: 139]
MALPVTALLLPLALLLHAARP
[SEQ ID NO: 140]
ATGGCGTTGCCGGTGACCGCGCTTTTGCTCCCTCTGGCCCTGCTCTT
GCACGCCGCACGACCG

### 3.1.2. Transmembrane Domain of a CAR

In certain embodiments, the transmembrane domain of the CAR comprises a hydrophobic alpha helix that spans at least a portion of the membrane. Different transmembrane domains result in different receptor stability. After antigen recognition, receptors cluster and a signal are transmitted to the cell. In accordance with the presently disclosed subject matter, the transmembrane domain of the CAR can comprise a native or modified transmembrane domain of CD8 or a fragment thereof, a native or modified transmembrane domain of CD28 or a fragment thereof, a native or modified transmembrane domain of CD3ζ or a fragment thereof, a native or modified transmembrane domain of CD4 or a fragment thereof, a native or modified transmembrane domain of 4-1BB or a fragment thereof, a native or modified transmembrane domain of OX40 or a fragment thereof, a native or modified transmembrane domain of ICOS or a fragment thereof, a native or modified transmembrane domain of CD84 or a fragment thereof, a native or modified transmembrane domain of CD166 or a fragment thereof, a native or modified transmembrane domain of CD8a or a fragment thereof, a native or modified transmembrane domain of CD8b or a fragment thereof, a native or modified transmembrane domain of ICAM-1 or a fragment thereof, a native or modified transmembrane domain of CTLA-4 or a fragment thereof, a native or modified transmembrane domain of CD27 or a fragment thereof, a native or modified transmembrane domain of CD40 or a fragment thereof, NKGD2 or a fragment thereof, or a combination thereof.

In certain embodiments, the transmembrane domain of a presently disclosed CAR comprises a CD28 polypeptide (e.g., a transmembrane domain of CD28 or a fragment thereof).

In certain embodiments, the transmembrane domain of the CAR comprises a CD28 polypeptide (e.g., a transmembrane domain of human CD28 or a fragment thereof). In certain embodiments, the CD28 polypeptide comprises or consists of an amino acid sequence that is at least about 85%, about 90%, about 95%, about 96%, about 97%, about 98%, about 99% or 100% homologous or identical to the amino acid sequence having a NCBI Reference No: NP_006130 (SEQ ID NO: 8) or a fragment thereof, and/or may optionally comprise up to one or up to two or up to three conservative amino acid substitutions. In certain embodiments, the CD28 polypeptide comprises or consists of an amino acid sequence that is a consecutive portion of SEQ ID NO: 141, which is at least 20, or at least 30, or at least 40, or at least 50, and up to 220 amino acids in length. In certain embodiments, the CD28 polypeptide comprises or consists of amino acids 1 to 220, 1 to 50, 50 to 100, 100 to 150, 150 to 200, 153 to 179, or 200 to 220 of SEQ ID NO: 141. In certain embodiments, the transmembrane domain of the CAR comprises a CD28 polypeptide comprising or consisting of amino acids 153 to 179 of SEQ ID NO: 141. SEQ ID NO: 141 is provided below:

[SEQ ID NO: 141]
MLRLLLALNLFPSIQVTGNKILVKQSPMLVAYDNAVNLSCKYSYNLFSR
EFRASLHKGLDSAVEVCVVYGNYSQQLQVYSKTGFNCDGKLGNESVTFY
LQNLYVNQTDIYFCKIEVMYPPPYLDNEKSNGTIIHVKGKHLCPSPLFP
GPSKPFWVLVVVGGVLACYSLLVTVAFIIFWVRSKRSRLLHSDYMNMTP
RRPGPTRKHYQPYAPPRDFAAYRS

An exemplary nucleotide sequence encoding amino acid 153 to 179 of SEQ ID NO: 141 is set forth in SEQ ID NO: 142, which is provided below.

[SEQ ID NO: 142]
TTCTGGGTACTGGTGGTGGTGGGCGGAGTTCTCGCATGTTATTCATTGC
TGGTAACGGTCGCATTCATTATATTCTGGGTG

In certain embodiments, the transmembrane domain of the CAR comprises a CD28 polypeptide (e.g., a transmembrane domain of mouse CD28 or a fragment thereof). In certain embodiments, the CD28 polypeptide comprises or consists of an amino acid sequence that is at least about 85%, about 90%, about 95%, about 96%, about 97%, about 98%, about 99% or 100% homologous or identical to the amino acid sequence having a NCBI Reference No: NP_031668.3 (SEQ ID NO: 143) or a fragment thereof, and/or may optionally comprise up to one or up to two or up to three conservative amino acid substitutions. In certain embodiments, the CD28 polypeptide comprises or consists of an amino acid sequence that is a consecutive portion of SEQ ID NO: 143, which is at least 20, or at least 30, or at least 40, or at least 50, and up to 218 amino acids in length. In certain embodiments, the CD28 polypeptide comprises or consists of amino acids 1 to 220, 1 to 50, 50 to 100, 100 to 150, 150 to 200, 151 to 177, or 200 to 218 of SEQ ID NO: 143. In certain embodiments, the transmembrane domain of the CAR comprises a CD28 polypeptide comprising or consisting of amino acids 151 to 177 of SEQ ID NO: 143. SEQ ID NO: 143 is provided below:

[SEQ ID NO: 143]
MTLRLLFLALNFFSVQVTENKILVKQSPLLVVDSNEVSLSCRYSYNLLA
KEFRASLYKGVNSDVEVCVGNGNFTYQPQFRSNAEFNCDGDFDNETVTF
RLWNLHVNHTDIYFCKIEFMYPPPYLDNERSNGTIIHIKEKHLCHTQSS
PKLFWALVVVAGVLFCYGLLVTVALCVIWTNSRRNRLLQSDYMNMTPRR
PGLTRKPYQPYAPARDFAAYRP

In certain embodiments, the transmembrane domain of the CAR comprises a CD8 polypeptide (e.g., a transmembrane domain of CD8 or a fragment thereof).

In certain embodiments, the transmembrane domain of the CAR comprises a CD8 polypeptide (e.g., a transmembrane domain of CD8 or a fragment thereof). In certain embodiments, the transmembrane domain of the CAR comprises a CD8 polypeptide (e.g., a transmembrane domain of human CD8 or a fragment thereof). In certain embodiments, the CD8 polypeptide comprises or consists of an amino acid sequence that is at least about 85%, about 90%, about 95%, about 96%, about 97%, about 98%, about 99% or about 100% homologous or identical to the amino acid sequence having a NCBI Reference No: NP_001139345.1 (SEQ ID NO: 144) or a fragment thereof, and/or may optionally comprise up to one or up to two or up to three conservative amino acid substitutions. In certain embodiments, the CD8 polypeptide comprises or consists of an amino acid sequence that is a consecutive portion of SEQ ID NO: 144, which is at least 20, or at least 30, or at least 40, or at least 50, and up to 235 amino acids in length. In certain embodiments, the CD8 polypeptide comprises or consists of amino acids 1 to 235, 1 to 50, 50 to 100, 100 to 150, 150 to 200, 137 to 209 or 200 to 235 of SEQ ID NO: 144. In certain embodiments, the transmembrane domain of the CAR comprises a CD8 polypeptide comprising or consisting of amino acids 137 to 209 of SEQ ID NO: 144. SEQ ID NO: 144 is provided below.

[SEQ ID NO: 144]
MALPVTALLLPLALLLHAARPSQFRVSPLDRTWNLGETVELKCQVLLSN
PTSGCSWLFQPRGAAASPTFLLYLSQNKPKAAEGLDTQRFSGKRLGDTF
VLTLSDFRRENEGYYFCSALSNSIMYFSHFVPVFLPAKPTTTPAPRPPT
PAPTIASQPLSLRPEACRPAAGGAVHTRGLDFACDIYIWAPLAGTCGVL
LLSLVITLYCNHRNRRRVCKCPRPVVKSGDKPSLSARYV

In certain embodiments, the transmembrane domain of the CAR comprises a CD8 polypeptide (e.g., a transmembrane domain of mouse CD8 or a fragment thereof). In certain embodiments, the CD8 polypeptide comprises or consists of an amino acid sequence that is at least about 85%, about 90%, about 95%, about 96%, about 97%, about 98%, about 99% or about 100% homologous or identical to the amino acid sequence having a NCBI Reference No: AAA92533.1 (SEQ ID NO: 145) or a fragment thereof, and/or may optionally comprise up to one or up to two or up to three conservative amino acid substitutions. In certain embodiments, the CD8 polypeptide comprises or consists of an amino acid sequence that is a consecutive portion of SEQ ID NO: 145, which is at least about 20, or at least about 30, or at least about 40, or at least about 50, or at least about 60, or at least about 70, or at least about 100, or at least about 200, and up to 247 amino acids in length. In certain embodiments, the CD8 polypeptide comprises or consists of amino acids 1 to 247, 1 to 50, 50 to 100, 100 to 150, 150 to 200, 151 to 219, or 200 to 247 of SEQ ID NO: 145. In certain embodiments, the transmembrane domain of the CAR comprises a CD8 polypeptide comprising or consisting of amino acids 151 to 219 of SEQ ID NO: 145. SEQ ID NO: 145 is provided below.

[SEQ ID NO: 145]
MASPLTRFLSLNLLLMGESIILGSGEAKPQAPELRIFPKKMDAELGQKV
DLVCEVLGSVSQGCSWLFQNSSSKLPQPTFVVYMASSHNKITWDEKLNS
SKLFSAVRDTNNKYVLTLNKFSKENEGYYFCSVISNSVMYFSSVVPVLQ
KVNSTTTKPVLRTPSPVHPTGTSQPQRPEDCRPRGSVKGTGLDFACDIY
IWAPLAGICVAPLLSLIITLICYHRSRKRVCKCPRPLVRQEGKPRPSEK
IV

In certain embodiments, the CAR further comprises a spacer region that links the extracellular antigen-binding domain to the transmembrane domain. The spacer region can be flexible enough to allow the antigen binding domain to orient in different directions to facilitate antigen recognition while preserving the activating activity of the CAR.

In certain embodiments, the hinge/spacer region of the CAR comprises a native or modified hinge region of CD8 or a fragment thereof, a native or modified hinge region of CD28 or a fragment thereof, a native or modified hinge region of CD3ζ or a fragment thereof, a native or modified hinge region of CD40 or a fragment thereof, a native or modified hinge region of 4-1BB or a fragment thereof, a native or modified hinge region of OX40 or a fragment thereof, a native or modified hinge region of CD84 or a fragment thereof, a native or modified hinge region of CD166 or a fragment thereof, a native or modified hinge region of CD8a or a fragment thereof, a native or modified hinge region of CD8b or a fragment thereof, a native or modified hinge region of ICOS or a fragment thereof, a native or modified hinge region of ICAM-1 or a fragment thereof, a native or modified hinge region of CTLA-4 or a fragment thereof, a native or modified hinge region of CD27 or a fragment thereof, a native or modified hinge region of CD40 or a fragment thereof, a native or modified hinge region of NKGD2 or a fragment thereof, a synthetic polypeptide (not based on a protein associated with the immune response), or a combination thereof. The hinge/spacer region can be the hinge region from IgG1, or the CH₂CH₃ region of immunoglobulin and portions of CD3, a portion of a CD28 polypeptide (e.g., a portion of SEQ ID NO: 8 or 10), a portion of a CD8 polypeptide (e.g., a portion of SEQ ID NO: 11 or 12), a variation of any of the foregoing which is at least about 80%, at least about 85%, at least about 90%, at least about 95%, or at least about 100% homologous or identical thereto, or a synthetic spacer sequence.

In certain embodiments, the hinge/spacer region of the CAR comprises a CD28 polypeptide. In certain embodiments, the hinge/spacer region of the CAR comprises a CD28 polypeptide comprising or consisting of amino acids 114 to 152 of SEQ ID NO: 141.

An exemplary nucleotide sequence encoding amino acid 114 to 152 of SEQ ID NO: 141 is set forth in SEQ ID NO: 146, which is provided below.

[SEQ ID NO: 146]
ATTGAAGTCATGTATCCTCCTCCGTACTTGGATAATGAAAAAAGTAATG
GGACCATAATTCACGTCAAAGGAAAACATTTGTGTCCAAGCCCCCTTTT
CCCTGGCCCGTCCAAGCCT

### 3.1.3. Intracellular Signaling Domain of a CAR

In certain embodiments, the CAR comprises an intracellular signaling domain. In certain embodiments, the intracellular signaling domain of the CAR comprises a CD3ζ polypeptide. CD3ζ can activate or stimulate a cell (e.g., a cell of the lymphoid lineage, e.g., a T cell). Wild type (“native”) CD3ζ comprises three functional immunoreceptor tyrosine-based activation motifs (ITAMs), three functional basic-rich stretch (BRS) regions (BRS1, BRS2 and BRS3). CD3ζ transmits an activation signal to the cell (e.g., a cell of the lymphoid lineage, e.g., a T cell) after antigen is bound. The intracellular signaling domain of the CD3ζ-chain is the primary transmitter of signals from endogenous TCRs.

In certain embodiments, the intracellular signaling domain of the CAR comprises a native CD3ζ. In certain embodiments, the native CD3ζ polypeptide comprises or consists of an amino acid sequence that is at least about 85%, about 90%, about 95%, about 96%, about 97%, about 98%, about 99% or about 100% homologous or identical to the amino acid sequence having a NCBI Reference No: NP_932170 (SEQ ID NO: 147) or a fragment thereof, and/or may optionally comprise up to one or up to two or up to three conservative amino acid substitutions. In certain embodiments, the native CD3ζ polypeptide comprises or consists of an amino acid sequence that is a consecutive portion of SEQ ID NO: 147, which is at least 20, or at least 30, or at least 40, or at least 50, and up to 164 amino acids in length. In certain embodiments, the native CD3ζ polypeptide comprises or consists of amino acids 1 to 164, 1 to 50, 50 to 100, 52 to 164, 100 to 150, or 150 to 164 of SEQ ID NO: 147. In certain embodiments, the intracellular signaling domain of the CAR comprises a CD3ζ polypeptide comprising or consisting of amino acids 52 to 164 of SEQ ID NO: 147. SEQ ID NO: 147 is provided below:

[SEQ ID NO: 147]
MKWKALFTAAILQAQLPITEAQSFGLLDPKLCYLLDGILFIYGVILTAL
FLRVKFSRSADAPAYQQGQNQLYNELNLGRREEYDVLDKRRGRDPEMGG
KPQRRKNPQEGLYNELQKDKMAEAYSEIGMKGERRRGKGHDGLYQGLST
ATKDTYDALHMQALPPR

In certain embodiments, the intracellular signaling domain of the CAR comprises a CD3ζ polypeptide comprising or consisting of the amino acid sequence set forth in SEQ ID NO: 148, which is provided below.

[SEQ ID NO: 148]
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

An exemplary nucleotide sequence encoding the amino acid sequence of SEQ ID NO: 148 is set forth in SEQ ID NO: 149, which is as provided below.

[SEQ ID NO: 149]
CGAGTAAAATTTTCCCGAAGTGCAGATGCGCCAGCTTATCAGCAAGGCCA
GAATCAGCTCTACAATGAGTTGAACCTCGGACGGCGCGAGGAATATGATG
TGCTCGATAAGCGCAGAGGTAGGGACCCTGAGATGGGGGGTAAACCAAGG
CGAAAGAATCCCCAAGAGGGGCTTTACAACGAATTGCAAAAAGACAAAAT
GGCAGAAGCCTATTCCGAAATTGGAATGAAGGGCGAACGGAGGCGAGGTA
AGGGCCACGACGGGCTCTACCAAGGCCTGAGCACAGCAACAAAAGACACG
TATGACGCATTGCACATGCAAGCTCTTCCGCCCCGG

In certain embodiments, the intracellular signaling domain of the CAR comprises a modified CD3ζ polypeptide. In certain embodiments, the modified CD3ζ polypeptide comprises one, two or three ITAMs. In certain embodiments, the modified CD3ζ polypeptide comprises a native ITAM1. In certain embodiments, the native ITAM1 comprises or consists of the amino acid sequence set forth in SEQ ID NO: 150.

[SEQ ID NO: 150]
QNQLYNELNLGRREEYDVLDKR

An exemplary nucleotide sequence encoding the amino acid sequence of SEQ ID NO: 150 is set forth in SEQ ID NO: 151, which is provided below.

[SEQ ID NO: 151]
CAGAACCAGCTCTATAACGAGCTCAATCTAGGACGAAGAGAGGAGTACGA
TGTTTTGGACAAGAGA

In certain embodiments, the modified CD3ζ polypeptide comprises an ITAM1 variant comprising one or more loss-of-function mutations. In certain embodiments, the ITAM1 variant comprises or consists of two loss-of-function mutations. In certain embodiments, each of the one or more (e.g., two) loss of function mutations comprises a mutation of a tyrosine residue in ITAM1. In certain embodiments, the ITAM1 variant consists of two loss-of-function mutations. In certain embodiments, the ITAM1 variant comprises or consists of the amino acid sequence set forth in SEQ ID NO: 152, which is provided below.

[SEQ ID NO: 152]
QNQLFNELNLGRREEFDVLDKR

An exemplary nucleotide sequence encoding the amino acid sequence of SEQ ID NO: 152 is set forth in SEQ ID NO: 153, which is provided below.

[SEQ ID NO: 153]
CAGAACCAGCTCTTTAACGAGCTCAATCTAGGACGAAGAGAGGAGTTCGA
TGTTTTGGACAAGAGA

In certain embodiments, the modified CD3ζ polypeptide comprises a native ITAM2. In certain embodiments, the native ITAM2 comprises or consists of the amino acid sequence set forth in SEQ ID NO: 154, which is provided below.

[SEQ ID NO: 154]
QEGLYNELQKDKMAEAYSEIGMK

An exemplary nucleotide sequence encoding the amino acid sequence of SEQ ID NO: 154 is set forth in SEQ ID NO: 155, which is provided below.

[SEQ ID NO: 155]
CAGGAAGGCCTGTACAATGAACTGCAGAAAGATAAGATGGCGGAGGCCTA
CAGTGAGATTGGGATGAAA

In certain embodiments, the modified CD3ζ polypeptide comprises an ITAM2 variant. In certain embodiments, the ITAM2 variant comprises or consists of one or more loss-of-function mutations. In certain embodiments, the ITAM2 variant comprises or consists of two loss-of-function mutations. In certain embodiments, each of the one or more (e.g., two) the loss of function mutations comprises a mutation of a tyrosine residue in ITAM2. In certain embodiments, the ITAM1 variant consists of two loss-of-function mutations. In certain embodiments, the ITAM2 variant comprises or consists of the amino acid sequence set forth in SEQ ID NO: 156, which is provided below.

[SEQ ID NO: 156]
QEGLFNELQKDKMAEAFSEIGMK

An exemplary nucleotide sequence encoding the amino acid sequence of SEQ ID NO: 156 is set forth in SEQ ID NO: 157, which is provided below.

[SEQ ID NO: 157]
CAGGAAGGCCTGTTCAATGAACTGCAGAAAGATAAGATGGCGGAGGCCTT
CAGTGAGATTGGGATGAAA

In certain embodiments, the modified CD3ζ polypeptide comprises a native ITAM3. In certain embodiments, the native ITAM3 comprises or consists of the amino acid sequence set forth in SEQ ID NO: 158, which is provided below.

[SEQ ID NO: 158]
HDGLYQGLSTATKDTYDALHMQ

An exemplary nucleotide sequence encoding the amino acid sequence of SEQ ID NO: 158 is set forth in SEQ ID NO: 159, which is provided below.

[SEQ ID NO: 159]
CACGATGGCCTTTACCAGGGTCTCAGTACAGCCACCAAGGACACCTACGA
CGCCCTTCACATGCAG

In certain embodiments, the modified CD3ζ polypeptide comprises an ITAM3 variant. In certain embodiments, the ITAM3 variant comprises or consists of two loss-of-function mutations. In certain embodiments, each of the one or more (e.g., two) the loss of function mutations comprises a mutation of a tyrosine residue in ITAM3. In certain embodiments, the ITAM3 variant comprises or consists of two loss-of-function mutations. In certain embodiments, the ITAM3 variant comprises or consists of the amino acid sequence set forth in SEQ ID NO: 160, which is provided below.

[SEQ ID NO: 160]
HDGLFQGLSTATKDTFDALHMQ

An exemplary nucleotide sequence encoding the amino acid sequence of SEQ ID NO: 160 is set forth in SEQ ID NO: 161, which is provided below.

[SEQ ID NO: 161]
CACGATGGCCTTTTCCAGGGGCTCAGTACAGCCACCAAGGACACCTTCGA
CGCCCTTCACATGCAG

Various modified CD3ζ polypeptides and CARs comprising modified CD3ζ polypeptides are disclosed in International Patent Application Publication No. WO2019/133969, which is incorporated by reference hereby in its entirety.

In certain embodiments, the intracellular signaling domain of the CAR comprises a modified CD3ζ polypeptide comprising a native ITAM1, an ITAM2 variant comprising or consisting of one or more (e.g., two) loss-of-function mutations, and an ITAM3 variant comprising or consisting of one or more (e.g., two) loss-of-function mutations. In certain embodiments, the intracellular signaling domain of the CAR comprises a modified CD3ζ polypeptide comprising a native ITAM1, an ITAM2 variant consisting of two loss-of-function mutations, and an ITAM3 variant consisting of two loss-of-function mutations. In certain embodiments, the intracellular signaling domain of the CAR comprises a modified CD3ζ polypeptide comprising a native ITAM1 consisting of the amino acid sequence set forth in SEQ ID NO: 150, an ITAM2 variant consisting of the amino acid sequence set forth in SEQ ID NO: 156, and an ITAM3 variant consisting of the amino acid sequence set forth in SEQ ID NO: 160. In certain embodiments, the modified CD3ζ polypeptide is designated as “1XX”. In certain embodiments, the modified CD3ζ polypeptide comprises or consists of the amino acid sequence set forth in SEQ ID NO: 162. SEQ ID NO: 162 is provided below.

[SEQ ID NO: 162]
RVKFSRSADAPAYQQGQNQLYNELNLGRREEYDVLDKRRGRDPEMGGKPR
RKNPQEGLFNELQKDKMAEAFSEIGMKGERRRGKGHDGLFQGLSTATKDT
FDALHMQALPPR

In certain embodiments, the intracellular signaling domain of the CAR comprises a modified CD3ζ polypeptide comprising or consisting of an amino acid sequence that is at least about 80%, at least about 85%, at least about 90%, at least about 95%, at least about 96%, at least about 97%, at least about 98%, or at least about 99%, at least about 100% identical to SEQ ID NO: 162 or a fragment thereof, and/or may optionally comprise up to one or up to two or up to three conservative amino acid substitutions.

An exemplary nucleotide sequence encoding the amino acid sequence of SEQ ID NO: 162 is set forth in SEQ ID NO: 163, which is provided below.

[SEQ ID NO: 163]
AGAGTGAAGTTCAGCAGGAGCGCAGACGCCCCCGCGTACCAGCAG
GGCCAGAACCAGCTCTATAACGAGCTCAATCTAGGACGAAGAGAG
GAGTACGATGTTTTGGACAAGAGACGTGGCCGGGACCCTGAGATG
GGGGGAAAGCCGAGAAGGAAGAACCCTCAGGAAGGCCTGTTCAAT
GAACTGCAGAAAGATAAGATGGCGGAGGCCTTCAGTGAGATTGGG
ATGAAAGGCGAGCGCCGGAGGGGCAAGGGGCACGATGGCCTTTTC
CAGGGGCTCAGTACAGCCACCAAGGACACCTTCGACGCCCTTCAC
ATGCAGGCCCTGCCCCCTCGC

In certain embodiments, the intracellular signaling domain of the CAR further comprises at least a co-stimulatory signaling region. In certain embodiments, the co-stimulatory signaling region comprises at least one co-stimulatory molecule or a fragment thereof. In certain embodiments, the co-stimulatory signaling region comprises an intracellular domain of at least one co-stimulatory molecule or a fragment thereof.

As used herein, a “co-stimulatory molecule” refers to a cell surface molecule other than antigen receptor or its ligand that can provide an efficient response of lymphocytes to an antigen. In certain embodiments, a co-stimulatory molecule can provide optimal lymphocyte activation. Non-limiting examples of co-stimulatory molecules include CD28, 4-1BB, OX40, ICOS, DAP-10, CD27, CD40, NKGD2, CD2, FN14, HVEM, LTBR, CD28H, TNFR1, TNFR2, BAFF-R, BCMA, TACI, TROY, RANK, CD40, CD27, CD30, EDAR, XEDAR, GITR, DR6, and NGFR, and combinations thereof. The co-stimulatory molecule can bind to a co-stimulatory ligand, which is a protein expressed on cell surface that upon binding to its receptor produces a co-stimulatory response, i.e., an intracellular response that effects the stimulation provided when an antigen-recognizing receptor (e.g., a chimeric antigen receptor (CAR)) binds to its target antigen. As one example, a 4-1BB ligand (i.e., 4-1BBL) may bind to 4-1BB for providing an intracellular signal that in combination with a CAR signal induces an effector cell function of the CAR⁺ T cell.

In certain embodiments, the intracellular signaling domain of the CAR comprises a co-stimulatory signaling region that comprises a CD28 polypeptide, e.g., an intracellular domain of CD28 or a fragment thereof. The CD28 polypeptide can comprise or have an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 141 or a fragment thereof, and/or may optionally comprise up to one or up to two or up to three conservative amino acid substitutions. In certain embodiments, the CD28 polypeptide comprises or consists of an amino acid sequence that is a consecutive portion of SEQ ID NO: 141, which is at least 20, or at least 30, or at least 40, or at least 50, and up to 220 amino acids in length. In certain embodiments, the CD28 polypeptide comprises or consists of amino acids 1 to 220, 1 to 50, 50 to 100, 100 to 150, 114 to 220, 150 to 200, 180 to 220, or 200 to 220 of SEQ ID NO: 141. In certain embodiments, the intracellular signaling domain of the CAR comprises a co-stimulatory signaling region that comprises a CD28 polypeptide comprising or consisting of amino acids 180 to 220 of SEQ ID NO: 141.

An exemplary nucleic acid sequence encoding amino acids 180 to 220 of SEQ ID NO: 141 is set forth in SEQ ID NO: 164, which is provided below.

[SEQ ID NO: 164]
CGATCAAAACGGAGTAGGCTTCTCCATAGTGACTATATGAACATG
ACGCCGAGGCGCCCAGGCCCAACGCGCAAACATTATCAACCATAC
GCTCCGCCACGAGATTTCGCAGCCTATAGATCC

In certain embodiments, the CD28 polypeptide comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 143 or a fragment thereof, and/or may optionally comprise up to one or up to two or up to three conservative amino acid substitutions. In certain embodiments, the CD28 polypeptide comprises or consists of an amino acid sequence that is a consecutive portion of SEQ ID NO: 143, which is at least about 20, or at least about 30, or at least about 40, or at least about 50, and up to 218 amino acids in length. In certain embodiments, the CD28 polypeptide comprises or consists of amino acids 1 to 218, 1 to 50, 50 to 100, 100 to 150, 150 to 218, 178 to 218, or 200 to 218 of SEQ ID NO: 143. In certain embodiments, the co-stimulatory signaling region of a presently disclosed CAR comprises a CD28 polypeptide that comprises or consists of the amino acids 178 to 218 of SEQ ID NO: 143.

In certain embodiments, the intracellular signaling domain of the CAR comprises a co-stimulatory signaling region that comprises a 4-1BB polypeptide, e.g., an intracellular domain of 4-1BB or a fragment thereof (e.g., an intracellular domain of human 4-1BB or a fragment thereof). The 4-1BB polypeptide can comprise or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence having a NCBI Ref. No.: NP_001552 (SEQ ID NO: 165) or a fragment thereof, and/or may optionally comprise up to one or up to two or up to three conservative amino acid substitutions. In certain embodiments, the 4-1BB polypeptide comprises or consists of an amino acid sequence that is a consecutive portion of SEQ ID NO: 165, which is at least 20, or at least 30, or at least 40, or at least 50, or at least 100, or at least 150, or at least 150, and up to 255 amino acids in length. In certain embodiments, the 4-1BB polypeptide comprises or consists of amino acids 1 to 255, 1 to 50, 50 to 100, 100 to 150, 150 to 200, or 200 to 255 of SEQ ID NO: 165. In certain embodiments, the intracellular signaling domain of the CAR comprises a co-stimulatory signaling region that comprises a 4-1BB polypeptide comprising or consisting of amino acids 214 to 255 of SEQ ID NO: 165. SEQ ID NO: 165 is provided below.

[SEQ ID NO: 165]
MGNSCYNIVATLLLVLNFERTRSLQDPCSNCPAGTFCDNNRNQIC
SPCPPNSESSAGGQRTCDICRQCKGVFRTRKECSSTSNAECDCTP
GFHCLGAGCSMCEQDCKQGQELTKKGCKDCCFGTENDQKRGICRP
WTNCSLDGKSVLVNGTKERDVVCGPSPADLSPGASSVTPPAPARE
PGHSPQIISFFLALTSTALLFLLFFLTLRFSVVKRGRKKLLYIFK
QPFMRPVQTTQEEDGCSCRFPEEEEGGCEL

In certain embodiments, the intracellular signaling domain of the CAR comprises a co-stimulatory signaling region that comprises intracellular domains of two or more co-stimulatory molecules or portions thereof, e.g., an intracellular domain of CD28 or a fragment thereof and an intracellular domain of 4-1BB or a fragment thereof, or an intracellular domain of CD28 or a fragment thereof and an intracellular domain of OX40 or a fragment thereof.

### 3.1.4. Exemplified CARs

In certain embodiments, the CAR is a L1CAM-targeted CAR. In certain embodiments, the CAR comprises (a) an extracellular antigen-binding domain comprising (i) a V_{H} that comprises a CDR1 comprising the amino acid sequence set forth in SEQ ID NO: 81, a CDR2 comprising the amino acid sequence set forth in SEQ ID NO: 82, and a V_{H} CDR3 comprising the amino acid sequence set forth in SEQ ID NO: 83, and (ii) a V_{L} that comprises a CDR1 comprising the amino acid sequence set forth in SEQ ID NO: 84, a CDR2 comprising the amino acid sequence set forth in SEQ ID NO: 85, and a V_{L} CDR3 comprising the amino acid sequence set forth in SEQ ID NO: 86; (b) a transmembrane domain comprising a CD28 polypeptide (e.g., a transmembrane domain of human CD28 or a fragment thereof), and (c) an intracellular signaling domain comprising (i) a CD3ζ polypeptide, and (ii) a co-stimulatory signaling region comprising a CD28 polypeptide (e.g., an intracellular domain of human CD28 or a fragment thereof). In certain embodiments, the V_{H} and V_{L} are linked via a linker comprising or consisting of the amino acid sequence set forth in SEQ ID NO: 11. In certain embodiments, the V_{H} and V_{L} are positioned from the N- to the C-terminus: V_{L}-V_{H}. In certain embodiments, the CAR is designed as “14A10_LH”. In certain embodiments, the CAR comprises the amino acid sequence set forth in SEQ ID NO: 166, which is provided below.

[SEQ ID NO: 166]
MALPVTALLLPLALLLHAARPDIVMTQTPLSSPVSLGQPASISCR
SSQSLVHSDGNTYLSWLQQRPGQPPRVLIYKISKRFSGVPDRESG
SGAGTDFTLKISRVEAEDVGVYYCMQATQFPFTFGPGTKVDIKGG
GGSGGGGSGGGGSEVQMVESGGGLVQPGGSLRLSCAAPGFIFSSY
NMNWVRQAPGKGLEWVSYISSSSSTTYYADSVKGRFTISRDNAKN
SLYLQMNSLRDEDTAVYYCASLYSGYDYYYYYGMDVWGQGTTVTV
SSAAAIEVMYPPPYLDNEKSNGTIIHVKGKHLCPSPLFPGPSKPF
WVLVVVGGVLACYSLLVTVAFIIFWVRSKRSRLLHSDYMNMTPRR
PGPTRKHYQPYAPPRDFAAYRSRVKFSRSADAPAYQQGQNQLYNE
LNLGRREEYDVLDKRRGRDPEMGGKPRRKNPQEGLYNELQKDKMA
EAYSEIGMKGERRRGKGHDGLYQGLSTATKDTYDALHMQALPPR

An exemplary nucleic acid sequence encoding the amino acid sequence of SEQ ID NO: 166 is set forth in SEQ ID NO: 167, which is provided below.

[SEQ ID NO: 167]
ATGGCGTTGCCGGTGACCGCGCTTTTGCTCCCTCTGGCCCTGCTC
TTGCACGCCGCACGACCGGATATTGTTATGACGCAAACGCCCTTG
TCATCACCTGTATCATTGGGTCAGCCGGCTTCCATATCCTGCCGA
TCTAGTCAATCCTTGGTGCACTCAGACGGGAATACCTACCTTTCA
TGGCTCCAGCAGAGACCCGGGCAACCACCGAGAGTGTTGATCTAT
AAAATTTCTAAACGGTTTAGCGGGGTCCCTGACAGATTCTCCGGT
TCCGGGGCTGGAACTGATTTTACACTTAAAATATCCCGAGTCGAA
GCGGAAGACGTGGGGGTGTATTATTGCATGCAAGCAACTCAATTT
CCCTTCACATTTGGGCCTGGTACAAAAGTGGACATCAAGGGCGGT
GGTGGCTCAGGCGGCGGAGGATCCGGGGGGGGGGGATCCGAAGTC
CAGATGGTGGAGTCAGGAGGGGGTCTGGTCCAACCGGGGGGTAGC
CTGAGGTTGTCCTGCGCGGCACCAGGATTTATCTTCTCAAGCTAT
AACATGAACTGGGTACGCCAAGCACCTGGCAAAGGACTGGAATGG
GTAAGTTACATTTCTTCCAGTTCATCAACAACTTACTATGCAGAC
AGTGTGAAAGGCCGGTTTACTATAAGCCGGGACAATGCTAAGAAC
TCACTCTATCTCCAAATGAACTCCCTCCGCGACGAGGACACAGCT
GTCTATTACTGTGCTAGTCTCTACTCTGGCTACGATTACTATTAC
TATTATGGAATGGATGTGTGGGGGCAAGGCACTACTGTCACAGTC
TCAAGCGCGGCCGCAATTGAAGTCATGTATCCTCCTCCGTACTTG
GATAATGAAAAAAGTAATGGGACCATAATTCACGTCAAAGGAAAA
CATTTGTGTCCAAGCCCCCTTTTCCCTGGCCCGTCCAAGCCTTTC
TGGGTACTGGTGGTGGTGGGCGGAGTTCTCGCATGTTATTCATTG
CTGGTAACGGTCGCATTCATTATATTCTGGGTGCGATCAAAACGG
AGTAGGCTTCTCCATAGTGACTATATGAACATGACGCCGAGGCGC
CCAGGCCCAACGCGCAAACATTATCAACCATACGCTCCGCCACGA
GATTTCGCAGCCTATAGATCCCGAGTAAAATTTTCCCGAAGTGCA
GATGCGCCAGCTTATCAGCAAGGCCAGAATCAGCTCTACAATGAG
TTGAACCTCGGACGGCGCGAGGAATATGATGTGCTCGATAAGCGC
AGAGGTAGGGACCCTGAGATGGGGGGTAAACCAAGGCGAAAGAAT
CCCCAAGAGGGGCTTTACAACGAATTGCAAAAAGACAAAATGGCA
GAAGCCTATTCCGAAATTGGAATGAAGGGCGAACGGAGGCGAGGT
AAGGGCCACGACGGGCTCTACCAAGGCCTGAGCACAGCAACAAAA
GACACGTATGACGCATTGCACATGCAAGCTCTTCCGCCCCGG

In certain embodiments, the CAR is a L1CAM-targeted CAR. In certain embodiments, the CAR comprises (a) an extracellular antigen-binding domain comprising (i) a V_{H} that comprises a CDR1 comprising the amino acid sequence set forth in SEQ ID NO: 93, a CDR2 comprising the amino acid sequence set forth in SEQ ID NO: 94, and a V_{H} CDR3 comprising the amino acid sequence set forth in SEQ ID NO: 95, and (ii) a V_{L} that comprises a CDR1 comprising the amino acid sequence set forth in SEQ ID NO: 96, a CDR2 comprising the amino acid sequence set forth in SEQ ID NO: 97, and a V_{L} CDR3 comprising the amino acid sequence set forth in SEQ ID NO: 98; (b) a transmembrane domain comprising a CD28 polypeptide (e.g., a transmembrane domain of human CD28 or a fragment thereof), and (c) an intracellular signaling domain comprising (i) a CD3ζ polypeptide, and (ii) a co-stimulatory signaling region comprising a CD28 polypeptide (e.g., an intracellular domain of human CD28 or a fragment thereof). In certain embodiments, the V_{H} and V_{L} are linked via a linker comprising or consisting of the amino acid sequence set forth in SEQ ID NO: 11. In certain embodiments, the V_{H} and V_{L} are positioned from the N- to the C-terminus: V_{H}-V_{L}. In certain embodiments, the CAR is designed as “76H05_HL”. In certain embodiments, the CAR comprises the amino acid sequence set forth in SEQ ID NO: 168, which is provided below.

[SEQ ID NO: 168]
MALPVTALLLPLALLLHAARPQVQLKESGPGLVAPSQSLSITCTI
SGESLTSYGVHWVRQPPGKGLEWLVVIWGDGSTSYNSALKSRLSI
SKDNSKSQVFLKMNSLQTDDTAMYFCATSTVGYAVDYWGQGTSVT
VSSGGGGSGGGGSGGGGSDIQMTQSPASLSVSVGETVTITCRASE
NIYSNLAWYQQRQGKSPQLLVYAATNLADGVPSRFSGSGSGTQYS
LKINSLQSEDFGSYYCQHFWGSPYTFGGGTKLEIKAAAIEVMYPP
PYLDNEKSNGTIIHVKGKHLCPSPLFPGPSKPFWVLVVVGGVLAC
YSLLVTVAFIIFWVRSKRSRLLHSDYMNMTPRRPGPTRKHYQPYA
PPRDFAAYRSRVKFSRSADAPAYQQGQNQLYNELNLGRREEYDVL
DKRRGRDPEMGGKPRRKNPQEGLYNELQKDKMAEAYSEIGMKGER
RRGKGHDGLYQGLSTATKDTYDALHMQALPPR

An exemplary nucleic acid sequence encoding the amino acid sequence of SEQ ID NO: 168 is set forth in SEQ ID NO: 169, which is provided below.

[SEQ ID NO: 169]
ATGGCGTTGCCGGTGACCGCGCTTTTGCTCCCTCTGGCCCTGCTC
TTGCACGCCGCACGACCGCAAGTCCAGCTCAAGGAGAGCGGTCCA
GGACTCGTAGCACCCTCTCAATCTTTGTCTATCACTTGTACCATA
AGCGGTTTTTCCTTGACAAGTTATGGCGTACATTGGGTGAGACAA
CCGCCAGGTAAAGGACTCGAATGGTTGGTTGTTATCTGGGGGGAT
GGGAGTACCAGCTACAACAGCGCACTCAAGTCTCGCCTTTCAATT
TCTAAGGACAACAGTAAATCACAGGTTTTTCTGAAGATGAATAGC
CTGCAAACCGACGACACGGCGATGTATTTCTGTGCGACTTCTACG
GTAGGTTATGCCGTCGACTATTGGGGTCAGGGTACGTCAGTTACC
GTAAGTAGCGGCGGTGGTGGCTCAGGCGGCGGAGGATCCGGGGGG
GGGGGATCCGACATCCAGATGACCCAGTCACCGGCCAGTTTGTCC
GTTTCCGTAGGCGAGACCGTAACCATCACATGCCGAGCAAGCGAG
AATATCTATTCCAACCTGGCCTGGTATCAGCAGAGACAAGGCAAA
AGTCCGCAACTTTTGGTTTATGCTGCCACTAATTTGGCCGATGGG
GTCCCGAGTCGATTTTCTGGCTCTGGCTCCGGGACGCAGTATTCC
CTGAAGATAAACTCTTTGCAGTCAGAAGATTTTGGTTCATATTAT
TGTCAGCATTTCTGGGGCAGCCCGTACACATTTGGAGGTGGGACC
AAATTGGAAATAAAAGCGGCCGCAATTGAAGTCATGTATCCTCCT
CCGTACTTGGATAATGAAAAAAGTAATGGGACCATAATTCACGTC
AAAGGAAAACATTTGTGTCCAAGCCCCCTTTTCCCTGGCCCGTCC
AAGCCTTTCTGGGTACTGGTGGTGGTGGGCGGAGTTCTCGCATGT
TATTCATTGCTGGTAACGGTCGCATTCATTATATTCTGGGTGCGA
TCAAAACGGAGTAGGCTTCTCCATAGTGACTATATGAACATGACG
CCGAGGCGCCCAGGCCCAACGCGCAAACATTATCAACCATACGCT
CCGCCACGAGATTTCGCAGCCTATAGATCCCGAGTAAAATTTTCC
CGAAGTGCAGATGCGCCAGCTTATCAGCAAGGCCAGAATCAGCTC
TACAATGAGTTGAACCTCGGACGGCGCGAGGAATATGATGTGCTC
GATAAGCGCAGAGGTAGGGACCCTGAGATGGGGGGTAAACCAAGG
CGAAAGAATCCCCAAGAGGGGCTTTACAACGAATTGCAAAAAGAC
AAAATGGCAGAAGCCTATTCCGAAATTGGAATGAAGGGCGAACGG
AGGCGAGGTAAGGGCCACGACGGGCTCTACCAAGGCCTGAGCACA
GCAACAAAAGACACGTATGACGCATTGCACATGCAAGCTCTTCCG
CCCCGG

In certain embodiments, the V_{H} and V_{L} are positioned from the N- to the C-terminus: V_{L}-V_{H}. In certain embodiments, the CAR is designed as “76H05_LH”. In certain embodiments, the CAR comprises the amino acid sequence set forth in SEQ ID NO: 170, which is provided below.

[SEQ ID NO: 170]
MALPVTALLLPLALLLHAARPDIQMTQSPASLSVSVGETVTITCR
ASENIYSNLAWYQQRQGKSPQLLVYAATNLADGVPSRESGSGSGT
QYSLKINSLQSEDFGSYYCQHFWGSPYTFGGGTKLEIKGGGGSGG
GGSGGGGSQVQLKESGPGLVAPSQSLSITCTISGFSLTSYGVHWV
RQPPGKGLEWLVVIWGDGSTSYNSALKSRLSISKDNSKSQVFLKM
NSLQTDDTAMYFCATSTVGYAVDYWGQGTSVTVSSAAAIEVMYPP
PYLDNEKSNGTIIHVKGKHLCPSPLFPGPSKPFWVLVVVGGVLAC
YSLLVTVAFIIFWVRSKRSRLLHSDYMNMTPRRPGPTRKHYQPYA
PPRDFAAYRSRVKESRSADAPAYQQGQNQLYNELNLGRREEYDVL
DKRRGRDPEMGGKPRRKNPQEGLYNELQKDKMAEAYSEIGMKGER
RRGKGHDGLYQGLSTATKDTYDALHMQALPPR

An exemplary nucleic acid sequence encoding the amino acid sequence of SEQ ID NO: 170 is set forth in SEQ ID NO: 171, which is provided below.

[SEQ ID NO: 171]
ATGGCGTTGCCGGTGACCGCGCTTTTGCTCCCTCTGGCCCTGCTC
TTGCACGCCGCACGACCGGACATCCAGATGACCCAGTCACCGGCC
AGTTTGTCCGTTTCCGTAGGCGAGACCGTAACCATCACATGCCGA
GCAAGCGAGAATATCTATTCCAACCTGGCCTGGTATCAGCAGAGA
CAAGGCAAAAGTCCGCAACTTTTGGTTTATGCTGCCACTAATTTG
GCCGATGGGGTCCCGAGTCGATTTTCTGGCTCTGGCTCCGGGACG
CAGTATTCCCTGAAGATAAACTCTTTGCAGTCAGAAGATTTTGGT
TCATATTATTGTCAGCATTTCTGGGGCAGCCCGTACACATTTGGA
GGTGGGACCAAATTGGAAATAAAAGGCGGTGGTGGCTCAGGCGGC
GGAGGATCCGGGGGGGGGGGATCCCAAGTCCAGCTCAAGGAGAGC
GGTCCAGGACTCGTAGCACCCTCTCAATCTTTGTCTATCACTTGT
ACCATAAGCGGTTTTTCCTTGACAAGTTATGGCGTACATTGGGTG
AGACAACCGCCAGGTAAAGGACTCGAATGGTTGGTTGTTATCTGG
GGGGATGGGAGTACCAGCTACAACAGCGCACTCAAGTCTCGCCTT
TCAATTTCTAAGGACAACAGTAAATCACAGGTTTTTCTGAAGATG
AATAGCCTGCAAACCGACGACACGGCGATGTATTTCTGTGCGACT
TCTACGGTAGGTTATGCCGTCGACTATTGGGGTCAGGGTACGTCA
GTTACCGTAAGTAGCGCGGCCGCAATTGAAGTCATGTATCCTCCT
CCGTACTTGGATAATGAAAAAAGTAATGGGACCATAATTCACGTC
AAAGGAAAACATTTGTGTCCAAGCCCCCTTTTCCCTGGCCCGTCC
AAGCCTTTCTGGGTACTGGTGGTGGTGGGCGGAGTTCTCGCATGT
TATTCATTGCTGGTAACGGTCGCATTCATTATATTCTGGGTGCGA
TCAAAACGGAGTAGGCTTCTCCATAGTGACTATATGAACATGACG
CCGAGGCGCCCAGGCCCAACGCGCAAACATTATCAACCATACGCT
CCGCCACGAGATTTCGCAGCCTATAGATCCCGAGTAAAATTTTCC
CGAAGTGCAGATGCGCCAGCTTATCAGCAAGGCCAGAATCAGCTC
TACAATGAGTTGAACCTCGGACGGCGCGAGGAATATGATGTGCTC
GATAAGCGCAGAGGTAGGGACCCTGAGATGGGGGGTAAACCAAGG
CGAAAGAATCCCCAAGAGGGGCTTTACAACGAATTGCAAAAAGAC
AAAATGGCAGAAGCCTATTCCGAAATTGGAATGAAGGGCGAACGG
AGGCGAGGTAAGGGCCACGACGGGCTCTACCAAGGCCTGAGCACA
GCAACAAAAGACACGTATGACGCATTGCACATGCAAGCTCTTCCG
CCCCGG

In certain embodiments, the CAR is a L1CAM-targeted CAR. In certain embodiments, the CAR comprises (a) an extracellular antigen-binding domain comprising (i) a V_{H} that comprises a CDR1 comprising the amino acid sequence set forth in SEQ ID NO: 105, a CDR2 comprising the amino acid sequence set forth in SEQ ID NO: 106, and a V_{H} CDR3 comprising the amino acid sequence set forth in SEQ ID NO: 107, and (ii) a V_{L} that comprises a CDR1 comprising the amino acid sequence set forth in SEQ ID NO: 108, a CDR2 comprising the amino acid sequence set forth in SEQ ID NO: 39, and a V_{L} CDR3 comprising the amino acid sequence set forth in SEQ ID NO: 109; (b) a transmembrane domain comprising a CD28 polypeptide (e.g., a transmembrane domain of human CD28 or a fragment thereof), and (c) an intracellular signaling domain comprising (i) a CD3ζ polypeptide, and (ii) a co-stimulatory signaling region comprising a CD28 polypeptide (e.g., an intracellular domain of human CD28 or a fragment thereof). In certain embodiments, the V_{H} and V_{L} are linked via a linker comprising or consisting of the amino acid sequence set forth in SEQ ID NO: 11. In certain embodiments, the V_{H} and V_{L} are positioned from the N- to the C-terminus: V_{L}-V_{H}. In certain embodiments, the CAR is designed as “18H05_LH”. In certain embodiments, the CAR comprises the amino acid sequence set forth in SEQ ID NO: 172, which is provided below.

[SEQ ID NO: 172]
MALPVTALLLPLALLLHAARPDIVMTQSPDSLAVSLGERATINCK
SSQSVLYSSNNKNYLAWYQQKPGQPPKLFIYWASTRESGVPDRES
GSGSGTDFTLTISSLQAEDVAVYYCQQYYSTPPTFGQGTKVEIKG
GGGSGGGGSGGGGSQVQLQESGPGLVKPSETLSLTCTVSGDSISS
YYWTWIRQPAGKGLEWIGRIYTSGTTNYNPSLKSRVTMSVDTSKN
QFSLKLSSVTAADTAVYYCASHSSGWYDAFDIWGQGTMVTVSSAA
AIEVMYPPPYLDNEKSNGTIIHVKGKHLCPSPLFPGPSKPFWVLV
VVGGVLACYSLLVTVAFIIFWVRSKRSRLLHSDYMNMTPRRPGPT
RKHYQPYAPPRDFAAYRSRVKESRSADAPAYQQGQNQLYNELNLG
RREEYDVLDKRRGRDPEMGGKPRRKNPQEGLYNELQKDKMAEAYS
EIGMKGERRRGKGHDGLYQGLSTATKDTYDALHMQALPPR

An exemplary nucleic acid sequence encoding the amino acid sequence of SEQ ID NO: 172 is set forth in SEQ ID NO: 173, which is provided below.

[SEQ ID NO: 173]
ATGGCGTTGCCGGTGACCGCGCTTTTGCTCCCTCTGGCCCTGCTC
TTGCACGCCGCACGACCGGACATCGTAATGACCCAATCACCTGAT
TCTCTGGCTGTGAGCCTTGGTGAGAGGGCAACAATAAACTGCAAG
AGCTCTCAGTCCGTACTTTATAGTAGCAACAACAAGAACTATTTG
GCGTGGTACCAGCAGAAGCCGGGTCAGCCGCCAAAGCTGTTTATA
TATTGGGCCTCTACCCGCGAATCTGGAGTACCGGACAGGTTTAGT
GGTAGTGGCTCAGGTACGGATTTTACGCTCACCATTAGCTCTCTG
CAAGCAGAGGACGTAGCCGTATATTATTGCCAGCAATATTATTCT
ACGCCACCCACCTTTGGTCAGGGGACGAAAGTTGAGATCAAGGGC
GGTGGTGGCTCAGGCGGCGGAGGATCCGGGGGGGGGGGATCCCAG
GTACAGCTTCAAGAAAGCGGTCCAGGACTTGTGAAGCCGAGCGAG
ACGCTTTCACTGACTTGCACAGTCAGTGGGGACAGTATTAGTTCC
TATTACTGGACATGGATACGACAACCAGCAGGGAAAGGTCTGGAA
TGGATTGGGCGCATTTACACCAGTGGAACTACAAATTACAACCCA
TCCCTCAAATCCAGGGTTACGATGAGCGTCGACACGAGTAAGAAT
CAGTTTTCCCTGAAGCTCTCCAGTGTGACGGCTGCAGATACTGCA
GTGTATTATTGCGCTTCCCACTCAAGTGGTTGGTACGATGCTTTC
GACATCTGGGGGCAGGGTACTATGGTGACCGTGTCCTCAGCGGCC
GCAATTGAAGTCATGTATCCTCCTCCGTACTTGGATAATGAAAAA
AGTAATGGGACCATAATTCACGTCAAAGGAAAACATTTGTGTCCA
AGCCCCCTTTTCCCTGGCCCGTCCAAGCCTTTCTGGGTACTGGTG
GTGGTGGGCGGAGTTCTCGCATGTTATTCATTGCTGGTAACGGTC
GCATTCATTATATTCTGGGTGCGATCAAAACGGAGTAGGCTTCTC
CATAGTGACTATATGAACATGACGCCGAGGCGCCCAGGCCCAACG
CGCAAACATTATCAACCATACGCTCCGCCACGAGATTTCGCAGCC
TATAGATCCCGAGTAAAATTTTCCCGAAGTGCAGATGCGCCAGCT
TATCAGCAAGGCCAGAATCAGCTCTACAATGAGTTGAACCTCGGA
CGGCGCGAGGAATATGATGTGCTCGATAAGCGCAGAGGTAGGGAC
CCTGAGATGGGGGGTAAACCAAGGCGAAAGAATCCCCAAGAGGGG
CTTTACAACGAATTGCAAAAAGACAAAATGGCAGAAGCCTATTCC
GAAATTGGAATGAAGGGCGAACGGAGGCGAGGTAAGGGCCACGAC
GGGCTCTACCAAGGCCTGAGCACAGCAACAAAAGACACGTATGAC
GCATTGCACATGCAAGCTCTTCCGCCCCGG

In certain embodiments, the V_{H} and V_{L} are positioned from the N- to the C-terminus: V_{H}-V_{L}. In certain embodiments, the CAR is designed as “18H05_HL”. In certain embodiments, the CAR comprises the amino acid sequence set forth in SEQ ID NO: 174, which is provided below.

[SEQ ID NO: 174]
MALPVTALLLPLALLLHAARPQVQLQESGPGLVKPSETLSLTCTV
SGDSISSYYWTWIRQPAGKGLEWIGRIYTSGTTNYNPSLKSRVTM
SVDTSKNQFSLKLSSVTAADTAVYYCASHSSGWYDAFDIWGQGTM
VTVSSGGGGSGGGGSGGGGSDIVMTQSPDSLAVSLGERATINCKS
SQSVLYSSNNKNYLAWYQQKPGQPPKLFIYWASTRESGVPDRESG
SGSGTDFTLTISSLQAEDVAVYYCQQYYSTPPTFGQGTKVEIKAA
AIEVMYPPPYLDNEKSNGTIIHVKGKHLCPSPLFPGPSKPFWVLV
VVGGVLACYSLLVTVAFIIFWVRSKRSRLLHSDYMNMTPRRPGPT
RKHYQPYAPPRDFAAYRSRVKESRSADAPAYQQGQNQLYNELNLG
RREEYDVLDKRRGRDPEMGGKPRRKNPQEGLYNELQKDKMAEAYS
EIGMKGERRRGKGHDGLYQGLSTATKDTYDALHMQALPPR

An exemplary nucleic acid sequence encoding the amino acid sequence of SEQ ID NO: 174 is set forth in SEQ ID NO: 175, which is provided below.

[SEQ ID NO: 175]
ATGGCGTTGCCGGTGACCGCGCTTTTGCTCCCTCTGGCCCTGCTC
TTGCACGCCGCACGACCGCAGGTACAGCTTCAAGAAAGCGGTCCA
GGACTTGTGAAGCCGAGCGAGACGCTTTCACTGACTTGCACAGTC
AGTGGGGACAGTATTAGTTCCTATTACTGGACATGGATACGACAA
CCAGCAGGGAAAGGTCTGGAATGGATTGGGCGCATTTACACCAGT
GGAACTACAAATTACAACCCATCCCTCAAATCCAGGGTTACGATG
AGCGTCGACACGAGTAAGAATCAGTTTTCCCTGAAGCTCTCCAGT
GTGACGGCTGCAGATACTGCAGTGTATTATTGCGCTTCCCACTCA
AGTGGTTGGTACGATGCTTTCGACATCTGGGGGCAGGGTACTATG
GTGACCGTGTCCTCAGGCGGTGGTGGCTCAGGCGGCGGAGGATCC
GGGGGGGGGGGATCCGACATCGTAATGACCCAATCACCTGATTCT
CTGGCTGTGAGCCTTGGTGAGAGGGCAACAATAAACTGCAAGAGC
TCTCAGTCCGTACTTTATAGTAGCAACAACAAGAACTATTTGGCG
TGGTACCAGCAGAAGCCGGGTCAGCCGCCAAAGCTGTTTATATAT
TGGGCCTCTACCCGCGAATCTGGAGTACCGGACAGGTTTAGTGGT
AGTGGCTCAGGTACGGATTTTACGCTCACCATTAGCTCTCTGCAA
GCAGAGGACGTAGCCGTATATTATTGCCAGCAATATTATTCTACG
CCACCCACCTTTGGTCAGGGGACGAAAGTTGAGATCAAGGCGGCC
GCAATTGAAGTCATGTATCCTCCTCCGTACTTGGATAATGAAAAA
AGTAATGGGACCATAATTCACGTCAAAGGAAAACATTTGTGTCCA
AGCCCCCTTTTCCCTGGCCCGTCCAAGCCTTTCTGGGTACTGGTG
GTGGTGGGCGGAGTTCTCGCATGTTATTCATTGCTGGTAACGGTC
GCATTCATTATATTCTGGGTGCGATCAAAACGGAGTAGGCTTCTC
CATAGTGACTATATGAACATGACGCCGAGGCGCCCAGGCCCAACG
CGCAAACATTATCAACCATACGCTCCGCCACGAGATTTCGCAGCC
TATAGATCCCGAGTAAAATTTTCCCGAAGTGCAGATGCGCCAGCT
TATCAGCAAGGCCAGAATCAGCTCTACAATGAGTTGAACCTCGGA
CGGCGCGAGGAATATGATGTGCTCGATAAGCGCAGAGGTAGGGAC
CCTGAGATGGGGGGTAAACCAAGGCGAAAGAATCCCCAAGAGGGG
CTTTACAACGAATTGCAAAAAGACAAAATGGCAGAAGCCTATTCC
GAAATTGGAATGAAGGGCGAACGGAGGCGAGGTAAGGGCCACGAC
GGGCTCTACCAAGGCCTGAGCACAGCAACAAAAGACACGTATGAC
GCATTGCACATGCAAGCTCTTCCGCCCCGG

In certain embodiments, a presently disclosed CAR further comprises an inducible promoter, for expressing nucleic acid sequences in human cells. Promoters for use in expressing CAR genes can be a constitutive promoter, such as ubiquitin C (UbiC) promoter.

### 3.2. TCR Like Fusion Molecules

In certain embodiments, the antigen-recognizing receptor is a TCR like fusion molecule. Non-limiting examples of TCR fusion molecules include HLA-Independent TCR-based Chimeric Antigen Receptor (also known as “HIT-CAR”, e.g., those disclosed in International Patent Application No. PCT/US19/017525, which is incorporated by reference in its entirety), and T cell receptor fusion constructs (TRuCs) (e.g., those disclosed in Baeuerle et al., “Synthetic TRuC receptors engaging the complete T cell receptor for potent anti-tumor response,” Nature Communications volume 10, Article number: 2087 (2019), which is incorporated by reference in its entirety).

In certain embodiments, the TCR like fusion molecule comprises an antigen binding chain that comprises an extracellular antigen-binding domain and a constant domain, wherein the TCR like fusion molecule binds to an antigen in an HLA-independent manner. In certain embodiments, the constant domain comprises a T cell receptor constant region selected from the group consisting of a native or modified TRAC peptide, a native or modified TRBC peptide, a native or modified TRDC peptide, a native or modified TRGC peptide and any variants or functional fragments thereof. In certain embodiments, the constant domain comprises a native or modified TRAC peptide. In certain embodiments, the constant domain comprises a native or modified TRBC peptide. In certain embodiments, the constant domain is capable of forming a homodimer or a heterodimer with another constant domain. In certain embodiments, the antigen binding chain is capable of associating with a CD3ζ polypeptide. In certain embodiments, the antigen binding chain, upon binding to an antigen, is capable of activating the CD3ζ polypeptide associated to the antigen binding chain. In certain embodiments, the activation of the CD3ζ polypeptide is capable of activating an immunoresponsive cell. In certain embodiments, the TCR like fusion molecule is capable of integrating with a CD3 complex and providing HLA-independent antigen recognition. In certain embodiments, the TCR like fusion molecule replaces an endogenous TCR in a CD3/TCR complex. In certain embodiments, the extracellular antigen-binding domain of the TCR like fusion molecule is capable of dimerizing with another extracellular antigen-binding domain. In certain embodiments, the extracellular antigen-binding domain of the TCR like fusion molecule comprises a ligand for a cell-surface receptor, a receptor for a cell surface ligand, an antigen binding portion of an antibody or a fragment thereof or an antigen binding portion of a TCR. In certain embodiments, the extracellular antigen-binding domain of the TCR like fusion molecule comprises one or two immunoglobulin variable region(s). In certain embodiments, the extracellular antigen-binding domain of the TCR like fusion molecule comprises a heavy chain variable region (V_{H}) of an antibody. In certain embodiments, the extracellular antigen-binding domain of the TCR like fusion molecule comprises a light chain variable region (V_{L}) of an antibody. In certain embodiments, the extracellular antigen-binding domain of the TCR like fusion molecule is capable of dimerizing with another extracellular antigen-binding domain. In certain embodiments, the extracellular antigen-binding domain of the TCR like fusion molecule comprises a V_{H} of an antibody, wherein the V_{H} is capable of dimerizing with another extracellular antigen-binding domain comprising a V_{L} of the antibody and form a fragment variable (Fv). In certain embodiments, the extracellular antigen-binding domain of the TCR like fusion molecule comprises a V_{L} of an antibody, wherein the V_{L} is capable of dimerizing with another extracellular antigen-binding domain comprising a V_{H} of the antibody and form a fragment variable (Fv).

The TCR like fusion molecule can bind to a tumor antigen or a pathogen antigen. In certain embodiments, the TCR like fusion molecule binds to a tumor antigen.

### 4. CELLS

The presently disclosed subject matter provides cells comprising a presently disclosed L1CAM-targeted antigen-recognizing receptor (e.g., one disclosed in Section 3). In certain embodiments, the cell is selected from the group consisting of cells of lymphoid lineage and cells of myeloid lineage. In certain embodiments, the cell is an immunoresponsive cell. In certain embodiments, the immunoresponsive cell is a cell of lymphoid lineage.

In certain embodiments, the cell is a cell of the lymphoid lineage. Cells of the lymphoid lineage can provide production of antibodies, regulation of cellular immune system, detection of foreign agents in the blood, detection of cells foreign to the host, and the like. Non-limiting examples of cells of the lymphoid lineage include T cells, Natural Killer (NK) cells, B cells, dendritic cells, stem cells from which lymphoid cells may be differentiated. In certain embodiments, the stem cell is a pluripotent stem cell. In certain embodiments, the pluripotent stem cell is an embryonic stem cell (ESC) or an induced pluripotent stem cell (iPSC).

In certain embodiments, the cell is a T cell. T cells can be lymphocytes that mature in the thymus and are chiefly responsible for cell-mediated immunity. T cells are involved in the adaptive immune system. The T cells of the presently disclosed subject matter can be any type of T cells, including, but not limited to, helper T cells, cytotoxic T cells, memory T cells (including central memory T cells, stem-cell-like memory T cells (or stem-like memory T cells), and two types of effector memory T cells: e.g., TEM cells and TEMRA cells, Regulatory T cells (also known as suppressor T cells), tumor-infiltrating lymphocyte (TIL), Natural killer T cells, Mucosal associated invariant T cells, and γδ T cells. Cytotoxic T cells (CTL or killer T cells) are a subset of T lymphocytes capable of inducing the death of infected somatic or tumor cells. A patient's own T cells may be genetically modified to target specific antigens through the introduction of an antigen-recognizing receptor, e.g., a CAR or a TCR like fusion molecule. In certain embodiments, the immunoresponsive cell is a T cell. The T cell can be a CD4⁺ T cell or a CD8⁺ T cell. In certain embodiments, the T cell is a CD4⁺ T cell. In certain embodiments, the T cell is a CD8⁺ T cell.

In certain embodiments, the cell is a NK cell. Natural killer (NK) cells can be lymphocytes that are part of cell-mediated immunity and act during the innate immune response. NK cells do not require prior activation in order to perform their cytotoxic effect on target cells.

Types of human lymphocytes of the presently disclosed subject matter include, without limitation, peripheral donor lymphocytes. e.g., those disclosed in Sadelain et al., Nat Rev Cancer (2003); 3:35-45 (disclosing peripheral donor lymphocytes genetically modified to express CARs), in Morgan, R. A., et al. 2006 Science 314:126-129 (disclosing peripheral donor lymphocytes genetically modified to express a full-length tumor antigen-recognizing T cell receptor complex comprising the α and β heterodimer), in Panelli et al., J Immunol (2000); 164:495-504; Panelli et al., J Immunol (2000); 164:4382-4392 (disclosing lymphocyte cultures derived from tumor infiltrating lymphocytes (TILs) in tumor biopsies), and in Dupont et al., Cancer Res (2005); 65:5417-5427; Papanicolaou et al., Blood (2003); 102:2498-2505 (disclosing selectively in vitro-expanded antigen-specific peripheral blood leukocytes employing artificial antigen-presenting cells (AAPCs) or pulsed dendritic cells).

The cells (e.g., T cells) can be autologous, non-autologous (e.g., allogeneic), or derived in vitro from engineered progenitor or stem cells.

The cells of the presently disclosed subject matter can be cells of the myeloid lineage. Non-limiting examples of cells of the myeloid lineage include monocytes, macrophages, neutrophils, dendritic cells, basophils, neutrophils, eosinophils, megakaryocytes, mast cell, erythrocyte, thrombocytes, and stem cells from which myeloid cells may be differentiated. In certain embodiments, the stem cell is a pluripotent stem cell (e.g., an embryonic stem cell or an induced pluripotent stem cell).

In certain embodiments, the presently disclosed cells are capable of modulating the tumor microenvironment. Tumors have a microenvironment that is hostile to the host immune response involving a series of mechanisms by malignant cells to protect themselves from immune recognition and elimination. This “hostile tumor microenvironment” comprises a variety of immune suppressive factors including infiltrating regulatory CD4⁺ T cells (Tregs), myeloid derived suppressor cells (MDSCs), tumor associated macrophages (TAMs), immune suppressive cytokines including TGF-β, and expression of ligands targeted to immune suppressive receptors expressed by activated T cells (CTLA-4 and PD-1). These mechanisms of immune suppression play a role in the maintenance of tolerance and suppressing inappropriate immune responses, however within the tumor microenvironment these mechanisms prevent an effective anti-tumor immune response. Collectively these immune suppressive factors can induce either marked anergy or apoptosis of adoptively transferred CAR modified T cells upon encounter with targeted tumor cells.

In certain embodiments, the cells can be transduced with the presently disclosed L1CAM-targeted antigen-recognizing receptor such that the cells express the antigen-recognizing receptor.

### 5. COMPOSITIONS AND VECTORS

The presently disclosed subject matter provides compositions comprising a presently disclosed L1CAM-targeted antigen-recognizing receptor (e.g., one disclosed in Section 3). Also provided are cells comprising such compositions.

In certain embodiments, the presently disclosed L1CAM-targeted antigen-recognizing receptor is operably linked to a promoter.

Furthermore, the present discloses subject matter provides nuclei acid compositions comprising a polynucleotide encoding a presently disclosed L1CAM-targeted antigen-recognizing receptor (e.g., one disclosed in Section 3). Also provided are cells comprising such nucleic acid compositions.

In certain embodiments, the nucleic acid composition further comprises a promoter that is operably linked to the presently disclosed L1CAM-targeted antigen-recognizing receptor.

In certain embodiments, the promoter is endogenous or exogenous. In certain embodiments, the exogenous promoter is selected from the group consisting of an elongation factor (EF)-1 promoter, a cytomegalovirus immediate-early promoter (CMV) promoter, a simian virus 40 early promoter (SV40) promoter, a phosphoglycerate kinase (PGK) promoter, a metallothionein promoter, and gamma retrovirus 5′ long-terminal repeat (LTR) promoter. In certain embodiments, the promoter is an inducible promoter. In certain embodiment, the inducible promoter is selected from the group consisting of a NFAT transcriptional response element (TRE) promoter, a CD69 promoter, a CD25 promoter, Nur77 promoter, and an IL-2 promoter.

The compositions and nucleic acid compositions can be administered to subjects or and/delivered into cells by art-known methods or as described herein. Genetic modification of a cell (e.g., a T cell or a NK cell) can be accomplished by transducing a substantially homogeneous cell composition with a recombinant DNA construct. In certain embodiments, a retroviral vector (e.g., gamma-retroviral vector or lentiviral vector) is employed for the introduction of the DNA construct into the cell. For example, a polynucleotide encoding an antigen-recognizing receptor can be cloned into a retroviral vector and expression can be driven from its endogenous promoter, from the retroviral long terminal repeat, or from a promoter specific for a target cell type of interest. Non-viral vectors may be used as well.

For initial genetic modification of a cell to include a presently disclosed L1CAM-targeted antigen-recognizing receptor (e.g., a CAR or a TCR like fusion molecule), a retroviral vector can be employed for transduction, however any other suitable viral vector or non-viral delivery system can be used. The antigen-recognizing receptor can be constructed in a single, multicistronic expression cassette, in multiple expression cassettes of a single vector, or in multiple vectors. Examples of elements that create polycistronic expression cassette include, but is not limited to, various viral and non-viral Internal Ribosome Entry Sites (IRES, e.g., FGF-1 IRES, FGF-2 IRES, VEGF IRES, IGF-II IRES, NF-κB IRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g., 2A peptides, e.g., P2A, T2A, E2A and F2A peptides). In certain embodiments, the multicistronic expression cassette comprises a P2A peptide. In certain embodiments, the multicistronic expression cassette comprises a T2A peptide. In certain embodiments, the multicistronic expression cassette comprises a P2A peptide and a T2A peptide. Combinations of retroviral vector and an appropriate packaging line are also suitable, where the capsid proteins will be functional for infecting human cells. Various amphotropic virus-producing cell lines are known, including, but not limited to, PA12 (Miller et al., (1985) Mol Cell Biol (1985); 5:431-437); PA317 (Miller, et al., Mol Cell Biol (1986); 6:2895-2902); and CRIP (Danos et al., Proc Natl Acad Sci USA (1988); 85:6460-6464). Non-amphotropic particles are suitable too, e.g., particles pseudotyped with VSVG, RD114 or GALV envelope and any other known in the art.

Possible methods of transduction also include direct co-culture of the cells with producer cells (Bregni et al., Blood (1992); 80:1418-1422), or culturing with viral supernatant alone or concentrated vector stocks with or without appropriate growth factors and polycations (Xu et al., Exp Hemat (1994); 22:223-230; and Hughes et al. J Clin Invest (1992); 89:1817).

Other transducing viral vectors can be used to modify a cell. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller, Human Gene Thera (1990); 15-14; Friedman, Science 244:1275-1281, 1989; Eglitis et al., BioTechniques (1988); 6:608-614; Tolstoshev et al., Cur Opin Biotechnol (1990); 1:55-61; Sharp, The Lancet (1991); 337:1277-78; Cornetta et al., Nucleic Acid Research and Molecular Biology 36:311-22, 1987; Anderson, Science (1984); 226:401-409; Moen, Blood Cells 17:407-16, 1991; Miller et al., Biotechnol (1989); 7:980-90; LeGal La Salle et al., Science (1993); 259:988-90; and Johnson, Chest (1995) 107:77S-83S). Retroviral vectors are particularly well developed and have been used in clinical settings (Rosenberg et al., N Engl J Med (1990); 323:370, 1990; Anderson et al., U.S. Pat. No. 5,399,346).

Non-viral approaches can also be employed for genetic modification of a cell. For example, a nucleic acid molecule can be introduced into a cell by administering the nucleic acid in the presence of lipofection (Feigner et al., Proc Natl Acad Sci U.S.A. (1987); 84:7413; Ono et al., Neurosci Lett (1990); 17:259; Brigham et al., Am J Med Sci (1989); 298:278; Staubinger et al., Methods in Enzymol (1983); 101:512, Wu et al., J Biol Chem (1988); 263:14621; Wu et al., J Biol Chem (1989); 264:16985), or by micro-injection under surgical conditions (Wolff et al., Science (1990); 247:1465). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell(s) (or its descendants) are injected into a targeted tissue or are injected systemically. Recombinant receptors can also be derived or obtained using transposases or targeted nucleases (e.g., Zinc finger nucleases, meganucleases, or TALE nucleases, CRISPR). Transient expression may be obtained by RNA electroporation.

Any targeted genome editing methods can also be used to deliver a presently disclosed antigen-recognizing receptor to a cell or a subject. In certain embodiments, a CRISPR system is used to deliver a presently disclosed antigen-recognizing receptor disclosed herein. In certain embodiments, zinc-finger nucleases are used to deliver the antigen-recognizing receptor. In certain embodiments, a TALEN system is used to deliver a presently disclosed antigen-recognizing receptor.

Clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. The crRNA needs to be designed for each application as this is the sequence that Cas9 uses to identify and directly bind to the target DNA in a cell. The repair template carrying CAR expression cassette need also be designed for each application, as it must overlap with the sequences on either side of the cut and code for the insertion sequence. Multiple crRNA's and the tracrRNA can be packaged together to form a single-guide RNA (sgRNA). This sgRNA can be joined together with the Cas9 gene and made into a plasmid in order to be transfected into cells.

A zinc-finger nuclease (ZFN) is an artificial restriction enzyme, which is generated by combining a zinc finger DNA-binding domain with a DNA-cleavage domain. A zinc finger domain can be engineered to target specific DNA sequences which allows a zinc-finger nuclease to target desired sequences within genomes. The DNA-binding domains of individual ZFNs typically contain a plurality of individual zinc finger repeats and can each recognize a plurality of basepairs. The most common method to generate new zinc-finger domain is to combine smaller zinc-finger “modules” of known specificity. The most common cleavage domain in ZFNs is the non-specific cleavage domain from the type IIs restriction endonuclease FokI. Using the endogenous homologous recombination (HR) machinery and a homologous DNA template carrying CAR expression cassette, ZFNs can be used to insert the CAR expression cassette into genome. When the targeted sequence is cleaved by ZFNs, the HR machinery searches for homology between the damaged chromosome and the homologous DNA template, and then copies the sequence of the template between the two broken ends of the chromosome, whereby the homologous DNA template is integrated into the genome.

Transcription activator-like effector nucleases (TALEN) are restriction enzymes that can be engineered to cut specific sequences of DNA. TALEN system operates on almost the same principle as ZFNs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome. cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g., the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

In certain embodiments, the delivery methods include use of colloids. As used herein, the term “colloid” refers to systems in which there are two or more phases, with one phase (e.g., the dispersed phase) distributed in the other phase (e.g., the continuous phase). Moreover, at least one of the phases has small dimensions (in the range of about 10⁻⁹ to about 10⁻⁶ m). Non-limiting examples of colloids encompassed by the presently disclosed subject matter include macromolecule complexes, nanocapsules, microspheres, beads, and lipid-based systems (e.g., micelles, liposomes, and lipid nanoparticles).

In certain embodiments, the delivery methods include use of liposomes. The term “liposome,” as used herein, refers to single- or multi-layered spherical lipid bilayer structures produced from lipids dissolved in organic solvents and then dispersed in aqueous media. Experimentally and therapeutically used for delivering an active pharmaceutical ingredient (e.g., nucleic acid compositions disclosed herein) to cells, liposomes fuse with cell membranes so the contents are transferred into the cytoplasm.

In certain embodiments, the delivery methods include use of lipid nanoparticles. As used herein, the term “lipid nanoparticle” refers to a particle having at least one dimension in the order of nanometers (e.g., from about 1 nm to about 1,000 nm) and including at least one lipid. In certain embodiments, the lipid nanoparticles can include an active pharmaceutical ingredient (e.g., nucleic acid compositions disclosed herein) for delivering to cells. The morphology of the lipid nanoparticles can be different from liposomes. While liposomes are characterized by a lipid bilayer surrounding a hydrophilic core, lipid nanoparticles have an electron-dense core where cationic lipids and/or ionizable lipids are organized into inverted micelles around an active pharmaceutical ingredient (e.g., nucleic acid compositions disclosed herein). Additional information on the morphology and properties of lipid nanoparticles and liposomes can be found in Wilczewska, et al., Pharmacological reports 64, no. 5 (2012): 1020-1037; Eygeris et al., Accounts of Chemical Research 55, no. 1 (2021): 2-12; Zhang et al., Chemical Reviews 121, no. 20 (2021): 12181-12277; and Fan et al., Journal of pharmaceutical and biomedical analysis 192 (2021): 113642.

In certain embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm.

In certain embodiments, the lipid nanoparticles can include a cationic lipid or an ionizable lipid. The term “cationic lipid” refers to lipids including a head group with permanent positive charges. Non-limiting examples of cationic lipids encompassed by the presently disclosed subject matter include 1,2-di-O-octadecenyl-3-trimethylammonium-propane (DOTMA), 1,2-dioleoyl-3-trimethylammonium-propane (DOTAP), 2,3-dioleyloxy-N-[2-(sperminecarboxamido)ethyl]-N,N-dimethyl-1-propanaminium trifluoroacetate (DOSPA), and ethylphosphatidylcholine (ePC).

As used herein, the term “ionizable lipid” refers to lipids that are protonated at low pH and are neutral at physiological pH. The pH-sensitivity of ionizable lipids is particularly beneficial for delivery in vivo (e.g., delivery of nucleic acid compositions disclosed herein), because neutral lipids have less interactions with the anionic membranes of blood cells and, thus, improve the biocompatibility of the lipid nanoparticles. Once trapped in endosomes, ionizable lipids are protonated and promote membrane destabilization to allow the endosomal escape of the nanoparticles. Non-limiting example of ionizable lipids encompassed by the presently disclosed subject matter include tetrakis(8-methylnonyl) 3,3′,3″,3′″-(((methylazanediyl) bis(propane-3,1 diyl))bis (azanetriyl))tetrapropionate; decyl (2-(dioctylammonio)ethyl) phosphate; ((4-hydroxybutyl)azanediyl)bis(hexane-6,1-diyl)bis(2-hexyldecanoate); bis(2-(dodecyldisulfanyl)ethyl) 3,3′-((3-methyl-9-oxo-10-oxa-13,14-dithia-3,6-diazahexacosyl)azanediyl)dipropionate; 1,1′-((2-(4-(2-((2-(bis(2-hydroxydodecyl)amino)ethyl)(2-hydroxydodecyl)amino)ethyl) piperazin-1-yl)ethyl)azanediyl) bis(dodecan-2-ol); cKK-E12, 3,6-bis(4-(bis(2-hydroxydodecyl)amino)butyl)piperazine-2,5-dione; (6Z,9Z,28Z,31Z)-heptatriaconta-6,9,28,31-tetraen-19-yl 4-(dimethylamino) butanoate; hexa(octan-3-yl) 9,9′,9″,9′″,9″″,9′″″-((((benzene-1,3,5-tricarbonyl)yris(azanediyl)) tris (propane-3,1-diyl)) tris(azanetriyl))hexanonanoate; heptadecan-9-yl 8-((2-hydroxyethyl)(6-oxo-6-(undecyloxy)hexyl)amino) octanoate; and (((3,6-dioxopipezine-2,5-diyl)bis(butane-4, 1-diyl))bis(azaneriyl))tetrakis(ethane-2,1-diyl) (9Z,9′Z,9″Z,9′″Z,12Z,12′Z,12″Z,12′″Z)-tetrakis (octadeca-9,12-dienoate).

Additionally, in certain embodiments, the lipid nanoparticles can include other lipids. For example, but without any limitation, the lipid nanoparticles of the presently disclosed subject matter can include phospholipids, cholesterol, polyethylene glycol (PEG)-functionalized lipids (PEG-lipids). These lipids can improve certain properties of the lipid nanoparticles (e.g., stability, biodistribution, etc.). For example, cholesterol enhances the stability of the lipid nanoparticles by modulating the integrity and rigidity. Non-limiting examples of other lipids present in lipid nanoparticles include cholesterol, DC-cholesterol, β-sitosterol, BHEM-cholesterol, ALC-0159, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoylphosphatidylethanolamine (DSPE), 16-O-monomethyl PE, 16-O-dimethyl PE, 18-1-trans PE, 1-stearoyl-2-oleoyl-phosphatidyethanol amine (SOPE), and 1,2-dielaidoyl-sn-glycero-3-phophoethanolamine (transDOPE).

In certain embodiments, the lipid nanoparticles can include a targeting moiety that binds to a ligand. The use of the targeting moieties allows selective delivery of an active pharmaceutical ingredient (e.g., nucleic acid compositions disclosed herein) to target cells expressing the ligand (e.g., T cells). In certain embodiments, the targeting moiety can be an antibody or antigen-binding fragment thereof that binds to a cell surface receptor. For example, but without any limitation, the targeting domain is an antibody or antigen-binding fragment thereof that binds to a receptor expressed on the surface of a T cell (e.g., CD3, CD4, CD8, CD16, CD40L, CD95, FasL, CTLA-4, OX40, GITR, LAG3, ICOS, and PD-1).

In certain embodiments, the delivery methods are in vivo delivery methods. In certain embodiments, the delivery methods are ex vivo delivery methods.

### 6. POLYPEPTIDES

The presently disclosed subject matter provides methods for optimizing an amino acid sequence or a nucleic acid sequence by producing an alteration in the sequence. Such alterations may include certain mutations, deletions, insertions, or post-translational modifications. The presently disclosed subject matter further includes analogs of any naturally occurring polypeptides disclosed herein (including, but not limited to, CD8, CD28, CD3ζ, etc.). Analogs can differ from a naturally occurring polypeptide disclosed herein by amino acid sequence differences, by post-translational modifications, or by both. Analogs can exhibit at least about 85%, about 90%, about 91%, about 92%, about 93%, about 94%, about 95%, about 96%, about 97%, about 98%, about 99% or more homologous or identical to all or part of a naturally occurring amino, acid sequence of the presently disclosed subject matter. The length of sequence comparison is at least 5, 10, 15 or 20 amino acid residues, e.g., at least 25, 50, or 75 amino acid residues, or more than 100 amino acid residues. Again, in an exemplary approach to determining the degree of identity, a BLAST program may be used, with a probability score between e⁻³ and e⁻¹⁰⁰ indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally occurring polypeptides by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., β or γ amino acids.

In addition to full-length polypeptides, the presently disclosed subject matter also provides fragments of any of the polypeptides disclosed herein. As used herein, the term “a fragment” means at least 5, 10, 13, or 15 amino acids. In certain embodiments, a fragment comprises at least 20 contiguous amino acids, at least 30 contiguous amino acids, or at least 50 contiguous amino acids. In certain embodiments, a fragment comprises at least 60 to 80, 100, 200, 300 or more contiguous amino acids. Fragments can be generated by methods known to those skilled in the art or may result from normal protein processing (e.g., removal of amino acids from the nascent polypeptide that are not required for biological activity or removal of amino acids by alternative mRNA splicing or alternative protein processing events).

### 7. FORMULATIONS AND ADMINISTRATION

The presently disclosed subject matter also provides compositions comprising the presently disclosed cells. Compositions comprising the presently disclosed cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the genetically modified cells in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the presently disclosed subject matter, however, any vehicle, diluent, or additive used would have to be compatible with the genetically modified cells.

The compositions can be isotonic, i.e., they can have the same osmotic pressure as blood and lacrimal fluid. The desired isotonicity of the compositions may be accomplished using sodium chloride, or other pharmaceutically acceptable agents such as dextrose, boric acid, sodium tartrate, propylene glycol or other inorganic or organic solutes. Sodium chloride can be particularly for buffers containing sodium ions.

Viscosity of the compositions, if desired, can be maintained at the selected level using a pharmaceutically acceptable thickening agent. For example, methylcellulose is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Compositions comprising the presently disclosed cells can be provided systemically or directly to a subject for inducing and/or enhancing an immune response to an antigen and/or treating and/or preventing a neoplasia. In certain embodiments, the presently disclosed cells or compositions comprising thereof are directly injected into an organ of interest (e.g., an organ affected by a neoplasia). Alternatively, the presently disclosed cells or compositions comprising thereof are provided indirectly to the organ of interest, for example, by administration into the circulatory system (e.g., the tumor vasculature). Expansion and differentiation agents can be provided prior to, during or after administration of the cells or compositions to increase production of cells (e.g., T cells or NK cells) in vitro or in vivo.

The presently disclosed cells can be administered in any physiologically acceptable vehicle, normally intravascularly, although they may also be introduced into bone or other convenient site where the cells may find an appropriate site for regeneration and differentiation (e.g., thymus).

The quantity of cells to be administered can vary for the subject being treated. In certain embodiments, between about 10⁴ and about 10¹⁰, between about 10⁴ and about 10⁷, between about 10⁵ and about 10⁷, between about 10⁵ and about 10⁹, or between about 10⁶ and about 10⁸ of the presently disclosed cells are administered to a subject. More effective cells may be administered in even smaller numbers. Usually, at least about 1×10⁵ cells will be administered, eventually reaching about 1×10¹⁰ or more. In certain embodiments, at least about 1×10⁵, 5×10⁵, 1×10⁶, about 5×10⁶, about 1×10⁷, about 5×10⁷, about 1×10⁸, or about 5×10⁸ of the presently disclosed cells are administered to a subject. In certain embodiments, about 1×10⁶ of the presently disclosed cells are administered to a subject. In certain embodiments, about 5×10⁶ of the presently disclosed cells are administered to a subject. The precise determination of what would be considered an effective dose can be based on factors individual to each subject, including their size, age, sex, weight, and condition of the particular subject. Dosages can be readily ascertained by those skilled in the art from this disclosure and the knowledge in the art.

The presently disclosed cells can comprise a purified population of cells. Those skilled in the art can readily determine the percentage of the presently disclosed cells in a population using various well-known methods, such as fluorescence activated cell sorting (FACS). Suitable ranges of purity in populations comprising the presently disclosed immunoresponsive cells are about 50% to about 55%, about 5% to about 60%, and about 65% to about 70%. In certain embodiments, the purity is about 70% to about 75%, about 75% to about 80%, or about 80% to about 85%. In certain embodiments, the purity is about 85% to about 90%, about 90% to about 95%, and about 95% to about 100%. Dosages can be readily adjusted by those skilled in the art (e.g., a decrease in purity may require an increase in dosage). The cells can be introduced by injection, catheter, or the like.

The skilled artisan can readily determine the amount of cells and optional additives, vehicles, and/or carrier in compositions and to be administered in methods. Typically, any additives (in addition to the active cell(s) and/or agent(s)) are present in an amount of 0.001 to 50% (weight) solution in phosphate buffered saline, and the active ingredient is present in the order of micrograms to milligrams, such as about 0.0001 to about 5 wt %, about 0.0001 to about 1 wt %, about 0.0001 to about 0.05 wt % or about 0.001 to about 20 wt %, about 0.01 to about 10 wt %, or about 0.05 to about 5 wt %. For any composition to be administered to an animal or human, the followings can be determined: toxicity such as by determining the lethal dose (LD) and LD50 in a suitable animal model e.g., rodent such as mouse; the dosage of the composition(s), concentration of components therein and timing of administering the composition(s), which elicit a suitable response. Such determinations do not require undue experimentation from the knowledge of the skilled artisan, this disclosure and the documents cited herein, and the time for sequential administrations can be ascertained without undue experimentation.

In certain embodiments, the composition is a pharmaceutical composition comprising the presently disclosed cells and a pharmaceutically acceptable carrier.

Administration of the compositions can be autologous or heterologous. For example, cells can be obtained from one subject, and administered to the same subject or a different, compatible subject. Peripheral blood derived cells or their progeny (e.g., in vivo, ex vivo or in vitro derived) can be administered. When administering a presently disclosed composition (e.g., a pharmaceutical composition comprising presently disclosed cells), it can be formulated in a unit dosage injectable form (solution, suspension, emulsion).

The presently disclosed cells and compositions can be administered by any method known in the art including, but not limited to, oral administration, intravenous administration, intravesicular administration, intracranial administration, intraocular administration, subcutaneous administration, intranodal administration, intratumoral administration, intrathecal administration, intrapleural administration, intraosseous administration, intraperitoneal administration, pleural administration, and direct administration to the subject.

### 8. METHODS OF TREATMENT

The presently disclosed subject cells and compositions comprising thereof can be used for treating and/or preventing a tumor or neoplasm. Such cells can be administered to a subject (e.g., a human subject) in need thereof for treatment and/or prevention of a tumor or neoplasm (e.g., metastatic cancer). In certain embodiments, the cell is a T cell. The T cell can be a CD4⁺ T cell or a CD8⁺ T cell. In certain embodiments, the T cell is a CD4⁺ T cell.

The presently disclosed subject matter provides methods for inducing and/or increasing an immune response in a subject in need thereof. The presently disclosed cells and compositions comprising thereof can be used in a therapy or medicament. The presently disclosed subject matter provides various methods of using the cells (e.g., T cells) or compositions comprising thereof. For example, the presently disclosed cells and compositions comprising thereof can be used for reducing tumor burden in a subject. The presently disclosed cell can reduce the number of tumor cells, reduce tumor size, and/or eradicate the tumor in the subject. The presently disclosed cells and compositions comprising thereof can be used for treating and/or preventing a tumor or neoplasm in a subject. The presently disclosed cells and compositions comprising thereof can be used for prolonging the survival of a subject suffering from a tumor or neoplasm. Such methods comprise administering the presently disclosed cells or a composition (e.g., a pharmaceutical composition) comprising thereof to achieve the desired effect, e.g., palliation of an existing condition or prevention of recurrence. For treatment, the amount administered is an amount effective in producing the desired effect. An effective amount can be provided in one or a series of administrations. An effective amount can be provided in a bolus or by continuous perfusion.

The presently disclosed subject matter provides various methods of using the cells (e.g., T cells) or compositions comprising thereof. For example, the presently disclosed subject matter provides methods of reducing tumor burden in a subject. In certain embodiments, the method of reducing tumor burden comprises administering the presently disclosed cells or a composition comprising thereof to the subject. The presently disclosed cell can reduce the number of tumor cells, reduce tumor size, and/or eradicate the tumor in the subject.

The presently disclosed subject matter also provides methods of increasing or lengthening survival of a subject having a tumor or neoplasm. In certain embodiments, the method of increasing or lengthening survival of a subject having a tumor or neoplasm comprises administering the presently disclosed immunoresponsive cells or a composition comprising thereof to the subject. The method can reduce or eradicate tumor burden in the subject. Additionally, the presently disclosed subject matter provides methods for increasing an immune response in a subject, comprising administering the presently disclosed cell or a composition comprising thereof to the subject. The presently disclosed subject matter further provides methods for treating and/or preventing a tumor or neoplasm in a subject, comprising administering the presently disclosed cells or a composition comprising thereof to the subject.

In certain embodiments, the tumor or neoplasm is associated with a L1CAM polypeptide. In certain embodiments, the tumor or neoplasm is associated with overexpression of a L1CAM polypeptide. In certain embodiments, the tumor is cancer. In certain embodiments, the cancer is a metastatic cancer. In certain embodiments, the metastatic cancer is an advanced metastatic cancer. Metastatic cancer is a cancer that has spread from the part of the body where it started (e.g., the primary site) to other parts of the body. When cancer cells break away from a tumor, they can travel to other parts of the body through the bloodstream or the lymph system. In certain non-limiting embodiments, the metastatic cancer localizes in at least one tissue selected from the group consisting of bone, liver, lung, brain, peritoneum, adrenal gland, skin, lymph nodes, pleura, and meninges.

The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

In certain embodiments, the presently disclosed subject matter also provides methods of reducing the risk of metastatic spread in a subject in need thereof. As used herein, “reducing the risk of metastatic spread” is relative to the risk of metastatic spread in a comparable control subject not treated with the presently disclosed cells.

In certain embodiments, the presently disclosed subject matter also provides methods of inhibiting metastatic spread of a primary cancer in a subject in need thereof. As used herein, the term “inhibiting metastatic spread of a primary cancer” means one or more of reducing the number, location(s), and/or size, of metastasis/es, and/or increasing the period of time to occurrence of metastasis/es, and/or prolonging survival, relative to a comparable control subject not treated with the presently disclosed cells.

In certain embodiments, the presently disclosed subject matter also provides methods of inhibiting progression of metastatic disease in a subject in need thereof. As used herein, “inhibiting progression of metastatic disease” means one or more of the following: decreasing the size of existing metastasis/es, reducing the rate of growth of existing metastasis/es, reducing the incidence of newly detectable metastasis/es, improving quality of life, and/or increasing time to recurrence, and/or prolonging survival, relative to a comparable control subject not treated with the presently disclosed cells.

As a consequence of surface expression of a presently disclosed L1CAM-targeted antigen-recognizing receptor, adoptively transferred cells (e.g., immunoresponsive cells, e.g., T cells or NK cells) are endowed with augmented and selective cytolytic activity at the tumor site. Furthermore, subsequent to their localization to tumor or viral infection and their proliferation, the cells turn the tumor or viral infection site into a highly conductive environment for a wide range of immune cells involved in the physiological anti-tumor or antiviral response (tumor infiltrating lymphocytes, NK-, NKT-cells, dendritic cells, and macrophages).

Further modification can be introduced to the presently disclosed cells (e.g., T cells) to avert or minimize the risks of immunological complications (known as “malignant T-cell transformation”), e.g., graft versus-host disease (GvHD), or when healthy tissues express the same target antigens as the tumor cells, leading to outcomes similar to GvHD. A potential solution to this problem is engineering a suicide gene into the presently disclosed cells. Suitable suicide genes include, but are not limited to, Herpes simplex virus thymidine kinase (hsv-tk), inducible Caspase 9 Suicide gene (iCasp-9), and a truncated human epidermal growth factor receptor (EGFRt) polypeptide. In certain embodiments, the suicide gene is an EGFRt polypeptide. The EGFRt polypeptide can enable T cell elimination by administering anti-EGFR monoclonal antibody (e.g., cetuximab). EGFRt can be covalently joined to the upstream of the antigen-recognizing receptor (e.g., CAR). The suicide gene can be included within the vector comprising nucleic acids encoding a presently disclosed antigen-recognizing receptor (e.g., CAR). In this way, administration of a prodrug designed to activate the suicide gene (e.g., a prodrug (e.g., AP1903 that can activate iCasp-9) during malignant T-cell transformation or T cell-mediated toxicity (e.g., GVHD) triggers apoptosis in the suicide gene-activated cells expressing the antigen-recognizing receptor (e.g., CAR). The incorporation of a suicide gene into the presently disclosed antigen-recognizing receptor (e.g., CAR) gives an added level of safety with the ability to eliminate the majority of receptor-expressing cells within a very short time period. A presently disclosed cell (e.g., a T cell) incorporated with a suicide gene can be pre-emptively eliminated at a given timepoint post the cell infusion, or eradicated at the earliest signs of toxicity.

In certain embodiments, the presently disclosed methods include administering the presently disclosed cells or a composition comprising thereof in combination with a second therapeutic agent. As used here, the term “in combination with” means that the presently disclosed cells or a composition comprising thereof, and one or more agents, e.g., a second therapeutic agent, are administered to a subject as part of a treatment regimen or plan.

In certain embodiments, the second therapeutic agent targets components of the immune system to fight cancer can be used with the presently disclosed methods. Non-limiting examples of second therapeutic agents include immune checkpoint inhibitors, dendritic cells, therapeutic antibodies (e.g., anti-CD33 antibodies, anti-CD11b antibodies), cancer vaccines, cytokines (e.g., IL-12, GM-CSF, IL-2, IFNβ, IFNγ, MIP-1, MCP-1, IL-8), Bacillus Calmette-Guérin (BCG), and any combinations thereof. In certain embodiments, the second therapeutic agent is an immune checkpoint inhibitor. In certain embodiments, the immune checkpoint inhibitor is selected from anti-PD1 antibodies, anti-PD-L1 antibodies, anti-CTLA-4 antibodies, anti-BTLA antibodies, anti-TIM3 antibodies, anti-LAG-3 antibodies, and any combinations thereof. For example, but without any limitation, the immune checkpoint inhibitor can be pembrolizumab (KEYTRUDA®), nivolumab (OPDIVO®), cemiplimab (LIBTAYO®), atezolizumab (TECENTRIQ®), avelumab (BAVENCIO®), durvalumab (IMFINZI®), or ipilimumab (YERVOY®).

In certain embodiments, the second therapeutic agent is a chemotherapy. For example, but without any limitation, chemotherapy includes CHOP (cyclophosphamide, doxorubicin, vincristie, prednisone), EPOCH (etoposide, vincristine, doxorubicin, cyclophosphamide, prednisone), or any other multidrug regimens.

In certain embodiments, the presently disclosed cells or a composition comprising thereof, and the second therapeutic agent can be administered to the subject as part of a treatment regimen. In certain embodiments, the presently disclosed cells or a composition comprising thereof, and the second therapeutic agent can be administered concurrently to the subject. In certain embodiments, the presently disclosed cells or a composition comprising thereof, and the second therapeutic agent can be administered at the same time. In certain embodiments, the presently disclosed cells or a composition comprising thereof and the second therapeutic agent can be administered sequentially in any order (e.g., the presently disclosed cells are administered to the subject after the second therapeutic agent is administered) or at different points in time (e.g., the presently disclosed cells or a composition comprising thereof and the second therapeutic agent are administered to the subject on the same day but different hours; or the presently disclosed cells or a composition comprising thereof and the second therapeutic agent are administered to the subject in the same week but on different days).

### 9. KITS

The presently disclosed subject matter provides kits for inducing and/or enhancing an immune response in a subject, treating and/or preventing a tumor or neoplasm in a subject, reducing tumor burden in a subject, and/or increasing or lengthening survival of a subject having a tumor or neoplasm in a subject. In certain embodiments, the kit comprises the presently disclosed cells or a composition comprising thereof. In certain embodiments, the kit comprises a sterile container; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. In certain embodiments, the kit includes a nucleic acid molecule encoding a presently disclosed L1CAM-targeted antigen-recognizing receptor (e.g., a CAR or a TCR like fusion molecule).

If desired, the cells and/or nucleic acid molecules are provided together with instructions for administering the cells or nucleic acid molecules to a subject having or at risk of developing a tumor or neoplasm. The instructions generally include information about the use of the composition for the treatment and/or prevention of a tumor or neoplasm. In certain embodiments, the instructions include at least one of the following: description of the therapeutic agent; dosage schedule and administration for treatment or prevention of a tumor or neoplasm; precautions; warnings; indications; counter-indications; over-dosage information; adverse reactions; animal pharmacology; clinical studies; and/or references. The instructions may be printed directly on the container (when present), or as a label applied to the container, or as a separate sheet, pamphlet, card, or folder supplied in or with the container.

### 10. EXEMPLARY EMBODIMENTS

Embodiment 1. An antigen-recognizing receptor, comprising an extracellular antigen-binding domain, a transmembrane domain, and an intracellular signaling domain, wherein the extracellular antigen-binding domain specifically binds to L1CAM and comprises a heavy chain variable region and a light chain variable region, wherein: a) the heavy chain variable region comprises a CDR1 comprising the amino acid sequence set forth in SEQ ID NO: 2 or a conservative modification thereof, a CDR2 comprising the amino acid sequence set forth in SEQ ID NO: 3 or a conservative modification thereof, and a CDR3 comprising the amino acid sequence set forth in SEQ ID NO: 4 or a conservative modification thereof, and the light chain variable region comprises a CDR1 comprising the amino acid sequence set forth in SEQ ID NO: 5 or a conservative modification thereof, a CDR2 comprising the amino acid sequence set forth in SEQ ID NO: 6 or a conservative modification thereof, and a CDR3 comprising the amino acid sequence set forth in SEQ ID NO: 7 or a conservative modification thereof; b) the heavy chain variable region comprises a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof; and the light chain variable region comprises a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof; c) the heavy chain variable region comprises a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 26 or a conservative modification thereof; and the light chain variable region comprises a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof; d) the heavy chain variable region comprises a CDR1 comprising the amino acid sequence set forth in SEQ ID NO: 35 or a conservative modification thereof, a CDR2 comprising the amino acid sequence set forth in SEQ ID NO: 36 or a conservative modification thereof, and a CDR3 comprising the amino acid sequence set forth in SEQ ID NO: 37 or a conservative modification thereof; and the light chain variable region comprises a CDR1 comprising the amino acid sequence set forth in SEQ ID NO: 38 or a conservative modification thereof, a CDR2 comprising the amino acid sequence set forth in SEQ ID NO: 39 or a conservative modification thereof, and a CDR3 comprising the amino acid sequence set forth in SEQ ID NO: 40 or a conservative modification thereof, e) the heavy chain variable region comprises a CDR1 comprising the amino acid sequence set forth in SEQ ID NO: 43 or a conservative modification thereof, a CDR2 comprising the amino acid sequence set forth in SEQ ID NO: 44 or a conservative modification thereof, and a CDR3 comprising the amino acid sequence set forth in SEQ ID NO: 45 or a conservative modification thereof; and the light chain variable region comprises a CDR1 comprising the amino acid sequence set forth in SEQ ID NO: 46 or a conservative modification thereof, a CDR2 comprising the amino acid sequence set forth in SEQ ID NO: 47 or a conservative modification thereof, and a CDR3 comprising the amino acid sequence set forth in SEQ ID NO: 48 or a conservative modification thereof; f) the heavy chain variable region comprises a CDR1 comprising the amino acid sequence set forth in SEQ ID NO: 43 or a conservative modification thereof, a CDR2 comprising the amino acid sequence set forth in SEQ ID NO: 44 or a conservative modification thereof, and a CDR3 comprising the amino acid sequence set forth in SEQ ID NO: 45 or a conservative modification thereof; and the light chain variable region comprises a CDR1 comprising the amino acid sequence set forth in SEQ ID NO: 51 or a conservative modification thereof, a CDR2 comprising the amino acid sequence set forth in SEQ ID NO: 52 or a conservative modification thereof, and a CDR3 comprising the amino acid sequence set forth in SEQ ID NO: 40 or a conservative modification thereof; g) the heavy chain variable region comprises a CDR1 comprising the amino acid sequence set forth in SEQ ID NO: 54 or a conservative modification thereof, a CDR2 comprising the amino acid sequence set forth in SEQ ID NO: 55 or a conservative modification thereof, and a CDR3 comprising the amino acid sequence set forth in SEQ ID NO: 56 or a conservative modification thereof; and the light chain variable region comprises a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 58 or a conservative modification thereof; h) the heavy chain variable region comprises a CDR1 comprising the amino acid sequence set forth in SEQ ID NO: 62 or a conservative modification thereof, a CDR2 comprising the amino acid sequence set forth in SEQ ID NO: 63 or a conservative modification thereof, and a CDR3 comprising the amino acid sequence set forth in SEQ ID NO: 64 or a conservative modification thereof; and the light chain variable region comprises a CDR1 comprising the amino acid sequence set forth in SEQ ID NO: 65 or a conservative modification thereof, a CDR2 comprising the amino acid sequence set forth in SEQ ID NO: 58 or a conservative modification thereof, and a CDR3 comprising the amino acid sequence set forth in SEQ ID NO: 66 or a conservative modification thereof; i) the heavy chain variable region comprises a CDR1 comprising the amino acid sequence set forth in SEQ ID NO: 69 or a conservative modification thereof, a CDR2 comprising the amino acid sequence set forth in SEQ ID NO: 70 or a conservative modification thereof, and a CDR3 comprising the amino acid sequence set forth in SEQ ID NO: 71 or a conservative modification thereof; and the light chain variable region comprises a CDR1 comprising the amino acid sequence set forth in SEQ ID NO: 72 or a conservative modification thereof, a CDR2 comprising the amino acid sequence set forth in SEQ ID NO: 73 or a conservative modification thereof, and a CDR3 comprising the amino acid sequence set forth in SEQ ID NO: 74 or a conservative modification thereof; j) the heavy chain variable region comprises a CDR1 comprising the amino acid sequence set forth in SEQ ID NO: 81 or a conservative modification thereof, a CDR2 comprising the amino acid sequence set forth in SEQ ID NO: 82 or a conservative modification thereof, and a CDR3 comprising the amino acid sequence set forth in SEQ ID NO: 83 or a conservative modification thereof; and the light chain variable region comprises a CDR1 comprising the amino acid sequence set forth in SEQ ID NO: 84 or a conservative modification thereof, a CDR2 comprising the amino acid sequence set forth in SEQ ID NO: 85 or a conservative modification thereof, and a CDR3 comprising the amino acid sequence set forth in SEQ ID NO: 86 or a conservative modification thereof; k) the heavy chain variable region comprises a CDR1 comprising the amino acid sequence set forth in SEQ ID NO: 93 or a conservative modification thereof, a CDR2 comprising the amino acid sequence set forth in SEQ ID NO: 94 or a conservative modification thereof, and a CDR3 comprising the amino acid sequence set forth in SEQ ID NO: 95 or a conservative modification thereof; and the light chain variable region comprises a CDR1 comprising the amino acid sequence set forth in SEQ ID NO: 96 or a conservative modification thereof, a CDR2 comprising the amino acid sequence set forth in SEQ ID NO: 97 or a conservative modification thereof, and a CDR3 comprising the amino acid sequence set forth in SEQ ID NO: 98 or a conservative modification thereof, l) the heavy chain variable region comprises a CDR1 comprising the amino acid sequence set forth in SEQ ID NO: 105 or a conservative modification thereof, a CDR2 comprising the amino acid sequence set forth in SEQ ID NO: 106 or a conservative modification thereof, and a CDR3 comprising the amino acid sequence set forth in SEQ ID NO: 107 or a conservative modification thereof; and the light chain variable region comprises a CDR1 comprising the amino acid sequence set forth in SEQ ID NO: 108 or a conservative modification thereof, a CDR2 comprising the amino acid sequence set forth in SEQ ID NO: 39 or a conservative modification thereof, and a CDR3 comprising the amino acid sequence set forth in SEQ ID NO: 109 or a conservative modification thereof, or m) the heavy chain variable region comprises a CDR1 comprising the amino acid sequence set forth in SEQ ID NO: 116 or a conservative modification thereof, a CDR2 comprising the amino acid sequence set forth in SEQ ID NO: 117 or a conservative modification thereof, and a CDR3 comprising the amino acid sequence set forth in SEQ ID NO: 118 or a conservative modification thereof; and the light chain variable region comprises a CDR1 comprising the amino acid sequence set forth in SEQ ID NO: 119 or a conservative modification thereof, a CDR2 comprising the amino acid sequence set forth in SEQ ID NO: 120 or a conservative modification thereof, and a CDR3 comprising the amino acid sequence set forth in SEQ ID NO: 121 or a conservative modification thereof.

Embodiment 2. The antigen-recognizing receptor of embodiment 1, wherein: a) the heavy chain variable region comprises a CDR1 comprising the amino acid sequence set forth in SEQ ID NO: 2, a CDR2 comprising the amino acid sequence set forth in SEQ ID NO: 3, and a CDR3 comprising the amino acid sequence set forth in SEQ ID NO: 4; and the light chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7; b) the heavy chain variable region comprises a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14; and the light chain variable region comprises a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17; c) the heavy chain variable region comprises a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 26; and the light chain variable region comprises a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17; d) the heavy chain variable region comprises a CDR1 comprising the amino acid sequence set forth in SEQ ID NO: 35, a CDR2 comprising the amino acid sequence set forth in SEQ ID NO: 36, and a CDR3 comprising the amino acid sequence set forth in SEQ ID NO: 37; and the light chain variable region comprises a CDR1 comprising the amino acid sequence set forth in SEQ ID NO: 38, a CDR2 comprising the amino acid sequence set forth in SEQ ID NO: 39, and a CDR3 comprising the amino acid sequence set forth in SEQ ID NO: 40; e) the heavy chain variable region comprises a CDR1 comprising the amino acid sequence set forth in SEQ ID NO: 43, a CDR2 comprising the amino acid sequence set forth in SEQ ID NO: 44, and a CDR3 comprising the amino acid sequence set forth in SEQ ID NO: 45; and the light chain variable region comprises a CDR1 comprising the amino acid sequence set forth in SEQ ID NO: 46, a CDR2 comprising the amino acid sequence set forth in SEQ ID NO: 47, and a CDR3 comprising the amino acid sequence set forth in SEQ ID NO: 48; f) the heavy chain variable region comprises a CDR1 comprising the amino acid sequence set forth in SEQ ID NO: 43, a CDR2 comprising the amino acid sequence set forth in SEQ ID NO: 44, and a CDR3 comprising the amino acid sequence set forth in SEQ ID NO: 45; and the light chain variable region comprises a CDR1 comprising the amino acid sequence set forth in SEQ ID NO: 51, a CDR2 comprising the amino acid sequence set forth in SEQ ID NO: 52, and a CDR3 comprising the amino acid sequence set forth in SEQ ID NO: 40; g) the heavy chain variable region comprises a CDR1 comprising the amino acid sequence set forth in SEQ ID NO: 54, a CDR2 comprising the amino acid sequence set forth in SEQ ID NO: 55, and a CDR3 comprising the amino acid sequence set forth in SEQ ID NO: 56; and the light chain variable region comprises a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 58; h) the heavy chain variable region comprises a CDR1 comprising the amino acid sequence set forth in SEQ ID NO: 62, a CDR2 comprising the amino acid sequence set forth in SEQ ID NO: 63, and a CDR3 comprising the amino acid sequence set forth in SEQ ID NO: 64; and the light chain variable region comprises a CDR1 comprising the amino acid sequence set forth in SEQ ID NO: 65, a CDR2 comprising the amino acid sequence set forth in SEQ ID NO: 58, and a CDR3 comprising the amino acid sequence set forth in SEQ ID NO: 66; i) the heavy chain variable region comprises a CDR1 comprising the amino acid sequence set forth in SEQ ID NO: 69, a CDR2 comprising the amino acid sequence set forth in SEQ ID NO: 70, and a CDR3 comprising the amino acid sequence set forth in SEQ ID NO: 71; and the light chain variable region comprises a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 74; j) the heavy chain variable region comprises a CDR1 comprising the amino acid sequence set forth in SEQ ID NO: 81, a CDR2 comprising the amino acid sequence set forth in SEQ ID NO: 82, and a CDR3 comprising the amino acid sequence set forth in SEQ ID NO: 83; and the light chain variable region comprises a CDR1 comprising the amino acid sequence set forth in SEQ ID NO: 84, a CDR2 comprising the amino acid sequence set forth in SEQ ID NO: 85, and a CDR3 comprising the amino acid sequence set forth in SEQ ID NO: 86; k) the heavy chain variable region comprises a CDR1 comprising the amino acid sequence set forth in SEQ ID NO: 93, a CDR2 comprising the amino acid sequence set forth in SEQ ID NO: 94, and a CDR3 comprising the amino acid sequence set forth in SEQ ID NO: 95; and the light chain variable region comprises a CDR1 comprising the amino acid sequence set forth in SEQ ID NO: 96, a CDR2 comprising the amino acid sequence set forth in SEQ ID NO: 97, and a CDR3 comprising the amino acid sequence set forth in SEQ ID NO: 98; l) the heavy chain variable region comprises a CDR1 comprising the amino acid sequence set forth in SEQ ID NO: 105, a CDR2 comprising the amino acid sequence set forth in SEQ ID NO: 106, and a CDR3 comprising the amino acid sequence set forth in SEQ ID NO: 107; and the light chain variable region comprises a CDR1 comprising the amino acid sequence set forth in SEQ ID NO: 108, a CDR2 comprising the amino acid sequence set forth in SEQ ID NO: 39, and a CDR3 comprising the amino acid sequence set forth in SEQ ID NO: 109; or m) the heavy chain variable region comprises a CDR1 comprising the amino acid sequence set forth in SEQ ID NO: 116, a CDR2 comprising the amino acid sequence set forth in SEQ ID NO: 117, and a CDR3 comprising the amino acid sequence set forth in SEQ ID NO: 118; and the light chain variable region comprises a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121.

Embodiment 3. The antigen-recognizing receptor of embodiment 1 or 2, wherein: a) the heavy chain variable region comprises a CDR1 comprising the amino acid sequence set forth in SEQ ID NO: 81, a CDR2 comprising the amino acid sequence set forth in SEQ ID NO: 82, and a CDR3 comprising the amino acid sequence set forth in SEQ ID NO: 83; and the light chain variable region comprises a CDR1 comprising the amino acid sequence set forth in SEQ ID NO: 84, a CDR2 comprising the amino acid sequence set forth in SEQ ID NO: 85, and a CDR3 comprising the amino acid sequence set forth in SEQ ID NO: 86; b) the heavy chain variable region comprises a CDR1 comprising the amino acid sequence set forth in SEQ ID NO: 93, a CDR2 comprising the amino acid sequence set forth in SEQ ID NO: 94, and a CDR3 comprising the amino acid sequence set forth in SEQ ID NO: 95; and the light chain variable region comprises a CDR1 comprising the amino acid sequence set forth in SEQ ID NO: 96, a CDR2 comprising the amino acid sequence set forth in SEQ ID NO: 97, and a CDR3 comprising the amino acid sequence set forth in SEQ ID NO: 98; or c) the heavy chain variable region comprises a CDR1 comprising the amino acid sequence set forth in SEQ ID NO: 105, a CDR2 comprising the amino acid sequence set forth in SEQ ID NO: 106, and a CDR3 comprising the amino acid sequence set forth in SEQ ID NO: 107; and the light chain variable region comprises a CDR1 comprising the amino acid sequence set forth in SEQ ID NO: 108, a CDR2 comprising the amino acid sequence set forth in SEQ ID NO: 39, and a CDR3 comprising the amino acid sequence set forth in SEQ ID NO: 109.

Embodiment 4. The antigen-recognizing receptor of any one of embodiments 1-3, wherein the extracellular antigen-binding domain is a single-chain variable fragment (scFv), a Fab, which is optionally crosslinked, or a F(ab)2.

Embodiment 5. The antigen-recognizing receptor of embodiment 4, wherein the extracellular antigen-binding domain is a humanized scFv.

Embodiment 6. The antigen-recognizing receptor of embodiment 4 or 5, wherein one or more of the scFv, Fab and F(ab)2 are comprised in a fusion protein with a heterologous sequence to form the extracellular antigen-binding domain.

Embodiment 7. The antigen-recognizing receptor of any one of embodiments 1-6, wherein the heavy chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 8, SEQ ID NO: 18, SEQ ID NO: 22, SEQ ID NO: 24, SEQ ID NO: 25, SEQ ID NO: 27, SEQ ID NO: 28, SEQ ID NO: 29, SEQ ID NO: 33, SEQ ID NO: 41, SEQ ID NO: 49, SEQ ID NO: 60, SEQ ID NO: 67, SEQ ID NO: 75, SEQ ID NO: 87, SEQ ID NO: 99, SEQ ID NO: 110, SEQ ID NO: 122, SEQ ID NO: 128, SEQ ID NO: 130, SEQ ID NO: 134, or SEQ ID NO: 135.

Embodiment 8. The antigen-recognizing receptor of any one of embodiments 1-7, wherein the light chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 9, SEQ ID NO: 19, SEQ ID NO: 23, SEQ ID NO: 30, SEQ ID NO: 31, SEQ ID NO: 32, SEQ ID NO: 34, SEQ ID NO: 42, SEQ ID NO: 50, SEQ ID NO: 53, SEQ ID NO: 61, SEQ ID NO: 68, SEQ ID NO: 76, SEQ ID NO: 88, SEQ ID NO: 100, SEQ ID NO: 111, SEQ ID NO: 123, SEQ ID NO: 129, SEQ ID NO: 131, SEQ ID NO: 132, SEQ ID NO: 133, or SEQ ID NO: 136.

Embodiment 9. The antigen-recognizing receptor of any one of embodiments 1-8, wherein a) the heavy chain variable region comprises the amino acid sequence set forth in SEQ ID NO: 8, and the light chain variable region comprises the amino acid sequence set forth in SEQ ID NO: 9; b) the heavy chain variable region comprises the amino acid sequence set forth in SEQ ID NO: 18, and the light chain variable region comprises the amino acid sequence set forth in SEQ ID NO: 19; c) the heavy chain variable region comprises the amino acid sequence set forth in SEQ ID NO: 22, and the light chain variable region comprises the amino acid sequence set forth in SEQ ID NO: 23; d) the heavy chain variable region comprises the amino acid sequence set forth in SEQ ID NO: 24, and the light chain variable region comprises the amino acid sequence set forth in SEQ ID NO: 23; e) the heavy chain variable region comprises the amino acid sequence set forth in SEQ ID NO: 25, and the light chain variable region comprises the amino acid sequence set forth in SEQ ID NO: 23; f) the heavy chain variable region comprises the amino acid sequence set forth in SEQ ID NO: 27, and the light chain variable region comprises the amino acid sequence set forth in SEQ ID NO: 23; g) the heavy chain variable region comprises the amino acid sequence set forth in SEQ ID NO: 28, and the light chain variable region comprises the amino acid sequence set forth in SEQ ID NO: 23; h) the heavy chain variable region comprises the amino acid sequence set forth in SEQ ID NO: 29, and the light chain variable region comprises the amino acid sequence set forth in SEQ ID NO: 23; i) the heavy chain variable region comprises the amino acid sequence set forth in SEQ ID NO: 22, and the light chain variable region comprises the amino acid sequence set forth in SEQ ID NO: 30; j) the heavy chain variable region comprises the amino acid sequence set forth in SEQ ID NO: 24, and the light chain variable region comprises the amino acid sequence set forth in SEQ ID NO: 30; k) the heavy chain variable region comprises the amino acid sequence set forth in SEQ ID NO: 25, and the light chain variable region comprises the amino acid sequence set forth in SEQ ID NO: 30; l) the heavy chain variable region comprises the amino acid sequence set forth in SEQ ID NO: 27, and the light chain variable region comprises the amino acid sequence set forth in SEQ ID NO: 30; m) the heavy chain variable region comprises the amino acid sequence set forth in SEQ ID NO: 28, and the light chain variable region comprises the amino acid sequence set forth in SEQ ID NO: 30; n) the heavy chain variable region comprises the amino acid sequence set forth in SEQ ID NO: 29, and the light chain variable region comprises the amino acid sequence set forth in SEQ ID NO: 30; o) the heavy chain variable region comprises the amino acid sequence set forth in SEQ ID NO: 22, and the light chain variable region comprises the amino acid sequence set forth in SEQ ID NO: 31; p) the heavy chain variable region comprises the amino acid sequence set forth in SEQ ID NO: 24, and the light chain variable region comprises the amino acid sequence set forth in SEQ ID NO: 31; q) the heavy chain variable region comprises the amino acid sequence set forth in SEQ ID NO: 25, and the light chain variable region comprises the amino acid sequence set forth in SEQ ID NO: 31; r) the heavy chain variable region comprises the amino acid sequence set forth in SEQ ID NO: 27, and the light chain variable region comprises the amino acid sequence set forth in SEQ ID NO: 31; s) the heavy chain variable region comprises the amino acid sequence set forth in SEQ ID NO: 28, and the light chain variable region comprises the amino acid sequence set forth in SEQ ID NO: 31; t) the heavy chain variable region comprises the amino acid sequence set forth in SEQ ID NO: 29, and the light chain variable region comprises the amino acid sequence set forth in SEQ ID NO: 31; u) the heavy chain variable region comprises the amino acid sequence set forth in SEQ ID NO: 22, and the light chain variable region comprises the amino acid sequence set forth in SEQ ID NO: 32; v) the heavy chain variable region comprises the amino acid sequence set forth in SEQ ID NO: 24, and the light chain variable region comprises the amino acid sequence set forth in SEQ ID NO: 32; w) the heavy chain variable region comprises the amino acid sequence set forth in SEQ ID NO: 25, and the light chain variable region comprises the amino acid sequence set forth in SEQ ID NO: 32; x) the heavy chain variable region comprises the amino acid sequence set forth in SEQ ID NO: 27, and the light chain variable region comprises the amino acid sequence set forth in SEQ ID NO: 32; y) the heavy chain variable region comprises the amino acid sequence set forth in SEQ ID NO: 28, and the light chain variable region comprises the amino acid sequence set forth in SEQ ID NO: 32; z) the heavy chain variable region comprises the amino acid sequence set forth in SEQ ID NO: 29, and the light chain variable region comprises the amino acid sequence set forth in SEQ ID NO: 32; aa) the heavy chain variable region comprises the amino acid sequence set forth in SEQ ID NO: 22, and the light chain variable region comprises the amino acid sequence set forth in SEQ ID NO: 19; ab) the heavy chain variable region comprises the amino acid sequence set forth in SEQ ID NO: 24, and the light chain variable region comprises the amino acid sequence set forth in SEQ ID NO: 19; ac) the heavy chain variable region comprises the amino acid sequence set forth in SEQ ID NO: 25, and the light chain variable region comprises the amino acid sequence set forth in SEQ ID NO: 19; ad) the heavy chain variable region comprises the amino acid sequence set forth in SEQ ID NO: 27, and the light chain variable region comprises the amino acid sequence set forth in SEQ ID NO: 19; ae) the heavy chain variable region comprises the amino acid sequence set forth in SEQ ID NO: 28, and the light chain variable region comprises the amino acid sequence set forth in SEQ ID NO: 19; af) the heavy chain variable region comprises the amino acid sequence set forth in SEQ ID NO: 29, and the light chain variable region comprises the amino acid sequence set forth in SEQ ID NO: 19; ag) the heavy chain variable region comprises the amino acid sequence set forth in SEQ ID NO: 33, and the light chain variable region comprises the amino acid sequence set forth in SEQ ID NO: 34; ah) the heavy chain variable region comprises the amino acid sequence set forth in SEQ ID NO: 41, and the light chain variable region comprises the amino acid sequence set forth in SEQ ID NO: 42; ai) the heavy chain variable region comprises the amino acid sequence set forth in SEQ ID NO: 49, and the light chain variable region comprises the amino acid sequence set forth in SEQ ID NO: 50; aj) the heavy chain variable region comprises the amino acid sequence set forth in SEQ ID NO: 49, and the light chain variable region comprises the amino acid sequence set forth in SEQ ID NO: 53; ak) the heavy chain variable region comprises the amino acid sequence set forth in SEQ ID NO: 60, and the light chain variable region comprises the amino acid sequence set forth in SEQ ID NO: 61; al) the heavy chain variable region comprises the amino acid sequence set forth in SEQ ID NO: 67, and the light chain variable region comprises the amino acid sequence set forth in SEQ ID NO: 68; am) the heavy chain variable region comprises the amino acid sequence set forth in SEQ ID NO: 75, and the light chain variable region comprises the amino acid sequence set forth in SEQ ID NO: 76; an) the heavy chain variable region comprises the amino acid sequence set forth in SEQ ID NO: 87, and the light chain variable region comprises the amino acid sequence set forth in SEQ ID NO: 88; ao) the heavy chain variable region comprises the amino acid sequence set forth in SEQ ID NO: 99, and the light chain variable region comprises the amino acid sequence set forth in SEQ ID NO: 100; ap) the heavy chain variable region comprises the amino acid sequence set forth in SEQ ID NO: 110, and the light chain variable region comprises the amino acid sequence set forth in SEQ ID NO: 111; aq) the heavy chain variable region comprises the amino acid sequence set forth in SEQ ID NO: 122, and the light chain variable region comprises the amino acid sequence set forth in SEQ ID NO: 123; ar) the heavy chain variable region comprises the amino acid sequence set forth in SEQ ID NO: 128, and the light chain variable region comprises the amino acid sequence set forth in SEQ ID NO: 129; as) the heavy chain variable region comprises the amino acid sequence set forth in SEQ ID NO: 130, and the light chain variable region comprises the amino acid sequence set forth in SEQ ID NO: 131; at) the heavy chain variable region comprises the amino acid sequence set forth in SEQ ID NO: 130, and the light chain variable region comprises the amino acid sequence set forth in SEQ ID NO: 132; au) the heavy chain variable region comprises the amino acid sequence set forth in SEQ ID NO: 130, and the light chain variable region comprises the amino acid sequence set forth in SEQ ID NO: 133; av) the heavy chain variable region comprises the amino acid sequence set forth in SEQ ID NO: 134, and the light chain variable region comprises the amino acid sequence set forth in SEQ ID NO: 131; aw) the heavy chain variable region comprises the amino acid sequence set forth in SEQ ID NO: 134, and the light chain variable region comprises the amino acid sequence set forth in SEQ ID NO: 132; ax) the heavy chain variable region comprises the amino acid sequence set forth in SEQ ID NO: 134, and the light chain variable region comprises the amino acid sequence set forth in SEQ ID NO: 133; ay) the heavy chain variable region comprises the amino acid sequence set forth in SEQ ID NO: 135, and the light chain variable region comprises the amino acid sequence set forth in SEQ ID NO: 131; az) the heavy chain variable region comprises the amino acid sequence set forth in SEQ ID NO: 135, and the light chain variable region comprises the amino acid sequence set forth in SEQ ID NO: 132; ba) the heavy chain variable region comprises the amino acid sequence set forth in SEQ ID NO: 135, and the light chain variable region comprises the amino acid sequence set forth in SEQ ID NO: 133; or bb) the heavy chain variable region comprises the amino acid sequence set forth in SEQ ID NO: 135, and the light chain variable region comprises the amino acid sequence set forth in SEQ ID NO: 136.

Embodiment 10. The antigen-recognizing receptor of any one of embodiments 1-9, wherein a) the heavy chain variable region comprises the amino acid sequence set forth in SEQ ID NO: 87, and the light chain variable region comprises the amino acid sequence set forth in SEQ ID NO: 88; b) the heavy chain variable region comprises the amino acid sequence set forth in SEQ ID NO: 99, and the light chain variable region comprises the amino acid sequence set forth in SEQ ID NO: 100; or c) the heavy chain variable region comprises the amino acid sequence set forth in SEQ ID NO: 110, and the light chain variable region comprises the amino acid sequence set forth in SEQ ID NO: 111.

Embodiment 11. The antigen-recognizing receptor of any one of embodiments 1-10, wherein the extracellular antigen-binding domain comprises a linker between the heavy chain variable region and the light chain variable region of the extracellular antigen-binding domain.

Embodiment 12. The antigen-recognizing receptor of embodiment 11, wherein the linker consists of the amino acid sequence set forth in SEQ ID NO: 10, SEQ ID NO: 11, SEQ ID NO: 20, SEQ ID NO: 21, SEQ ID NO: 137, or SEQ ID NO: 138.

Embodiment 13. The antigen-recognizing receptor of embodiment 12, wherein the linker consists of the amino acid sequence set forth in SEQ ID NO: 11.

Embodiment 14. The antigen-recognizing receptor of any one of embodiments 1-13, wherein a signal peptide is covalently joined to the 5′ terminus of the extracellular antigen-binding domain.

Embodiment 15. The antigen-recognizing receptor of any one of embodiments 1-14, wherein the heavy chain variable region and the light chain variable region are positioned from the N- to the C-terminus: VH-VL.

Embodiment 16. The antigen-recognizing receptor of any one of embodiments 1-14, wherein the heavy chain variable region and the light chain variable region are positioned from the N- to the C-terminus: VL-VH.

Embodiment 17. An antigen-recognizing receptor, comprising an extracellular antigen-binding domain, a transmembrane domain, and an intracellular signaling domain, wherein the extracellular antigen-binding domain specifically binds to L1CAM and comprises a heavy chain variable region and a light chain variable region, wherein the heavy chain variable region comprises a CDR1 comprising the amino acid sequence set forth in SEQ ID NO: 81, a CDR2 comprising the amino acid sequence set forth in SEQ ID NO: 82, and a CDR3 comprising the amino acid sequence set forth in SEQ ID NO: 83; and the light chain variable region comprises a CDR1 comprising the amino acid sequence set forth in SEQ ID NO: 84, a CDR2 comprising the amino acid sequence set forth in SEQ ID NO: 85, and a CDR3 comprising the amino acid sequence set forth in SEQ ID NO: 86.

Embodiment 18. An antigen-recognizing receptor, comprising an extracellular antigen-binding domain, a transmembrane domain, and an intracellular signaling domain, wherein the extracellular antigen-binding domain specifically binds to L1CAM and comprises a heavy chain variable region and a light chain variable region, wherein the heavy chain variable region comprises a CDR1 comprising the amino acid sequence set forth in SEQ ID NO: 93, a CDR2 comprising the amino acid sequence set forth in SEQ ID NO: 94, and a CDR3 comprising the amino acid sequence set forth in SEQ ID NO: 95; and the light chain variable region comprises a CDR1 comprising the amino acid sequence set forth in SEQ ID NO: 96, a CDR2 comprising the amino acid sequence set forth in SEQ ID NO: 97, and a CDR3 comprising the amino acid sequence set forth in SEQ ID NO: 98.

Embodiment 19. An antigen-recognizing receptor, comprising an extracellular antigen-binding domain, a transmembrane domain, and an intracellular signaling domain, wherein the extracellular antigen-binding domain specifically binds to L1CAM and comprises a heavy chain variable region and a light chain variable region, wherein the heavy chain variable region comprises a CDR1 comprising the amino acid sequence set forth in SEQ ID NO: 105, a CDR2 comprising the amino acid sequence set forth in SEQ ID NO: 106, and a CDR3 comprising the amino acid sequence set forth in SEQ ID NO: 107; and the light chain variable region comprises a CDR1 comprising the amino acid sequence set forth in SEQ ID NO: 108, a CDR2 comprising the amino acid sequence set forth in SEQ ID NO: 39, and a CDR3 comprising the amino acid sequence set forth in SEQ ID NO: 109.

Embodiment 20. An antigen-recognizing receptor, comprising an extracellular antigen-binding domain, a transmembrane domain, and an intracellular signaling domain, wherein the extracellular antigen-binding domain specifically binds to L1CAM and comprises a heavy chain variable region and a light chain variable region, wherein the heavy chain variable region comprises a CDR1, a CDR2, and a CDR3 of a heavy chain variable region comprising the amino acid sequence set forth in SEQ ID NO: 87, and the light chain variable region comprises a CDR1, a CDR2, and a CDR3 of a light chain variable region comprising the amino acid sequence set forth in SEQ ID NO: 88.

Embodiment 21. An antigen-recognizing receptor, comprising an extracellular antigen-binding domain, a transmembrane domain, and an intracellular signaling domain, wherein the extracellular antigen-binding domain specifically binds to L1CAM and comprises a heavy chain variable region and a light chain variable region, wherein the heavy chain variable region comprises a CDR1, a CDR2, and a CDR3 of a heavy chain variable region comprising the amino acid sequence set forth in SEQ ID NO: 99, and the light chain variable region comprises a CDR1, a CDR2, and a CDR3 of a light chain variable region comprising the amino acid sequence set forth in SEQ ID NO: 100.

Embodiment 22. An antigen-recognizing receptor, comprising an extracellular antigen-binding domain, a transmembrane domain, and an intracellular signaling domain, wherein the extracellular antigen-binding domain specifically binds to L1CAM and comprises a heavy chain variable region and a light chain variable region, wherein the heavy chain variable region comprises a CDR1, a CDR2, and a CDR3 of a heavy chain variable region comprising the amino acid sequence set forth in SEQ ID NO: 110, and the light chain variable region comprises a CDR1, a CDR2, and a CDR3 of a light chain variable region comprising the amino acid sequence set forth in SEQ ID NO: 111.

Embodiment 23. An antigen-recognizing receptor, comprising an extracellular antigen-binding domain, a transmembrane domain, and an intracellular signaling domain, wherein the extracellular antigen-binding domain specifically binds to L1CAM and comprises a single chain a single chain variable region (scFv) comprising the amino acid sequence set forth in SEQ ID NO: 77.

Embodiment 24. An antigen-recognizing receptor, comprising an extracellular antigen-binding domain, a transmembrane domain, and an intracellular signaling domain, wherein the extracellular antigen-binding domain specifically binds to L1CAM and comprises a single chain a single chain variable region (scFv) comprising the amino acid sequence set forth in SEQ ID NO: 78.

Embodiment 25. An antigen-recognizing receptor, comprising an extracellular antigen-binding domain, a transmembrane domain, and an intracellular signaling domain, wherein the extracellular antigen-binding domain specifically binds to L1CAM and comprises a single chain a single chain variable region (scFv) comprising the amino acid sequence set forth in SEQ ID NO: 89.

Embodiment 26. An antigen-recognizing receptor, comprising an extracellular antigen-binding domain, a transmembrane domain, and an intracellular signaling domain, wherein the extracellular antigen-binding domain specifically binds to L1CAM and comprises a single chain a single chain variable region (scFv) comprising the amino acid sequence set forth in SEQ ID NO: 90.

Embodiment 27. An antigen-recognizing receptor, comprising an extracellular antigen-binding domain, a transmembrane domain, and an intracellular signaling domain, wherein the extracellular antigen-binding domain specifically binds to L1CAM and comprises a single chain a single chain variable region (scFv) comprising the amino acid sequence set forth in SEQ ID NO: 101.

Embodiment 28. An antigen-recognizing receptor, comprising an extracellular antigen-binding domain, a transmembrane domain, and an intracellular signaling domain, wherein the extracellular antigen-binding domain specifically binds to L1CAM and comprises a single chain a single chain variable region (scFv) comprising the amino acid sequence set forth in SEQ ID NO: 102.

Embodiment 29. The antigen-recognizing receptor of any one of embodiments 1-28, wherein the transmembrane domain comprises a CD8 polypeptide, a CD28 polypeptide, a CD3ζ polypeptide, a CD4 polypeptide, a 4-1BB polypeptide, an OX40 polypeptide, an ICOS polypeptide, a CTLA-4 polypeptide, a PD-1 polypeptide, a LAG-3 polypeptide, a 2B4 polypeptide, a BTLA polypeptide, or a combination thereof.

Embodiment 30. The antigen-recognizing receptor of embodiment 29, wherein the transmembrane domain comprises a CD28 polypeptide.

Embodiment 31. The antigen-recognizing receptor of any one of embodiments 1-30, wherein the intracellular signaling domain comprises a CD3ζ polypeptide.

Embodiment 32. The antigen-recognizing receptor of any one of embodiments 1-31, wherein the intracellular signaling domain further comprises at least one co-stimulatory signaling region.

Embodiment 33. The antigen-recognizing receptor of embodiment 32, wherein the at least one co-stimulatory signaling region comprises a CD28 polypeptide, a 4-1BB polypeptide, an OX40 polypeptide, an ICOS polypeptide, a DAP-10 polypeptide, or a combination thereof.

Embodiment 34. The antigen-recognizing receptor of embodiment 33, wherein the at least one co-stimulatory signaling region comprises a CD28 polypeptide.

Embodiment 35. The antigen-recognizing receptor of any one of embodiments 1-34, wherein the antigen-recognizing receptor is a chimeric antigen receptor (CAR) or a T-cell receptor (TCR) like fusion protein.

Embodiment 36. The antigen-recognizing receptor of embodiment 35, wherein the antigen-recognizing receptor is a CAR.

Embodiment 37. The antigen-recognizing receptor of any one of embodiments 1-36, wherein the antigen-recognizing receptor is recombinantly expressed or expressed from a vector.

Embodiment 38. A cell comprising the antigen-recognizing receptor of any one of embodiments 1-37.

Embodiment 39. The cell of embodiment 38, wherein the antigen-recognizing receptor is constitutively expressed on the surface of the cell.

Embodiment 40. The cell of embodiment 38 or 39, wherein the cell is an immunoresponsive cell.

Embodiment 41. The cell of any one of embodiments 38-40, wherein the cell is a cell of the lymphoid lineage or a cell of the myeloid lineage.

Embodiment 42. The cell of any one of embodiments 38-41, wherein the cell is selected from the group consisting of a T cell, a Natural Killer (NK) cell, and a stem cell from which a lymphoid cell may be differentiated.

Embodiment 43. The cell of embodiment 42, wherein the cell is a T cell.

Embodiment 44. The cell of embodiment 43, wherein the T cell is a cytotoxic T lymphocyte (CTL) or a regulatory T cell.

Embodiment 45. The cell of embodiment 42, wherein the stem cell is a pluripotent stem cell.

Embodiment 46. The cell of embodiment 45, wherein the pluripotent stem cell is an embryoid stem cell or an induced pluripotent stem cell.

Embodiment 47. A nucleic acid molecule encoding the antigen-recognizing receptor of any one of embodiments 1-37.

Embodiment 48. A vector comprising the nucleic acid molecule of embodiment 47.

Embodiment 49. The vector of embodiment 48, wherein the vector is a retroviral vector.

Embodiment 50. The vector of embodiment 49, wherein the retroviral vector is a 7-retroviral vector or a lentiviral vector.

Embodiment 51. A host cell expressing the nucleic acid molecule of embodiment 47.

Embodiment 52. The host cell of embodiment 51, wherein the host cell is a T cell.

Embodiment 53. A composition comprising the cell of any one of embodiments 38-46.

Embodiment 54. The composition of embodiment 53, which is a pharmaceutical composition further comprising a pharmaceutically acceptable carrier.

Embodiment 55. A lipid nanoparticle comprising the nucleic acid of embodiment 47.

Embodiment 56. A composition comprising the lipid nanoparticle of embodiment 55.

Embodiment 57. The composition of embodiment 56, which is a pharmaceutical composition further comprising a pharmaceutically acceptable carrier.

Embodiment 58. A method of treating and/or preventing a tumor in a subject in need thereof, the method comprising administering to the subject an effective amount of the cell of any one of embodiments 38-46 or the composition of any one of embodiments 53, 54, 56, or 57.

Embodiment 59. The method of embodiment 58, wherein the method reduces the number of tumor cells, reduces tumor size, and/or eradicates the tumor in the subject.

Embodiment 60. A method of increasing or lengthening survival of a subject having a tumor, comprising administering to the subject an effective amount of the cell of any one of embodiments 38-46 or the composition of any one of embodiments 53, 54, 56, or 57.

Embodiment 61. A method of reducing the risk of metastatic spread in a subject in need thereof, comprising administering to the subject an effective amount of the cell of any one of embodiments 38-46 or the composition of any one of embodiments 53, 54, 56, or 57.

Embodiment 62. A method of inhibiting metastatic spread of a primary cancer in a subject in need thereof, comprising administering to the subject an effective amount of the cell of any one of embodiments 38-46 or the composition of any one of embodiments 53, 54, 56, or 57.

Embodiment 63. A method of inhibiting progression of metastatic disease in a subject in need thereof, comprising administering to the subject an effective amount of the cell of any one of embodiments 38-46 or the composition of any one of embodiments 53, 54, 56, or 57 Embodiment 64. The method of any one of embodiments 46-51, wherein the tumor is associated with L1CAM.

Embodiment 65. The method of any one of embodiments 46-52, wherein the tumor is a cancer.

Embodiment 66. The method of any one of embodiments 58-65, wherein the cancer is a metastatic cancer.

Embodiment 67. The method of embodiment 66, wherein metastatic cancer localizes in at least one tissue selected from the group consisting of bone, liver, lung, brain, peritoneum, adrenal gland, skin, lymph nodes, pleura, and meninges.

Embodiment 68. A method for producing a cell comprising an antigen-recognizing receptor of any one of embodiments 1-37, comprising introducing into the cell a nucleic acid molecule that encodes the antigen-recognizing receptor.

Embodiment 69. A kit for reducing tumor burden in a subject, treating and/or preventing a tumor in a subject, and/or increasing or lengthening survival of a subject having a tumor, comprising the cell of any one of embodiments 38-46.

Embodiment 70. The kit of embodiment 69, wherein the kit further comprises written instructions for using the cell for reducing tumor burden in a subject, treating and/or preventing a tumor or neoplasm in a subject, and/or increasing or lengthening survival of a subject having a tumor.

Embodiment 71. The cell of any one of embodiments 38-46 or the composition of any one of embodiments 53, 54, 56, or 57 for use in treating and/or preventing a tumor in a subject in need thereof, increasing or lengthening survival of a subject having a tumor, reducing the risk of metastatic spread in a subject in need thereof, inhibiting metastatic spread of a primary cancer in a subject in need thereof, and/or inhibiting progression of metastatic disease in a subject in need thereof.

Embodiment 72. The cell of any one of embodiments 38-46 or the composition of any one of embodiments 53, 54, 56, or 57 for treating and/or preventing a tumor in a subject in need thereof, increasing or lengthening survival of a subject having a tumor, reducing the risk of metastatic spread in a subject in need thereof, inhibiting metastatic spread of a primary cancer in a subject in need thereof, and/or inhibiting progression of metastatic disease in a subject in need thereof.

Embodiment 73. Use of the cell of any one of embodiments 38-46 or the composition of any one of embodiments 53, 54, 56, or 57 for the manufacture of a medicament for treating and/or preventing a tumor in a subject in need thereof, increasing or lengthening survival of a subject having a tumor, reducing the risk of metastatic spread in a subject in need thereof, inhibiting metastatic spread of a primary cancer in a subject in need thereof, and/or inhibiting progression of metastatic disease in a subject in need thereof.

### EXAMPLES

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides disclosed herein, and, as such, may be considered in making and practicing the presently disclosed subject matter. Particularly useful techniques for particular embodiments will be discussed in the sections that follow.

The following examples are put forth so as to provide those of ordinary skill in the art with a complete disclosure and description of how to make and use the presently disclosed cells and compositions and are not intended to limit the scope of the present disclosure.

### Example 1—Antibody Generation

This example describes the generation of antibodies that bind to L1CAM. Antibody generation was carried out by immunizing mice with recombinant L1CAM proteins that were sourced commercially. Hybridoma supernatants were screened for antibodies that bind L1CAM in solution and on cells. Antibodies purified from the hybridoma supernatants were tested for internalization, followed by subcloning and sequencing of the top hits.

### Antibody Generation

Immunogens. Recombinant L1CAM proteins were sourced commercially for immunizations and hybridoma screening. The extracellular domain of human L1CAM (isoform 1, residues 1-1120) fused to a poly-histidine tag via its carboxy terminus was obtained from Creative Biomart [L1CAM-His]. In addition, the extracellular portions of human L1CAM (isoform 1, residues 20-1120 with a heterologous signal sequence at the amino terminus) or mouse L1CAM (residues 1-1123), each with a human Fc fused to the carboxy terminus, were procured from R&D Systems [L1CAM-Fc]. All recombinant proteins were produced in mammalian cells.

Immunizations. Two cohorts of 6 mice each (Balb/c and A/J strains) and 8 AlivaMab® mice with a human immunoglobulin repertoire were primed with a mixture of human and mouse L1CAM-Fc, followed by 3 booster injections with human L1CAM-His over the course of 8-10 weeks. To further stimulate antigen presentation, 3 mice in each group were given anti-mouse CD40 (FGK5 clone, BioXcell) the day of immunogen administration and 3 days after. TiterMax® Gold (Sigma-Aldrich) was used as adjuvant for all doses. Immunogens were administered subcutaneously. Serum was collected periodically to assess the immune response to L1CAM. After the third dose (prime+2 boosts), the serum from most mice had mid-point titers of 1:10,000 or greater when analyzed by enzyme-linked immunosorbent assay (ELISA) using recombinant human L1CAM-His. In addition, most sera demonstrated strong binding to stably transfected HEK293T cells overexpressing human L1CAM.

Mice with high serum mid-point titers from each strain were selected for an acute boost with human L1CAM-His. Three days later, splenocytes were collected and fused with mouse myeloma cells using polyethylene glycol to generate hybridomas, which were plated for screening.

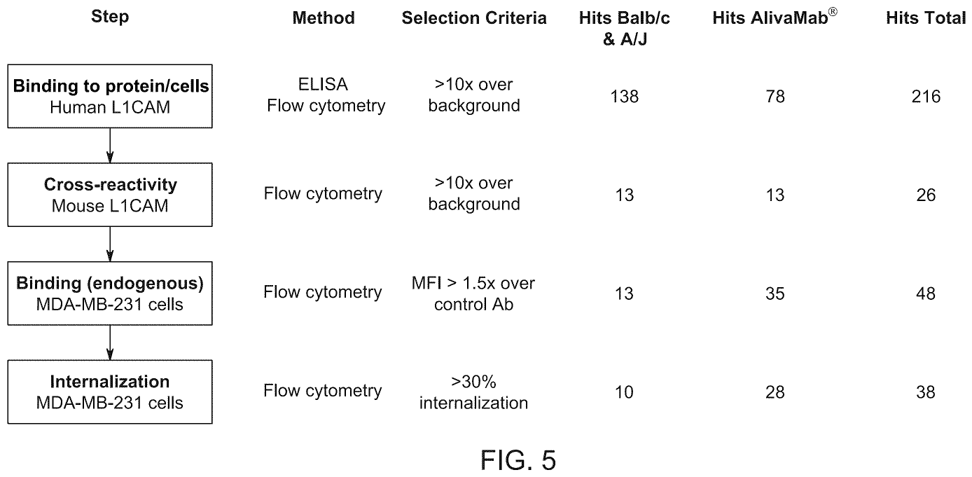

Hybridoma Screening. Hybridoma supernatants were analyzed using a screening funnel shown in FIG. 5. Binding to human L1CAM protein was tested by ELISA and to a HEK293T cell line overexpressing human L1CAM by flow cytometry. Supernatants that showed binding to human L1CAM on cells were further tested for their ability to bind to HEK293FT cells transiently transfected with mouse L1CAM. Cross-reactivity to cynomolgus L1CAM was not assessed at this point, as it is >99% homologous to human L1CAM. The top hits, including subsets of antibodies that did not cross-react with mouse L1CAM, were purified from hybridoma supernatants. The antibodies were tested for binding to MDA-MB-231 cells, a breast cancer cell line that endogenously expresses L1CAM, followed by analysis of their internalization efficacy in MDA-MB-231 cells.

The primary screen by ELISA using soluble, His-tagged human L1CAM and by flow cytometry using HEK293T cells overexpressing human L1CAM resulted in 216 confirmed hits. Of these, 26 were cross-reactive with mouse L1CAM expressed by transiently transfected HEK293FT cells. Furthermore, 76 antibodies with a strong signal over background in the binding assays (ELISA, flow cytometry) were purified from hybridoma supernatant to test for binding to MDA-MB-231 cells. The top 48 binders were tested for internalization in MDA-MB-231 cells, leading to the final hit list with 38 antibodies that fulfilled the criteria defined by the screening funnel.

Subcloning and Sequencing. A total of 32 hybridomas were subcloned by limiting dilution. Clonal supernatants were tested for binding to human L1CAM by ELISA and flow cytometry. Three mAb subclones were selected for each parent, and hybridoma cells were expanded and frozen. The variable regions of the antibodies were sequenced, resulting in 26 unique sequences (10 wild-type mice, 16 AlivaMab®) with good diversity in both the heavy and light chains.

Binding Analysis. To identify lead candidate antibodies, the binding profile of seven mAbs that showed strong internalization was characterized in detail using material purified from the supernatant of hybridoma clones. Dissociation constants (K_{D}) for binding to soluble human and cynomolgus monkey L1CAM-His were determined using biolayer interferometry (BLI). In addition, EC50 values for binding to cell-bound L1CAM were established by flow cytometry using HEK293T/FT cells overexpressing human or mouse L1CAM, MCF-7 cells, and MDA-MB-231 cells. In addition, the epitope regions of the mAbs were determined by BLI interaction analysis with L1CAM subdomains (Ig and FN) expressed individually, and by a BLI competition experiment to identify epitope bins. The results are summarized in Table 55.

TABLE 55
Binding profile of L1CAM mAbs.

| | | | | huL1CAM | cyL1CAM | huL1CAM | moL1CAM | MCF-7 | MDA-231 |
|---|---|---|---|---|---|---|---|---|---|
| | | Epitope | Epitope | Protein | Protein | 293T cells | 293FT cells | Cells | Cells |
| mAb | Mouse | Bin | Domain | K_{D} [nM] | K_{D} [nM] | EC₅₀ [nM] | EC₅₀ [nM] | EC₅₀ [nM] | EC₅₀ [nM] |
| 13F04 | WT | A | Ig | 1.13 | 19.8 | 4 | NB | 1.2 | 1.7 |
| 143G03 | WT | C | FN | 0.12 | 0.34 | 1.0 | NB | 0.35 | 0.9 |
| 04B06 | AMM | D | FN | 0.07 | 0.6 | 2.3 | NB | 1.1 | 9.6 |
| 12D10 | AMM | C | FN | 0.15 | 1.9 | 1.2 | NB | 0.77 | 2 |
| 13G04 | AMM | D | FN | <0.02* | 3.2 | 4.8 | NB | 4.8 | 6.7 |
| 14A10 | AMM | B | Ig | 1.2 | 3.2 | 3.5 | 5.6 | 5.3 | 8.9 |
| 15G02 | AMM | E | FN | 0.58 | 0.93 | 2.6 | NB | 1.0 | 0.98 |

WT: wild-type mice;
AMM: AlivaMab ® Mice
Ig: immunoglobulin domain;
FN: fibronectin domain
NB: no binding
huL1CAM: human L1CAM;
cyL1CAM: cynomolgus L1CAM;
moL1CAM: mouse L1CAM
*off-rate below detection limit

The panel of lead candidate mAbs, identified in both wild-type and AlivaMab® mice, showed good epitope diversity, with five distinct bins (A to E) spread across the two subdomains in the extracellular portion of L1CAM (Ig and FN). All mAbs bound human L1CAM protein at high affinity, with dissociation constants (KD) ranging from low single-digit nM to sub-nM. Cross-reactivity with cynomolgus monkey L1CAM protein was observed for all mAbs. However, only 3 mAbs had dissociation constants within 3-fold of the human protein. The EC50 values for binding to HEK293T cells overexpressing human L1CAM or cells endogenously expressing L1CAM (MCF-7 and MDA-MB-231) were in the single-digit nM or sub-nM range for all mAbs. Notably, 14A10, which is in a separate epitope bin (B), was the only mAb that cross-reacted with mouse L1CAM, as shown with transiently transfected HEK293FT cells.

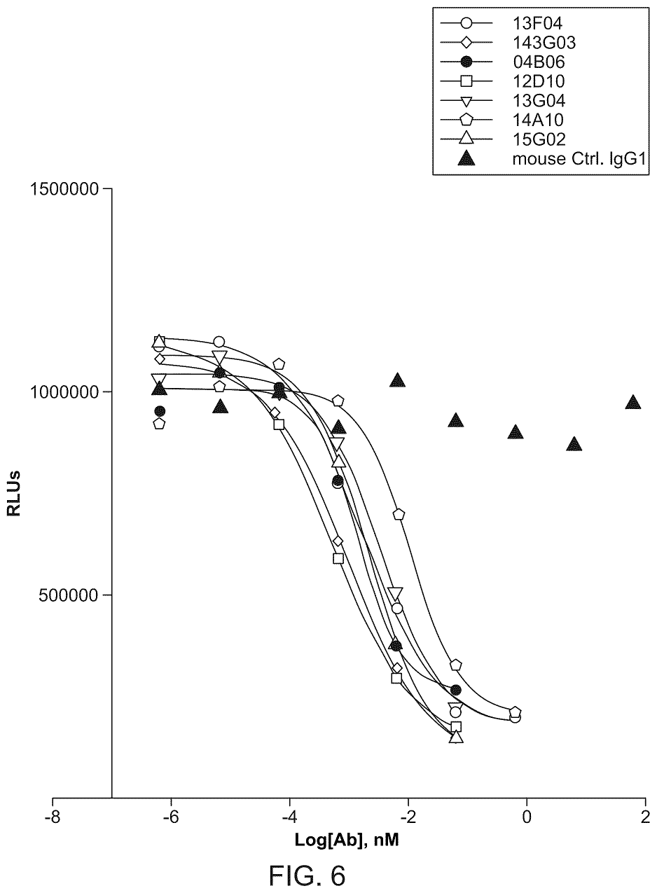

Internalization. Dose-response curves were established for the internalization potency of the lead candidate mAbs using a Fab-ZAP assay. Saporin (a plant ribosome inhibitor) conjugated to a Fab fragment that binds to the Fc portion of mouse IgG was pre-incubated with the L1CAM mAbs, followed by addition of the complex to HEK293T cells overexpressing human L1CAM. Five days later, the viability of the cells was analyzed using a colorimetric assay that detects the cellular metabolic activities (FIG. 6).

A Fab-ZAP assay was used to determine the potency of internalization for the L1CAM lead candidate mAbs in HEK293T cells overexpressing human L1CAM. The L1CAM mAbs and a mouse IgG1 isotype control were pre-incubated at various concentrations with a saporin-conjugated Fab fragment that binds mouse Fc. Five days later, cell viability was measured and plotted against mAb concentrations.

All L1CAM lead candidate mAbs were internalized potently, confirming the results from the screening with antibodies purified from the hybridoma supernatants. Their EC50 values for killing were in the sub-nM range for all mAbs within ˜10× from each other.

Full dose-response curves could not be established for MDA-MB-231 cells, which express substantially lower amounts of L1CAM on the surface than HEK293T cells overexpressing exogenous L1CAM. The one-step mix-and-read format of the Fab-ZAP assay resulted in a “hook effect” at higher L1CAM antibody concentrations prior to reaching the lower plateau.

Based on the binding and internalization data for the seven lead candidate mAbs, 143G03 and 14A10 were selected for antibody-drug conjugate formatting and testing. 143G03 was selected based on its high-affinity binding profile to proteins and cells, and its potent internalization in L1CAM-expressing cells. 14A10 was advanced due its ability to cross-react with mouse L1CAM, enabling the assessment of on-target toxicities in mice, and testing in syngeneic tumor models. 15G02 was identified as the primary backup candidate but was not explored beyond this point.

### Example 2—Humanization of Murine Antibody 76H12

To humanize the variable regions of murine mAb 76H12, the complementarity-determining regions (CDR) were grafted onto human VH and VL acceptor frameworks and select human-to-murine framework substitutions were introduced as needed to fully recapitulate L1CAM binding affinity. IGHV1-46 and IGKV1-39 human germline acceptor frameworks were selected based on sequence identity to the VH and VL regions of 76H12 and on their frequency of usage in natural human immune repertoires. For VH and VL sequence downstream of CDR3, human IGHJ4 and IGKJ2 J-region sequences were used in humanized designs. Four humanized VH sequences (Table 56) and four humanized VL sequences (Table 57) were designed.

TABLE 56
VH sequences of 76H12 and humanized variants

| VH | |
|---|---|
| Identifier | Sequence |
| 76H12_H | QVQLQQPGSDLVRPGTSVKLSCKASGYTFTSYWMHWVKQRPGQGLEWIGN |
| | VYPGSGSTKYDEKFKTKATLTVDTSSNTAYMELSSLTSEDSAVYYCTRPG |
| | NYEGFAYWGQGTLVTVSA [SEQ ID NO: 122] |
| h76H12_Hv0 | QVQLVQSGAEVKKPGASVKVSCKASGYTFTSYWMHWVRQAPGQGLEWMGN |
| | VYPGSGSTKYAQKFQGRVTMTRDTSTSTVYMELSSLRSEDTAVYYCARPG |
| | NYEGFAYWGQGTLVTVSS [SEQ ID NO: 128] |
| h76H12_Hv1 | QVQLVQSGAEVKKPGASVKVSCKASGYTFTSYWMHWVRQAPGQGLEWIGN |
| | VYPGSGSTKYAQKFQGRATLTVDTSTSTVYMELSSLRSEDTAVYYCARPG |
| | NYEGFAYWGQGTLVTVSS [SEQ ID NO: 130] |
| h76H12_Hv2 | QVQLVQSGAEVKKPGASVKVSCKASGYTFTSYWMHWVRQAPGQGLEWIGN |
| | VYPGSGSTKYAQKFQGRATLTVDTSTSTAYMELSSLRSEDTAVYYCTRPG |
| | NYEGFAYWGQGTLVTVSS [SEQ ID NO: 134] |
| h76H12_Hv3 | QVQLVQSGAEVKKPGASVKVSCKASGYTFTSYWMHWVRQAPGQGLEWIGN |
| | VYPGSGSTKYAQKFQGRATLTVDTSTNTAYMELSSLRSEDTAVYYCTRPG |
| | NYEGFAYWGQGTLVTVSS [SEQ ID NO: 135] |

76H12_H corresponds to the sequence of VH from parental (murine) 76H12.
Underlined residues represent CDRs as defined for the purpose of humanization. This is a custom CDR definition distinct from the IMGT definition used elsewhere.
Bold residues in humanized variants represent framework point mutations to match the 76H12_H sequence.

TABLE 57
VL sequences of 76H12 and humanized variants

| VL | |
|---|---|
| Identifier | Sequence |
| 76H12_L | NIVLTQSPASLAVSLGQRATISCRASESVDSYGSSFMHWYQQKPGQPPKL |
| | LIYLASNLESGVPARFSGSGSRTDFTLTIDPVEADDAATYYCQQNNEDPW |
| | TFGGGTKLEIK [SEQ ID NO: 123] |
| h76H12_Lv0 | DIQMTQSPSSLSASVGDRVTITCRASESVDSYGSSFMHWYQQKPGKAPKL |
| | LIYLASNLESGVPSRFSGSGSGTDFTLTISSLQPEDFATYYCQQNNEDPW |
| | TFGQGTKLEIK [SEQ ID NO: 136] |
| h76H12_Lv1 | DIQLTQSPSSLSASVGDRVTITCRASESVDSYGSSFMHWYQQKPGKPPKL |
| | LIYLASNLESGVPSRESGSGSGTDFTLTISSLQPEDFATYYCQQNNEDPW |
| | TFGQGTKLEIK [SEQ ID NO: 131] |
| h76H12_Lv2 | DIQLTQSPSSLSASVGDRVTITCRASESVDSYGSSFMHWYQQKPGKPPKL |
| | LIYLASNLESGVPSRFSGSGSRTDFTLTISSLQPEDFATYYCQQNNEDPW |
| | TFGQGTKLEIK [SEQ ID NO: 132] |
| h76H12_Lv3 | NIQLTQSPSSLSASVGDRVTITCRASESVDSYGSSFMHWYQQKPGKPPKL |
| | LIYLASNLESGVPSRFSGSGSGTDFTLTISSLQPEDFATYYCQQNNEDPW |
| | TFGQGTKLEIK [SEQ ID NO: 133] |

76H12_L corresponds to the sequence of VL from parental (murine) 76H12.
Underlined residues represent CDRs as defined for the purpose of humanization. This is a custom CDR definition distinct from the IMGT definition used elsewhere.
Bold residues in humanized variants represent framework point mutations to match the 76H12_L sequence.

To evaluate monovalent binding by humanized 76H12 variants to L1CAM, Fab proteins were generated (Table 58) with VH sequences fused to CH1 from human IgG1 and VL fused to the constant region of human kappa light chain.

TABLE 58
Design of chimeric and humanized 76H12 Fab variants

| Fab Construct | VH sequence | VL sequence |
|---|---|---|
| 1 (parental mouse | 76H12_H | 76H12_L |
| 76H12 V-regions) | | |
| 2 | h76H12_Hv0 | h76H12_Lv3 |
| 3 | h76H12_Hv1 | h76H12_Lv1 |
| 4 | h76H12_Hv1 | h76H12_Lv2 |
| 5 | h76H12_Hv1 | h76H12_Lv3 |
| 6 | h76H12_Hv2 | h76H12_Lv1 |
| 7 | h76H12_Hv2 | h76H12_Lv2 |
| 8 | h76H12_Hv2 | h76H12_Lv3 |
| 9 | h76H12_Hv3 | h76H12_Lv1 |
| 10 | h76H12_Hv3 | h76H12_Lv2 |
| 11 | h76H12_Hv3 | h76H12_Lv3 |
| 12 | h76H12_Hv3 | h76H12_Lv0 |

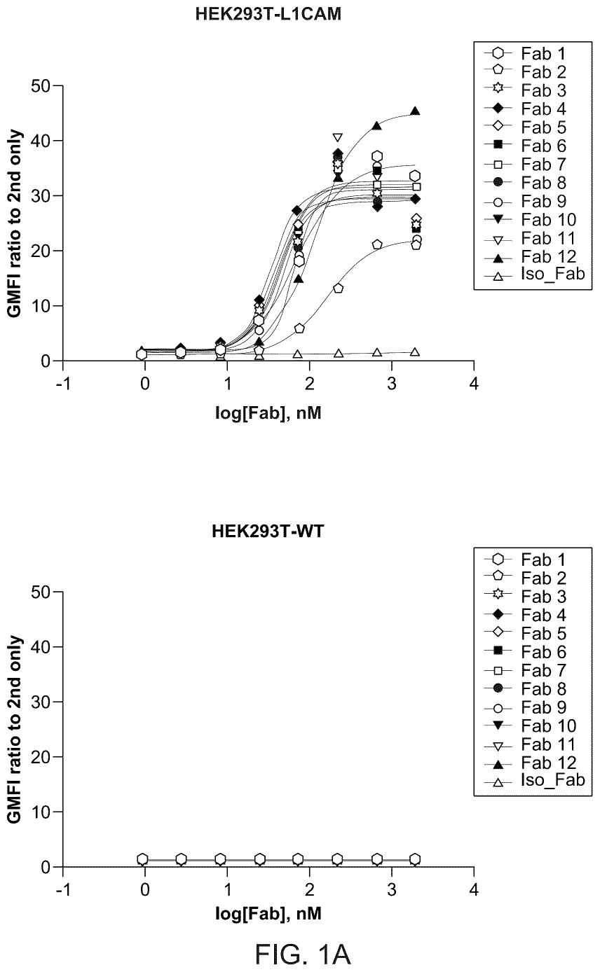

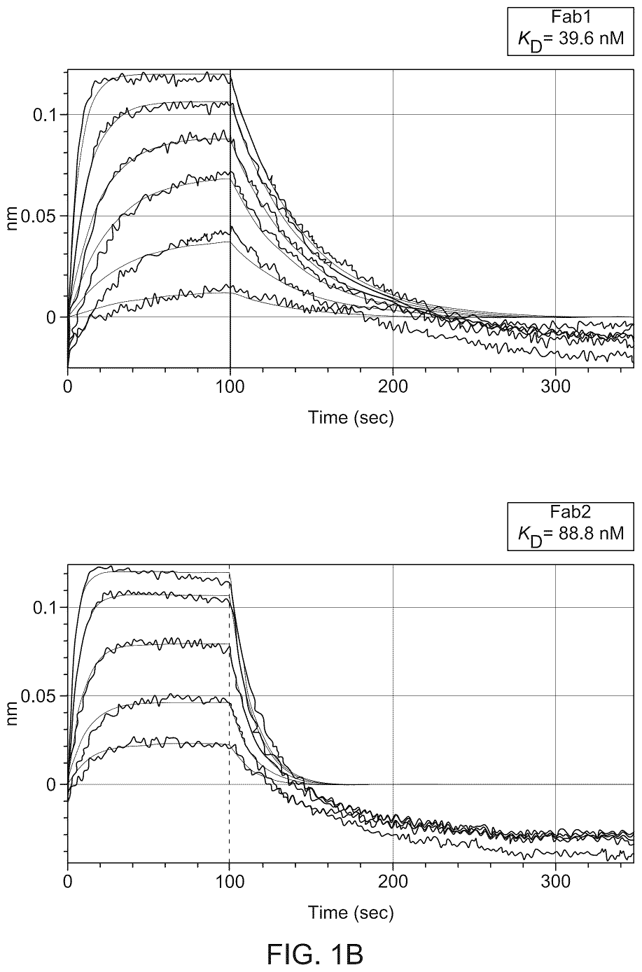

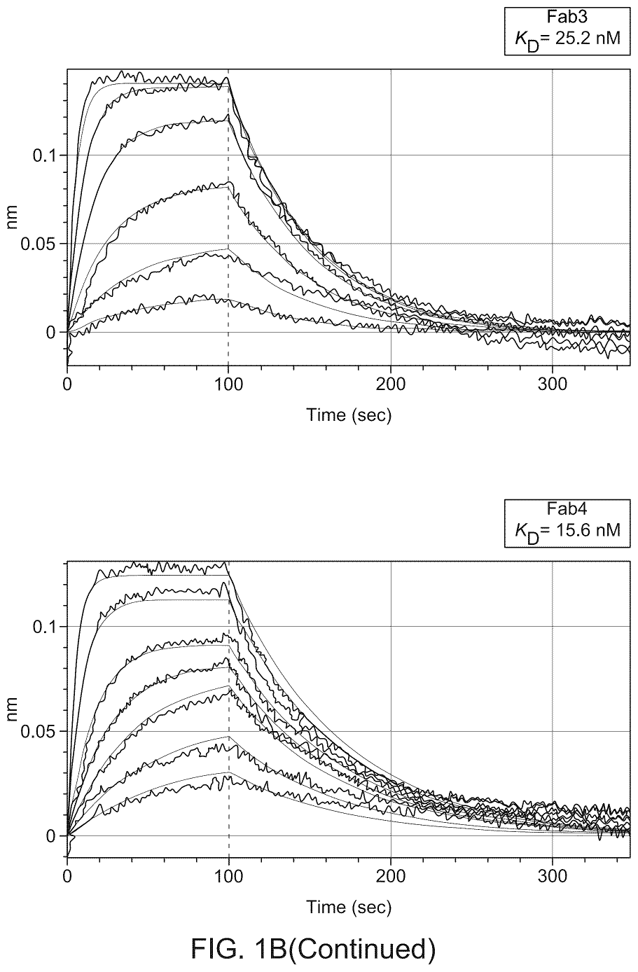

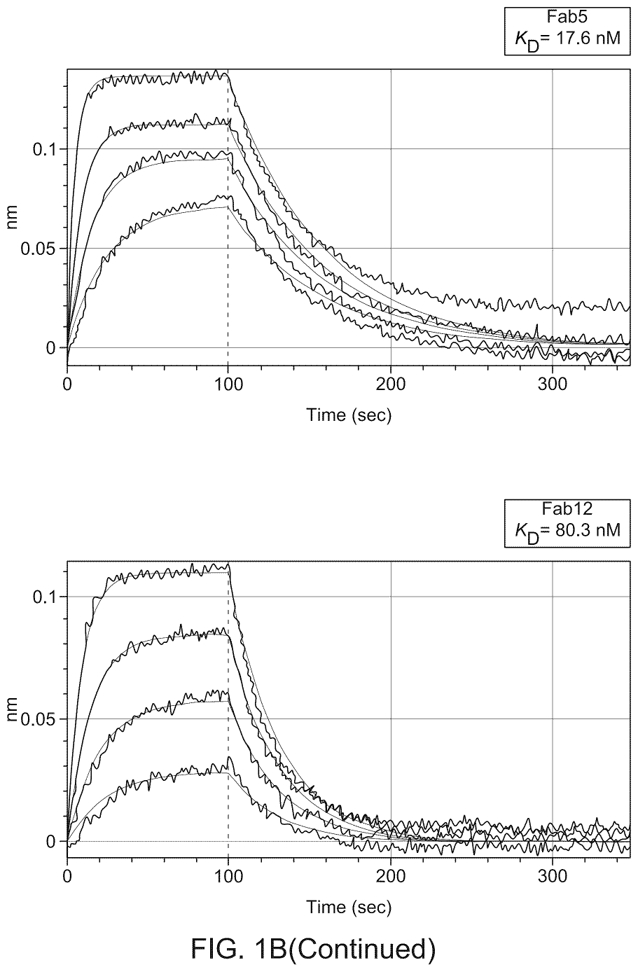

A total of 11 humanized 76H12 Fab variants (Fabs 2-12) were generated in addition to a chimeric 76H12 Fab (Fab 1) containing the parental murine VH and VL sequences. The chimeric Fab served as the benchmark for comparative assessment of humanized Fab variants. Humanized Fabs were first assessed by flow cytometry for binding to 293T cells engineered to overexpress L1CAM (FIG. 1A). All humanized variants bound to the L1CAM overexpressing cells, with most binding comparably to the chimeric 76H12 Fab. None of the Fabs bound to parental 293T cells that do not express L1CAM. Based on these data, the chimeric Fab and a subset of humanized variants were further assessed for binding to recombinant L1CAM protein by bilayer interferometry (FIG. 1B). Here, recombinant L1CAM-Fc fusion protein was immobilized onto anti-Human IgG Fc Capture (AHC) biosensors and binding of Fabs was measured kinetically over a 3-200 nM concentration range. Kinetic data were fitted to a 1:1 binding model to compute association (kₒₙ) and dissociation (k_{off}) rate constants, in addition to equilibrium dissociation constants (K_{D}) as shown in Table 59. Three of the humanized Fab constructs (Fabs 3, 4 and 5) met the criteria of binding to L1CAM with equivalent or better affinity than the chimeric Fab (Fab 1). From these, Fab 3 represents the preferred humanized variant given higher identity to human germline IGHV1-46 and IGKV1-39 sequences resulting from fewer human-to-murine framework back mutations.

TABLE 59
Binding of chimeric and humanized 76H12 Fab variants to L1CAM by BLI.

| Fab Construct | VH sequence | VL sequence | kₒₙ (M⁻¹s⁻¹) | k_{off} (s⁻¹) | K_{D} (M) |
|---|---|---|---|---|---|
| 1 (chimeric) | 76H12_H | 76H12_L | 5.86E+05 | 2.32E−02 | 3.96E−08 |
| 2 (humanized) | h76H12_Hv0 | h76H12_Lv3 | 7.97E+05 | 7.06E−02 | 8.88E−08 |
| 3 (humanized) | h76H12_Hv1 | h76H12_Lv1 | 7.81E+05 | 1.97E−02 | 2.52E−08 |
| 4 (humanized) | h76H12_Hv1 | h76H12_Lv2 | 9.22E+05 | 1.43E−02 | 1.56E−08 |
| 5 (humanized) | h76H12_Hv1 | h76H12_Lv3 | 9.57E+05 | 1.68E−02 | 1.76E−08 |
| 12 (humanized) | h76H12_Hv3 | h76H12_Lv0 | 3.95E+05 | 3.18E−02 | 8.03E−08 |

### Example 3—Generation of CAR T Cells

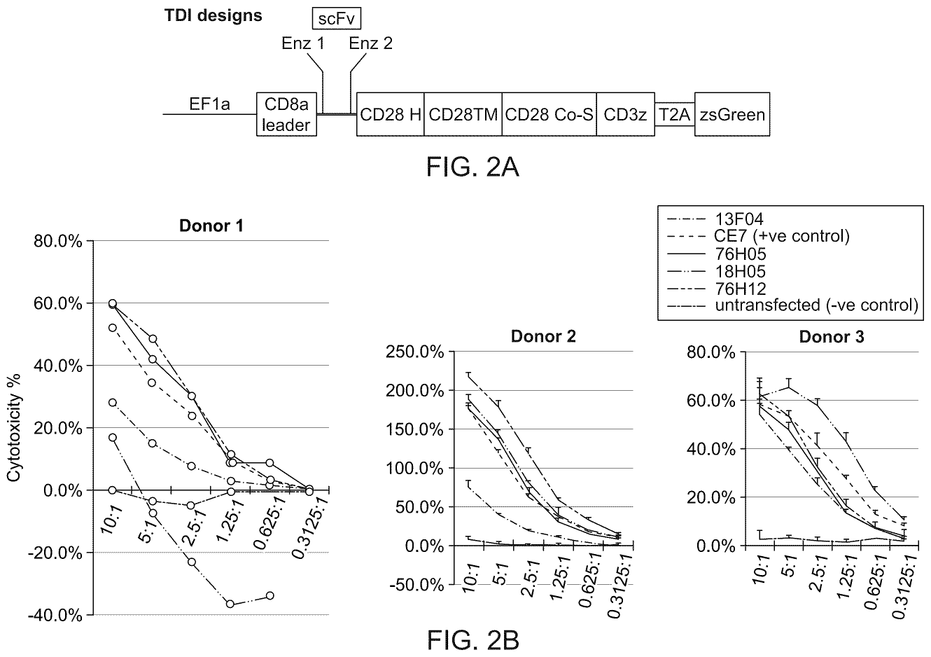

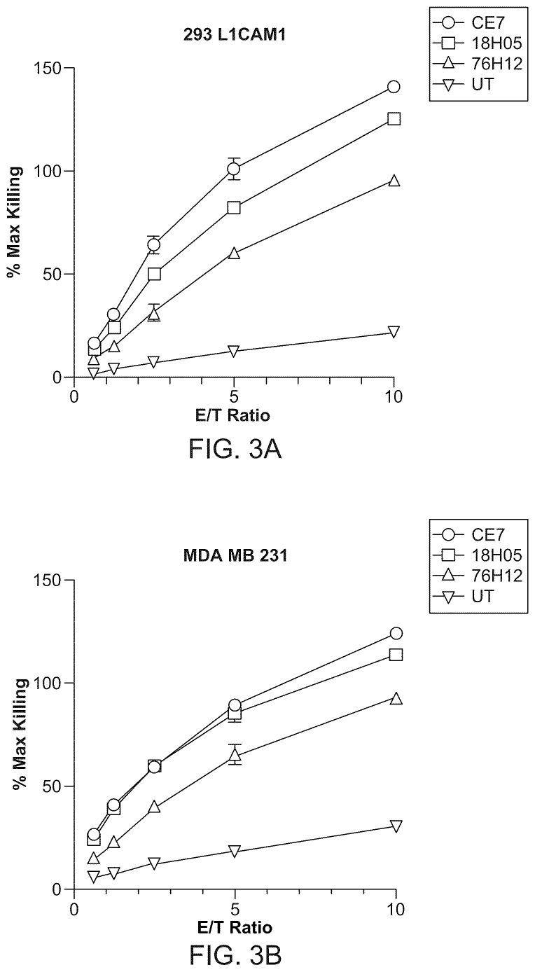

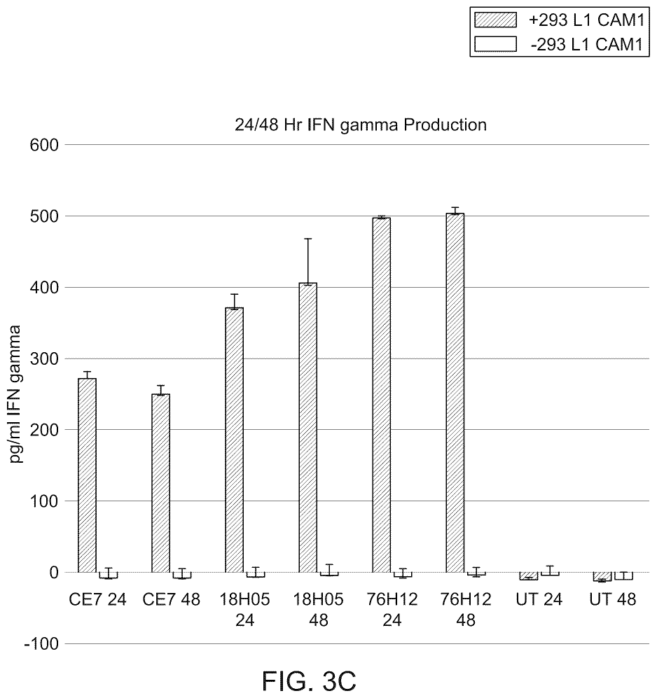

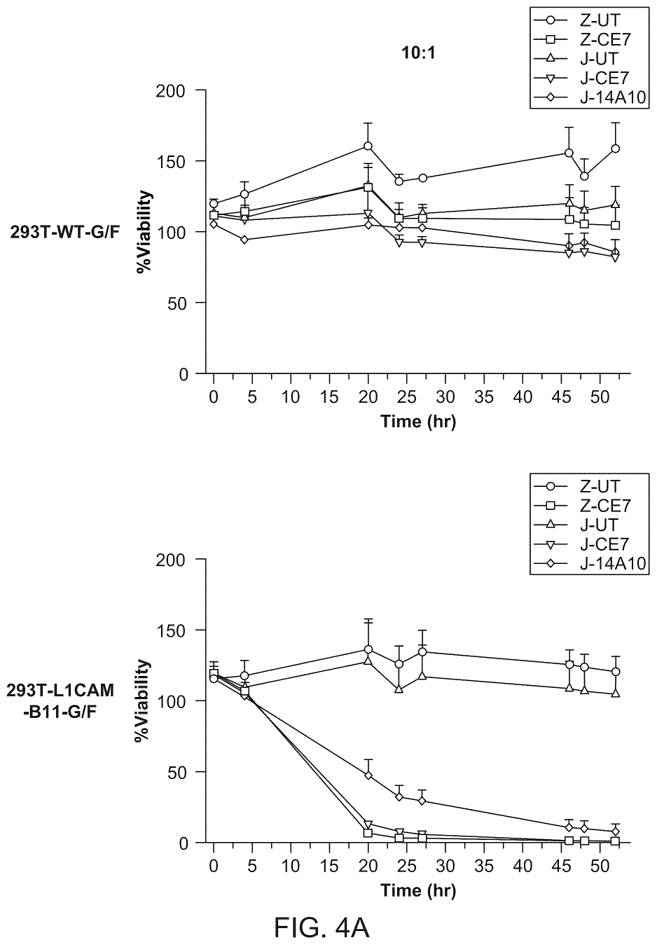

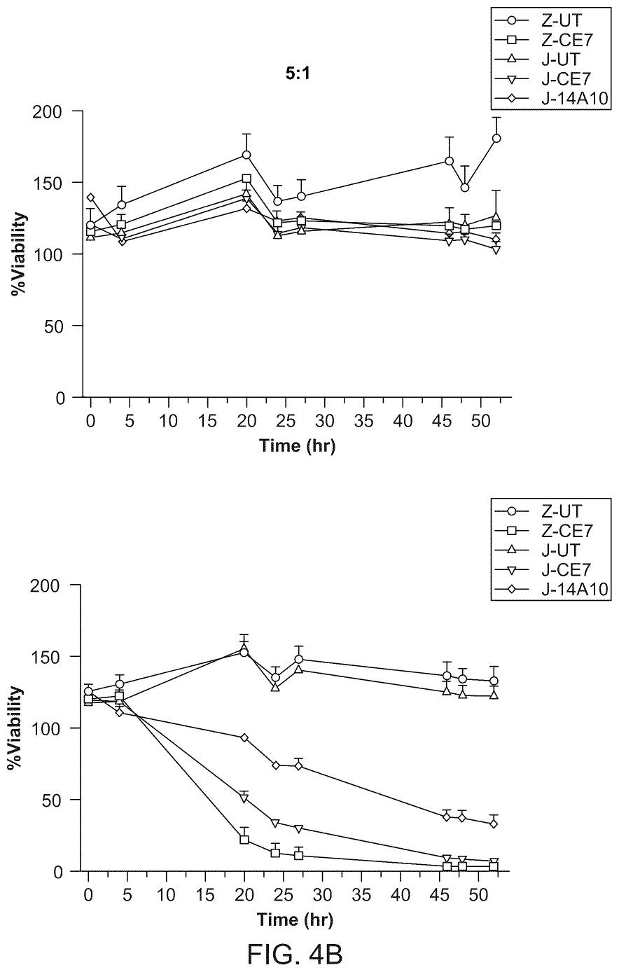

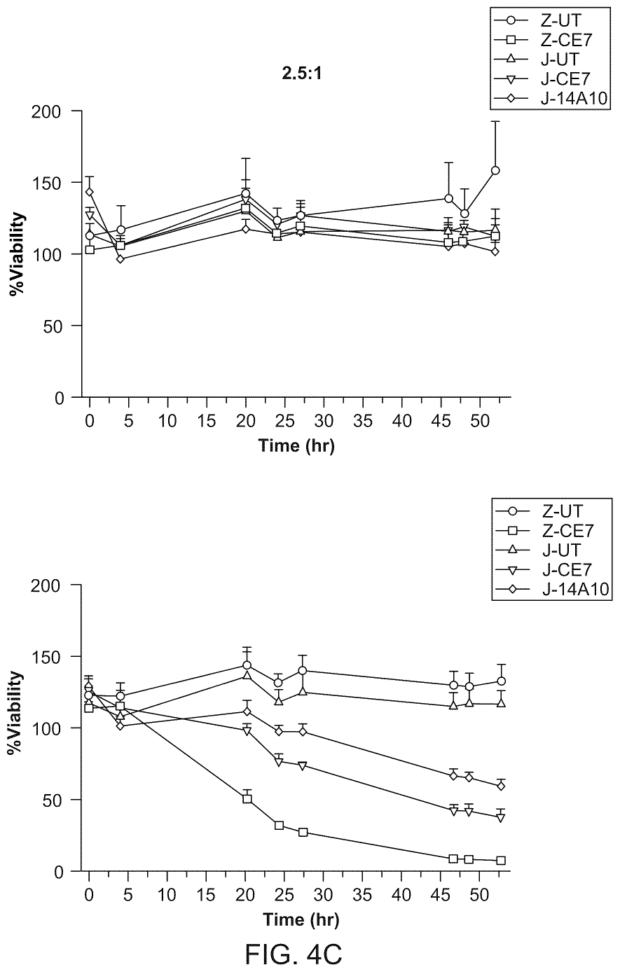

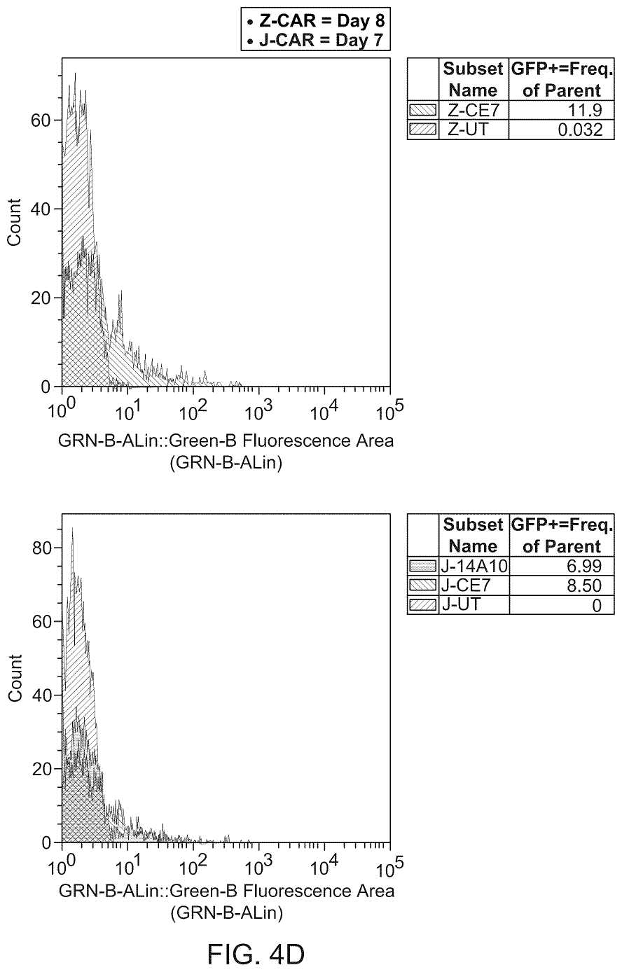

Human T cells were genetically modified to include a chimeric antigen receptor (CAR) as depicted in FIG. 2A. To determine whether CAR T cells targeting L1CAM were effective in cell killing, coculture of different CAR T cell clones with MDA-MB-231 parental cells expressing L1CAM were established. As seen in FIG. 2B, the tested clones induced cytotoxicity of the target cells comparable to the CE7 control (previously described in Giordano and Cavallaro, Journal of Clinical Medicine 9, no. 5 (2020): 1502). Next, CAR including the 18H05 and 76H12 scFvs were selected for further validation. As seen in FIGS. 3A and 3B, both CARs were capable of inducing killing of 293 L1CAM1 cells as well as MDA MB 231 cells even at lower E/T ratios. Notably, when cytokine release was measured, both CARs did not display tonic signaling. Next, the 14A10 scFv was tested to determine whether it could be a good candidate for CAR T cells. FIGS. 4A-4D show that CAR constructs including the 14A10 scFv were efficient in targeting 293T-L1CAM cells at different EFT ratios.

Collectively, these data indicate that the tested scFv are good candidates for the generation of CAR T cells.

### Example 4—Functional Assessment of Humanized 76H12 CAR T Cells

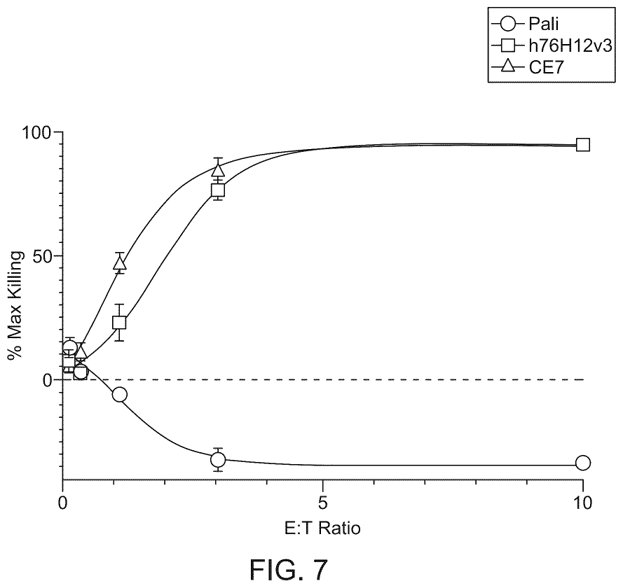

CAR constructs against L1CAM were generated by cloning scFvs derived from humanized 76H12 antibody (76H12v3) and the reference antibody CE7 into an scFv-CD28-CD3z CAR cassette. In addition, a negative control CAR was constructed by cloning an scFv derived from palivizumab into the same cassette. Lentiviruses encoding each of these CARs were used to transduce PBMCs from healthy donors after first activating the T cells according to standard protocols. T cells expressing each of the three transduced CARs constructs were then evaluated for their ability to induce lysis of L1CAM+MDA MB231 breast cancer cells after co-culturing for 48 hours. As shown in FIG. 7, the humanized 76H12 CAR exhibited similar lytic activity as CE7, while the palivizumab negative control had no effect.

### EMBODIMENTS OF THE PRESENTLY DISCLOSED SUBJECT MATTER

From the foregoing description, it will be apparent that variations and modifications may be made to the presently disclosed subject matter to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1.-73. (canceled)

74. An antigen-recognizing receptor, comprising an extracellular antigen-binding domain, a transmembrane domain, and an intracellular signaling domain, wherein the extracellular antigen-binding domain specifically binds to L1CAM and comprises a heavy chain variable region and a light chain variable region, wherein:
a) the heavy chain variable region comprises a CDR1 comprising the amino acid sequence set forth in SEQ ID NO: 2, a CDR2 comprising the amino acid sequence set forth in SEQ ID NO: 3, and a CDR3 comprising the amino acid sequence set forth in SEQ ID NO: 4; and the light chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7;
b) the heavy chain variable region comprises a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14; and the light chain variable region comprises a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17;
c) the heavy chain variable region comprises a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 26; and the light chain variable region comprises a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17;
d) the heavy chain variable region comprises a CDR1 comprising the amino acid sequence set forth in SEQ ID NO: 35, a CDR2 comprising the amino acid sequence set forth in SEQ ID NO: 36, and a CDR3 comprising the amino acid sequence set forth in SEQ ID NO: 37; and the light chain variable region comprises a CDR1 comprising the amino acid sequence set forth in SEQ ID NO: 38, a CDR2 comprising the amino acid sequence set forth in SEQ ID NO: 39, and a CDR3 comprising the amino acid sequence set forth in SEQ ID NO: 40;
e) the heavy chain variable region comprises a CDR1 comprising the amino acid sequence set forth in SEQ ID NO: 43, a CDR2 comprising the amino acid sequence set forth in SEQ ID NO: 44, and a CDR3 comprising the amino acid sequence set forth in SEQ ID NO: 45; and the light chain variable region comprises a CDR1 comprising the amino acid sequence set forth in SEQ ID NO: 46, a CDR2 comprising the amino acid sequence set forth in SEQ ID NO: 47, and a CDR3 comprising the amino acid sequence set forth in SEQ ID NO: 48;
f) the heavy chain variable region comprises a CDR1 comprising the amino acid sequence set forth in SEQ ID NO: 43, a CDR2 comprising the amino acid sequence set forth in SEQ ID NO: 44, and a CDR3 comprising the amino acid sequence set forth in SEQ ID NO: 45; and the light chain variable region comprises a CDR1 comprising the amino acid sequence set forth in SEQ ID NO: 51, a CDR2 comprising the amino acid sequence set forth in SEQ ID NO: 52, and a CDR3 comprising the amino acid sequence set forth in SEQ ID NO: 40;
g) the heavy chain variable region comprises a CDR1 comprising the amino acid sequence set forth in SEQ ID NO: 54, a CDR2 comprising the amino acid sequence set forth in SEQ ID NO: 55, and a CDR3 comprising the amino acid sequence set forth in SEQ ID NO: 56; and the light chain variable region comprises a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 58;
h) the heavy chain variable region comprises a CDR1 comprising the amino acid sequence set forth in SEQ ID NO: 62, a CDR2 comprising the amino acid sequence set forth in SEQ ID NO: 63, and a CDR3 comprising the amino acid sequence set forth in SEQ ID NO: 64; and the light chain variable region comprises a CDR1 comprising the amino acid sequence set forth in SEQ ID NO: 65, a CDR2 comprising the amino acid sequence set forth in SEQ ID NO: 58, and a CDR3 comprising the amino acid sequence set forth in SEQ ID NO: 66;
i) the heavy chain variable region comprises a CDR1 comprising the amino acid sequence set forth in SEQ ID NO: 69, a CDR2 comprising the amino acid sequence set forth in SEQ ID NO: 70, and a CDR3 comprising the amino acid sequence set forth in SEQ ID NO: 71; and the light chain variable region comprises a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 74;
j) the heavy chain variable region comprises a CDR1 comprising the amino acid sequence set forth in SEQ ID NO: 81, a CDR2 comprising the amino acid sequence set forth in SEQ ID NO: 82, and a CDR3 comprising the amino acid sequence set forth in SEQ ID NO: 83; and the light chain variable region comprises a CDR1 comprising the amino acid sequence set forth in SEQ ID NO: 84, a CDR2 comprising the amino acid sequence set forth in SEQ ID NO: 85, and a CDR3 comprising the amino acid sequence set forth in SEQ ID NO: 86;
k) the heavy chain variable region comprises a CDR1 comprising the amino acid sequence set forth in SEQ ID NO: 93, a CDR2 comprising the amino acid sequence set forth in SEQ ID NO: 94, and a CDR3 comprising the amino acid sequence set forth in SEQ ID NO: 95; and the light chain variable region comprises a CDR1 comprising the amino acid sequence set forth in SEQ ID NO: 96, a CDR2 comprising the amino acid sequence set forth in SEQ ID NO: 97, and a CDR3 comprising the amino acid sequence set forth in SEQ ID NO: 98;
l) the heavy chain variable region comprises a CDR1 comprising the amino acid sequence set forth in SEQ ID NO: 105, a CDR2 comprising the amino acid sequence set forth in SEQ ID NO: 106, and a CDR3 comprising the amino acid sequence set forth in SEQ ID NO: 107; and the light chain variable region comprises a CDR1 comprising the amino acid sequence set forth in SEQ ID NO: 108, a CDR2 comprising the amino acid sequence set forth in SEQ ID NO: 39, and a CDR3 comprising the amino acid sequence set forth in SEQ ID NO: 109; or
m) the heavy chain variable region comprises a CDR1 comprising the amino acid sequence set forth in SEQ ID NO: 116, a CDR2 comprising the amino acid sequence set forth in SEQ ID NO: 117, and a CDR3 comprising the amino acid sequence set forth in SEQ ID NO: 118; and the light chain variable region comprises a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121.

75. The antigen-recognizing receptor of claim 74, wherein:
a) the heavy chain variable region comprises a CDR1 comprising the amino acid sequence set forth in SEQ ID NO: 81, a CDR2 comprising the amino acid sequence set forth in SEQ ID NO: 82, and a CDR3 comprising the amino acid sequence set forth in SEQ ID NO: 83; and the light chain variable region comprises a CDR1 comprising the amino acid sequence set forth in SEQ ID NO: 84, a CDR2 comprising the amino acid sequence set forth in SEQ ID NO: 85, and a CDR3 comprising the amino acid sequence set forth in SEQ ID NO: 86;
b) the heavy chain variable region comprises a CDR1 comprising the amino acid sequence set forth in SEQ ID NO: 93, a CDR2 comprising the amino acid sequence set forth in SEQ ID NO: 94, and a CDR3 comprising the amino acid sequence set forth in SEQ ID NO: 95; and the light chain variable region comprises a CDR1 comprising the amino acid sequence set forth in SEQ ID NO: 96, a CDR2 comprising the amino acid sequence set forth in SEQ ID NO: 97, and a CDR3 comprising the amino acid sequence set forth in SEQ ID NO: 98; or
c) the heavy chain variable region comprises a CDR1 comprising the amino acid sequence set forth in SEQ ID NO: 105, a CDR2 comprising the amino acid sequence set forth in SEQ ID NO: 106, and a CDR3 comprising the amino acid sequence set forth in SEQ ID NO: 107; and the light chain variable region comprises a CDR1 comprising the amino acid sequence set forth in SEQ ID NO: 108, a CDR2 comprising the amino acid sequence set forth in SEQ ID NO: 39, and a CDR3 comprising the amino acid sequence set forth in SEQ ID NO: 109.

76. The antigen-recognizing receptor of claim 74, wherein the extracellular antigen-binding domain is a single-chain variable fragment (scFv), a Fab, which is optionally crosslinked, or a F(ab)₂.

77. The antigen-recognizing receptor of claim 74, wherein:
a) the heavy chain variable region comprises the amino acid sequence set forth in SEQ ID NO: 8, and the light chain variable region comprises the amino acid sequence set forth in SEQ ID NO: 9;
b) the heavy chain variable region comprises the amino acid sequence set forth in SEQ ID NO: 18, and the light chain variable region comprises the amino acid sequence set forth in SEQ ID NO: 19;
c) the heavy chain variable region comprises the amino acid sequence set forth in SEQ ID NO: 22, and the light chain variable region comprises the amino acid sequence set forth in SEQ ID NO: 23;
d) the heavy chain variable region comprises the amino acid sequence set forth in SEQ ID NO: 24, and the light chain variable region comprises the amino acid sequence set forth in SEQ ID NO: 23;
e) the heavy chain variable region comprises the amino acid sequence set forth in SEQ ID NO: 25, and the light chain variable region comprises the amino acid sequence set forth in SEQ ID NO: 23;
f) the heavy chain variable region comprises the amino acid sequence set forth in SEQ ID NO: 27, and the light chain variable region comprises the amino acid sequence set forth in SEQ ID NO: 23;
g) the heavy chain variable region comprises the amino acid sequence set forth in SEQ ID NO: 28, and the light chain variable region comprises the amino acid sequence set forth in SEQ ID NO: 23;
h) the heavy chain variable region comprises the amino acid sequence set forth in SEQ ID NO: 29, and the light chain variable region comprises the amino acid sequence set forth in SEQ ID NO: 23;
i) the heavy chain variable region comprises the amino acid sequence set forth in SEQ ID NO: 22, and the light chain variable region comprises the amino acid sequence set forth in SEQ ID NO: 30;
j) the heavy chain variable region comprises the amino acid sequence set forth in SEQ ID NO: 24, and the light chain variable region comprises the amino acid sequence set forth in SEQ ID NO: 30;
k) the heavy chain variable region comprises the amino acid sequence set forth in SEQ ID NO: 25, and the light chain variable region comprises the amino acid sequence set forth in SEQ ID NO: 30;
l) the heavy chain variable region comprises the amino acid sequence set forth in SEQ ID NO: 27, and the light chain variable region comprises the amino acid sequence set forth in SEQ ID NO: 30;
m) the heavy chain variable region comprises the amino acid sequence set forth in SEQ ID NO: 28, and the light chain variable region comprises the amino acid sequence set forth in SEQ ID NO: 30;
n) the heavy chain variable region comprises the amino acid sequence set forth in SEQ ID NO: 29, and the light chain variable region comprises the amino acid sequence set forth in SEQ ID NO: 30;
o) the heavy chain variable region comprises the amino acid sequence set forth in SEQ ID NO: 22, and the light chain variable region comprises the amino acid sequence set forth in SEQ ID NO: 31;
p) the heavy chain variable region comprises the amino acid sequence set forth in SEQ ID NO: 24, and the light chain variable region comprises the amino acid sequence set forth in SEQ ID NO: 31;
q) the heavy chain variable region comprises the amino acid sequence set forth in SEQ ID NO: 25, and the light chain variable region comprises the amino acid sequence set forth in SEQ ID NO: 31;
r) the heavy chain variable region comprises the amino acid sequence set forth in SEQ ID NO: 27, and the light chain variable region comprises the amino acid sequence set forth in SEQ ID NO: 31;
s) the heavy chain variable region comprises the amino acid sequence set forth in SEQ ID NO: 28, and the light chain variable region comprises the amino acid sequence set forth in SEQ ID NO: 31;
t) the heavy chain variable region comprises the amino acid sequence set forth in SEQ ID NO: 29, and the light chain variable region comprises the amino acid sequence set forth in SEQ ID NO: 31;
u) the heavy chain variable region comprises the amino acid sequence set forth in SEQ ID NO: 22, and the light chain variable region comprises the amino acid sequence set forth in SEQ ID NO: 32;
v) the heavy chain variable region comprises the amino acid sequence set forth in SEQ ID NO: 24, and the light chain variable region comprises the amino acid sequence set forth in SEQ ID NO: 32;
w) the heavy chain variable region comprises the amino acid sequence set forth in SEQ ID NO: 25, and the light chain variable region comprises the amino acid sequence set forth in SEQ ID NO: 32;
x) the heavy chain variable region comprises the amino acid sequence set forth in SEQ ID NO: 27, and the light chain variable region comprises the amino acid sequence set forth in SEQ ID NO: 32;
y) the heavy chain variable region comprises the amino acid sequence set forth in SEQ ID NO: 28, and the light chain variable region comprises the amino acid sequence set forth in SEQ ID NO: 32;
z) the heavy chain variable region comprises the amino acid sequence set forth in SEQ ID NO: 29, and the light chain variable region comprises the amino acid sequence set forth in SEQ ID NO: 32;
aa) the heavy chain variable region comprises the amino acid sequence set forth in SEQ ID NO: 22, and the light chain variable region comprises the amino acid sequence set forth in SEQ ID NO: 19;
ab) the heavy chain variable region comprises the amino acid sequence set forth in SEQ ID NO: 24, and the light chain variable region comprises the amino acid sequence set forth in SEQ ID NO: 19;
ac) the heavy chain variable region comprises the amino acid sequence set forth in SEQ ID NO: 25, and the light chain variable region comprises the amino acid sequence set forth in SEQ ID NO: 19;
ad) the heavy chain variable region comprises the amino acid sequence set forth in SEQ ID NO: 27, and the light chain variable region comprises the amino acid sequence set forth in SEQ ID NO: 19;
ae) the heavy chain variable region comprises the amino acid sequence set forth in SEQ ID NO: 28, and the light chain variable region comprises the amino acid sequence set forth in SEQ ID NO: 19;
af) the heavy chain variable region comprises the amino acid sequence set forth in SEQ ID NO: 29, and the light chain variable region comprises the amino acid sequence set forth in SEQ ID NO: 19;
ag) the heavy chain variable region comprises the amino acid sequence set forth in SEQ ID NO: 33, and the light chain variable region comprises the amino acid sequence set forth in SEQ ID NO: 34;
ah) the heavy chain variable region comprises the amino acid sequence set forth in SEQ ID NO: 41, and the light chain variable region comprises the amino acid sequence set forth in SEQ ID NO: 42;
ai) the heavy chain variable region comprises the amino acid sequence set forth in SEQ ID NO: 49, and the light chain variable region comprises the amino acid sequence set forth in SEQ ID NO: 50;
aj) the heavy chain variable region comprises the amino acid sequence set forth in SEQ ID NO: 49, and the light chain variable region comprises the amino acid sequence set forth in SEQ ID NO: 53;
ak) the heavy chain variable region comprises the amino acid sequence set forth in SEQ ID NO: 60, and the light chain variable region comprises the amino acid sequence set forth in SEQ ID NO: 61;
al) the heavy chain variable region comprises the amino acid sequence set forth in SEQ ID NO: 67, and the light chain variable region comprises the amino acid sequence set forth in SEQ ID NO: 68;
am) the heavy chain variable region comprises the amino acid sequence set forth in SEQ ID NO: 75, and the light chain variable region comprises the amino acid sequence set forth in SEQ ID NO: 76;
an) the heavy chain variable region comprises the amino acid sequence set forth in SEQ ID NO: 87, and the light chain variable region comprises the amino acid sequence set forth in SEQ ID NO: 88;
ao) the heavy chain variable region comprises the amino acid sequence set forth in SEQ ID NO: 99, and the light chain variable region comprises the amino acid sequence set forth in SEQ ID NO: 100;
ap) the heavy chain variable region comprises the amino acid sequence set forth in SEQ ID NO: 110, and the light chain variable region comprises the amino acid sequence set forth in SEQ ID NO: 111;
aq) the heavy chain variable region comprises the amino acid sequence set forth in SEQ ID NO: 122, and the light chain variable region comprises the amino acid sequence set forth in SEQ ID NO: 123;
ar) the heavy chain variable region comprises the amino acid sequence set forth in SEQ ID NO: 128, and the light chain variable region comprises the amino acid sequence set forth in SEQ ID NO: 129;
as) the heavy chain variable region comprises the amino acid sequence set forth in SEQ ID NO: 130, and the light chain variable region comprises the amino acid sequence set forth in SEQ ID NO: 131;
at) the heavy chain variable region comprises the amino acid sequence set forth in SEQ ID NO: 130, and the light chain variable region comprises the amino acid sequence set forth in SEQ ID NO: 132;
au) the heavy chain variable region comprises the amino acid sequence set forth in SEQ ID NO: 130, and the light chain variable region comprises the amino acid sequence set forth in SEQ ID NO: 133;
av) the heavy chain variable region comprises the amino acid sequence set forth in SEQ ID NO: 134, and the light chain variable region comprises the amino acid sequence set forth in SEQ ID NO: 131;
aw) the heavy chain variable region comprises the amino acid sequence set forth in SEQ ID NO: 134, and the light chain variable region comprises the amino acid sequence set forth in SEQ ID NO: 132;
ax) the heavy chain variable region comprises the amino acid sequence set forth in SEQ ID NO: 134, and the light chain variable region comprises the amino acid sequence set forth in SEQ ID NO: 133;
ay) the heavy chain variable region comprises the amino acid sequence set forth in SEQ ID NO: 135, and the light chain variable region comprises the amino acid sequence set forth in SEQ ID NO: 131;
az) the heavy chain variable region comprises the amino acid sequence set forth in SEQ ID NO: 135, and the light chain variable region comprises the amino acid sequence set forth in SEQ ID NO: 132;
ba) the heavy chain variable region comprises the amino acid sequence set forth in SEQ ID NO: 135, and the light chain variable region comprises the amino acid sequence set forth in SEQ ID NO: 133; or
bb) the heavy chain variable region comprises the amino acid sequence set forth in SEQ ID NO: 135, and the light chain variable region comprises the amino acid sequence set forth in SEQ ID NO: 136.

78. The antigen-recognizing receptor of claim 74, wherein
a) the heavy chain variable region comprises the amino acid sequence set forth in SEQ ID NO: 87, and the light chain variable region comprises the amino acid sequence set forth in SEQ ID NO: 88;
b) the heavy chain variable region comprises the amino acid sequence set forth in SEQ ID NO: 99, and the light chain variable region comprises the amino acid sequence set forth in SEQ ID NO: 100; or
c) the heavy chain variable region comprises the amino acid sequence set forth in SEQ ID NO: 110, and the light chain variable region comprises the amino acid sequence set forth in SEQ ID NO: 111.

79. The antigen-recognizing receptor of claim 74, wherein the extracellular antigen-binding domain comprises a linker between the heavy chain variable region and the light chain variable region of the extracellular antigen-binding domain.

80. The antigen-recognizing receptor of claim 74, wherein the linker consists of the amino acid sequence set forth in SEQ ID NO: 10, SEQ ID NO: 11, SEQ ID NO: 20, SEQ ID NO: 21, SEQ ID NO: 137, or SEQ ID NO: 138.

81. The antigen-recognizing receptor of claim 74, wherein (a) the heavy chain variable region and the light chain variable region are positioned from the N- to the C-terminus: V_{H}-V_{L}, or (b) the heavy chain variable region and the light chain variable region are positioned from the N- to the C-terminus: V_{L}-V_{H}.

82. An antigen-recognizing receptor, comprising an extracellular antigen-binding domain, a transmembrane domain, and an intracellular signaling domain, wherein the extracellular antigen-binding domain specifically binds to L1CAM and comprises:
(a) a heavy chain variable region and a light chain variable region, wherein the heavy chain variable region comprises a CDR1, a CDR2, and a CDR3 of a heavy chain variable region comprising the amino acid sequence set forth in SEQ ID NO: 87, and the light chain variable region comprises a CDR1, a CDR2, and a CDR3 of a light chain variable region comprising the amino acid sequence set forth in SEQ ID NO: 88;
(b) a heavy chain variable region and a light chain variable region, wherein the heavy chain variable region comprises a CDR1, a CDR2, and a CDR3 of a heavy chain variable region comprising the amino acid sequence set forth in SEQ ID NO: 99, and the light chain variable region comprises a CDR1, a CDR2, and a CDR3 of a light chain variable region comprising the amino acid sequence set forth in SEQ ID NO: 100; or
(c) a heavy chain variable region and a light chain variable region, wherein the heavy chain variable region comprises a CDR1, a CDR2, and a CDR3 of a heavy chain variable region comprising the amino acid sequence set forth in SEQ ID NO: 110, and the light chain variable region comprises a CDR1, a CDR2, and a CDR3 of a light chain variable region comprising the amino acid sequence set forth in SEQ ID NO: 111.

83. An antigen-recognizing receptor, comprising an extracellular antigen-binding domain, a transmembrane domain, and an intracellular signaling domain, wherein the extracellular antigen-binding domain specifically binds to L1CAM and comprises a single chain a single chain variable region (scFv) comprising the amino acid sequence set forth in SEQ ID NO: 77, SEQ ID NO: 78, SEQ ID NO: 89, SEQ ID NO: 90, SEQ ID NO: 101, or SEQ ID NO: 102.

84. The antigen-recognizing receptor of claim 74, wherein the transmembrane domain comprises a CD8 polypeptide, a CD28 polypeptide, a CD3ζ polypeptide, a CD4 polypeptide, a 4-1BB polypeptide, an OX40 polypeptide, an ICOS polypeptide, a CTLA-4 polypeptide, a PD-1 polypeptide, a LAG-3 polypeptide, a 2B4 polypeptide, a BTLA polypeptide, or a combination thereof.

85. The antigen-recognizing receptor of claim 74, wherein the intracellular signaling domain comprises a CD3ζ polypeptide.

86. The antigen-recognizing receptor of claim 74, wherein the intracellular signaling domain further comprises at least one co-stimulatory signaling region comprising a CD28 polypeptide, a 4-1BB polypeptide, an OX40 polypeptide, an ICOS polypeptide, a DAP-10 polypeptide, or a combination thereof.

87. The antigen-recognizing receptor of claim 74, wherein the antigen-recognizing receptor is a chimeric antigen receptor (CAR) or a T-cell receptor (TCR) like fusion protein.

88. The antigen-recognizing receptor of claim 87, wherein the antigen-recognizing receptor is a CAR.

89. A cell comprising the antigen-recognizing receptor of claim 74.

90. The cell of claim 89, wherein the cell is selected from the group consisting of a T cell, a Natural Killer (NK) cell, and a stem cell from which a lymphoid cell may be differentiated.

91. The cell of claim 90, wherein the cell is a T cell.

92. The cell of claim 91, wherein the T cell is a cytotoxic T lymphocyte (CTL) or a regulatory T cell.

93. A nucleic acid molecule encoding the antigen-recognizing receptor of claim 74.

94. A vector comprising the nucleic acid molecule of claim 93.

95. A host cell expressing the nucleic acid molecule of claim 93.

96. A composition comprising the cell of claim 89.

97. The composition of claim 96, which is a pharmaceutical composition further comprising a pharmaceutically acceptable carrier.

98. A lipid nanoparticle comprising the nucleic acid of claim 93.

99. A composition comprising the lipid nanoparticle of claim 98.

100. A method of treating and/or preventing a tumor in a subject in need thereof, increasing or lengthening survival of a subject having a tumor, reducing the risk of metastatic spread in a subject in need thereof, inhibiting metastatic spread of a primary cancer in a subject in need thereof, or inhibiting progression of metastatic disease in a subject in need thereof, the method comprising administering to the subject an effective amount of the cell of claim 89.

101. The method of claim 100, wherein the tumor is associated with L1CAM.

102. The method of claim 101, wherein the tumor is a cancer.

103. The method of claim 102, wherein the cancer is a metastatic cancer that localizes in at least one tissue selected from the group consisting of bone, liver, lung, brain, peritoneum, adrenal gland, skin, lymph nodes, pleura, and meninges.

104. A method for producing a cell comprising an antigen-recognizing receptor of claim 74, comprising introducing into the cell a nucleic acid molecule that encodes the antigen-recognizing receptor.

105. A kit for reducing tumor burden in a subject, treating and/or preventing a tumor in a subject, and/or increasing or lengthening survival of a subject having a tumor, comprising the cell of claim 89.


SEQUENCE LISTING (175 sequences)

SEQ ID NO: 1 - protein, 1257 aa, Homo sapiens
MVVALRYVWPLLLCSPCLLIQIPEEYEGHHVMEPPVITEQSPRRLVVFPTDDISLKCEASGKPEVQFRWTRDGVHFKPKEELGVTVYQSPHSGSFTITGNNSNFAQRFQGIYRCFASNKLGTAMSHEIRLMAEGAPKWPKETVKPVEVEEGESVVLPCNPPPSAEPLRIYWMNSKILHIKQDERVTMGQNGNLYFANVLTSDNHSDYICHAHFPGTRTIIQKEPIDLRVKATNSMIDRKPRLLFPTNSSSHLVALQGQPLVLECIAEGFPTPTIKWLRPSGPMPADRVTYQNHNKTLQLLKVGEEDDGEYRCLAENSLGSARHAYYVTVEAAPYWLHKPQSHLYGPGETARLDCQVQGRPQPEVTWRINGIPVEELAKDQKYRIQRGALILSNVQPSDTMVTQCEARNRHGLLLANAYIYVVQLPAKILTADNQTYMAVQGSTAYLLCKAFGAPVPSVQWLDEDGTTVLQDERFFPYANGTLGIRDLQANDTGRYFCLAANDQNNVTIMANLKVKDATQITQGPRSTIEKKGSRVTFTCQASFDPSLQPSITWRGDGRDLQELGDSDKYFIEDGRLVIHSLDYSDQGNYSCVASTELDVVESRAQLLVVGSPGPVPRLVLSDLHLLTQSQVRVSWSPAEDHNAPIEKYDIEFEDKEMAPEKWYSLGKVPGNQTSTTLKLSPYVHYTFRVTAINKYGPGEPSPVSETVVTPEAAPEKNPVDVKGEGNETTNMVITWKPLRWMDWNAPQVQYRVQWRPQGTRGPWQEQIVSDPFLVVSNTSTFVPYEIKVQAVNSQGKGPEPQVTIGYSGEDYPQAIPELEGIEILNSSAVLVKWRPVDLAQVKGHLRGYNVTYWREGSQRKHSKRHIHKDHVVVPANTTSVILSGLRPYSSYHLEVQAFNGRGSGPASEFTFSTPEGVPGHPEALHLECQSNTSLLLRWQPPLSHNGVLTGYVLSYHPLDEGGKGQLSFNLRDPELRTHNLTDLSPHLRYRFQLQATTKEGPGEAIVREGGTMALSGISDFGNISATAGENYSVVSWVPKEGQCNFRFHILFKALGEEKGGASLSPQYVSYNQSSYTQWDLQPDTDYEIHLFKERMFRHQMAVKTNGTGRVRLPPAGFATEGWFIGFVSAIILLLLVLLILCFIKRSKGGKYSVKDKEDTQVDSEARPMKDETFGEYRSLESDNEEKAFGSSQPSLNGDIKPLGSDDSLADYGGSVDVQFNEDGSFIGQYSGKKEKEAAGGNDSSGATSPINPAVALE

SEQ ID NO: 2 - protein, 8 aa, synthetic construct
GFIFSSYN

SEQ ID NO: 3 - protein, 8 aa, synthetic construct
ISSSSSTT

SEQ ID NO: 4 - protein, 17 aa, synthetic construct
ASLYSGYDYYYYYGMDV

SEQ ID NO: 5 - protein, 11 aa, synthetic construct
QSLVHSDGNTY

SEQ ID NO: 6 - skipped

SEQ ID NO: 7 - protein, 9 aa, synthetic construct
MQATQFPFT

SEQ ID NO: 8 - protein, 124 aa, synthetic construct
EVQMVESGGGLVQPGGSLRLSCAAPGFIFSSYNMNWVRQAPGKGLEWVSYISSSSSTTYYADSVKGRFTISRDNAKNSLYLQMNSLRDEDTAVYYCASLYSGYDYYYYYGMDVWGQGTTVTVSS

SEQ ID NO: 9 - protein, 112 aa, synthetic construct
DIVMTQTPLSSPVSLGQPASISCRSSQSLVHSDGNTYLSWLQQRPGQPPRVLIYKISKRFSGVPDRFSGSGAGTDFTLKISRVEAEDVGVYYCMQATQFPFTFGPGTKVDIK

SEQ ID NO: 10 - protein, 19 aa, synthetic construct
GGGGSGGGGSGGGSGGGGS

SEQ ID NO: 11 - protein, 15 aa, synthetic construct
GGGGSGGGGSGGGGS

SEQ ID NO: 12 - protein, 8 aa, synthetic construct
GYTFTNYY

SEQ ID NO: 13 - protein, 8 aa, synthetic construct
IGPGSGSP

SEQ ID NO: 14 - protein, 9 aa, synthetic construct
ARWLLPFDY

SEQ ID NO: 15 - protein, 5 aa, synthetic construct
SSVNY

SEQ ID NO: 16 - skipped

SEQ ID NO: 17 - protein, 9 aa, synthetic construct
QQFSSDPLT

SEQ ID NO: 18 - protein, 116 aa, synthetic construct
QVQLVQSGAEVKKPGSSVKVSCKASGYTFTNYYIHWVRQAPGQGLEWMGKIGPGSGSPYYQGMFKDRVTITADESTSTAYMELSSLRSEDTAVYYCARWLLPFDYWGQGTLVTVSS

SEQ ID NO: 19 - protein, 106 aa, synthetic construct
EIVLTQSPGTLSLSPGERATLSCRASSSVNYMHWYQQKPGQAPRLLIYATSILASGIPDRFSGSGSGTDYTLTISRLEPEDFAVYYCQQFSSDPLTFGGGTKVEIK

SEQ ID NO: 20 - protein, 24 aa, synthetic construct
GGGGSGGGGSGGGGSGGGSGGGGS

SEQ ID NO: 21 - protein, 29 aa, synthetic construct
GGGGSGGGGSGGGGSGGGGSGGGSGGGGS

SEQ ID NO: 22 - protein, 116 aa, synthetic construct
QVQLVQSGAEVKKPGASVKVSCKASGYTFTNYYIHWVRQAPGQGLEWMGKIGPGSGSPYYNGMFKDRVTMTRDTSTSTVYMELSSLRSEDTAVYYCARWLLPFDYWGQGTLVTVSS

SEQ ID NO: 23 - protein, 106 aa, synthetic construct
EIVLTQSPATLSLSPGERATLSCRASSSVNYMHWYQQKPGQAPRLLIYATSILASGIPARFSGSGSGTDYTLTISSLEPEDFAVYYCQQFSSDPLTFGGGTKVEIK

SEQ ID NO: 24 - protein, 116 aa, synthetic construct
QVQLVQSGAEVKKPGASVKVSCKASGYTFTNYYIHWVRQAPGQGLEWMGKIGPGSGSPYYNGMFKDRVTSTRDTSISTAYMELSRLRSDDTVVYYCARWLLPFDYWGQGTLVTVSS

SEQ ID NO: 25 - protein, 116 aa, synthetic construct
QVQLVESGGGVVQPGRSLRLSCAASGYTFTNYYIHWVRQAPGKGLEWVAKIGPGSGSPYYNGMFKDRFTISRDNSKNTLYLQMNSLRAEDTAVYYCARWLLPFDYWGQGTLVTVSS

SEQ ID NO: 26 - protein, 9 aa, synthetic construct
AKWLLPFDY

SEQ ID NO: 27 - protein, 116 aa, synthetic construct
EVQLLESGGGLVQPGGSLRLSCAASGYTFTNYYIHWVRQAPGKGLEWVSKIGPGSGSPYYNGMFKDRFTISVDNSKNTLYLQMNSLRAEDTAVYYCAKWLLPFDYWGQGTLVTVSS

SEQ ID NO: 28 - protein, 116 aa, synthetic construct
QVQLVQSGAEVKKPGSSVKVSCKASGYTFTNYYIHWVRQAPGQGLEWMGKIGPGSGSPYYNGMFKDRVTITADESTSTAYMELSSLRSEDTAVYYCARWLLPFDYWGQGTLVTVSS

SEQ ID NO: 29 - protein, 116 aa, synthetic construct
EVQLLESGGGLVQPGGSLRLSCAASGYTFTNYYIHWVRQAPGKGLEWVSKIGPGSGSPYYADSVKGRFTISVDNSKNTLYLQMNSLRAEDTAVYYCAKWLLPFDYWGQGTLVTVSS

SEQ ID NO: 30 - protein, 106 aa, synthetic construct
EIVMTQSPATLSVSPGERATLSCRASSSVNYMHWYQQKPGQAPRLLIYATSILASGIPARFSGSGSGTEYTLTISSLQSEDFAVYYCQQFSSDPLTFGGGTKVEIK

SEQ ID NO: 31 - protein, 106 aa, synthetic construct
DIQMTQSPSTLSASVGDRVTITCRASSSVNYMHWYQQKPGKAPKLLIYATSILASGVPSRFSGSGSGTEYTLTISSLQPDDFATYYCQQFSSDPLTFGGGTKVEIK

SEQ ID NO: 32 - protein, 106 aa, synthetic construct
DIQMTQSPSSVSASVGDRVTITCRASSSVNYMHWYQQKPGKAPKPLIYATSILASGVPSRFSGSGSGTDYTLTISSLQPEDFATYYCQQFSSDPLTFGGGTKVEIK

SEQ ID NO: 33 - protein, 116 aa, synthetic construct
QVQLRQSGAELVRPGTSVKMSCKASGYTFTNYYIHWVKQRPGQGLEWIGKIGPGSGSPYYNGMFKDKATLTVDTSSSTAYMQLSSLTSEDSAVYFCARWLLPFDYWGQGTTLTVSS

SEQ ID NO: 34 - protein, 106 aa, synthetic construct
QIVLSQSPGILSASPGEKVTMTCRASSSVNYMHWYQQKSGSSPKPWIFATSILASGVPPRFSGSGSGTSYSLTISSVEAEDTATYYCQQFSSDPLTFGAGTKLELK

SEQ ID NO: 35 - protein, 8 aa, synthetic construct
GGSINSYY

SEQ ID NO: 36 - protein, 7 aa, synthetic construct
FYTSGST

SEQ ID NO: 37 - protein, 16 aa, synthetic construct
AREWSGSFYGGDAFDI

SEQ ID NO: 38 - protein, 12 aa, synthetic construct
QSVLNSSNNKNY

SEQ ID NO: 39 - skipped

SEQ ID NO: 40 - protein, 9 aa, synthetic construct
QQYYSTPYT

SEQ ID NO: 41 - protein, 122 aa, synthetic construct
QVQLQESGPGLVKPSETLSLTCTVSGGSINSYYWSWIRQPAGKGLEWIGRFYTSGSTNHNPSLKSRVTMSVDTSKNQFSLKLTSVTAADTAVYYCAREWSGSFYGGDAFDIWGQGTMVTVSS

SEQ ID NO: 42 - protein, 113 aa, synthetic construct
DIVMTQSPDSLAVSLGERATINCKSSQSVLNSSNNKNYLTWYQQRPGQPPNLLIYWASTRESGVPDRFSGSGSGTDFTLTISSLQAEDVAVYYCQQYYSTPYTFGQGTKLEIE

SEQ ID NO: 43 - protein, 8 aa, synthetic construct
GYPFTGYY

SEQ ID NO: 44 - protein, 8 aa, synthetic construct
INPNSGDT

SEQ ID NO: 45 - protein, 13 aa, synthetic construct
AREMGTTKGYFDL

SEQ ID NO: 46 - protein, 6 aa, synthetic construct
QGISSY

SEQ ID NO: 47 - skipped

SEQ ID NO: 48 - protein, 9 aa, synthetic construct
QQLNSYPDT

SEQ ID NO: 49 - protein, 120 aa, synthetic construct
QVQLVQSGAEVKKPGASVRVSCKASGYPFTGYYMHWVRQAPGQGLEWMGWINPNSGDTDYVQKFQGRVTMTRDTSISTAYMELSRLRSDDTAVYYCAREMGTTKGYFDLWGRGTLVTVSS

SEQ ID NO: 50 - protein, 107 aa, synthetic construct
DIQLTQSPSFLSASVGDRVTITCRASQGISSYLAWFQQKPGKAPRLLIYAASTLQSGVPSRFSGSRSGTEFTLTISSLQPEDFATYYCQQLNSYPDTFGQGTKVDIK

SEQ ID NO: 51 - protein, 12 aa, synthetic construct
QTILYSPNNKNY

SEQ ID NO: 52 - skipped

SEQ ID NO: 53 - protein, 113 aa, synthetic construct
DIVMTQSPDSLAVSLGERATINCKSSQTILYSPNNKNYLAWYQQKPGQPPKLLIYLTSTRESGVPDRFSGSGSGTDFTLTISSLQAEDVAVYYCQQYYSTPYTFGQGTKLEIK

SEQ ID NO: 54 - protein, 8 aa, synthetic construct
GYTFIGYY

SEQ ID NO: 55 - protein, 8 aa, synthetic construct
INPNSGGT

SEQ ID NO: 56 - protein, 13 aa, synthetic construct
ARGGLERRDAFDI

SEQ ID NO: 57 - protein, 6 aa, synthetic construct
QSISNW

SEQ ID NO: 58 - skipped

SEQ ID NO: 59 - protein, 8 aa, synthetic construct
QQYDTYRT

SEQ ID NO: 60 - protein, 120 aa, synthetic construct
QVQLVQSGAEVKKPGASVKVSCKASGYTFIGYYLHWVRQAPGQGLEWMGWINPNSGGTNSAQKFQGRVTMTRDTSISTTYMDLSRLRSDDTAVYYCARGGLERRDAFDIWGQGTLVTVSS

SEQ ID NO: 61 - protein, 106 aa, synthetic construct
DIQMTQSPSTLSASVGDRVTITCRASQSISNWLAWYQQKPGKAPNLLIYKASSLESGVPSRFSGSGSGTEFTLTISSLQPDDFATYYCQQYDTYRTFGQGTKVEIK

SEQ ID NO: 62 - protein, 8 aa, synthetic construct
GFTFSHYW

SEQ ID NO: 63 - protein, 8 aa, synthetic construct
IMQDGSEK

SEQ ID NO: 64 - protein, 12 aa, synthetic construct
ARDRGVTTLFDY

SEQ ID NO: 65 - protein, 6 aa, synthetic construct
QSISSW

SEQ ID NO: 66 - protein, 8 aa, synthetic construct
QQYYSYGT

SEQ ID NO: 67 - protein, 119 aa, synthetic construct
EVQLVESGGGLVQPGGSLRLSCGVSGFTFSHYWMNWVRQAPGKGLEWVANIMQDGSEKYYVDSVKGRFTISRDNAKNSLYLQMNSLRVEDTAVYYCARDRGVTTLFDYWGQGILVTVSS

SEQ ID NO: 68 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYYSYGTFGQGTKVEIK

SEQ ID NO: 69 - protein, 8 aa, synthetic construct
GYIFISYW

SEQ ID NO: 70 - protein, 8 aa, synthetic construct
INPRNGRT

SEQ ID NO: 71 - protein, 12 aa, synthetic construct
ARGGDYGGSFDY

SEQ ID NO: 72 - protein, 10 aa, synthetic construct
ESVDSYGNSF

SEQ ID NO: 73 - skipped

SEQ ID NO: 74 - protein, 9 aa, synthetic construct
QQSNEDPWT

SEQ ID NO: 75 - protein, 119 aa, synthetic construct
QVQLQQPGAELVKPGASVKLSCKASGYIFISYWMHWVKQRPGQGLEWIGEINPRNGRTNYNEKFKSKATLTVDKSSSTAYMQLTSLTSEDSAVYYCARGGDYGGSFDYWGQGTTLTVSS

SEQ ID NO: 76 - protein, 111 aa, synthetic construct
DIVLTQSPASLAVSLGQRATISCRASESVDSYGNSFMHWYQQKPGQPPKLLIYRASNLESGIPARFSGSGSRTDFTLTVNPVEADDVATYYCQQSNEDPWTFGGGTKLEIK

SEQ ID NO: 77 - protein, 245 aa, synthetic construct
QVQLQQPGAELVKPGASVKLSCKASGYIFISYWMHWVKQRPGQGLEWIGEINPRNGRTNYNEKFKSKATLTVDKSSSTAYMQLTSLTSEDSAVYYCARGGDYGGSFDYWGQGTTLTVSSGGGGSGGGGSGGGGSDIVLTQSPASLAVSLGQRATISCRASESVDSYGNSFMHWYQQKPGQPPKLLIYRASNLESGIPARFSGSGSRTDFTLTVNPVEADDVATYYCQQSNEDPWTFGGGTKLEIK

SEQ ID NO: 78 - protein, 245 aa, synthetic construct
DIVLTQSPASLAVSLGQRATISCRASESVDSYGNSFMHWYQQKPGQPPKLLIYRASNLESGIPARFSGSGSRTDFTLTVNPVEADDVATYYCQQSNEDPWTFGGGTKLEIKGGGGSGGGGSGGGGSQVQLQQPGAELVKPGASVKLSCKASGYIFISYWMHWVKQRPGQGLEWIGEINPRNGRTNYNEKFKSKATLTVDKSSSTAYMQLTSLTSEDSAVYYCARGGDYGGSFDYWGQGTTLTVSS

SEQ ID NO: 79 - DNA, 735 bp, synthetic construct
  source 1..735: mol_type = other DNA
caggtacagctccagcaaccgggggcggagttggtaaagccgggtgcgtccgtaaaattgtcatgcaaggccagcggctatatcttcattagctactggatgcactgggttaagcaacggcccggccagggtcttgagtggatcggggaaattaatccgaggaacggccggacgaactacaacgaaaaattcaaatctaaagctactctcaccgtggataagtcttcttctacagcttatatgcagttgaccagtctcaccagtgaagacagtgcggtatattactgtgcaaggggtggagactatgggggctcattcgattattgggggcaaggtactacactgactgtatcttccggcggtggtggctcaggcggcggaggatccgggggggggggatccgacatagtactcactcagagcccagcttcactggctgtgtctttgggacaacgcgcaactatatcctgccgggcgtcagaatctgtagatagctatggaaacagttttatgcattggtatcagcaaaaacccggtcagccccctaagttgctcatttatagagcctcaaacctcgaatcagggattccggcacgcttcagtgggtctggtagtagaacggatttcacacttacagtcaatccagtcgaggccgacgatgtggctacatactactgccagcaatccaatgaagatccttggacatttggtggcgggacaaagctggaaataaag

SEQ ID NO: 80 - DNA, 735 bp, synthetic construct
  source 1..735: mol_type = other DNA
gacatagtactcactcagagcccagcttcactggctgtgtctttgggacaacgcgcaactatatcctgccgggcgtcagaatctgtagatagctatggaaacagttttatgcattggtatcagcaaaaacccggtcagccccctaagttgctcatttatagagcctcaaacctcgaatcagggattccggcacgcttcagtgggtctggtagtagaacggatttcacacttacagtcaatccagtcgaggccgacgatgtggctacatactactgccagcaatccaatgaagatccttggacatttggtggcgggacaaagctggaaataaagggcggtggtggctcaggcggcggaggatccgggggggggggatcccaggtacagctccagcaaccgggggcggagttggtaaagccgggtgcgtccgtaaaattgtcatgcaaggccagcggctatatcttcattagctactggatgcactgggttaagcaacggcccggccagggtcttgagtggatcggggaaattaatccgaggaacggccggacgaactacaacgaaaaattcaaatctaaagctactctcaccgtggataagtcttcttctacagcttatatgcagttgaccagtctcaccagtgaagacagtgcggtatattactgtgcaaggggtggagactatgggggctcattcgattattgggggcaaggtactacactgactgtatcttcc

SEQ ID NO: 81 - protein, 8 aa, synthetic construct
GFIFSSYN

SEQ ID NO: 82 - protein, 8 aa, synthetic construct
ISSSSSTT

SEQ ID NO: 83 - protein, 17 aa, synthetic construct
ASLYSGYDYYYYYGMDV

SEQ ID NO: 84 - protein, 11 aa, synthetic construct
QSLVHSDGNTY

SEQ ID NO: 85 - skipped

SEQ ID NO: 86 - protein, 9 aa, synthetic construct
MQATQFPFT

SEQ ID NO: 87 - protein, 124 aa, synthetic construct; identical to SEQ ID NO: 8

SEQ ID NO: 88 - protein, 112 aa, synthetic construct; identical to SEQ ID NO: 9

SEQ ID NO: 89 - protein, 251 aa, synthetic construct
EVQMVESGGGLVQPGGSLRLSCAAPGFIFSSYNMNWVRQAPGKGLEWVSYISSSSSTTYYADSVKGRFTISRDNAKNSLYLQMNSLRDEDTAVYYCASLYSGYDYYYYYGMDVWGQGTTVTVSSGGGGSGGGGSGGGGSDIVMTQTPLSSPVSLGQPASISCRSSQSLVHSDGNTYLSWLQQRPGQPPRVLIYKISKRFSGVPDRFSGSGAGTDFTLKISRVEAEDVGVYYCMQATQFPFTFGPGTKVDIK

SEQ ID NO: 90 - protein, 252 aa, synthetic construct
DIVMTQTPLSSPVSLGQPASISCRSSQSLVHSDGNTYLSWLQQRPGQPPRVLIYKISKRFSGVPDRFSGSGAGTDFTLKISRVEAEDVGVYYCMQATQFPFTFGPGTKVDIKGGGGSGGGGSGGGGSEEVQMVESGGGLVQPGGSLRLSCAAPGFIFSSYNMNWVRQAPGKGLEWVSYISSSSSTTYYADSVKGRFTISRDNAKNSLYLQMNSLRDEDTAVYYCASLYSGYDYYYYYGMDVWGQGTTVTVSS

SEQ ID NO: 91 - DNA, 753 bp, synthetic construct
  source 1..753: mol_type = other DNA
gaagtccagatggtggagtcaggagggggtctggtccaaccggggggtagcctgaggttgtcctgcgcggcaccaggatttatcttctcaagctataacatgaactgggtacgccaagcacctggcaaaggactggaatgggtaagttacatttcttccagttcatcaacaacttactatgcagacagtgtgaaaggccggtttactataagccgggacaatgctaagaactcactctatctccaaatgaactccctccgcgacgaggacacagctgtctattactgtgctagtctctactctggctacgattactattactattatggaatggatgtgtgggggcaaggcactactgtcacagtctcaagcggcggtggtggctcaggcggcggaggatccgggggggggggatccgatattgttatgacgcaaacgcccttgtcatcacctgtatcattgggtcagccggcttccatatcctgccgatctagtcaatccttggtgcactcagacgggaatacctacctttcatggctccagcagagacccgggcaaccaccgagagtgttgatctataaaatttctaaacggtttagcggggtccctgacagattctccggttccggggctggaactgattttacacttaaaatatcccgagtcgaagcggaagacgtgggggtgtattattgcatgcaagcaactcaatttcccttcacatttgggcctggtacaaaagtggacatcaag

SEQ ID NO: 92 - DNA, 753 bp, synthetic construct
  source 1..753: mol_type = other DNA
gatattgttatgacgcaaacgcccttgtcatcacctgtatcattgggtcagccggcttccatatcctgccgatctagtcaatccttggtgcactcagacgggaatacctacctttcatggctccagcagagacccgggcaaccaccgagagtgttgatctataaaatttctaaacggtttagcggggtccctgacagattctccggttccggggctggaactgattttacacttaaaatatcccgagtcgaagcggaagacgtgggggtgtattattgcatgcaagcaactcaatttcccttcacatttgggcctggtacaaaagtggacatcaagggcggtggtggctcaggcggcggaggatccgggggggggggatccgaagtccagatggtggagtcaggagggggtctggtccaaccggggggtagcctgaggttgtcctgcgcggcaccaggatttatcttctcaagctataacatgaactgggtacgccaagcacctggcaaaggactggaatgggtaagttacatttcttccagttcatcaacaacttactatgcagacagtgtgaaaggccggtttactataagccgggacaatgctaagaactcactctatctccaaatgaactccctccgcgacgaggacacagctgtctattactgtgctagtctctactctggctacgattactattactattatggaatggatgtgtgggggcaaggcactactgtcacagtctcaagc

SEQ ID NO: 93 - protein, 8 aa, synthetic construct
GFSLTSYG

SEQ ID NO: 94 - protein, 7 aa, synthetic construct
IWGDGST

SEQ ID NO: 95 - protein, 11 aa, synthetic construct
ATSTVGYAVDY

SEQ ID NO: 96 - protein, 6 aa, synthetic construct
ENIYSN

SEQ ID NO: 97 - skipped

SEQ ID NO: 98 - protein, 9 aa, synthetic construct
QHFWGSPYT

SEQ ID NO: 99 - protein, 117 aa, synthetic construct
QVQLKESGPGLVAPSQSLSITCTISGFSLTSYGVHWVRQPPGKGLEWLVVIWGDGSTSYNSALKSRLSISKDNSKSQVFLKMNSLQTDDTAMYFCATSTVGYAVDYWGQGTSVTVSS

SEQ ID NO: 100 - protein, 107 aa, synthetic construct
DIQMTQSPASLSVSVGETVTITCRASENIYSNLAWYQQRQGKSPQLLVYAATNLADGVPSRFSGSGSGTQYSLKINSLQSEDFGSYYCQHFWGSPYTFGGGTKLEIK

SEQ ID NO: 101 - protein, 239 aa, synthetic construct
QVQLKESGPGLVAPSQSLSITCTISGFSLTSYGVHWVRQPPGKGLEWLVVIWGDGSTSYNSALKSRLSISKDNSKSQVFLKMNSLQTDDTAMYFCATSTVGYAVDYWGQGTSVTVSSGGGGSGGGGSGGGGSDIQMTQSPASLSVSVGETVTITCRASENIYSNLAWYQQRQGKSPQLLVYAATNLADGVPSRFSGSGSGTQYSLKINSLQSEDFGSYYCQHFWGSPYTFGGGTKLEIK

SEQ ID NO: 102 - protein, 239 aa, synthetic construct
DIQMTQSPASLSVSVGETVTITCRASENIYSNLAWYQQRQGKSPQLLVYAATNLADGVPSRFSGSGSGTQYSLKINSLQSEDFGSYYCQHFWGSPYTFGGGTKLEIKGGGGSGGGGSGGGGSQVQLKESGPGLVAPSQSLSITCTISGFSLTSYGVHWVRQPPGKGLEWLVVIWGDGSTSYNSALKSRLSISKDNSKSQVFLKMNSLQTDDTAMYFCATSTVGYAVDYWGQGTSVTVSS

SEQ ID NO: 103 - DNA, 717 bp, synthetic construct
  source 1..717: mol_type = other DNA
caagtccagctcaaggagagcggtccaggactcgtagcaccctctcaatctttgtctatcacttgtaccataagcggtttttccttgacaagttatggcgtacattgggtgagacaaccgccaggtaaaggactcgaatggttggttgttatctggggggatgggagtaccagctacaacagcgcactcaagtctcgcctttcaatttctaaggacaacagtaaatcacaggtttttctgaagatgaatagcctgcaaaccgacgacacggcgatgtatttctgtgcgacttctacggtaggttatgccgtcgactattggggtcagggtacgtcagttaccgtaagtagcggcggtggtggctcaggcggcggaggatccgggggggggggatccgacatccagatgacccagtcaccggccagtttgtccgtttccgtaggcgagaccgtaaccatcacatgccgagcaagcgagaatatctattccaacctggcctggtatcagcagagacaaggcaaaagtccgcaacttttggtttatgctgccactaatttggccgatggggtcccgagtcgattttctggctctggctccgggacgcagtattccctgaagataaactctttgcagtcagaagattttggttcatattattgtcagcatttctggggcagcccgtacacatttggaggtgggaccaaattggaaataaaa

SEQ ID NO: 104 - DNA, 717 bp, synthetic construct
  source 1..717: mol_type = other DNA
gacatccagatgacccagtcaccggccagtttgtccgtttccgtaggcgagaccgtaaccatcacatgccgagcaagcgagaatatctattccaacctggcctggtatcagcagagacaaggcaaaagtccgcaacttttggtttatgctgccactaatttggccgatggggtcccgagtcgattttctggctctggctccgggacgcagtattccctgaagataaactctttgcagtcagaagattttggttcatattattgtcagcatttctggggcagcccgtacacatttggaggtgggaccaaattggaaataaaaggcggtggtggctcaggcggcggaggatccgggggggggggatcccaagtccagctcaaggagagcggtccaggactcgtagcaccctctcaatctttgtctatcacttgtaccataagcggtttttccttgacaagttatggcgtacattgggtgagacaaccgccaggtaaaggactcgaatggttggttgttatctggggggatgggagtaccagctacaacagcgcactcaagtctcgcctttcaatttctaaggacaacagtaaatcacaggtttttctgaagatgaatagcctgcaaaccgacgacacggcgatgtatttctgtgcgacttctacggtaggttatgccgtcgactattggggtcagggtacgtcagttaccgtaagtagc

SEQ ID NO: 105 - protein, 8 aa, synthetic construct
GDSISSYY

SEQ ID NO: 106 - protein, 7 aa, synthetic construct
IYTSGTT

SEQ ID NO: 107 - protein, 13 aa, synthetic construct
ASHSSGWYDAFDI

SEQ ID NO: 108 - protein, 12 aa, synthetic construct
QSVLYSSNNKNY

SEQ ID NO: 109 - protein, 9 aa, synthetic construct
QQYYSTPPT

SEQ ID NO: 110 - protein, 119 aa, synthetic construct
QVQLQESGPGLVKPSETLSLTCTVSGDSISSYYWTWIRQPAGKGLEWIGRIYTSGTTNYNPSLKSRVTMSVDTSKNQFSLKLSSVTAADTAVYYCASHSSGWYDAFDIWGQGTMVTVSS

SEQ ID NO: 111 - protein, 113 aa, synthetic construct
DIVMTQSPDSLAVSLGERATINCKSSQSVLYSSNNKNYLAWYQQKPGQPPKLFIYWASTRESGVPDRFSGSGSGTDFTLTISSLQAEDVAVYYCQQYYSTPPTFGQGTKVEIK

SEQ ID NO: 112 - protein, 247 aa, synthetic construct
QVQLQESGPGLVKPSETLSLTCTVSGDSISSYYWTWIRQPAGKGLEWIGRIYTSGTTNYNPSLKSRVTMSVDTSKNQFSLKLSSVTAADTAVYYCASHSSGWYDAFDIWGQGTMVTVSSGGGGSGGGGSGGGGSDIVMTQSPDSLAVSLGERATINCKSSQSVLYSSNNKNYLAWYQQKPGQPPKLFIYWASTRESGVPDRFSGSGSGTDFTLTISSLQAEDVAVYYCQQYYSTPPTFGQGTKVEIK

SEQ ID NO: 113 - protein, 247 aa, synthetic construct
DIVMTQSPDSLAVSLGERATINCKSSQSVLYSSNNKNYLAWYQQKPGQPPKLFIYWASTRESGVPDRFSGSGSGTDFTLTISSLQAEDVAVYYCQQYYSTPPTFGQGTKVEIKGGGGSGGGGSGGGGSQVQLQESGPGLVKPSETLSLTCTVSGDSISSYYWTWIRQPAGKGLEWIGRIYTSGTTNYNPSLKSRVTMSVDTSKNQFSLKLSSVTAADTAVYYCASHSSGWYDAFDIWGQGTMVTVSS

SEQ ID NO: 114 - DNA, 741 bp, synthetic construct
  source 1..741: mol_type = other DNA
caggtacagcttcaagaaagcggtccaggacttgtgaagccgagcgagacgctttcactgacttgcacagtcagtggggacagtattagttcctattactggacatggatacgacaaccagcagggaaaggtctggaatggattgggcgcatttacaccagtggaactacaaattacaacccatccctcaaatccagggttacgatgagcgtcgacacgagtaagaatcagttttccctgaagctctccagtgtgacggctgcagatactgcagtgtattattgcgcttcccactcaagtggttggtacgatgctttcgacatctgggggcagggtactatggtgaccgtgtcctcaggcggtggtggctcaggcggcggaggatccgggggggggggatccgacatcgtaatgacccaatcacctgattctctggctgtgagccttggtgagagggcaacaataaactgcaagagctctcagtccgtactttatagtagcaacaacaagaactatttggcgtggtaccagcagaagccgggtcagccgccaaagctgtttatatattgggcctctacccgcgaatctggagtaccggacaggtttagtggtagtggctcaggtacggattttacgctcaccattagctctctgcaagcagaggacgtagccgtatattattgccagcaatattattctacgccacccacctttggtcaggggacgaaagttgagatcaag

SEQ ID NO: 115 - DNA, 741 bp, synthetic construct
  source 1..741: mol_type = other DNA
gacatcgtaatgacccaatcacctgattctctggctgtgagccttggtgagagggcaacaataaactgcaagagctctcagtccgtactttatagtagcaacaacaagaactatttggcgtggtaccagcagaagccgggtcagccgccaaagctgtttatatattgggcctctacccgcgaatctggagtaccggacaggtttagtggtagtggctcaggtacggattttacgctcaccattagctctctgcaagcagaggacgtagccgtatattattgccagcaatattattctacgccacccacctttggtcaggggacgaaagttgagatcaagggcggtggtggctcaggcggcggaggatccgggggggggggatcccaggtacagcttcaagaaagcggtccaggacttgtgaagccgagcgagacgctttcactgacttgcacagtcagtggggacagtattagttcctattactggacatggatacgacaaccagcagggaaaggtctggaatggattgggcgcatttacaccagtggaactacaaattacaacccatccctcaaatccagggttacgatgagcgtcgacacgagtaagaatcagttttccctgaagctctccagtgtgacggctgcagatactgcagtgtattattgcgcttcccactcaagtggttggtacgatgctttcgacatctgggggcagggtactatggtgaccgtgtcctca

SEQ ID NO: 116 - protein, 8 aa, synthetic construct
GYTFTSYW

SEQ ID NO: 117 - protein, 8 aa, synthetic construct
VYPGSGST

SEQ ID NO: 118 - protein, 11 aa, synthetic construct
TRPGNYEGFAY

SEQ ID NO: 119 - protein, 10 aa, synthetic construct
ESVDSYGSSF

SEQ ID NO: 120 - skipped

SEQ ID NO: 121 - protein, 9 aa, synthetic construct
QQNNEDPWT

SEQ ID NO: 122 - protein, 118 aa, synthetic construct
QVQLQQPGSDLVRPGTSVKLSCKASGYTFTSYWMHWVKQRPGQGLEWIGNVYPGSGSTKYDEKFKTKATLTVDTSSNTAYMELSSLTSEDSAVYYCTRPGNYEGFAYWGQGTLVTVSA

SEQ ID NO: 123 - protein, 111 aa, synthetic construct
NIVLTQSPASLAVSLGQRATISCRASESVDSYGSSFMHWYQQKPGQPPKLLIYLASNLESGVPARFSGSGSRTDFTLTIDPVEADDAATYYCQQNNEDPWTFGGGTKLEIK

SEQ ID NO: 124 - protein, 244 aa, synthetic construct
QVQLQQPGSDLVRPGTSVKLSCKASGYTFTSYWMHWVKQRPGQGLEWIGNVYPGSGSTKYDEKFKTKATLTVDTSSNTAYMELSSLTSEDSAVYYCTRPGNYEGFAYWGQGTLVTVSAGGGGSGGGGSGGGGSNIVLTQSPASLAVSLGQRATISCRASESVDSYGSSFMHWYQQKPGQPPKLLIYLASNLESGVPARFSGSGSRTDFTLTIDPVEADDAATYYCQQNNEDPWTFGGGTKLEIK

SEQ ID NO: 125 - protein, 244 aa, synthetic construct
NIVLTQSPASLAVSLGQRATISCRASESVDSYGSSFMHWYQQKPGQPPKLLIYLASNLESGVPARFSGSGSRTDFTLTIDPVEADDAATYYCQQNNEDPWTFGGGTKLEIKGGGGSGGGGSGGGGSQVQLQQPGSDLVRPGTSVKLSCKASGYTFTSYWMHWVKQRPGQGLEWIGNVYPGSGSTKYDEKFKTKATLTVDTSSNTAYMELSSLTSEDSAVYYCTRPGNYEGFAYWGQGTLVTVSA

SEQ ID NO: 126 - DNA, 732 bp, synthetic construct
  source 1..732: mol_type = other DNA
caggtgcaacttcagcagcccggctctgatcttgtgcgccctggtacttctgtaaagcttagctgtaaagcttctggttatacgttcacgagttattggatgcactgggtaaagcagcggcctggacagggattggagtggatcggaaacgtgtaccctggcagtggttctacaaagtatgacgagaaatttaagacaaaagcaacactcactgtcgatacgtcttcaaacacagcgtacatggagttgagttcattgacctccgaggacagcgctgtatattactgcacacgccctggaaactacgaaggtttcgcgtattgggggcaggggaccttggtcacagtgtcagccggcggtggtggctcaggcggcggaggatccgggggggggggatccaacatagtccttacgcaaagtccagcttcactggcagtatctctgggtcaacgcgctaccattagttgtcgcgcaagcgaatccgtcgatagctatgggtcttcctttatgcactggtatcagcagaagccgggacagccgcccaagctgctgatctacctcgcgtccaacttggagtcaggcgttcccgcacggtttagtggaagcggaagtagaactgattttactcttacgattgatcctgtcgaggctgatgatgctgctacatattattgtcagcagaataatgaggatccttggaccttcggcgggggaaccaaattggaaattaaa

SEQ ID NO: 127 - DNA, 732 bp, synthetic construct
  source 1..732: mol_type = other DNA
aacatagtccttacgcaaagtccagcttcactggcagtatctctgggtcaacgcgctaccattagttgtcgcgcaagcgaatccgtcgatagctatgggtcttcctttatgcactggtatcagcagaagccgggacagccgcccaagctgctgatctacctcgcgtccaacttggagtcaggcgttcccgcacggtttagtggaagcggaagtagaactgattttactcttacgattgatcctgtcgaggctgatgatgctgctacatattattgtcagcagaataatgaggatccttggaccttcggcgggggaaccaaattggaaattaaaggcggtggtggctcaggcggcggaggatccgggggggggggatcccaggtgcaacttcagcagcccggctctgatcttgtgcgccctggtacttctgtaaagcttagctgtaaagcttctggttatacgttcacgagttattggatgcactgggtaaagcagcggcctggacagggattggagtggatcggaaacgtgtaccctggcagtggttctacaaagtatgacgagaaatttaagacaaaagcaacactcactgtcgatacgtcttcaaacacagcgtacatggagttgagttcattgacctccgaggacagcgctgtatattactgcacacgccctggaaactacgaaggtttcgcgtattgggggcaggggaccttggtcacagtgtcagcc

SEQ ID NO: 128 - protein, 118 aa, synthetic construct
QVQLVQSGAEVKKPGASVKVSCKASGYTFTSYWMHWVRQAPGQGLEWMGNVYPGSGSTKYAQKFQGRVTMTRDTSTSTVYMELSSLRSEDTAVYYCARPGNYEGFAYWGQGTLVTVSS

SEQ ID NO: 129 - protein, 111 aa, synthetic construct
NIQLTQSPSSLSASVGDRVTITCRASESVDSYGSSFMHWYQQKPGKPPKLLIYLASNLESGVPSRFSGSGSGTDFTLTISSLQPEDFATYYCQQNNEDPWTFGQGTKLEIK

SEQ ID NO: 130 - protein, 118 aa, synthetic construct
QVQLVQSGAEVKKPGASVKVSCKASGYTFTSYWMHWVRQAPGQGLEWIGNVYPGSGSTKYAQKFQGRATLTVDTSTSTVYMELSSLRSEDTAVYYCARPGNYEGFAYWGQGTLVTVSS

SEQ ID NO: 131 - protein, 111 aa, synthetic construct
DIQLTQSPSSLSASVGDRVTITCRASESVDSYGSSFMHWYQQKPGKPPKLLIYLASNLESGVPSRFSGSGSGTDFTLTISSLQPEDFATYYCQQNNEDPWTFGQGTKLEIK

SEQ ID NO: 132 - protein, 111 aa, synthetic construct
DIQLTQSPSSLSASVGDRVTITCRASESVDSYGSSFMHWYQQKPGKPPKLLIYLASNLESGVPSRFSGSGSRTDFTLTISSLQPEDFATYYCQQNNEDPWTFGQGTKLEIK

SEQ ID NO: 133 - protein, 111 aa, synthetic construct; identical to SEQ ID NO: 129

SEQ ID NO: 134 - protein, 118 aa, synthetic construct
QVQLVQSGAEVKKPGASVKVSCKASGYTFTSYWMHWVRQAPGQGLEWIGNVYPGSGSTKYAQKFQGRATLTVDTSTSTAYMELSSLRSEDTAVYYCTRPGNYEGFAYWGQGTLVTVSS

SEQ ID NO: 135 - protein, 118 aa, synthetic construct
QVQLVQSGAEVKKPGASVKVSCKASGYTFTSYWMHWVRQAPGQGLEWIGNVYPGSGSTKYAQKFQGRATLTVDTSTNTAYMELSSLRSEDTAVYYCTRPGNYEGFAYWGQGTLVTVSS

SEQ ID NO: 136 - protein, 111 aa, synthetic construct
DIQMTQSPSSLSASVGDRVTITCRASESVDSYGSSFMHWYQQKPGKAPKLLIYLASNLESGVPSRFSGSGSGTDFTLTISSLQPEDFATYYCQQNNEDPWTFGQGTKLEIK

SEQ ID NO: 137 - protein, 5 aa, synthetic construct
GGGGS

SEQ ID NO: 138 - protein, 10 aa, synthetic construct
GGGGSGGGGS

SEQ ID NO: 139 - protein, 21 aa, synthetic construct
MALPVTALLLPLALLLHAARP

SEQ ID NO: 140 - DNA, 63 bp, synthetic construct
  source 1..63: mol_type = other DNA
atggcgttgccggtgaccgcgcttttgctccctctggccctgctcttgcacgccgcacgaccg

SEQ ID NO: 141 - protein, 220 aa, synthetic construct
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 142 - DNA, 81 bp, synthetic construct
  source 1..81: mol_type = other DNA
ttctgggtactggtggtggtgggcggagttctcgcatgttattcattgctggtaacggtcgcattcattatattctgggtg

SEQ ID NO: 143 - protein, 218 aa, Mus musculus
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 144 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 145 - protein, 247 aa, Mus musculus
MASPLTRFLSLNLLLMGESIILGSGEAKPQAPELRIFPKKMDAELGQKVDLVCEVLGSVSQGCSWLFQNSSSKLPQPTFVVYMASSHNKITWDEKLNSSKLFSAVRDTNNKYVLTLNKFSKENEGYYFCSVISNSVMYFSSVVPVLQKVNSTTTKPVLRTPSPVHPTGTSQPQRPEDCRPRGSVKGTGLDFACDIYIWAPLAGICVAPLLSLIITLICYHRSRKRVCKCPRPLVRQEGKPRPSEKIV

SEQ ID NO: 146 - DNA, 117 bp, synthetic construct
  source 1..117: mol_type = other DNA
attgaagtcatgtatcctcctccgtacttggataatgaaaaaagtaatgggaccataattcacgtcaaaggaaaacatttgtgtccaagcccccttttccctggcccgtccaagcct

SEQ ID NO: 147 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 148 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 149 - DNA, 336 bp, synthetic construct
  source 1..336: mol_type = other DNA
cgagtaaaattttcccgaagtgcagatgcgccagcttatcagcaaggccagaatcagctctacaatgagttgaacctcggacggcgcgaggaatatgatgtgctcgataagcgcagaggtagggaccctgagatggggggtaaaccaaggcgaaagaatccccaagaggggctttacaacgaattgcaaaaagacaaaatggcagaagcctattccgaaattggaatgaagggcgaacggaggcgaggtaagggccacgacgggctctaccaaggcctgagcacagcaacaaaagacacgtatgacgcattgcacatgcaagctcttccgccccgg

SEQ ID NO: 150 - protein, 22 aa, synthetic construct
QNQLYNELNLGRREEYDVLDKR

SEQ ID NO: 151 - DNA, 66 bp, synthetic construct
  source 1..66: mol_type = other DNA
cagaaccagctctataacgagctcaatctaggacgaagagaggagtacgatgttttggacaagaga

SEQ ID NO: 152 - protein, 22 aa, synthetic construct
QNQLFNELNLGRREEFDVLDKR

SEQ ID NO: 153 - DNA, 66 bp, synthetic construct
  source 1..66: mol_type = other DNA
cagaaccagctctttaacgagctcaatctaggacgaagagaggagttcgatgttttggacaagaga

SEQ ID NO: 154 - protein, 23 aa, synthetic construct
QEGLYNELQKDKMAEAYSEIGMK

SEQ ID NO: 155 - DNA, 69 bp, synthetic construct
  source 1..69: mol_type = other DNA
caggaaggcctgtacaatgaactgcagaaagataagatggcggaggcctacagtgagattgggatgaaa

SEQ ID NO: 156 - protein, 23 aa, synthetic construct
QEGLFNELQKDKMAEAFSEIGMK

SEQ ID NO: 157 - DNA, 69 bp, synthetic construct
  source 1..69: mol_type = other DNA
caggaaggcctgttcaatgaactgcagaaagataagatggcggaggccttcagtgagattgggatgaaa

SEQ ID NO: 158 - protein, 22 aa, synthetic construct
HDGLYQGLSTATKDTYDALHMQ

SEQ ID NO: 159 - DNA, 66 bp, synthetic construct
  source 1..66: mol_type = other DNA
cacgatggcctttaccagggtctcagtacagccaccaaggacacctacgacgcccttcacatgcag

SEQ ID NO: 160 - protein, 22 aa, synthetic construct
HDGLFQGLSTATKDTFDALHMQ

SEQ ID NO: 161 - DNA, 66 bp, synthetic construct
  source 1..66: mol_type = other DNA
cacgatggccttttccaggggctcagtacagccaccaaggacaccttcgacgcccttcacatgcag

SEQ ID NO: 162 - protein, 112 aa, synthetic construct
RVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 163 - DNA, 336 bp, synthetic construct
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccaggggctcagtacagccaccaaggacaccttcgacgcccttcacatgcaggccctgccccctcgc

SEQ ID NO: 164 - DNA, 123 bp, synthetic construct
  source 1..123: mol_type = other DNA
cgatcaaaacggagtaggcttctccatagtgactatatgaacatgacgccgaggcgcccaggcccaacgcgcaaacattatcaaccatacgctccgccacgagatttcgcagcctatagatcc

SEQ ID NO: 165 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 166 - protein, 494 aa, synthetic construct
MALPVTALLLPLALLLHAARPDIVMTQTPLSSPVSLGQPASISCRSSQSLVHSDGNTYLSWLQQRPGQPPRVLIYKISKRFSGVPDRFSGSGAGTDFTLKISRVEAEDVGVYYCMQATQFPFTFGPGTKVDIKGGGGSGGGGSGGGGSEVQMVESGGGLVQPGGSLRLSCAAPGFIFSSYNMNWVRQAPGKGLEWVSYISSSSSTTYYADSVKGRFTISRDNAKNSLYLQMNSLRDEDTAVYYCASLYSGYDYYYYYGMDVWGQGTTVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 167 - DNA, 1482 bp, synthetic construct
  source 1..1482: mol_type = other DNA
atggcgttgccggtgaccgcgcttttgctccctctggccctgctcttgcacgccgcacgaccggatattgttatgacgcaaacgcccttgtcatcacctgtatcattgggtcagccggcttccatatcctgccgatctagtcaatccttggtgcactcagacgggaatacctacctttcatggctccagcagagacccgggcaaccaccgagagtgttgatctataaaatttctaaacggtttagcggggtccctgacagattctccggttccggggctggaactgattttacacttaaaatatcccgagtcgaagcggaagacgtgggggtgtattattgcatgcaagcaactcaatttcccttcacatttgggcctggtacaaaagtggacatcaagggcggtggtggctcaggcggcggaggatccgggggggggggatccgaagtccagatggtggagtcaggagggggtctggtccaaccggggggtagcctgaggttgtcctgcgcggcaccaggatttatcttctcaagctataacatgaactgggtacgccaagcacctggcaaaggactggaatgggtaagttacatttcttccagttcatcaacaacttactatgcagacagtgtgaaaggccggtttactataagccgggacaatgctaagaactcactctatctccaaatgaactccctccgcgacgaggacacagctgtctattactgtgctagtctctactctggctacgattactattactattatggaatggatgtgtgggggcaaggcactactgtcacagtctcaagcgcggccgcaattgaagtcatgtatcctcctccgtacttggataatgaaaaaagtaatgggaccataattcacgtcaaaggaaaacatttgtgtccaagcccccttttccctggcccgtccaagcctttctgggtactggtggtggtgggcggagttctcgcatgttattcattgctggtaacggtcgcattcattatattctgggtgcgatcaaaacggagtaggcttctccatagtgactatatgaacatgacgccgaggcgcccaggcccaacgcgcaaacattatcaaccatacgctccgccacgagatttcgcagcctatagatcccgagtaaaattttcccgaagtgcagatgcgccagcttatcagcaaggccagaatcagctctacaatgagttgaacctcggacggcgcgaggaatatgatgtgctcgataagcgcagaggtagggaccctgagatggggggtaaaccaaggcgaaagaatccccaagaggggctttacaacgaattgcaaaaagacaaaatggcagaagcctattccgaaattggaatgaagggcgaacggaggcgaggtaagggccacgacgggctctaccaaggcctgagcacagcaacaaaagacacgtatgacgcattgcacatgcaagctcttccgccccgg

SEQ ID NO: 168 - protein, 482 aa, synthetic construct
MALPVTALLLPLALLLHAARPQVQLKESGPGLVAPSQSLSITCTISGFSLTSYGVHWVRQPPGKGLEWLVVIWGDGSTSYNSALKSRLSISKDNSKSQVFLKMNSLQTDDTAMYFCATSTVGYAVDYWGQGTSVTVSSGGGGSGGGGSGGGGSDIQMTQSPASLSVSVGETVTITCRASENIYSNLAWYQQRQGKSPQLLVYAATNLADGVPSRFSGSGSGTQYSLKINSLQSEDFGSYYCQHFWGSPYTFGGGTKLEIKAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 169 - DNA, 1446 bp, synthetic construct
  source 1..1446: mol_type = other DNA
atggcgttgccggtgaccgcgcttttgctccctctggccctgctcttgcacgccgcacgaccgcaagtccagctcaaggagagcggtccaggactcgtagcaccctctcaatctttgtctatcacttgtaccataagcggtttttccttgacaagttatggcgtacattgggtgagacaaccgccaggtaaaggactcgaatggttggttgttatctggggggatgggagtaccagctacaacagcgcactcaagtctcgcctttcaatttctaaggacaacagtaaatcacaggtttttctgaagatgaatagcctgcaaaccgacgacacggcgatgtatttctgtgcgacttctacggtaggttatgccgtcgactattggggtcagggtacgtcagttaccgtaagtagcggcggtggtggctcaggcggcggaggatccgggggggggggatccgacatccagatgacccagtcaccggccagtttgtccgtttccgtaggcgagaccgtaaccatcacatgccgagcaagcgagaatatctattccaacctggcctggtatcagcagagacaaggcaaaagtccgcaacttttggtttatgctgccactaatttggccgatggggtcccgagtcgattttctggctctggctccgggacgcagtattccctgaagataaactctttgcagtcagaagattttggttcatattattgtcagcatttctggggcagcccgtacacatttggaggtgggaccaaattggaaataaaagcggccgcaattgaagtcatgtatcctcctccgtacttggataatgaaaaaagtaatgggaccataattcacgtcaaaggaaaacatttgtgtccaagcccccttttccctggcccgtccaagcctttctgggtactggtggtggtgggcggagttctcgcatgttattcattgctggtaacggtcgcattcattatattctgggtgcgatcaaaacggagtaggcttctccatagtgactatatgaacatgacgccgaggcgcccaggcccaacgcgcaaacattatcaaccatacgctccgccacgagatttcgcagcctatagatcccgagtaaaattttcccgaagtgcagatgcgccagcttatcagcaaggccagaatcagctctacaatgagttgaacctcggacggcgcgaggaatatgatgtgctcgataagcgcagaggtagggaccctgagatggggggtaaaccaaggcgaaagaatccccaagaggggctttacaacgaattgcaaaaagacaaaatggcagaagcctattccgaaattggaatgaagggcgaacggaggcgaggtaagggccacgacgggctctaccaaggcctgagcacagcaacaaaagacacgtatgacgcattgcacatgcaagctcttccgccccgg

SEQ ID NO: 170 - protein, 482 aa, synthetic construct
MALPVTALLLPLALLLHAARPDIQMTQSPASLSVSVGETVTITCRASENIYSNLAWYQQRQGKSPQLLVYAATNLADGVPSRFSGSGSGTQYSLKINSLQSEDFGSYYCQHFWGSPYTFGGGTKLEIKGGGGSGGGGSGGGGSQVQLKESGPGLVAPSQSLSITCTISGFSLTSYGVHWVRQPPGKGLEWLVVIWGDGSTSYNSALKSRLSISKDNSKSQVFLKMNSLQTDDTAMYFCATSTVGYAVDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 171 - DNA, 1446 bp, synthetic construct
  source 1..1446: mol_type = other DNA
atggcgttgccggtgaccgcgcttttgctccctctggccctgctcttgcacgccgcacgaccggacatccagatgacccagtcaccggccagtttgtccgtttccgtaggcgagaccgtaaccatcacatgccgagcaagcgagaatatctattccaacctggcctggtatcagcagagacaaggcaaaagtccgcaacttttggtttatgctgccactaatttggccgatggggtcccgagtcgattttctggctctggctccgggacgcagtattccctgaagataaactctttgcagtcagaagattttggttcatattattgtcagcatttctggggcagcccgtacacatttggaggtgggaccaaattggaaataaaaggcggtggtggctcaggcggcggaggatccgggggggggggatcccaagtccagctcaaggagagcggtccaggactcgtagcaccctctcaatctttgtctatcacttgtaccataagcggtttttccttgacaagttatggcgtacattgggtgagacaaccgccaggtaaaggactcgaatggttggttgttatctggggggatgggagtaccagctacaacagcgcactcaagtctcgcctttcaatttctaaggacaacagtaaatcacaggtttttctgaagatgaatagcctgcaaaccgacgacacggcgatgtatttctgtgcgacttctacggtaggttatgccgtcgactattggggtcagggtacgtcagttaccgtaagtagcgcggccgcaattgaagtcatgtatcctcctccgtacttggataatgaaaaaagtaatgggaccataattcacgtcaaaggaaaacatttgtgtccaagcccccttttccctggcccgtccaagcctttctgggtactggtggtggtgggcggagttctcgcatgttattcattgctggtaacggtcgcattcattatattctgggtgcgatcaaaacggagtaggcttctccatagtgactatatgaacatgacgccgaggcgcccaggcccaacgcgcaaacattatcaaccatacgctccgccacgagatttcgcagcctatagatcccgagtaaaattttcccgaagtgcagatgcgccagcttatcagcaaggccagaatcagctctacaatgagttgaacctcggacggcgcgaggaatatgatgtgctcgataagcgcagaggtagggaccctgagatggggggtaaaccaaggcgaaagaatccccaagaggggctttacaacgaattgcaaaaagacaaaatggcagaagcctattccgaaattggaatgaagggcgaacggaggcgaggtaagggccacgacgggctctaccaaggcctgagcacagcaacaaaagacacgtatgacgcattgcacatgcaagctcttccgccccgg

SEQ ID NO: 172 - protein, 490 aa, synthetic construct
MALPVTALLLPLALLLHAARPDIVMTQSPDSLAVSLGERATINCKSSQSVLYSSNNKNYLAWYQQKPGQPPKLFIYWASTRESGVPDRFSGSGSGTDFTLTISSLQAEDVAVYYCQQYYSTPPTFGQGTKVEIKGGGGSGGGGSGGGGSQVQLQESGPGLVKPSETLSLTCTVSGDSISSYYWTWIRQPAGKGLEWIGRIYTSGTTNYNPSLKSRVTMSVDTSKNQFSLKLSSVTAADTAVYYCASHSSGWYDAFDIWGQGTMVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 173 - DNA, 1470 bp, synthetic construct
  source 1..1470: mol_type = other DNA
atggcgttgccggtgaccgcgcttttgctccctctggccctgctcttgcacgccgcacgaccggacatcgtaatgacccaatcacctgattctctggctgtgagccttggtgagagggcaacaataaactgcaagagctctcagtccgtactttatagtagcaacaacaagaactatttggcgtggtaccagcagaagccgggtcagccgccaaagctgtttatatattgggcctctacccgcgaatctggagtaccggacaggtttagtggtagtggctcaggtacggattttacgctcaccattagctctctgcaagcagaggacgtagccgtatattattgccagcaatattattctacgccacccacctttggtcaggggacgaaagttgagatcaagggcggtggtggctcaggcggcggaggatccgggggggggggatcccaggtacagcttcaagaaagcggtccaggacttgtgaagccgagcgagacgctttcactgacttgcacagtcagtggggacagtattagttcctattactggacatggatacgacaaccagcagggaaaggtctggaatggattgggcgcatttacaccagtggaactacaaattacaacccatccctcaaatccagggttacgatgagcgtcgacacgagtaagaatcagttttccctgaagctctccagtgtgacggctgcagatactgcagtgtattattgcgcttcccactcaagtggttggtacgatgctttcgacatctgggggcagggtactatggtgaccgtgtcctcagcggccgcaattgaagtcatgtatcctcctccgtacttggataatgaaaaaagtaatgggaccataattcacgtcaaaggaaaacatttgtgtccaagcccccttttccctggcccgtccaagcctttctgggtactggtggtggtgggcggagttctcgcatgttattcattgctggtaacggtcgcattcattatattctgggtgcgatcaaaacggagtaggcttctccatagtgactatatgaacatgacgccgaggcgcccaggcccaacgcgcaaacattatcaaccatacgctccgccacgagatttcgcagcctatagatcccgagtaaaattttcccgaagtgcagatgcgccagcttatcagcaaggccagaatcagctctacaatgagttgaacctcggacggcgcgaggaatatgatgtgctcgataagcgcagaggtagggaccctgagatggggggtaaaccaaggcgaaagaatccccaagaggggctttacaacgaattgcaaaaagacaaaatggcagaagcctattccgaaattggaatgaagggcgaacggaggcgaggtaagggccacgacgggctctaccaaggcctgagcacagcaacaaaagacacgtatgacgcattgcacatgcaagctcttccgccccgg

SEQ ID NO: 174 - protein, 490 aa, synthetic construct
MALPVTALLLPLALLLHAARPQVQLQESGPGLVKPSETLSLTCTVSGDSISSYYWTWIRQPAGKGLEWIGRIYTSGTTNYNPSLKSRVTMSVDTSKNQFSLKLSSVTAADTAVYYCASHSSGWYDAFDIWGQGTMVTVSSGGGGSGGGGSGGGGSDIVMTQSPDSLAVSLGERATINCKSSQSVLYSSNNKNYLAWYQQKPGQPPKLFIYWASTRESGVPDRFSGSGSGTDFTLTISSLQAEDVAVYYCQQYYSTPPTFGQGTKVEIKAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 175 - DNA, 1470 bp, synthetic construct
  source 1..1470: mol_type = other DNA
atggcgttgccggtgaccgcgcttttgctccctctggccctgctcttgcacgccgcacgaccgcaggtacagcttcaagaaagcggtccaggacttgtgaagccgagcgagacgctttcactgacttgcacagtcagtggggacagtattagttcctattactggacatggatacgacaaccagcagggaaaggtctggaatggattgggcgcatttacaccagtggaactacaaattacaacccatccctcaaatccagggttacgatgagcgtcgacacgagtaagaatcagttttccctgaagctctccagtgtgacggctgcagatactgcagtgtattattgcgcttcccactcaagtggttggtacgatgctttcgacatctgggggcagggtactatggtgaccgtgtcctcaggcggtggtggctcaggcggcggaggatccgggggggggggatccgacatcgtaatgacccaatcacctgattctctggctgtgagccttggtgagagggcaacaataaactgcaagagctctcagtccgtactttatagtagcaacaacaagaactatttggcgtggtaccagcagaagccgggtcagccgccaaagctgtttatatattgggcctctacccgcgaatctggagtaccggacaggtttagtggtagtggctcaggtacggattttacgctcaccattagctctctgcaagcagaggacgtagccgtatattattgccagcaatattattctacgccacccacctttggtcaggggacgaaagttgagatcaaggcggccgcaattgaagtcatgtatcctcctccgtacttggataatgaaaaaagtaatgggaccataattcacgtcaaaggaaaacatttgtgtccaagcccccttttccctggcccgtccaagcctttctgggtactggtggtggtgggcggagttctcgcatgttattcattgctggtaacggtcgcattcattatattctgggtgcgatcaaaacggagtaggcttctccatagtgactatatgaacatgacgccgaggcgcccaggcccaacgcgcaaacattatcaaccatacgctccgccacgagatttcgcagcctatagatcccgagtaaaattttcccgaagtgcagatgcgccagcttatcagcaaggccagaatcagctctacaatgagttgaacctcggacggcgcgaggaatatgatgtgctcgataagcgcagaggtagggaccctgagatggggggtaaaccaaggcgaaagaatccccaagaggggctttacaacgaattgcaaaaagacaaaatggcagaagcctattccgaaattggaatgaagggcgaacggaggcgaggtaagggccacgacgggctctaccaaggcctgagcacagcaacaaaagacacgtatgacgcattgcacatgcaagctcttccgccccgg